(19) United States
(12) Patent Application Publication
(10) Pub. No.: US 2023/0181620 A1
(43) Pub. Date: Jun. 15, 2023

(54) COMPOSITIONS FOR TRANSLATION AND METHODS OF USE THEREOF

(71) Applicant: Flagship Pioneering Innovations VI, LLC, Cambridge, MA (US)

(72) Inventors: Avak KAHVEJIAN, Lexington, MA (US); Alexandra Sophie DE BOER, Somerville, MA (US); Nicholas McCartney PLUGIS, Duxbury, MA (US); Ki Young PAEK, Brighton, MA (US)

(21) Appl. No.: 17/795,915

(22) PCT Filed: Jan. 29, 2021
(86) PCT No.: PCT/US2021/015742

§ 371 Date: Jul. 28, 2022

Related U.S. Application Data
(60) Provisional application No. 62/967,547, filed on Jan. 29, 2020.

(51) Int. Cl. A61K 31/7115; A61K 31/7105; C12N 15/67; C12N 15/88

(52) U.S. Cl. CPC A61K 31/7115; A61K 31/7105; C12N 15/67; C12N 15/88

(57) ABSTRACT

This invention relates generally to pharmaceutical compositions and preparations of polyribonucleotides and circular polyribonucleotides and uses thereof.

### CROSS-REFERENCE TO RELATED APPLICATIONS

The present application claims priority to and benefit from U.S. Provisional Application No. 62/967,547, filed Jan. 29, 2020, the entire contents of which is herein incorporated by reference.

### BACKGROUND

Certain circular polyribonucleotides are ubiquitously present in human tissues and cells, including tissues and cells of healthy individuals.

### SUMMARY

The present disclosure generally relates to compositions of a polyribonucleotide comprising a 5′ modified guanosine cap and a circular polyribonucleotide. In some embodiments, the compositions as decribed herein are pharmaceutical compositions further comprising a pharmaceutically acceptable excipient. The present disclosure further relates to methods of translation of an expression sequence of the circular polyribonucleotide using a composition comprising a polyribonucleotide comprising a 5′ modified guanosine cap and a circular polyribonucleotide. In some embodiments, the compositions comprising a polyribonucleotide comprising a 5′ modified guanosine cap and a circular polyribonucleotide have increased translation of an expression sequence of the circular polyribonucleotide than translation of an expression sequence of the circular polyribonucleotide in a composition of the circular polyribonucleotide alone. In some embodiments, the compositions comprising a polyribonucleotide comprising a 5′ modified guanosine cap and a circular polyribonucleotide have prolonged translation of an expression sequence of the circular polyribonucleotide than translation of an expression sequence of the circular polyribonucleotide in a composition of the circular polyribonucleotide alone.

In a first aspect, the invention features a pharmaceutical composition comprising: (a) a polyribonucleotide comprising a 5′ modified guanosine cap and a first binding region; (b) a circular polyribonucleotide; and (c) a pharmaceutically acceptable excipient.

In some embodiments, the circular polyribonucleotide comprises a second binding region. In some embodiments, the first binding region specifically binds to the second binding region. In some embodiments, the polyribonucleotide comprising the 5′ modified guanosine cap drives expression of the expression sequence in the circular polyribonucleotide when the polyribonucleotide is bound to the circular polyribonucleotide. In some embodiments, the polyribonucleotide is bound to the circular polyribonucleotide by indirect binding. In some embodiments, the polyribonucleotide is bound to the circular polyribonucleotide by direct binding. In some embodiments, the polyribonucleotide is bound to the circular polyribonucleotide by covalent binding. In some embodiments, the polyribonucleotide is bound to the circular polyribonucleotide by noncovalent binding. In some embodiments, the first binding region is complementary to the second binding region.

In some embodiments, the polyribonucleotide recruits a ribosome. In some embodiments, the 5′ modified guanosine cap of the polyribonucleotide recruits the ribosome. In some embodiments, the circular polyribonucleotide comprises an expression sequence. In some embodiments, the polyribonucleotide comprising the 5′ modified guanosine cap drives expression of the expression sequence in the circular polyribonucleotide.

In some embodiments, the polyribonucleotide further comprises a UTR. In some embodiments, the polyribonucleotide comprises a 5′ UTR. In some embodiments, the polyribonucleotide comprises a 3′ UTR. In some embodiments, the polyribonucleotide comprises a poly A region. In some embodiments, the first binding region is a binding region that is 3′ of a UTR. In some embodiments, the first binding region comprises from 5 to 100 nucleotides in length.

In some embodiments, the 5′ modified guanosine cap is a 7-methylguanylate cap. In some embodiments, the 5′ modified guanosine cap is an anti-reverse cap analog. In some embodiments, the polyribonucleotide comprises one or more of the 5′ modified guanosine cap. In some embodiments, the polyribonucleotide is linear.

In some embodiments, the polyribonucleotide comprises from 5 to 1100 nucleotides in length. In some embodiments, the circular polyribonucleotide is an unmodified circular polyribonucleotide. In some embodiments, the circular polyribunucleotide comprises a UTR. In some embodiments, the circular polyribunucleotide comprises a poly A region. In some embodiments, the circular polyribonucleotide comprises an IRES. In some embodiments, the circular polyribunucleotide lacks an IRES. In some embodiments, the second binding region comprises from 5 to 100 nucleotides in length. In some embodiments, the circular polyribonucleotide comprises a stop codon. In some embodiments, the circular polyribonucleotide comprises the second binding region located in an untranslated region between the stop and a start codon. In some embodiments, the circular polyribonucleotide comprises an encryptogen, regulatory element, replication element, or quasi-double stranded secondary structure. In some embodiments, the circular polyribonucleotide comprises a stagger element. In some embodiments, the circular polyribonucleotide comprises a stop codon between the second binding region and the stagger element. In some embodiments, the circular polyribonucleotide comprises a protein translation initiation site. In some embodiments, the protein translation initiation site comprises a Kozak sequence. In some embodiments, the circular polyribonucleotide comprises from 50 to 20000 nucleotides in length.

In a second aspect, the invention features a pharmaceutical composition comprising (a) a first polyribonucleotide comprising a 5′ modified guanosine cap and a first binding region; (b) a second polyribonucleotide comprising a 5′ modified guanosine cap and a third binding region; (c) a circular polyribonucleotide; and (d) a pharmaceutically acceptable excipient.

In some embodiments, the circular polyribonucleotide comprises a second binding region and a fourth binding region. In some embodiments, the first binding region specifically binds to the second binding region, and the third binding region specifically binds to the fourth binding region. In some embodiments, the first polyribonucleotide and the second polyribonucleotide drive expression of an expression sequence in the circular polyribonucleotide when the polyribonucleotides are bound to the circular polyribonucleotide. In some embodiments, the first polyribonucleotide and the second polyribonucleotide drive increased expression of an expression sequence in the circular polyribonucleotide when the first polyribonucleotide and the second polyribonucleotide are bound to the circular polyribonucleotide compared to expression of an expression sequence in the circular polyribonucleotide when the first polyribonucleotide is bound to the circular polyribonucleotide or compared to expression of an expression sequence in the circular polyribonucleotide when the second polyribonucleotide is bound to the circular polyribonucleotide.

In a third aspect, the invention features a polyribonucleotide comprising a 5′ modified guanosine cap and a first binding region, wherein the first binding region specifically binds to a second binding region of a circular polyribonucleotide.

In a fourth aspect, the invention features a circular polyribonucleotide comprising a second binding region, wherein the second binding region specifically binds to a first binding region of a polyribonucleotide and wherein the polyribonucleotide comprises a 5′ modified guanosine cap.

In a fifth aspect, the invention features a complex comprising the polyribonucleotide of any one of the preceding embodiments; and the circular polyribonucleotide of any one of the preceding embodiments, wherein the first binding region of the polyribonucleotide is bound to the second binding region of the circular polyribonucleotide.

In a sixth aspect, the invention features a method of producing a complex comprising binding the first binding region of the polyribonucleotide of any one of the preceding embodiments to the second binding region of the circular polyribonucleotide any one of the preceding embodiments, thereby producing the complex.

In a seventh aspect, the invention features a method of expressing an expression sequence from a circular polyribonucleotide in a cell, comprising delivering the complex of any one of the preceding embodiments to the cell, wherein the circular polyribonucleotide of the complex comprises an expression sequence.

In an eighth aspect, the invention features the phamaceutical composition of any one of the preceding embodiments for use in a method of treatment of a human or animal body by therapy.

In a ninth aspect, the invention features the complex of any one of the preceding embodiments for use as a medicament or a pharmaceutical.

In a tenth aspect, the invention features the complex of any one of the preceding embodiments for use in a method of treatment of a human or animal body by therapy.

In an eleventh aspect, the invention features a use of the complex of any one of the preceding embodiments, or the polyribonucleotide of any one of the preceding embodiments and the circular polyribonucleotide of any one of the preceding embodiments, in the manufacture of a medicament or a pharmaceutical.

In a twelfth aspect, the invention features a use of the complex of any one of the preceding embodiments, or the polyribonucleotide of any one of the preceding embodiments and the circular polyribonucleotide of any one of the preceding embodiments, in the manufacture of a medicament or a pharmaceutical for treating a human or animal body by therapy.

### Definitions

The present invention will be described with respect to particular embodiments and with reference to certain figures but the invention is not limited thereto but only by the claims. Terms as set forth hereinafter are generally to be understood in their common sense unless indicated otherwise.

The term “pharmaceutical composition” is intended to disclose that the circular polyribonucleotide comprised within a pharmaceutical composition can be used for the treatment of the human or animal body by therapy. It is thus meant to be equivalent to the “a circular polyribonucleotide for use in therapy”.

As used herein, the terms “circRNA” or “circular polyribonucleotide” or “circular RNA” are used interchangeably and mean a polyribonucleotide that has a structure having no free ends (i.e. no free 3′ and/or 5′ ends), for example a polyribonucleotide molecule that forms a circular structure through covalent or non-covalent bonds.

As used herein, the term “encryptogen” is a nucleic acid sequence or structure of the circular polyribonucleotide that aids in reducing, evading, and/or avoiding detection by an immune cell and/or reduces induction of an immune response against the circular polyribonucleotide.

As used herein, the term “expression sequence” is a nucleic acid sequence that encodes a product, e.g., a peptide or polypeptide, or a regulatory nucleic acid. An exemplary expression sequence that codes for a peptide or polypeptide can comprise a plurality of nucleotide triads, each of which can code for an amino acid and is termed as a “codon”.

As used herein, the term “modified ribonucleotide” means any ribonucleotide analog or derivative that has one or more chemical modifications to the chemical composition of an unmodified natural ribonucleotide, such as a natural unmodified nucleotide adenosine (A), uridine (U), guanine (G), cytidine (C). In some embodiments, the chemical modifications of the modified ribonucleotide are modifications to any one or more functional groups of the ribonucleotide, such as, the sugar the nucleobase, or the internucleoside linkage (e.g. to a linking phosphate / to a phosphodiester linkage / to the phosphodiester backbone).

As used herein, the phrase “quasi-helical structure” is a higher order structure of the circular polyribonucleotide, wherein at least a portion of the circular polyribonucleotide folds into a helical structure.

As used herein, the phrase “quasi-double-stranded secondary structure” is a higher order structure of the circular polyribonucleotide, wherein at least a portion of the circular polyribonucleotide creates an internal double strand.

As used herein, the term “regulatory element” is a moiety, such as a nucleic acid sequence, that modifies expression of an expression sequence within the circular polyribonucleotide.

As used herein, the term “repetitive nucleotide sequence” is a repetitive nucleic acid sequence within a stretch of DNA or RNA or throughout a genome. In some embodiments, the repetitive nucleotide sequence includes poly CA or poly TG (UG) sequences. In some embodiments, the repetitive nucleotide sequence includes repeated sequences in the Alu family of introns.

As used herein, the term “replication element” is a sequence and/or motifs useful for replication or that initiate transcription of the circular polyribonucleotide.

As used herein, the term “stagger element” is a moiety, such as a nucleotide sequence, that induces ribosomal pausing during translation. In some embodiments, the stagger element is a non-conserved sequence of amino-acids with a strong alpha-helical propensity followed by the consensus sequence -D(V/I)ExNPG P, where x= any amino acid. In some embodiments, the stagger element may include a chemical moiety, such as glycerol, a non nucleic acid linking moiety, a chemical modification, a modified nucleic acid, or any combination thereof.

As used herein, the term “substantially resistant” refers to one that has at least 50%, 55%, 60%, 65%, 70%, 75%, 80%, 85%, 90%, 95%, 96%, 97%, 98% or 99% resistance as compared to a reference.

As used herein, the term “translation initiation sequence” is a nucleic acid sequence that initiates translation of an expression sequence in the circular polyribonucleotide.

As used herein, the term “termination element” is a moiety, such as a nucleic acid sequence, that terminates translation of the expression sequence in the circular polyribonucleotide.

As used herein, the term “translation efficiency” is a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., an in vitro translation system like rabbit reticulocyte lysate, or an in vivo translation system like a eukaryotic cell or a prokaryotic cell.

As used herein, the term “circularization efficiency” is a measurement of resultant circular polyribonucleotide versus its starting material.

As used herein, the term “immunogenic” is a potential to induce a response to a substance in a particular immune response assay above a pre-determined threshold. The assay can be, e.g., expression of certain inflammatory markers, production of antibodies, or an assay for immunogenicity as described herein. In some embodiments, an immune response may be induced when an immune system of an organism or a certain type of immune cells is exposed to an immunogenic substance.

An immunogenic response may be assessed by evaluating the antibodies in the plasma or serum of a subject using a total antibody assay, a confirmatory test, titration and isotyping of the antibodies, and neutralizing antibody assessment. A total antibody assay measures the antibodies generated as part of the immune response in the serum or plasma of a subject that has been administered the immunogenic substance. The most commonly used test to detect antibodies is an ELISA (enzyme-linked immunosorbent assay), which detects antibodies in the tested serum that bind to the antibody of interest, including IgM, IgD, IgG, IgA, and IgE. An immunogenic response can be further assessed by a confirmatory assay. Following a total antibody assessment, a confirmatory assay may be used to confirm the results of the total antibody assay. A competition assay may be used to confirm that antibody is specifically binding to target and that the positive finding in the screening assay is not a result of nonspecific interactions of the test serum or detection reagent with other materials in the assay.

An immunogenic response can be assessed by isotyping and titration. An isotyping assay may be used to assess only the relevant antibody isotypes. For example, the expected isotypes may be IgM and IgG which may be specifically detected and quantified by isotyping and titration, and then compared to the total antibodies present.

An immunogenic response can be assessed by a neutralizing antibody assay (nAb). A neutralizing antibody assay (nAb) may be used to determine if the antibodies produced in response to the immunogenic substance neutralized the immunogenic substance thereby inhibiting the immunogenic substance from having an effect on the target and leading to abnormal pharmacokinetic behaviors. An nAb assay is often a cell-based assay where the target cells are incubated with the antibody. A variety of cell based nAb assays may be used including but not limited to cell proliferation, viability, antibody-dependent cell-mediated cytotoxicity (ADCC), complement-dependent cytotoxicity (CDC), cytopathic effect inhibition (CPE), apoptosis, ligand stimulated cell signaling, enzyme activity, reporter gene assays, protein secretion, metabolic activity, stress and mitochondrial function. Detection readouts include absorbance, fluorescence, luminescence, Ccemiluminescence, or flow cytometry A ligand-binding assay may also be used to measure the binding affinity of an immunogen and an antibody in vitro to evaluate neutralization efficacy.

Furthermore, induction of a cellular immune response may be assessed by measuring T cell activation in a subject using cellular markers on T cells obtained from the subject. A blood sample, lymph node biopsy, or tissue sample can be collected from a subject and T cells from the sample evaluated for one or more (e.g., 2, 3, 4 or more) activation markers: CD25, CD71, CD26, CD27, CD28, CD30, CD154, CD40L, CD134, CD69, CD62L or CD44. T cell activation can also be assessed using the same methods in an in vivo animal model. This assay can also be performed by adding an immunogenenic substance to T cells in vitro (e.g., T cells obtained from a subject, animal model, repository, or commercial source) and measuring the aforementioned markers to evaluate T cell activation. Similar approaches can be used to assess the effect of an on activation of other immune cells, such as eosinophils (markers: CD35, CD11b, CD66, CD69 and CD81), dendritic cells (markers: IL-8, MHC class II, CD40, CD80, CD83, and CD86), basophils (CD63, CD13, CD4, and CD203c), and neutrophils (CD11b, CD35, CD66b and CD63). These markers can be assessed using flow cytometry, immunohistochemistry, in situ hybridization, and other assays that allow for measurement of cellular markers. Comparing results from before and after administration of an immunogenic substance can be used to determine its effect.

The term “non-immunogenic” is a lack of or absence of an immune response above a predetermined threshold when measured by a particular immune response assay. For example, when an innate immune response assay is used to measure an innate immune response against a circular polyribonucleotide (such as measuring inflammatory markers), a non-immunogenic polyribonucleotide as provided herein can lead to production of an innate immune response at a level lower than a predetermined threshold. The predetermined threshold can be, for instance, at most 1.5 times, 2 times, 3 times, 4 times, 5 times, 6 times, 7 times, 8 times, 9 times, or 10 times the level of a marker(s) produced by an innate immune response for a control reference.

As used herein, the term “direct binding” is an association between at least two moieties (e.g., chemical or biochemical) that have an affinity for one another. Examples include covalent binding of two moieties, binding by click chemistry, noncovalent binding canonical Watson-Crick base pairing or noncanoical base pairing, or electrostatic interactions, such as ionic interactions, a hydrogen bonding and a halogen bonding, π-effects, van der Waals forces, and hydrophobic effects.

As used herein, the term “indirect binding” is to an association between at least two moieties through an intermediary moiety, wherein the intermediary moiety has an affinity for the at least two moieties. Examples include co-binding partners, such as chemicals, small molecules, proteins, peptides, agents, or factors, each of which bind to the at least two moieties, respectively.

As used herein, the term “carrier” means a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride- modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the term “naked delivery” means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that comprises a circular polyribonucleotide without covalent modification and is free from a carrier

The term “diluent” means vehicle comprising an inactive solvent in which a composition described herein (e.g., a composition comprising a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3- butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, or powdered sugar.

As used herein, the term “parenterally acceptable diluent” is a diluent used for parenteral administration of a composition (e.g., a composition comprising a circular polyribonucleotide).

As used herein, the term “linear counterpart” is a polyribonucleotide molecule (and its fragments) having the same or similar nucleotide sequence (e.g., 100%, 95%, 90%, 85%, 80%, 75%, or any percentage therebetween of sequence similarity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence similarity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween of sequence similarity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further comprises a 5′ cap. In some embodiments, the linear counterpart further comprises a poly adenosine tail. In some embodiments, the linear counterpart further comprises a 3′ UTR. In some embodiments, the linear counterpart further comprises a 5′ UTR.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 shows a schematic of an exemplary in vitro production process of a circular RNA that contains a start-codon, an ORF (open reading frame) coding for GFP, a stagger element (2A), an encryptogen (optional), and an IRES (internal ribosome entry site).

FIG. 2 shows a schematic of an exemplary in vivo production process of a circular RNA.

FIG. 3A and FIG. 3B are schematics demonstrating in vivo stoichiometric protein expression of two different circular RNAs.

FIG. 4 is a schematic demonstrating transcription, self-cleavage, and ligation of an exemplary self-replicable circular RNA.

FIG. 5A shows a schematic of an exemplary circular RNA with a 2A stagger element, annealing region, and Kozak NLuc ORF.

FIG. 5B shows a schematic of an exemplary polyribonucleotide comprising 5′ cap, 5′ UTR and anti-sense annealing sequence annealed to the exemplary circular RNA of FIG. 5A.

FIG. 5C is a graph showing that annealing of capped polyribonucleotides to circular RNAs increases translation of functional a NanoLuc® luciferase (nLuc) in BJ Fibroblasts.

FIG. 5D is a graph showing that annealing of capped polyribonucleotides to circular RNAs increases translation of functional a NanoLuc® luciferase (nLuc) in SV40 MEFs.

FIG. 6A shows a schematic of an exemplary circular RNA with a 2A stagger element, 3X stop codon, annealing region, and Kozak NLuc ORF.

FIG. 6B shows a schematic of an exemplary polyribonucleotide comprising 5′ cap, 5′ UTR and anti-sense annealing sequence annealed to the exemplary circular RNA of FIG. 6A.

FIG. 6C is a graph showing that annealing of capped polyribonucleotides to circular RNAs increases translation of functional a NanoLuc® luciferase (nLuc) in BJ Fibroblasts.

FIG. 6D is a graph showing that annealing of capped polyribonucleotides to circular RNAs increases translation of functional a NanoLuc® luciferase (nLuc) in SV40 MEFs.

FIG. 7A shows a schematic of an exemplary circular RNA with an ORF encoding a Gaussia luciferase (GLuc ORF) and a stop codon.

FIG. 7B shows a schematic of an exemplary polyribonucleotide comprising a 5′ cap and a 3′ annealing sequence complementary to the annealing region of the circular RNA (Oligo #0) annealed to the exemplary circular RNA of FIG. 7A.

FIG. 7C shows a schematic of an exemplary polyribonucleotide comprising a 5′ cap and a 3′ annealing sequence complementary to 44 nucleotides upstream of the stop codon of the Gluc ORF (Oligo #9) annealed to the exemplary circular RNA of FIG. 7A.

FIG. 7D is a graph showing that circular RNA annealed with the capped polyribonucleotide exhibited greater GLuc expression than the circular RNA only counterpart.

FIG. 8A shows a schematic of an exemplary circular RNA with an ORF encoding Gaussia luciferase (GLuc ORF), an annealing region, and stop codon.

FIG. 8B shows a schematic of an exemplary polyribonucleotide comprising a 5′ cap and a 3′ annealing sequence complementary to the annealing region of the circular RNA (Oligo #0) annealed to the exemplary circular RNA of FIG. 8A.

FIG. 8C shows a schematic of an exemplary polyribonucleotide comprising comprising a 5′ cap and a 3′ annealing sequence complementary to nucleotides upstream of the stop codon of the Gluc ORF (Oligo #9) annealed to the exemplary circular RNA of FIG. 8A.

FIG. 8D shows a schematic of an exemplary capped polyribonucleotide of FIG. 8B and an exemplary capped polyribonucleotide of FIG. 8C annealed to the exemplary circular RNA of FIG. 5A.

FIG. 8E is a graph showing that annealing of capped polyribonucleotides to circular RNAs increases translation of functional a NanoLuc® luciferase (nLuc) in SV40 MEFs.

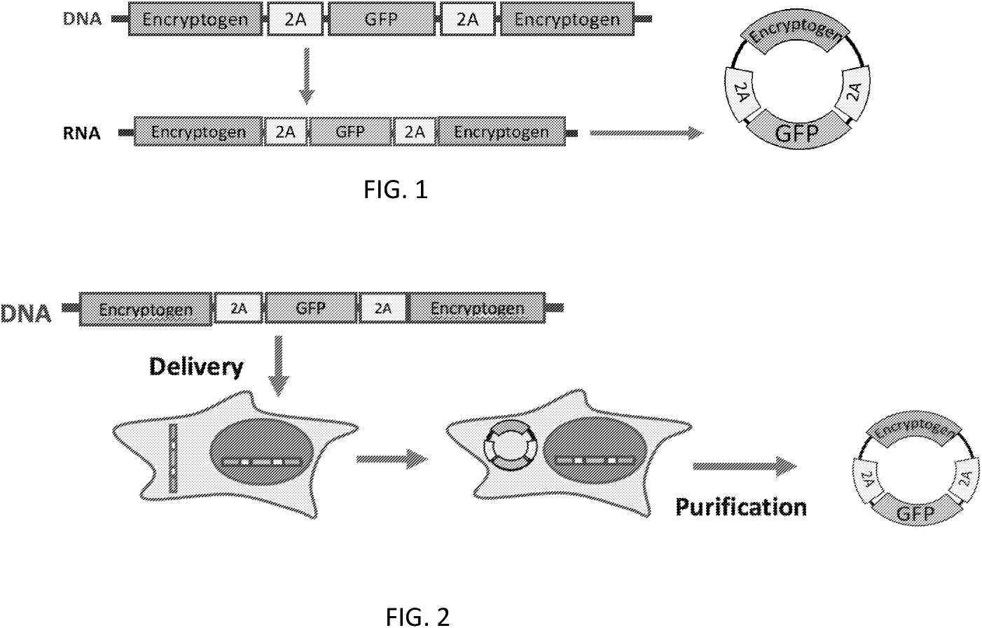

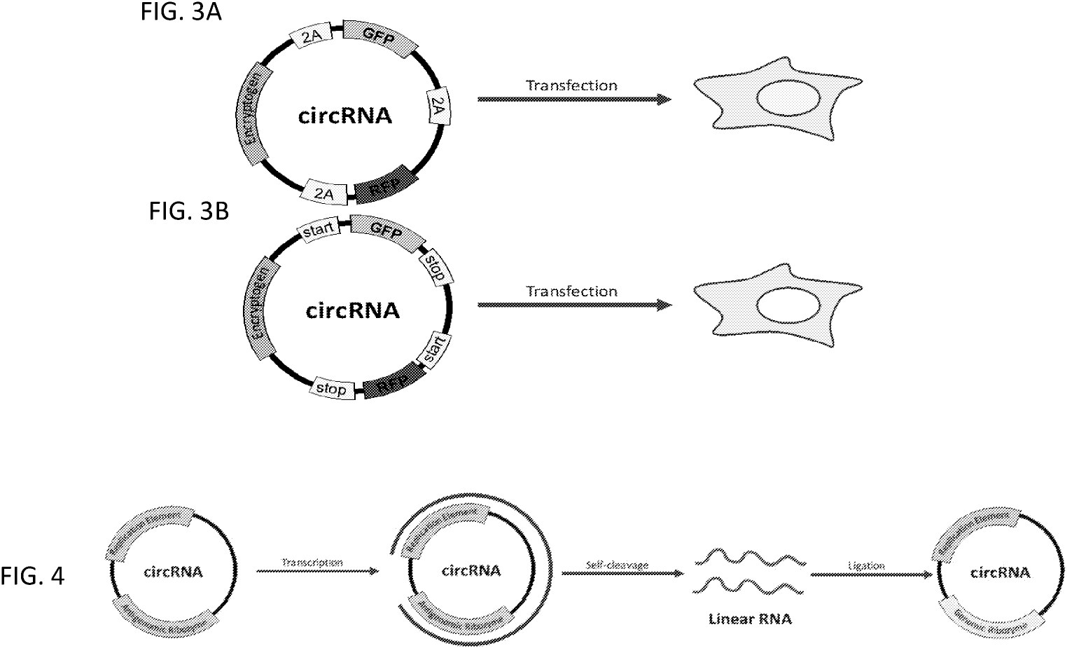

### DETAILED DESCRIPTION

This invention relates generally to pharmaceutical compositions and preparations of circular polyribonucleotides and polyribonucleotides comprising a 5′ cap, and uses thereof.

The present invention described herein includes compositions of a polyribonucleotide comprising a 5′ modified guanosine cap (referred to herein as a capped polyribonucleotide) and a circular polyribonucleotide. The circular polyribonucleotide can further comprise an expression sequence. Sometimes, the compositions as decribed herein are pharmaceutical compositions further comprising a pharmaceutically acceptable excipient.

In some embodiments, the capped polyribonucleotide further comprises a binding region that binds to the circular polyribonucleotide. In some embodiments, the circular polyribonucleotide further comprises a binding region that binds to the capped polyribonucleotide. The binding region of the capped polyribonucleotide can comprise a sequence that is antisense to a sequence of the binding region of the circular polyribonucleotide.

The invention described herein can further comprise a complex formed by the capped polyribonucleotide and the circular polyribonucleotide. The capped polyribonucleotide can form a complex with the circular polyribonucleotide, wherein the binding region of the capped polyribonucleotide binds to the binding region of the circular polyribonucleotide.

The compositions as decribed herein are used in methods of translation of an expression sequence of the circular polyribonucleotide.Applicants have discovered that the compositions have increased translation of an expression sequence of the circular polyribonucleotide than a composition of the circular polyribonucleotide alone (e.g., a composition lacking the capped polyribonucleotide). Applicants have find that the compositions have increased expression of an expression sequence of the circular polyribonucleotide leading to increased protein production compared to a composition of the circular polyribonucleotide alone (e.g., a composition lacking the capped polyribonucleotide) or a linear counterpart of the circular polyribonucleotide. Applicants have also find that the compositions have prolonged translation of an expression sequence of the circular polyribonucleotide than a composition of the circular polyribonucleotide alone (e.g., a composition lacking the capped polyribonucleotide). In certain stress conditions, cap-dependent translation is a preferred method of translation (e.g., preferred over translation methods using an IRES).

### Capped Polyribonucleotides

The polyribonucleotides as described herein comprise a 5′ modified guanosine cap, which is also referred to herein as a capped polyribonucleotide. In some embodiments, the polyribonucleotide of the capped polyribonucleotide further comprises a binding region that binds to a circular polyribonucleotide. The binding region of the capped polyribonucleotide can comprise a sequence that is antisense to a sequence of a binding region of a circular polyribonucleotide. The polyribonucleotide of the capped polyribonucleotide can further comprise a UTR. The polyribonucleotide of the capped polyribonucleotide can further comprise a poly-A region. The capped polyribonucleotide can form a complex with a circular polyribonucleotide, wherein the circular polyribonucleotide comprises an expression sequence. The capped polyribonucleotide complexed with the circular polyribonucleotide can recruit a ribosome for initation of translation of an expression sequence in the circular polyribonucleotide. In some embodiments, the capped polynucleotides as described herein are a plurality of capped polynucleotides. In some embodiments, the plurality of capped polynucleotides comprises at least two of the same capped polynucleotides. In some embodiments, the plurality of the capped polynucleotides comprises one or more different capped polynucleotides.

### Cap

In some embodiments, the polyribonucleotide comprises a 5′-terminal cap, which is referred to as a capped polyribonucleotide. In some embodiments, the polyribonucleotide comprises a 5′ modified guanosine cap. In some embodiments, the polyribonucleotide comprises one or more 5′ modified guanosine caps. In some embodiments, the 5′ modified guanosine cap is a 7-methylguanylate cap. In some embodiments, the polyribonucleotide comprises a physiological 5′ modified guanosine cap. In some embodiments, the polyribonucleotide comprises a synthetic 5′-terminal cap analog. In some embodiments, the polyribonucleotide comprises a 5′ modified guanosine cap structure generated using co-transcriptional capping with anti-reverse cap analog (ARCA). In some embodiments, the 5′ modified guanosine cap is an anti-reverse cap analog. For example, in some embodiments, the polyribonucleotide comprises an m⁷Gp₃G. For another example, in some embodiments, the polyribonucleotide comprises an m⁷3′dGp₃G, m₂^{7,3′-O}Gp₃G, m₂^{7,2′-O}Gp₃G, m⁷2′dGp₃G, m⁷2′dGp₄G, m₂ ^{7,2′-O}Gp₄G, m₂^{7,3′-O}Gp₄G, m⁷Gp₅G, m₂^{7,3′-0}Gp₅G, m⁷Gp₄G, or m⁷Gp₅G. In some embodiments, the polyribonucleotide comprises exemplary embodiments of synthetic 5′-terminal cap analogs or a 5′ modified guanosine cap structure generated using co-transcriptional capping with anti-reverse cap analog (ARCA) as described by Jemielity J. et al. (RNA. 2003;9(9): 1108-22) or by Kowalska, J. et al. (RNA 2008;14: 1119 -1131). In some embodiments, the 5′ modified guanosine cap of the polyribonucleotide recruits a ribosome. In some embodiments, the 5′ modified guanosine cap of the polyribonucleotide binds to the ribosome. In some embodiments, the recruitment of the ribosome initates translation of an expression sequence.

### Polyribonucleotide of the Capped Polyribonucleotide

The polyribonucleotide of the capped polyribonucleotide can be any contiguous stretch of ribonucleic acids. In some embodiments, the polyribonucleotide is an unmodified polyribonucleotide. In some embodiments, the polyribonucleotide is a modified polyribonucleotide. The polyribonucleotide of the capped polyribonucleotide can be a linear polyribonucleotide. In some embodiments, the polyribonucleotide is an oligopolyribonucleotide. In some embodiments, the polyribonucleotide is a single stranded polyribonucleotide. In some embodiments, the polyribonucleotide is pseudo-double stranded (e.g., a portion of the single stranded polyribonucleotide self-hybridizes). In some embodiments, the polynucleotides of the capped polynucleotides comprises a plurality of polynucleotides. In some embodiments, the plurality polynucleotides comprises at least two of the same polynucleotide. In some embodiments, the plurality of the polynucleotides comprises one or more different polynucleotides.

In some embodiments, the polyribonucleotide of the capped polyribonucleotide comprises from 5 to 1100 nucleotides in length. In some embodiments, the polyribonucleotide comprises from 5 to 1150 nucleotides in length. In some embodiments, the polyribonucleotide comprises from 5 to 1000 nucleotides in length. In some embodiments, the polyribonucleotide comprises from 5 to 950 nucleotides in length. In some embodiments, the polyribonucleotide comprises from 5 to 900 nucleotides in length. In some embodiments, the polyribonucleotide comprises from 5 to 850 nucleotides in length. In some embodiments, the polyribonucleotide comprises from 5 to 800 nucleotides in length. In some embodiments, the polyribonucleotide comprises from 5 to 750 nucleotides in length. In some embodiments, the polyribonucleotide comprises from 5 to 700 nucleotides in length. In some embodiments, the polyribonucleotide comprises from 5 to 650 nucleotides in length. In some embodiments, the polyribonucleotide comprises from 5 to 600 nucleotides in length. In some embodiments, the polyribonucleotide comprises from 5 to 550 nucleotides in length. In some embodiments, the polyribonucleotide comprises from 5 to 500 nucleotides in length. In some embodiments, the polyribonucleotide comprises from 10 to 450 nucleotides in length. In some embodiments, the polyribonucleotide comprises from 10 to 400 nucleotides in length. In some embodiments, the polyribonucleotide comprises from 5 to 350 nucleotides in length. In some embodiments, the polyribonucleotide comprises from 5 to 300 nucleotides in length. In some embodiments, the polyribonucleotide comprises from 5 to 250 nucleotides in length. In some embodiments, the polyribonucleotide comprises from 5 to 200 nucleotides in length. In some embodiments, the polyribonucleotide comprises from 5 to 150 nucleotides in length. In some embodiments, the polyribonucleotide comprises from 5 to 100 nucleotides in length. In some embodiments, the polyribonucleotide comprises from 5 to 95 nucleotides in length. In some embodiments, the polyribonucleotide comprises from 5 to 90 nucleotides in length. In some embodiments, the polyribonucleotide comprises from 5 to 85 nucleotides in length. In some embodiments, the polyribonucleotide comprises from 5 to 80 nucleotides in length. In some embodiments, the polyribonucleotide comprises from 5 to 75 nucleotides in length. In some embodiments, the polyribonucleotide comprises from 5 to 70 nucleotides in length. In some embodiments, the polyribonucleotide comprises from 5 to 65 nucleotides in length. In some embodiments, the polyribonucleotide comprises from 5 to 60 nucleotides in length. In some embodiments, the polyribonucleotide comprises from 5 to 55 nucleotides in length. In some embodiments, the polyribonucleotide comprises from 5 to 50 nucleotides in length. In some embodiments, the polyribonucleotide comprises from 5 to 45 nucleotides in length. In some embodiments, the polyribonucleotide comprises from 5 to 40 nucleotides in length. In some embodiments, the polyribonucleotide comprises from 5 to 35 nucleotides in length. In some embodiments, the polyribonucleotide comprises from 5 to 30 nucleotides in length. In some embodiments, the polyribonucleotide comprises from 5 to 25 nucleotides in length. In some embodiments, the polyribonucleotide comprises from 5 to 20 nucleotides in length. In some embodiments, the polyribonucleotide comprises from 5 to 15 nucleotides in length. In some embodiments, the polyribonucleotide comprises from 5 to 10 nucleotides in length.

In some embodiments, the polyribonucleotide of the capped polyribonucleotide comprises from 10 to 1100 nucleotides in length. In some embodiments, the polyribonucleotide comprises from 15 to 1100 nucleotides in length. In some embodiments, the polyribonucleotide comprises from 20 to 1100 nucleotides in length. In some embodiments, the polyribonucleotide comprises from 25 to 1100 nucleotides in length. In some embodiments, the polyribonucleotide comprises from 30 to 1100 nucleotides in length. In some embodiments, the polyribonucleotide comprises from 35 to 1100 nucleotides in length. In some embodiments, the polyribonucleotide comprises from 40 to 1100 nucleotides in length. In some embodiments, the polyribonucleotide comprises from 45 to 1100 nucleotides in length. In some embodiments, the polyribonucleotide comprises from 50 to 1100 nucleotides in length. In some embodiments, the polyribonucleotide comprises from 55 to 1100 nucleotides in length. In some embodiments, the polyribonucleotide comprises from 60 to 1100 nucleotides in length. In some embodiments, the polyribonucleotide comprises from 65 to 1100 nucleotides in length. In some embodiments, the polyribonucleotide comprises from 70 to 1100 nucleotides in length. In some embodiments, the polyribonucleotide comprises from 75 to 1100 nucleotides in length. In some embodiments, the polyribonucleotide comprises from 80 to 1100 nucleotides in length. In some embodiments, the polyribonucleotide comprises from 85 to 1100 nucleotides in length. In some embodiments, the polyribonucleotide comprises from 90 to 1100 nucleotides in length. In some embodiments, the polyribonucleotide comprises from 95 to 1100 nucleotides in length. In some embodiments, the polyribonucleotide comprises from 100 to 1100 nucleotides in length. In some embodiments, the polyribonucleotide comprises from 150 to 1100 nucleotides in length. In some embodiments, the polyribonucleotide comprises from 200 to 1100 nucleotides in length. In some embodiments, the polyribonucleotide comprises from 250 to 1100 nucleotides in length. In some embodiments, the polyribonucleotide comprises from 300 to 1100 nucleotides in length. In some embodiments, the polyribonucleotide comprises from 350 to 1100 nucleotides in length. In some embodiments, the polyribonucleotide comprises from 400 to 1100 nucleotides in length. In some embodiments, the polyribonucleotide comprises from 450 to 1100 nucleotides in length. In some embodiments, the polyribonucleotide comprises from 500 to 1100 nucleotides in length. In some embodiments, the polyribonucleotide comprises from 550 to 1100 nucleotides in length. In some embodiments, the polyribonucleotide comprises from 600 to 1100 nucleotides in length. In some embodiments, the polyribonucleotide comprises from 650 to 1100 nucleotides in length. In some embodiments, the polyribonucleotide comprises from 700 to 1100 nucleotides in length. In some embodiments, the polyribonucleotide comprises from 750 to 1100 nucleotides in length. In some embodiments, the polyribonucleotide comprises from 800 to 1100 nucleotides in length. In some embodiments, the polyribonucleotide comprises from 850 to 1100 nucleotides in length. In some embodiments, the polyribonucleotide comprises from 900 to 1100 nucleotides in length. In some embodiments, the polyribonucleotide comprises from 950 to 1100 nucleotides in length. In some embodiments, the polyribonucleotide comprises from 1000 to 1100 nucleotides in length. In some embodiments, the polyribonucleotide comprises from 1050 to 1100 nucleotides in length.

In some embodiments, the polyribonucleotide of the capped polyribonucleotide comprises at least 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, 100 nt, 120 nt, 140 nt, 160 nt, 180 nt, 200 nt, 250 nt, 300 nt, 350 nt, 400 nt, 450 nt, or 500 nt. In some embodiments, the polyribonucleotide of the capped polyribonucleotide comprises at least 10 nt. In some embodiments, the polyribonucleotide of the capped polyribonucleotide comprises at least 15 nt. In some embodiments, the polyribonucleotide of the capped polyribonucleotide comprises at least 20 nt. In some embodiments, the polyribonucleotide of the capped polyribonucleotide comprises at least 25 nt. In some embodiments, the polyribonucleotide of the capped polyribonucleotide comprises at least 30 nt. In some embodiments, the polyribonucleotide of the capped polyribonucleotide comprises at least 35 nt. In some embodiments, the polyribonucleotide of the capped polyribonucleotide comprises at least 40 nt. In some embodiments, the polyribonucleotide of the capped polyribonucleotide comprises at least 45 nt. In some embodiments, the polyribonucleotide of the capped polyribonucleotide comprises at least 50 nt. In some embodiments, the polyribonucleotide of the capped polyribonucleotide comprises at least 55 nt. In some embodiments, the polyribonucleotide of the capped polyribonucleotide comprises at least 60 nt. In some embodiments, the polyribonucleotide of the capped polyribonucleotide comprises at least 65 nt. In some embodiments, the polyribonucleotide of the capped polyribonucleotide comprises at least 70 nt. In some embodiments, the polyribonucleotide of the capped polyribonucleotide comprises at least 75 nt. In some embodiments, the polyribonucleotide of the capped polyribonucleotide comprises at least 80 nt. In some embodiments, the polyribonucleotide of the capped polyribonucleotide comprises at least 85 nt. In some embodiments, the polyribonucleotide of the capped polyribonucleotide comprises at least 90 nt. In some embodiments, the polyribonucleotide of the capped polyribonucleotide comprises at least 95 nt. In some embodiments, the polyribonucleotide of the capped polyribonucleotide comprises at least 100 nt. In some embodiments, the polyribonucleotide of the capped polyribonucleotide comprises at least 120 nt. In some embodiments, the polyribonucleotide of the capped polyribonucleotide comprises at least 140 nt. In some embodiments, the polyribonucleotide of the capped polyribonucleotide comprises at least 160 nt. In some embodiments, the polyribonucleotide of the capped polyribonucleotide comprises at least 180 nt. In some embodiments, the polyribonucleotide of the capped polyribonucleotide comprises at least 200 nt. In some embodiments, the polyribonucleotide of the capped polyribonucleotide comprises at least 250 nt. In some embodiments, the polyribonucleotide of the capped polyribonucleotide comprises at least 300 nt. In some embodiments, the polyribonucleotide of the capped polyribonucleotide comprises at least 350 nt. In some embodiments, the polyribonucleotide of the capped polyribonucleotide comprises at least 400 nt. In some embodiments, the polyribonucleotide of the capped polyribonucleotide comprises at least 450 nt. In some embodiments, the polyribonucleotide of the capped polyribonucleotide comprises at least 500 nt. In some embodiments, the polyribonucleotide comprises 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, 100 nt, 120 nt, 140 nt, 160 nt, 180 nt, 200 nt, 250 nt, 300 nt, 350 nt, 400 nt, 450 nt, or 500 nt. In some embodiments, the polyribonucleotide comprises 10 nt. In some embodiments, the polyribonucleotide comprises 15 nt. In some embodiments, the polyribonucleotide comprises 20 nt. In some embodiments, the polyribonucleotide comprises 25 nt. In some embodiments, the polyribonucleotide comprises 30 nt. In some embodiments, the polyribonucleotide comprises 35 nt. In some embodiments, the polyribonucleotide comprises 40 nt. In some embodiments, the polyribonucleotide comprises 45 nt. In some embodiments, the polyribonucleotide comprises 50 nt. In some embodiments, the polyribonucleotide comprises 55 nt. In some embodiments, the polyribonucleotide comprises 60 nt. In some embodiments, the polyribonucleotide comprises 65 nt. In some embodiments, the polyribonucleotide comprises 70 nt. In some embodiments, the polyribonucleotide comprises 75 nt. In some embodiments, the polyribonucleotide comprises 80 nt. In some embodiments, the polyribonucleotide comprises 85 nt. In some embodiments, the polyribonucleotide comprises 90 nt. In some embodiments, the polyribonucleotide comprises 95 nt. In some embodiments, the polyribonucleotide comprises 100 nt. In some embodiments, the polyribonucleotide comprises 120 nt. In some embodiments, the polyribonucleotide comprises 140 nt. In some embodiments, the polyribonucleotide comprises 160 nt. In some embodiments, the polyribonucleotide comprises 180 nt. In some embodiments, the polyribonucleotide comprises 200 nt. In some embodiments, the polyribonucleotide comprises 250 nt. In some embodiments, the polyribonucleotide comprises 300 nt. In some embodiments, the polyribonucleotide comprises 350 nt. In some embodiments, the polyribonucleotide comprises 400 nt. In some embodiments, the polyribonucleotide comprises 450 nt. In some embodiments, the polyribonucleotide comprises 500 nt. In some embodiments, the polyribonucleotide comprises at least 50 nt, 51 nt, 52 nt, 53 nt, 54 nt, 55 nt, 56 nt, 57 nt, 58 nt, 59 nt, 60 nt, 61 nt, 62 nt, 63 nt, 64 nt, 65 nt, 66 nt, 67 nt, 68 nt, 69 nt, 70 nt, 71 nt, 72 nt, 73 nt, 74 nt, 75 nt, 76 nt, 77 nt, 78 nt, 79 nt, 80 nt, 81 nt, 82 nt, 83 nt, 84 nt, or 85 nt. In some embodiments, the polyribonucleotide comprises at least 50 nt. In some embodiments, the polyribonucleotide comprises at least 51 nt. In some embodiments, the polyribonucleotide comprises at least 52 nt. In some embodiments, the polyribonucleotide comprises at least 53 nt. In some embodiments, the polyribonucleotide comprises at least 54 nt. In some embodiments, the polyribonucleotide comprises at least 55 nt. In some embodiments, the polyribonucleotide comprises at least 56 nt. In some embodiments, the polyribonucleotide comprises at least 57 nt. In some embodiments, the polyribonucleotide comprises at least 58 nt. In some embodiments, the polyribonucleotide comprises at least 59 nt. In some embodiments, the polyribonucleotide comprises at least 60 nt. In some embodiments, the polyribonucleotide comprises at least 61 nt. In some embodiments, the polyribonucleotide comprises at least 62 nt. In some embodiments, the polyribonucleotide comprises at least 63 nt. In some embodiments, the polyribonucleotide comprises at least 64 nt. In some embodiments, the polyribonucleotide comprises at least 65 nt. In some embodiments, the polyribonucleotide comprises at least 66 nt. In some embodiments, the polyribonucleotide comprises at least 67 nt. In some embodiments, the polyribonucleotide comprises at least 68 nt. In some embodiments, the polyribonucleotide comprises at least 69 nt. In some embodiments, the polyribonucleotide comprises at least 70 nt. In some embodiments, the polyribonucleotide comprises at least 71 nt. In some embodiments, the polyribonucleotide comprises at least 72 nt. In some embodiments, the polyribonucleotide comprises at least 73 nt. In some embodiments, the polyribonucleotide comprises at least 74 nt. In some embodiments, the polyribonucleotide comprises at least 75 nt. In some embodiments, the polyribonucleotide comprises at least 76 nt. In some embodiments, the polyribonucleotide comprises at least 77 nt. In some embodiments, the polyribonucleotide comprises at least 78 nt. In some embodiments, the polyribonucleotide comprises at least 79 nt. In some embodiments, the polyribonucleotide comprises at least 80 nt. In some embodiments, the polyribonucleotide comprises at least 81 nt. In some embodiments, the polyribonucleotide comprises at least 82 nt. In some embodiments, the polyribonucleotide comprises at least 83 nt. In some embodiments, the polyribonucleotide comprises at least 84 nt. In some embodiments, the polyribonucleotide comprises at least or 85 nt. In some embodiments, the polyribonucleotide comprises 50 nt, 51 nt, 52 nt, 53 nt, 54 nt, 55 nt, 56 nt, 57 nt, 58 nt, 59 nt, 60 nt, 61 nt, 62 nt, 63 nt, 64 nt, 65 nt, 66 nt, 67 nt, 68 nt, 69 nt, 70 nt, 71 nt, 72 nt, 73 nt, 74 nt, 75 nt, 76 nt, 77 nt, 78 nt, 79 nt, 80 nt, 81 nt, 82 nt, 83 nt, 84 nt, or 85 nt. In some embodiments, the polyribonucleotide comprises 50 nt. In some embodiments, the polyribonucleotide comprises 51 nt. In some embodiments, the polyribonucleotide comprises 52 nt. In some embodiments, the polyribonucleotide comprises 53 nt. In some embodiments, the polyribonucleotide comprises 54 nt. In some embodiments, the polyribonucleotide comprises 55 nt. In some embodiments, the polyribonucleotide comprises 56 nt. In some embodiments, the polyribonucleotide comprises 57 nt. In some embodiments, the polyribonucleotide comprises 58 nt. In some embodiments, the polyribonucleotide comprises 59 nt. In some embodiments, the polyribonucleotide comprises 60 nt. In some embodiments, the polyribonucleotide comprises 61 nt. In some embodiments, the polyribonucleotide comprises 62 nt. In some embodiments, the polyribonucleotide comprises 63 nt. In some embodiments, the polyribonucleotide comprises 64 nt. In some embodiments, the polyribonucleotide comprises 65 nt. In some embodiments, the polyribonucleotide comprises 66 nt. In some embodiments, the polyribonucleotide comprises 67 nt. In some embodiments, the polyribonucleotide comprises 68 nt. In some embodiments, the polyribonucleotide comprises 69 nt. In some embodiments, the polyribonucleotide comprises 70 nt. In some embodiments, the polyribonucleotide comprises 71 nt. In some embodiments, the polyribonucleotide comprises 72 nt. In some embodiments, the polyribonucleotide comprises 73 nt. In some embodiments, the polyribonucleotide comprises 74 nt. In some embodiments, the polyribonucleotide comprises 75 nt. In some embodiments, the polyribonucleotide comprises 76 nt. In some embodiments, the polyribonucleotide comprises 77 nt. In some embodiments, the polyribonucleotide comprises 78 nt. In some embodiments, the polyribonucleotide comprises 79 nt. In some embodiments, the polyribonucleotide comprises 80 nt. In some embodiments, the polyribonucleotide comprises 81 nt. In some embodiments, the polyribonucleotide comprises 82 nt. In some embodiments, the polyribonucleotide comprises 83 nt. In some embodiments, the polyribonucleotide comprises 84 nt. In some embodiments, the polyribonucleotide comprises 85 nt.

### UTR

In some embodiments, the polyribonucleotide of the capped polyribonucleotide comprises a UTR (untranslated region). UTRs of a genomic region comprising a gene may be transcribed but not translated. A UTR can be involved in translation regulation, influence localization and stability of the polyribonucleotide, and can comprise binding sites for regulatory proteins and microRNAs. In some embodiments, the UTR comprises a ribosome binding site.

In some embodiments, the UTR comprises secondary structures, such as a hairpin loop, that regulates translation. In some embodiments, the polyribonucleotide of the capped polyribonucleotide comprises a UTR with one or more stretches of Adenosines and Uridines embedded within. These AU rich signatures are may increase turnover rates of the expression product.

Introduction, removal or modification of UTR AU rich elements (AREs) may be useful to modulate the stability or immunogenicity of the polyribonucleotide. When engineering specific polyribonucleotides, one or more copies of an ARE may be introduced to the polyribonucleotide and the copies of an ARE may modulate translation and/or production of an expression product. Likewise, AREs may be identified and removed or engineered into the polyribonucleotide to modulate the intracellular stability and thus affect translation and production of the resultant protein.

It should be understood that any UTR from any gene may be incorporated into the respective flanking regions of the polyribonucleotide. As a non-limiting example, the UTR or a fragment thereof which may be incorporated is a UTR listed in U.S. Provisional Application Nos. US 61/775,509 and US 61/829,372, or in International Patent Application No. PCT/US2014/021522; the contents of each of which are herein incorporated by reference in its entirety. Furthermore, multiple wild-type UTRs of any known gene may be utilized. It is also within the scope of the present invention to provide artificial UTRs which are not variants of wild type genes. These UTRs or portions thereof may be placed in the same orientation as in the transcript from which they were selected or may be altered in orientation or location. Hence a 5′ or 3′ UTR may be inverted, shortened, lengthened, made chimeric with one or more other 5′ UTRs or 3′ UTRs. As used herein, the term “altered” as it relates to a UTR sequence, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In one embodiment, a double, triple or quadruple UTR, such as a 5′ or 3′ UTR, may be used. As used herein, a “double” UTR is one in which two copies of the same UTR are encoded either in series or substantially in series. For example, a double beta- globin 3′ UTR may be used as described in U.S. Pat. Publication 20100129877, the contents of which are incorporated herein by reference in its entirety.

In some embodiments, the polyribonucleotide of the capped polyribonucleotide comprises a 5′ UTR. The 5′ UTR can be 5′ to a binding region of the polyribonucleotide, wherein the binding region binds to a circular polyribonucleotide. In some embodiments, a polyribonucleotide of the capped polyribonucleotide comprises a poly-A region. The 5′ UTR can be 5′ of the poly-A region of the polyribonucleotide of the capped polyribonucleotide. In some embodiments, the polyribonucleotide of the capped polyribonucleotide comprises a 3′ UTR. The 3′ UTR can be 3′ to a binding region of the polyribonucleotide, wherein the binding region binds to a circular polyribonucleotide. In some embodiments, the polyribonucleotide of the capped polyribonucleotide lacks a UTR.

### PolyA Region

The polyribonucleotide of the capped polyribonucleotide can comprise a poly-A region. In some embodiments, the length of a poly-A region is greater than 10 nucleotides in length. In one embodiment, the poly-A region is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A region is from about 10 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000). In some embodiments, the poly-A region is 15 nucleotides in length. In some embodiments, the poly-A region is 10 nucleotides in length. In some embodiments, the poly-A region is 15 nucleotides in length. In some embodiments, the poly-A region is 20 nucleotides in length. In some embodiments, the poly-A region is 25 nucleotides in length. In some embodiments, the poly-A region is 30 nucleotides in length. In some embodiments, the poly-A region is 35 nucleotides in length. In some embodiments, the poly-A region is 40 nucleotides in length. In some embodiments, the poly-A region is 45 nucleotides in length. In some embodiments, the poly-A region is 50 nucleotides in length. In some embodiments, the poly-A region is 55 nucleotides in length. In some embodiments, the poly-A region is 60 nucleotides in length. In some embodiments, the poly-A region is 70 nucleotides in length. In some embodiments, the poly-A region is 80 nucleotides in length. In some embodiments, the poly-A region is 90 nucleotides in length. In some embodiments, the poly-A region is 100 nucleotides in length. In some embodiments, the poly-A region is 120 nucleotides in length. In some embodiments, the poly-A region is 140 nucleotides in length. In some embodiments, the poly-A region is 160 nucleotides in length. In some embodiments, the poly-A region is 180 nucleotides in length. In some embodiments, the poly-A region is 200 nucleotides in length. In some embodiments, the poly-A region is 250 nucleotides in length. In some embodiments, the poly-A region is 300 nucleotides in length. In some embodiments, the poly-A region is 350 nucleotides in length. In some embodiments, the poly-A region is 400 nucleotides in length. In some embodiments, the poly-A region is 450 nucleotides in length. In some embodiments, the poly-A region is 500 nucleotides in length. In some embodiments, the poly-A region is 600 nucleotides in length. In some embodiments, the poly-A region is 700 nucleotides in length. In some embodiments, the poly-A region is 800 nucleotides in length. In some embodiments, the poly-A region is 900 nucleotides in length. In some embodiments, the poly-A region is 1,000 nucleotides in length. In some embodiments, the poly-A region is 1,100 nucleotides in length. In some embodiments, the poly-A region is 1,200 nucleotides in length. In some embodiments, the poly-A region is 1,300 nucleotides in length. In some embodiments, the poly-A region is 1,400 nucleotides in length. In some embodiments, the poly-A region is 1,500 nucleotides in length. In some embodiments, the poly-A region is 1,600 nucleotides in length. In some embodiments, the poly-A region is 1,700 nucleotides in length. In some embodiments, the poly-A region is 1,800 nucleotides in length. In some embodiments, the poly-A region is 1,900 nucleotides in length. In some embodiments, the poly-A region is 2,000 nucleotides in length. In some embodiments, the poly-A region is 2,500 nucleotides in length. In some embodiments, the poly-A region is 3,000 nucleotides.

In one embodiment, the poly-A region is designed relative to the length of the overall polyribonucleotide. This design may be based on the length of the coding region, the length of a particular feature or region (such as the first or flanking regions). In this context, the poly-A region may be 10, 20, 30, 40, 50, 60, 70, 80, 90, or 100% greater in length than the circular polyribonucleotide or a feature thereof. The poly-A region may also be designed as a fraction of the polyribonucleotide to which it belongs. In this context, the poly-A region may be 10, 20, 30, 40, 50, 60, 70, 80, or 90% or more of the total length of the construct or the total length of the construct minus the poly-A region. Further, engineered binding sites and conjugation of the polyribonucleotide for Poly-A binding protein may enhance expression.

In one embodiment, the polyribonucleotide is designed to include a polyA-G quartet. The G-quartet is a cyclic hydrogen bonded array of four guanine nucleotides that can be formed by G-rich sequences in both DNA and RNA. In one embodiment, the G-quartet is incorporated at the end of the poly-A sequence. The resultant polyribonucleotide construct is assayed for stability, protein production and/or other parameters including half-life at various time points. In some embodiments, the polyA-G quartet results in protein production equivalent to at least 75% of that seen using a poly-A sequence of 120 nucleotides alone.

In some embodiments, the polyribonucleotide comprises a polyA. In some embodiments, the polyribonucleotide lacks a polyA. In some embodiments, the polyribonucleotide has a modified polyA to modulate one or more characteristics of the polyribonucleotide. In some embodiments, the polyribonucleotide lacking a polyA or having modified polyA improves one or more functional characteristics, e.g., immunogenicity, half-life, expression efficiency, etc.

### Binding Region

A polyriboncleotide of the capped polyribonucleotide as described herein can comprise a binding region that binds to a circular polyribonucleotide as described herein. The binding region can be 3′ of a UTR in the polyribonucleotide. The binding region can be 5′ of a UTR in the polyribonucleotide. The binding region can be 5′ of a poly-A region. Often, the binding region is first binding region that comprises a sequence that is antisense to the sequence of second binding region, wherein a circular polyribonucleotide comprises the second binding region.

In some embodiments, the first binding region of the polyribonucleotide of the capped polyribonucleotide comprises from 5 to 100 nucleotides in length. In some embodiments, the first binding region comprises from 5 to 90 nucleotides in length. In some embodiments, the first binding region comprises from 5 to 85 nucleotides in length. In some embodiments, the first binding region comprises from 5 to 80 nucleotides in length. In some embodiments, the first binding region comprises from 5 to 75 nucleotides in length. In some embodiments, the first binding region comprises from 5 to 70 nucleotides in length. In some embodiments, the first binding region comprises from 5 to 65 nucleotides in length. In some embodiments, the first binding region comprises from 5 to 60 nucleotides in length. In some embodiments, the first binding region comprises from 5 to 55 nucleotides in length. In some embodiments, the first binding region comprises from 5 to 50 nucleotides in length. In some embodiments, the first binding region comprises from 5 to 45 nucleotides in length. In some embodiments, the first binding region comprises from 5 to 40 nucleotides in length. In some embodiments, the first binding region comprises from 5 to 35 nucleotides in length. In some embodiments, the first binding region comprises from 5 to 30 nucleotides in length. In some embodiments, the first binding region comprises from 5 to 25 nucleotides in length. In some embodiments, the first binding region comprises from 5 to 20 nucleotides in length. In some embodiments, the first binding region comprises from 5 to 15 nucleotides in length. In some embodiments, the first binding region comprises from 5 to 10 nucleotides in length.

In some embodiments, the first binding region comprises from 5 to 95 nucleotides in length. In some embodiments, the first binding region comprises from 10 to 95 nucleotides in length. In some embodiments, the first binding region comprises from 15 to 95 nucleotides in length. In some embodiments, the first binding region comprises from 20 to 95 nucleotides in length. In some embodiments, the first binding region comprises from 25 to 95 nucleotides in length. In some embodiments, the first binding region comprises from 30 to 95 nucleotides in length. In some embodiments, the first binding region comprises from 35 to 95 nucleotides in length. In some embodiments, the first binding region comprises from 40 to 95 nucleotides in length. In some embodiments, the first binding region comprises from 45 to 95 nucleotides in length. In some embodiments, the first binding region comprises from 50 to 95 nucleotides in length. In some embodiments, the first binding region comprises from 55 to 95 nucleotides in length. In some embodiments, the first binding region comprises from 60 to 95 nucleotides in length. In some embodiments, the first binding region comprises from 65 to 95 nucleotides in length. In some embodiments, the first binding region comprises from 70 to 95 nucleotides in length. In some embodiments, the first binding region comprises from 75 to 95 nucleotides in length. In some embodiments, the first binding region comprises from 80 to 95 nucleotides in length. In some embodiments, the first binding region comprises from 85 to 95 nucleotides in length. In some embodiments, the first binding region comprises from 90 to 95 nucleotides in length.

In some embodiments, the first binding region comprises from 10 to 95 nucleotides in length. In some embodiments, the first binding region comprises from 15 to 90 nucleotides in length. In some embodiments, the first binding region comprises from 20 to 85 nucleotides in length. In some embodiments, the first binding region comprises from 25 to 80 nucleotides in length. In some embodiments, the first binding region comprises from 30 to 75 nucleotides in length. In some embodiments, the first binding region comprises from 35 to 70 nucleotides in length. In some embodiments, the first binding region comprises from 40 to 65 nucleotides in length. In some embodiments, the first binding region comprises from 45 to 60 nucleotides in length. In some embodiments, the first binding region comprises from 50 to 55 nucleotides in length.

In some embodiments, the first binding region comprises at least 5 nucleotides (nt), 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt. In some embodiments, the first binding region comprises 5 nt, 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt. In some embodiments, the first binding region comprises at least 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or 35 nt. In some embodiments, the first binding region comprises 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or 35 nt.

In some embodiments, the first binding region comprises 5 nucleotides. In some embodiments, the first binding region comprises 10 nt. In some embodiments, the first binding region comprises 15 nt. In some embodiments, the first binding region comprises 20 nt. In some embodiments, the first binding region comprises 25 nt. In some embodiments, the first binding region comprises 30 nt. In some embodiments, the first binding region comprises 35 nt. In some embodiments, the first binding region comprises 40 nt. In some embodiments, the first binding region comprises 45 nt. In some embodiments, the first binding region comprises 50 nt. In some embodiments, the first binding region comprises 55 nt. In some embodiments, the first binding region comprises 60 nt. In some embodiments, the first binding region comprises 65 nt. In some embodiments, the first binding region comprises 70 nt. In some embodiments, the first binding region comprises 75 nt. In some embodiments, the first binding region comprises 80 nt. In some embodiments, the first binding region comprises 85 nt. In some embodiments, the first binding region comprises 90 nt. In some embodiments, the first binding region comprises 95 nt. In some embodiments, the first binding region comprises 100 nt.

In some embodiments, the first binding region specifically binds to a second binding region of a circular polyribonucleotide. Often, the binding region is first binding region that comprises a sequence that is antisense to the sequence of second binding region, wherein a circular polyribonucleotide comprises the second binding region. In some embodiments, the first binding region of the polyribonucleotide is complementary to the second binding region of the circular polyribonucleotide, which allows the base pairing between the polyribonucleotide and the circular polyribonucleotide. In some embodiments, the first binding region of the polyribonucleotide is 100% complementary to the second binding region of the circular polyribonucleotide. In some embodiments, the first binding region is at least 99%, 98%, 97%, 96%, 95%, 94%, 93%, 92%, 91%, 90%, 85%, 80%, 75%, 70%, 65%, 60%, 55%, 50%, 45%, 40%, 35%, 30%, or more complementary to the second binding region. In some embodiments, the first binding region of the polyribonucleotide is 100% complementary to the second binding region of the circular polyribonucleotide. In some embodiments, the first binding region is at least 99% complementary to the second binding region of the circular polyribonucleotide. In some embodiments, the first binding region is at least 98% complementary to the second binding region of the circular polyribonucleotide. In some embodiments, the first binding region is at least 97% complementary to the second binding region of the circular polyribonucleotide. In some embodiments, the first binding region is at least 96% complementary to the second binding region of the circular polyribonucleotide. In some embodiments, the first binding region is at least 95% complementary to the second binding region of the circular polyribonucleotide. In some embodiments, the first binding region is at least 94% complementary to the second binding region of the circular polyribonucleotide. In some embodiments, the first binding region is at least 93% complementary to the second binding region of the circular polyribonucleotide. In some embodiments, the first binding region is at least 92% complementary to the second binding region of the circular polyribonucleotide. In some embodiments, the first binding region is at least 91% complementary to the second binding region of the circular polyribonucleotide. In some embodiments, the first binding region is at least 90% complementary to the second binding region of the circular polyribonucleotide. In some embodiments, the first binding region is at least 85% complementary to the second binding region of the circular polyribonucleotide. In some embodiments, the first binding region is at least 80% complementary to the second binding region of the circular polyribonucleotide. In some embodiments, the first binding region is at least 75% complementary to the second binding region of the circular polyribonucleotide. In some embodiments, the first binding region is at least 70% complementary to the second binding region of the circular polyribonucleotide. In some embodiments, the first binding region is at least 65% complementary to the second binding region of the circular polyribonucleotide. In some embodiments, the first binding region is at least 60% complementary to the second binding region of the circular polyribonucleotide. In some embodiments, the first binding region is at least 55% complementary to the second binding region of the circular polyribonucleotide. In some embodiments, the first binding region is at least 50% complementary to the second binding region of the circular polyribonucleotide. In some embodiments, the first binding region is at least 45% complementary to the second binding region of the circular polyribonucleotide. In some embodiments, the first binding region is at least 40% complementary to the second binding region of the circular polyribonucleotide. In some embodiments, the first binding region is at least 35% complementary to the second binding region of the circular polyribonucleotide. In some embodiments, the first binding region is at least 30% complementary to the second binding region. In some embodiments, the first binding region is 99%, 98%, 97%, 96%, 95%, 94%, 93%, 92%, 91%, 90%, 85%, 80%, 75%, 70%, 65%, 60%, 55%, 50%, 45%, 40%, 35%, 30%, or more complementary to the second binding region. In some embodiments, the first binding region is 99% complementary to the second binding region. In some embodiments, the first binding region is 98% complementary to the second binding region. In some embodiments, the first binding region is 97% complementary to the second binding region. In some embodiments, the first binding region is 96% complementary to the second binding region. In some embodiments, the first binding region is 95% complementary to the second binding region. In some embodiments, the first binding region is 94% complementary to the second binding region. In some embodiments, the first binding region is 93% complementary to the second binding region. In some embodiments, the first binding region is 92% complementary to the second binding region. In some embodiments, the first binding region is 91% complementary to the second binding region. In some embodiments, the first binding region is 90% complementary to the second binding region. In some embodiments, the first binding region is 85% complementary to the second binding region. In some embodiments, the first binding region is 80% complementary to the second binding region. In some embodiments, the first binding region is 75% complementary to the second binding region. In some embodiments, the first binding region is 70% complementary to the second binding region. In some embodiments, the first binding region is 65% complementary to the second binding region. In some embodiments, the first binding region is 60% complementary to the second binding region. In some embodiments, the first binding region is 55% complementary to the second binding region. In some embodiments, the first binding region is 50% complementary to the second binding region. In some embodiments, the first binding region is 45% complementary to the second binding region. In some embodiments, the first binding region is 40% complementary to the second binding region. In some embodiments, the first binding region is 35% complementary to the second binding region. In some embodiments, the first binding region is 30% complementary to the second binding region.

In some embodiments, the capped polynucleotides as described herein are a plurality of capped polynucleotides. In some embodiments, the plurality of the capped polynucleotides comprises one or more different capped polynucleotides. In some embodiments, the one or more different capped polynucleotides comprise a different binding region. For example, a third capped polynucleotide comprises a third binding region that binds to a fourth binding region of a circular polynucleotide and a fourth capped polynucleotide comprises a third binding region that binds to the circular polyribonucleotide.

In some embodiments, the third binding region of the polyribonucleotide of the capped polyribonucleotide comprises from 5 to 100 nucleotides in length. In some embodiments, the third binding region comprises from 5 to 90 nucleotides in length. In some embodiments, the third binding region comprises from 5 to 85 nucleotides in length. In some embodiments, the third binding region comprises from 5 to 80 nucleotides in length. In some embodiments, the third binding region comprises from 5 to 75 nucleotides in length. In some embodiments, the third binding region comprises from 5 to 70 nucleotides in length. In some embodiments, the third binding region comprises from 5 to 65 nucleotides in length. In some embodiments, the third binding region comprises from 5 to 60 nucleotides in length. In some embodiments, the third binding region comprises from 5 to 55 nucleotides in length. In some embodiments, the third binding region comprises from 5 to 50 nucleotides in length. In some embodiments, the third binding region comprises from 5 to 45 nucleotides in length. In some embodiments, the third binding region comprises from 5 to 40 nucleotides in length. In some embodiments, the third binding region comprises from 5 to 35 nucleotides in length. In some embodiments, the third binding region comprises from 5 to 30 nucleotides in length. In some embodiments, the third binding region comprises from 5 to 25 nucleotides in length. In some embodiments, the third binding region comprises from 5 to 20 nucleotides in length. In some embodiments, the third binding region comprises from 5 to 15 nucleotides in length. In some embodiments, the third binding region comprises from 5 to 10 nucleotides in length.

In some embodiments, the third binding region comprises from 5 to 95 nucleotides in length. In some embodiments, the third binding region comprises from 10 to 95 nucleotides in length. In some embodiments, the third binding region comprises from 15 to 95 nucleotides in length. In some embodiments, the third binding region comprises from 20 to 95 nucleotides in length. In some embodiments, the third binding region comprises from 25 to 95 nucleotides in length. In some embodiments, the third binding region comprises from 30 to 95 nucleotides in length. In some embodiments, the third binding region comprises from 35 to 95 nucleotides in length. In some embodiments, the third binding region comprises from 40 to 95 nucleotides in length. In some embodiments, the third binding region comprises from 45 to 95 nucleotides in length. In some embodiments, the third binding region comprises from 50 to 95 nucleotides in length. In some embodiments, the third binding region comprises from 55 to 95 nucleotides in length. In some embodiments, the third binding region comprises from 60 to 95 nucleotides in length. In some embodiments, the third binding region comprises from 65 to 95 nucleotides in length. In some embodiments, the third binding region comprises from 70 to 95 nucleotides in length. In some embodiments, the third binding region comprises from 75 to 95 nucleotides in length. In some embodiments, the third binding region comprises from 80 to 95 nucleotides in length. In some embodiments, the third binding region comprises from 85 to 95 nucleotides in length. In some embodiments, the third binding region comprises from 90 to 95 nucleotides in length.

In some embodiments, the third binding region comprises from 10 to 95 nucleotides in length. In some embodiments, the third binding region comprises from 15 to 90 nucleotides in length. In some embodiments, the third binding region comprises from 20 to 85 nucleotides in length. In some embodiments, the third binding region comprises from 25 to 80 nucleotides in length. In some embodiments, the third binding region comprises from 30 to 75 nucleotides in length. In some embodiments, the third binding region comprises from 35 to 70 nucleotides in length. In some embodiments, the third binding region comprises from 40 to 65 nucleotides in length. In some embodiments, the third binding region comprises from 45 to 60 nucleotides in length. In some embodiments, the third binding region comprises from 50 to 55 nucleotides in length.

In some embodiments, the third binding region comprises at least 5 nucleotides (nt), 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt. In some embodiments, the third binding region comprises 5 nt, 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt. In some embodiments, the third binding region comprises at least 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or 35 nt. In some embodiments, the third binding region comprises 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or 35 nt.

In some embodiments, the third binding region comprises 5 nucleotides. In some embodiments, the third binding region comprises 10 nt. In some embodiments, the third binding region comprises 15 nt. In some embodiments, the third binding region comprises 20 nt. In some embodiments, the third binding region comprises 25 nt. In some embodiments, the third binding region comprises 30 nt. In some embodiments, the third binding region comprises 35 nt. In some embodiments, the third binding region comprises 40 nt. In some embodiments, the third binding region comprises 45 nt. In some embodiments, the third binding region comprises 50 nt. In some embodiments, the third binding region comprises 55 nt. In some embodiments, the third binding region comprises 60 nt. In some embodiments, the third binding region comprises 65 nt. In some embodiments, the third binding region comprises 70 nt. In some embodiments, the third binding region comprises 75 nt. In some embodiments, the third binding region comprises 80 nt. In some embodiments, the third binding region comprises 85 nt. In some embodiments, the third binding region comprises 90 nt. In some embodiments, the third binding region comprises 95 nt. In some embodiments, the third binding region comprises 100 nt.

In some embodiments, the third binding region specifically binds to a fourth binding region of a circular polyribonucleotide. Often, the binding region is third binding region that comprises a sequence that is antisense to the sequence of fourth binding region, wherein a circular polyribonucleotide comprises the fourth binding region. In some embodiments, the third binding region of the polyribonucleotide is complementary to the fourth binding region of the circular polyribonucleotide, which allows the base pairing between the polyribonucleotide and the circular polyribonucleotide. In some embodiments, the third binding region of the polyribonucleotide is 100% complementary to the fourth binding region of the circular polyribonucleotide. In some embodiments, the third binding region is at least 99%, 98%, 97%, 96%, 95%, 94%, 93%, 92%, 91%, 90%, 85%, 80%, 75%, 70%, 65%, 60%, 55%, 50%, 45%, 40%, 35%, 30%, or more complementary to the fourth binding region. In some embodiments, the third binding region of the polyribonucleotide is 100% complementary to the fourth binding region of the circular polyribonucleotide. In some embodiments, the third binding region is at least 99% complementary to the fourth binding region of the circular polyribonucleotide. In some embodiments, the third binding region is at least 98% complementary to the fourth binding region of the circular polyribonucleotide. In some embodiments, the third binding region is at least 97% complementary to the fourth binding region of the circular polyribonucleotide. In some embodiments, the third binding region is at least 96% complementary to the fourth binding region of the circular polyribonucleotide. In some embodiments, the third binding region is at least 95% complementary to the fourth binding region of the circular polyribonucleotide. In some embodiments, the third binding region is at least 94% complementary to the fourth binding region of the circular polyribonucleotide. In some embodiments, the third binding region is at least 93% complementary to the fourth binding region of the circular polyribonucleotide. In some embodiments, the third binding region is at least 92% complementary to the fourth binding region of the circular polyribonucleotide. In some embodiments, the third binding region is at least 91% complementary to the fourth binding region of the circular polyribonucleotide. In some embodiments, the third binding region is at least 90% complementary to the fourth binding region of the circular polyribonucleotide. In some embodiments, the third binding region is at least 85% complementary to the fourth binding region of the circular polyribonucleotide. In some embodiments, the third binding region is at least 80% complementary to the fourth binding region of the circular polyribonucleotide. In some embodiments, the third binding region is at least 75% complementary to the fourth binding region of the circular polyribonucleotide. In some embodiments, the third binding region is at least 70% complementary to the fourth binding region of the circular polyribonucleotide. In some embodiments, the third binding region is at least 65% complementary to the fourth binding region of the circular polyribonucleotide. In some embodiments, the third binding region is at least 60% complementary to the fourth binding region of the circular polyribonucleotide. In some embodiments, the third binding region is at least 55% complementary to the fourth binding region of the circular polyribonucleotide. In some embodiments, the third binding region is at least 50% complementary to the fourth binding region of the circular polyribonucleotide. In some embodiments, the third binding region is at least 45% complementary to the fourth binding region of the circular polyribonucleotide. In some embodiments, the third binding region is at least 40% complementary to the fourth binding region of the circular polyribonucleotide. In some embodiments, the third binding region is at least 35% complementary to the fourth binding region of the circular polyribonucleotide. In some embodiments, the third binding region is at least 30% complementary to the fourth binding region. In some embodiments, the third binding region is 99%, 98%, 97%, 96%, 95%, 94%, 93%, 92%, 91%, 90%, 85%, 80%, 75%, 70%, 65%, 60%, 55%, 50%, 45%, 40%, 35%, 30%, or more complementary to the fourth binding region. In some embodiments, the third binding region is 99% complementary to the fourth binding region. In some embodiments, the third binding region is 98% complementary to the fourth binding region. In some embodiments, the third binding region is 97% complementary to the fourth binding region. In some embodiments, the third binding region is 96% complementary to the fourth binding region. In some embodiments, the third binding region is 95% complementary to the fourth binding region. In some embodiments, the third binding region is 94% complementary to the fourth binding region. In some embodiments, the third binding region is 93% complementary to the fourth binding region. In some embodiments, the third binding region is 92% complementary to the fourth binding region. In some embodiments, the third binding region is 91% complementary to the fourth binding region. In some embodiments, the third binding region is 90% complementary to the fourth binding region. In some embodiments, the third binding region is 85% complementary to the fourth binding region. In some embodiments, the third binding region is 80% complementary to the fourth binding region. In some embodiments, the third binding region is 75% complementary to the fourth binding region. In some embodiments, the third binding region is 70% complementary to the fourth binding region. In some embodiments, the third binding region is 65% complementary to the fourth binding region. In some embodiments, the third binding region is 60% complementary to the fourth binding region. In some embodiments, the third binding region is 55% complementary to the fourth binding region. In some embodiments, the third binding region is 50% complementary to the fourth binding region. In some embodiments, the third binding region is 45% complementary to the fourth binding region. In some embodiments, the third binding region is 40% complementary to the fourth binding region. In some embodiments, the third binding region is 35% complementary to the fourth binding region. In some embodiments, the third binding region is 30% complementary to the fourth binding region.

In some embodiments, the first binding region and the third binding region are the same. In some embodiments, the first binding region and the third binding region are different.

In some embodiments, the polyribonucleotide of the capped polyribonucleotide is bound to the circular polyribonucleotide by direct binding. In some embodiments, the polyribonucleotide is bound to the circular polyribonucleotide by covalent binding. For example, the polyribonucleotide is bound to the circular polyribonucleotide by click chemistry. In some embodiments, the polyribonucleotide is bound to the circular polyribonucleotide by noncovalent binding. For example, the polyribonucleotide is bound to the circular polyribonucleotide by the canonical Watson-Crick base pairing or non-canoical base pairing. As another example, the polyribonucleotide is bound to the circular polyribonucleotide by electrostatic interactions, such as ionic interactions, a hydrogen bonding and a halogen bonding, π-effects, van der Waals forces, and hydrophobic effects.

In some embodiments, the polyribonucleotide is bound to the circular polyribonucleotide by indirect binding. For example, in some embodiments, the polyribonucleotide is bound to the circular polyribonucleotide through the interaction between co-binding partners, such as chemicals, small molecules, proteins, peptides, agents, or factors, each of which bind to the polyribonucleotide and the circular polyribonucleotide, respectively.

In some embodiments, the polyribonucleotide comprises a 5′ modified guanosine cap and a first binding region, wherein the first binding region specifically binds to a second binding region of a circular polyribonucleotide. For example, in some embodiments, the polyribonucleotide of the capped polyribonucleotide is a linear RNA oligonucleotide encoded with a human alpha globin 5′UTR and a 3′ binding region (can also be referred to as an annealing region) complementary to binding region (the annealing region) of the circular RNA. In some embodiments, the polyribonucleotide comprises the sequence as represented by SEQ ID NO: 4. In some embodiments, the polyribonucleotide comprises the sequence as represented by SEQ ID NO: 5. In some embodiments, the polyribonucleotide of the capped polyribonucleotide comprises the sequence as represented by SEQ ID NO: 1. In some embodiments, the the polyribonucleotide of the capped polyribonucleotide is the sequence as represented by SEQ ID NO: 1.

### Ribosome Recruitment

In some embodiments, the capped polyribonucleotide recruits a ribosome. In some embodiments, the capped polyribonucleotide comprises ribosome-binding moieties. In some embodiments, the capped polyribonucleotide comprises moieties that recruit a ribosome. In some embodiments, the ribosome-binding moieties recruit a ribosome.

### Circular Polyribonucleotides

The circular polyribonucleotides as described herein comprise binding region that specifically binds a capped polyribonucleotide as described herein. The binding region of the circular polyribonucleotide can comprise a sequence that is sense to a sequence of a binding region of a capped polyribonucleotide. In some embodiments, the circular polyribonucleotide further comprises an expression sequence. The circular polyribonucleotide can further comprise a UTR. The circular polyribonucleotide can further comprise a poly-A region. In some embodiments, the circular polyribonucleotide is an unmodified circular polyribonucleotide. In some embodiments, the circular polyribonucleotide is a modified circular polyribonucleotide. The circular polyribonucleotide can form a complex with a capped polyribonucleotide. The cap of the capped polyribonucleotide complexed with the circular polyribonucleotide can recruit a ribosome for initation of translation of an expression sequence in the circular polyribonucleotide. In some embodiments, the circular polyribonucleotide binds to a plurality of capped polyribonucleotides. In some embodiments, the plurality of capped polynucleotides comprises at least two of the same capped polynucleotide. In some embodiments, the plurality of the capped polynucleotides comprises one or more different capped polynucleotides. In some embodiments, the circular polyribonucleotide comprises one or more binding regions that specifically bind one or more binding regions of one or more capped polyribonucleotides. For example, a circular polyribonucleotide comprises a second binding region and a fourth binding region, wherein the second binding region binds to a first binding region of a first capped polyribonucleotide and the fourth binding region binds to a third binding region of a second capped polyribonucleotide. In some embodiments, the second binding region and the fourth binding region are the same. In some embodiments, the second binding region and the fourth binding region are different.

In some embodiments, the circular polyribonucleotide comprises any feature or any combination of features as disclosed in WO2019/118919 and WO2020/023655, which are each hereby incorporated by reference in their entirety.

The circular polyribonucleotide as described herein can comprise from 50 to 20000 nucleotides in length. In some embodiments, the circular polyribonucleotide comprises from 50 to 19000 nucleotides in length. In some embodiments, the circular polyribonucleotide comprises from 50 to 18500 nucleotides in length. In some embodiments, the circular polyribonucleotide comprises from 50 to 18000 nucleotides in length. In some embodiments, the circular polyribonucleotide comprises from 50 to 17500 nucleotides in length. In some embodiments, the circular polyribonucleotide comprises from 50 to 17000 nucleotides in length. In some embodiments, the circular polyribonucleotide comprises from 50 to 16500 nucleotides in length. In some embodiments, the circular polyribonucleotide comprises from 50 to 16000 nucleotides in length. In some embodiments, the circular polyribonucleotide comprises from 50 to 15500 nucleotides in length. In some embodiments, the circular polyribonucleotide comprises from 50 to 15000 nucleotides in length. In some embodiments, the circular polyribonucleotide comprises from 50 to 14500 nucleotides in length. In some embodiments, the circular polyribonucleotide comprises from 50 to 14000 nucleotides in length. In some embodiments, the circular polyribonucleotide comprises from 50 to 13500 nucleotides in length. In some embodiments, the circular polyribonucleotide comprises from 50 to 13000 nucleotides in length. In some embodiments, the circular polyribonucleotide comprises from 50 to 12500 nucleotides in length. In some embodiments, the circular polyribonucleotide comprises from 50 to 12000 nucleotides in length. In some embodiments, the circular polyribonucleotide comprises from 50 to 11500 nucleotides in length. In some embodiments, the circular polyribonucleotide comprises from 50 to 11000 nucleotides in length. In some embodiments, the circular polyribonucleotide comprises from 50 to 11500 nucleotides in length.

In some embodiments, the circular polyribonucleotide comprises from 50 to 10000 nucleotides in length. In some embodiments, the circular polyribonucleotide comprises from 50 to 9500 nucleotides in length. In some embodiments, the circular polyribonucleotide comprises from 50 to 9000 nucleotides in length. In some embodiments, the circular polyribonucleotide comprises from 50 to 8500 nucleotides in length. In some embodiments, the circular polyribonucleotide comprises from 50 to 8000 nucleotides in length. In some embodiments, the circular polyribonucleotide comprises from 50 to 7500 nucleotides in length. In some embodiments, the circular polyribonucleotide comprises from 50 to 7000 nucleotides in length. In some embodiments, the circular polyribonucleotide comprises from 50 to 6500 nucleotides in length. In some embodiments, the circular polyribonucleotide comprises from 50 to 6000 nucleotides in length. In some embodiments, the circular polyribonucleotide comprises from 50 to 5500 nucleotides in length. In some embodiments, the circular polyribonucleotide comprises from 50 to 5000 nucleotides in length. In some embodiments, the circular polyribonucleotide comprises from 50 to 4500 nucleotides in length. In some embodiments, the circular polyribonucleotide comprises from 50 to 4000 nucleotides in length. In some embodiments, the circular polyribonucleotide comprises from 50 to 3500 nucleotides in length. In some embodiments, the circular polyribonucleotide comprises from 50 to 3000 nucleotides in length. In some embodiments, the circular polyribonucleotide comprises from 50 to 2500 nucleotides in length. In some embodiments, the circular polyribonucleotide comprises from 50 to 2000 nucleotides in length. In some embodiments, the circular polyribonucleotide comprises from 50 to 1500 nucleotides in length. In some embodiments, the circular polyribonucleotide comprises from 50 to 1000 nucleotides in length. In some embodiments, the circular polyribonucleotide comprises from 50 to 950 nucleotides in length. In some embodiments, the circular polyribonucleotide comprises from 50 to 900 nucleotides in length. In some embodiments, the circular polyribonucleotide comprises from 50 to 850 nucleotides in length. In some embodiments, the circular polyribonucleotide comprises from 50 to 800 nucleotides in length. In some embodiments, the circular polyribonucleotide comprises from 50 to 750 nucleotides in length. In some embodiments, the circular polyribonucleotide comprises from 50 to 700 nucleotides in length. In some embodiments, the circular polyribonucleotide comprises from 50 to 650 nucleotides in length. In some embodiments, the circular polyribonucleotide comprises from 50 to 600 nucleotides in length. In some embodiments, the circular polyribonucleotide comprises from 50 to 550 nucleotides in length. In some embodiments, the circular polyribonucleotide comprises from 50 to 500 nucleotides in length. In some embodiments, the circular polyribonucleotide comprises from 50 to 450 nucleotides in length. In some embodiments, the circular polyribonucleotide comprises from 50 to 400 nucleotides in length. In some embodiments, the circular polyribonucleotide comprises from 50 to 350 nucleotides in length.

In some embodiments, the circular polyribonucleotide comprises from 100 to 20000 nucleotides in length. In some embodiments, the circular polyribonucleotide comprises from 150 to 20000 nucleotides in length. In some embodiments, the circular polyribonucleotide comprises from 200 to 20000 nucleotides in length. In some embodiments, the circular polyribonucleotide comprises from 250 to 20000 nucleotides in length. In some embodiments, the circular polyribonucleotide comprises from 300 to 20000 nucleotides in length. In some embodiments, the circular polyribonucleotide comprises from 350 to 20000 nucleotides in length. In some embodiments, the circular polyribonucleotide comprises from 400 to 20000 nucleotides in length. In some embodiments, the circular polyribonucleotide comprises from 450 to 20000 nucleotides in length. In some embodiments, the circular polyribonucleotide comprises from 500 to 20000 nucleotides in length. In some embodiments, the circular polyribonucleotide comprises from 550 to 20000 nucleotides in length. In some embodiments, the circular polyribonucleotide comprises from 600 to 20000 nucleotides in length. In some embodiments, the circular polyribonucleotide comprises from 650 to 20000 nucleotides in length. In some embodiments, the circular polyribonucleotide comprises from 700 to 20000 nucleotides in length. In some embodiments, the circular polyribonucleotide comprises from 750 to 20000 nucleotides in length. In some embodiments, the circular polyribonucleotide comprises from 800 to 20000 nucleotides in length. In some embodiments, the circular polyribonucleotide comprises from 850 to 20000 nucleotides in length. In some embodiments, the circular polyribonucleotide comprises from 900 to 20000 nucleotides in length. In some embodiments, the circular polyribonucleotide comprises from 950 to 20000 nucleotides in length. In some embodiments, the circular polyribonucleotide comprises from 1000 to 20000 nucleotides in length. In some embodiments, the circular polyribonucleotide comprises from 1500 to 20000 nucleotides in length. In some embodiments, the circular polyribonucleotide comprises from 2000 to 20000 nucleotides in length. In some embodiments, the circular polyribonucleotide comprises from 2500 to 20000 nucleotides in length. In some embodiments, the circular polyribonucleotide comprises from 3000 to 20000 nucleotides in length. In some embodiments, the circular polyribonucleotide comprises from 3500 to 20000 nucleotides in length. In some embodiments, the circular polyribonucleotide comprises from 4000 to 20000 nucleotides in length. In some embodiments, the circular polyribonucleotide comprises from 4500 to 20000 nucleotides in length. In some embodiments, the circular polyribonucleotide comprises from 5000 to 20000 nucleotides in length. In some embodiments, the circular polyribonucleotide comprises from 5500 to 20000 nucleotides in length. In some embodiments, the circular polyribonucleotide comprises from 6000 to 20000 nucleotides in length. In some embodiments, the circular polyribonucleotide comprises from 6500 to 20000 nucleotides in length. In some embodiments, the circular polyribonucleotide comprises from 7000 to 20000 nucleotides in length. In some embodiments, the circular polyribonucleotide comprises from 7500 to 20000 nucleotides in length. In some embodiments, the circular polyribonucleotide comprises from 8000 to 20000 nucleotides in length. In some embodiments, the circular polyribonucleotide comprises from 8500 to 20000 nucleotides in length. In some embodiments, the circular polyribonucleotide comprises from 9000 to 20000 nucleotides in length. In some embodiments, the circular polyribonucleotide comprises from 9500 to 20000 nucleotides in length.

In some embodiments, the circular polyribonucleotide comprises from 10000 to 20000 nucleotides in length. In some embodiments, the circular polyribonucleotide comprises from 10050 to 20000 nucleotides in length. In some embodiments, the circular polyribonucleotide comprises from 10100 to 20000 nucleotides in length. In some embodiments, the circular polyribonucleotide comprises from 10150 to 20000 nucleotides in length. In some embodiments, the circular polyribonucleotide comprises from 10200 to 20000 nucleotides in length. In some embodiments, the circular polyribonucleotide comprises from 10250 to 20000 nucleotides in length. In some embodiments, the circular polyribonucleotide comprises from 10300 to 20000 nucleotides in length. In some embodiments, the circular polyribonucleotide comprises from 10350 to 20000 nucleotides in length. In some embodiments, the circular polyribonucleotide comprises from 10400 to 20000 nucleotides in length. In some embodiments, the circular polyribonucleotide comprises from 10450 to 20000 nucleotides in length. In some embodiments, the circular polyribonucleotide comprises from 10500 to 20000 nucleotides in length. In some embodiments, the circular polyribonucleotide comprises from 10550 to 20000 nucleotides in length. In some embodiments, the circular polyribonucleotide comprises from 10600 to 20000 nucleotides in length. In some embodiments, the circular polyribonucleotide comprises from 10650 to 20000 nucleotides in length. In some embodiments, the circular polyribonucleotide comprises from 10700 to 20000 nucleotides in length. In some embodiments, the circular polyribonucleotide comprises from 10750 to 20000 nucleotides in length. In some embodiments, the circular polyribonucleotide comprises from 10800 to 20000 nucleotides in length. In some embodiments, the circular polyribonucleotide comprises from 10850 to 20000 nucleotides in length. In some embodiments, the circular polyribonucleotide comprises from 10900 to 20000 nucleotides in length. In some embodiments, the circular polyribonucleotide comprises from 10950 to 20000 nucleotides in length. In some embodiments, the circular polyribonucleotide comprises from 11000 to 20000 nucleotides in length. In some embodiments, the circular polyribonucleotide comprises from 11500 to 20000 nucleotides in length. In some embodiments, the circular polyribonucleotide comprises from 12000 to 20000 nucleotides in length. In some embodiments, the circular polyribonucleotide comprises from 12500 to 20000 nucleotides in length. In some embodiments, the circular polyribonucleotide comprises from 13000 to 20000 nucleotides in length. In some embodiments, the circular polyribonucleotide comprises from 13500 to 20000 nucleotides in length. In some embodiments, the circular polyribonucleotide comprises from 14000 to 20000 nucleotides in length. In some embodiments, the circular polyribonucleotide comprises from 14500 to 20000 nucleotides in length. In some embodiments, the circular polyribonucleotide comprises from 15000 to 20000 nucleotides in length. In some embodiments, the circular polyribonucleotide comprises from 15500 to 20000 nucleotides in length. In some embodiments, the circular polyribonucleotide comprises from 16000 to 20000 nucleotides in length. In some embodiments, the circular polyribonucleotide comprises from 16500 to 20000 nucleotides in length. In some embodiments, the circular polyribonucleotide comprises from 17000 to 20000 nucleotides in length. In some embodiments, the circular polyribonucleotide comprises from 17500 to 20000 nucleotides in length. In some embodiments, the circular polyribonucleotide comprises from 18000 to 20000 nucleotides in length. In some embodiments, the circular polyribonucleotide comprises from 18500 to 20000 nucleotides in length. In some embodiments, the circular polyribonucleotide comprises from 19000 to 20000 nucleotides in length. In some embodiments, the circular polyribonucleotide comprises from 19500 to 20000 nucleotides in length.

In some embodiments, the circular polyribonucleotide comprises at least 600 nt, 605 nt, 610 nt, 615 nt, 620 nt, 625 nt, 630 nt, 635 nt, 640 nt, 645 not, 650 nt, 6650 nt, 660 nt, 665 nt, 670 nt, 675 nt, 680 nt, 685 nt, 690 nt, 695 nt, or 700 nt. In some embodiments, the circular polyribonucleotide comprises 600 nt, 605 nt, 610 nt, 615 nt, 620 nt, 625 nt, 630 nt, 635 nt, 640 nt, 645 not, 650 nt, 6650 nt, 660 nt, 665 nt, 670 nt, 675 nt, 680 nt, 685 nt, 690 nt, 695 nt, or 700 nt. In some embodiments, the circular polyribonucleotide comprises at least 620 nt, 621 nt, 622 nt, 623 nt, 624 nt, 625 nt, 626 nt, 627 nt, 628 nt, 629 nt, 630 nt, 631 nt, 632 nt, 633 nt, 634 nt, 635 nt, 636 nt, 637 nt, 638 nt, 639 nt, 640 nt, 641 nt, 642 nt, 643 nt, 644 nt, 645 nt, 646 nt, 647 nt, 648 nt, 649 nt, 650 nt, 651 nt, 652 nt, 653 nt, 654 nt, 655 nt, 656 nt, 657 nt, 658 nt, 659 nt, 660 nt, 661 nt, 662 nt, 663 nt, 664 nt, 665 nt, 666 nt, 667 nt, 668 nt, 669 nt, 670 nt, 671 nt, 672 nt, 673 nt, 674 nt, or 675 nt. In some embodiments, the circular polyribonucleotide comprises 620 nt, 621 nt, 622 nt, 623 nt, 624 nt, 625 nt, 626 nt, 627 nt, 628 nt, 629 nt, 630 nt, 631 nt, 632 nt, 633 nt, 634 nt, 635 nt, 636 nt, 637 nt, 638 nt, 639 nt, 640 nt, 641 nt, 642 nt, 643 nt, 644 nt, 645 nt, 646 nt, 647 nt, 648 nt, 649 nt, 650 nt, 651 nt, 652 nt, 653 nt, 654 nt, 655 nt, 656 nt, 657 nt, 658 nt, 659 nt, 660 nt, 661 nt, 662 nt, 663 nt, 664 nt, 665 nt, 666 nt, 667 nt, 668 nt, 669 nt, 670 nt, 671 nt, 672 nt, 673 nt, 674 nt, or 675 nt.

In some embodiments, the circular polyribonucleotide may be of a sufficient size to accommodate a binding site for a ribosome. One of skill in the art can appreciate that the maximum size of a circular polyribonucleotide can be as large as is within the technical constraints of producing a circular polyribonucleotide, and/or using the circular polyribonucleotide. While not being bound by theory, it is possible that multiple segments of RNA may be produced from DNA and their 5′ and 3′ free ends annealed to produce a “string” of RNA, which ultimately may be circularized when only one 5′ and one 3′ free end remains. In some embodiments, the maximum size of a circular polyribonucleotide may be limited by the ability of packaging and delivering the RNA to a target. In some embodiments, the size of a circular polyribonucleotide is a length sufficient to encode useful polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least t 400 nucleotides, at least 300 nucleotides, at least 200 nucleotides, at least 100 nucleotides may be useful.

In some embodiments, the circular polyribonucleotide as described herein is non-immunogenic in a mammal, e.g., a human. In some embodiments, the circular polyribonucleotide is capable of replicating or replicates in a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a livestock animal (horses, cows, pigs, chickens etc.), a human cell, cultured cells, primary cells or cell lines, stem cells, progenitor cells, differentiated cells, germ cells, cancer cells (e.g., tumorigenic, metastic), non-tumorigenic cells (normal cells), fetal cells, embryonic cells, adult cells, mitotic cells, non-mitotic cells, or any combination thereof. In some embodiments, the invention includes a cell comprising the circular polyribonucleotide described herein, wherein the cell is a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a farm or working animal (horses, cows, pigs, chickens etc.), a human cell, a cultured cell, a primary cell or a cell line, a stem cell, a progenitor cell, a differentiated cell, a germ cell, a cancer cell (e.g., tumorigenic, metastic), a non-tumorigenic cell (normal cells), a fetal cell, an embryonic cell, an adult cell, a mitotic cell, a non-mitotic cell, or any combination thereof. In some embodiments, the cell is modified to comprise the circular polyribonucleotide.

In some embodiments, the circular polyribonucleotide has a half-life of at least that of a linear counterpart, e.g., linear expression sequence, or linear circular polyribonucleotide. In some embodiments, the circular polyribonucleotide has a half-life that is increased over that of a linear counterpart. In some embodiments, the half-life is increased by about 5%, 10%, 15%, 20%, 25%, 30%, 35%, 40%, 45%, 50%, or greater. In some embodiments, the circular polyribonucleotide has a half-life or persistence in a cell for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the circular polyribonucleotide has a half-life or persistence in a cell for no more than about 10 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween. In some embodiments, the circular polyribonucleotide has a half-life or persistence in a cell while the cell is dividing. In some embodiments, the circular polyribonucleotide has a half-life or persistence in a cell post division. In certain embodiments, the circular polyribonucleotide has a half-life or persistence in a dividing cell for greater than about 10 minutes to about 30 days, or at least about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In some embodiments, the circular polyribonucleotide modulates a cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the circular polyribonucleotide comprises one or more elements described elsewhere herein. In some embodiments, the elements may be separated from one another by a spacer sequence or linker. In some embodiments, the elements may be separated from one another by 1 ribonucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1kb, at least about 1000 nucleotides, any amount of nucleotides therebetween. In some embodiments, one or more elements are contiguous with one another, e.g., lacking a spacer element. In some embodiments, one or more elements in the circular polyribonucleotide is conformationally flexible. In some embodiments, the conformational flexibility is due to the sequence being substantially free of a secondary structure. In some embodiments, the circular polyribonucleotide comprises a secondary or tertiary structure that accommodates one or more desired functions or characteristics described herein, e.g., accommodate a binding site for a ribosome, e.g., translation, e.g., rolling circle translation.

In some embodiments, the circular polyribonucleotide comprises particular sequence characteristics. For example, the circular polyribonucleotide may comprise a particular nucleotide composition. In some such embodiments, the circular polyribonucleotide may include one or more purine rich regions (adenine or guanosine). In some such embodiments, the circular polyribonucleotide may include one or more purine rich regions (adenine or guanosine). In some embodiments, the circular polyribonucleotide may include one or more AU rich regions or elements (AREs). In some embodiments, the circular polyribonucleotide may include one or more adenine rich regions.

In some embodiments, the circular polyribonucleotide may include one or more repetitive elements described elsewhere herein.

In some embodiments, the circular polyribonucleotide comprises one or more modifications described elsewhere herein.

In some embodiments, the circular polyribonucleotides are those known in the art (e.g., U.S. Pat. Publication 20150079630, and CN Patent publication 106222174, the contents of which are incorporated herein by reference in its entirety). For example, in some embodiments, the cyclic RNA encodes a protein, has a full-length number of bases that is equal to or greater than 102 and is a multiple of 3, has at least one start codon, does not have a stop codon in the same reading frame as the start codon, and does not contain an internal ribosome entry site (IRES). In some embodiments, the full-length number of bases of the cyclic RNA is 561 or less. In some embodiments, the cyclic RNA has a Kozak sequence upstream from the start codon. In some embodiments, the cyclic RNA is used for a method for producing protein in a eukaryotic cell expression system as a template. In some embodiments, the cyclic RNA is introduced into eukaryotic cells to express the protein encoded by the cyclic RNA. In some embodiments, the cyclic RNA is added to a cell-free expression system derived from eukaryotic cells to express the protein encoded by the cyclic RNA. In some embodiments, the cyclic RNA encodes a protein, has a full-length number of bases that is from 102 to 360 and is a multiple of 3, has at least one IRES and one start codon within 1 to 20 bases downstream from the IRES, and does not have a stop codon in the same reading frame as the start codon. In some embodiments, the cyclic RNA is used for a method for producing protein in a prokaryotic cell expression system.

### Binding Region

A circular polyribonucleotide as described herein can comprise a binding region that binds to a capped polyribonucleotide as described herein. The binding region can be in a UTR between a stop and start codon in the circular polyribonucleotide. In some embodiments, a stop codon is between the binding region and a stagger element. Often, the binding region of the circular polyribonucleotide is a second binding region that comprises a sequence that is sense to the sequence of a first binding region, wherein a capped polyribonucleotide comprises the first binding region. A circular polyribonucleotide can comprise a plurality of binding regions. For example, a circular polyribonucleotide comprises 2 binding regions. In some embodiments, a circular polyribonucleotide comprises 3 binding regions. In some embodiments, a circular polyribonucleotide comprises 4 binding regions. In some embodiments, a circular polyribonucleotide comprises 5 binding regions. In some embodiments, a circular polyribonucleotide comprises 6 binding regions. In some embodiments, a circular polyribonucleotide comprises 7 binding regions. In some embodiments, a circular polyribonucleotide comprises 8 binding regions. In some embodiments, a circular polyribonucleotide comprises 9 binding regions. In some embodiments, a circular polyribonucleotide comprises 10 binding regions. In some embodiments, a circular polyribonucleotide comprises 15 binding regions. In some embodiments, a circular polyribonucleotide comprises 20 binding regions. In some embodiments, a circular polyribonucleotide comprises 30 binding regions. In some embodiments, a circular polyribonucleotide comprises 40 binding regions. In some embodiments, a circular polyribonucleotide comprises 50 binding regions. In some embodiments, a circular polyribonucleotide comprises 60 binding regions. In some embodiments, a circular polyribonucleotide comprises 70 binding regions. In some embodiments, a circular polyribonucleotide comprises 80 binding regions. In some embodiments, a circular polyribonucleotide comprises 90 binding regions. In some embodiments, a circular polyribonucleotide comprises 100 binding regions. In some embodiments, a circular polyribonucleotide comprises 200.

In some embodiments, the second binding region comprises from 5 to 100 nucleotides in length. In some embodiments, the second binding region comprises from 5 to 95 nucleotides in length. In some embodiments, the second binding region comprises from 5 to 90 nucleotides in length. In some embodiments, the second binding region comprises from 5 to 85 nucleotides in length. In some embodiments, the second binding region comprises from 5 to 80 nucleotides in length. In some embodiments, the second binding region comprises from 5 to 75 nucleotides in length. In some embodiments, the second binding region comprises from 5 to 70 nucleotides in length. In some embodiments, the second binding region comprises from 5 to 65 nucleotides in length. In some embodiments, the second binding region comprises from 5 to 60 nucleotides in length. In some embodiments, the second binding region comprises from 5 to 55 nucleotides in length. In some embodiments, the second binding region comprises from 5 to 50 nucleotides in length. In some embodiments, the second binding region comprises from 5 to 45 nucleotides in length. In some embodiments, the second binding region comprises from 5 to 40 nucleotides in length. In some embodiments, the second binding region comprises from 5 to 35 nucleotides in length. In some embodiments, the second binding region comprises from 5 to 30 nucleotides in length. In some embodiments, the second binding region comprises from 5 to 25 nucleotides in length. In some embodiments, the second binding region comprises from 5 to 20 nucleotides in length. In some embodiments, the second binding region comprises from 5 to 15 nucleotides in length. In some embodiments, the second binding region comprises from 5 to 10 nucleotides in length.

In some embodiments, the second binding region comprises from 5 to 95 nucleotides in length. In some embodiments, the second binding region comprises from 10 to 95 nucleotides in length. In some embodiments, the second binding region comprises from 15 to 95 nucleotides in length. In some embodiments, the second binding region comprises from 20 to 95 nucleotides in length. In some embodiments, the second binding region comprises from 25 to 95 nucleotides in length. In some embodiments, the second binding region comprises from 30 to 95 nucleotides in length. In some embodiments, the second binding region comprises from 35 to 95 nucleotides in length. In some embodiments, the second binding region comprises from 40 to 95 nucleotides in length. In some embodiments, the second binding region comprises from 45 to 95 nucleotides in length. In some embodiments, the second binding region comprises from 50 to 95 nucleotides in length. In some embodiments, the second binding region comprises from 55 to 95 nucleotides in length. In some embodiments, the second binding region comprises from 60 to 95 nucleotides in length. In some embodiments, the second binding region comprises from 65 to 95 nucleotides in length. In some embodiments, the second binding region comprises from 70 to 95 nucleotides in length. In some embodiments, the second binding region comprises from 75 to 95 nucleotides in length. In some embodiments, the second binding region comprises from 80 to 95 nucleotides in length. In some embodiments, the second binding region comprises from 85 to 95 nucleotides in length. In some embodiments, the second binding region comprises from 90 to 95 nucleotides in length.

In some embodiments, the second binding region comprises from 10 to 95 nucleotides in length. In some embodiments, the second binding region comprises from 15 to 90 nucleotides in length. In some embodiments, the second binding region comprises from 20 to 85 nucleotides in length. In some embodiments, the second binding region comprises from 25 to 80 nucleotides in length. In some embodiments, the second binding region comprises from 30 to 75 nucleotides in length. In some embodiments, the second binding region comprises from 35 to 70 nucleotides in length. In some embodiments, the second binding region comprises from 40 to 65 nucleotides in length. In some embodiments, the second binding region comprises from 45 to 60 nucleotides in length. In some embodiments, the second binding region comprises from 50 to 55 nucleotides in length.

In some embodiments, the second binding region comprises at least 5 nt, 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt. In some embodiments, the second binding region comprises 5 nt, 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt. In some embodiments, the second binding region comprises at least 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or 35 nt. In some embodiments, the second binding region comprises 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or 35 nt. In some embodiments, the second binding region comprises at least 5 nt. In some embodiments, the second binding region comprises at least 10 nt. In some embodiments, the second binding region comprises at least 15 nt. In some embodiments, the second binding region comprises at least 20 nt. In some embodiments, the second binding region comprises at least 25 nt. In some embodiments, the second binding region comprises at least 30 nt. In some embodiments, the second binding region comprises at least 35 nt. In some embodiments, the second binding region comprises at least 40 nt. In some embodiments, the second binding region comprises at least 45 nt. In some embodiments, the second binding region comprises at least 50 nt. In some embodiments, the second binding region comprises at least 55 nt. In some embodiments, the second binding region comprises at least 60 nt. In some embodiments, the second binding region comprises at least 65 nt. In some embodiments, the second binding region comprises at least 70 nt. In some embodiments, the second binding region comprises at least 75 nt. In some embodiments, the second binding region comprises at least 80 nt. In some embodiments, the second binding region comprises at least 85 nt. In some embodiments, the second binding region comprises at least 90 nt. In some embodiments, the second binding region comprises at least 95 nt. In some embodiments, the second binding region comprises at least 100 nt. In some embodiments, the second binding region comprises 5 nt. In some embodiments, the second binding region comprises 10 nt. In some embodiments, the second binding region comprises 15 nt. In some embodiments, the second binding region comprises 20 nt. In some embodiments, the second binding region comprises 25 nt. In some embodiments, the second binding region comprises 30 nt. In some embodiments, the second binding region comprises 35 nt. In some embodiments, the second binding region comprises 40 nt. In some embodiments, the second binding region comprises 45 nt. In some embodiments, the second binding region comprises 50 nt. In some embodiments, the second binding region comprises 55 nt. In some embodiments, the second binding region comprises 60 nt. In some embodiments, the second binding region comprises 65 nt. In some embodiments, the second binding region comprises 70 nt. In some embodiments, the second binding region comprises 75 nt. In some embodiments, the second binding region comprises 80 nt. In some embodiments, the second binding region comprises 85 nt. In some embodiments, the second binding region comprises 90 nt. In some embodiments, the second binding region comprises 95 nt. In some embodiments, the second binding region comprises 100 nt.

In some embodiments, the fourth binding region comprises from 5 to 100 nucleotides in length. In some embodiments, the fourth binding region comprises from 5 to 95 nucleotides in length. In some embodiments, the fourth binding region comprises from 5 to 90 nucleotides in length. In some embodiments, the fourth binding region comprises from 5 to 85 nucleotides in length. In some embodiments, the fourth binding region comprises from 5 to 80 nucleotides in length. In some embodiments, the fourth binding region comprises from 5 to 75 nucleotides in length. In some embodiments, the fourth binding region comprises from 5 to 70 nucleotides in length. In some embodiments, the fourth binding region comprises from 5 to 65 nucleotides in length. In some embodiments, the fourth binding region comprises from 5 to 60 nucleotides in length. In some embodiments, the fourth binding region comprises from 5 to 55 nucleotides in length. In some embodiments, the fourth binding region comprises from 5 to 50 nucleotides in length. In some embodiments, the fourth binding region comprises from 5 to 45 nucleotides in length. In some embodiments, the fourth binding region comprises from 5 to 40 nucleotides in length. In some embodiments, the fourth binding region comprises from 5 to 35 nucleotides in length. In some embodiments, the fourth binding region comprises from 5 to 30 nucleotides in length. In some embodiments, the fourth binding region comprises from 5 to 25 nucleotides in length. In some embodiments, the fourth binding region comprises from 5 to 20 nucleotides in length. In some embodiments, the fourth binding region comprises from 5 to 15 nucleotides in length. In some embodiments, the fourth binding region comprises from 5 to 10 nucleotides in length.

In some embodiments, the fourth binding region comprises from 5 to 95 nucleotides in length. In some embodiments, the fourth binding region comprises from 10 to 95 nucleotides in length. In some embodiments, the fourth binding region comprises from 15 to 95 nucleotides in length. In some embodiments, the fourth binding region comprises from 20 to 95 nucleotides in length. In some embodiments, the fourth binding region comprises from 25 to 95 nucleotides in length. In some embodiments, the fourth binding region comprises from 30 to 95 nucleotides in length. In some embodiments, the fourth binding region comprises from 35 to 95 nucleotides in length. In some embodiments, the fourth binding region comprises from 40 to 95 nucleotides in length. In some embodiments, the fourth binding region comprises from 45 to 95 nucleotides in length. In some embodiments, the fourth binding region comprises from 50 to 95 nucleotides in length. In some embodiments, the fourth binding region comprises from 55 to 95 nucleotides in length. In some embodiments, the fourth binding region comprises from 60 to 95 nucleotides in length. In some embodiments, the fourth binding region comprises from 65 to 95 nucleotides in length. In some embodiments, the fourth binding region comprises from 70 to 95 nucleotides in length. In some embodiments, the fourth binding region comprises from 75 to 95 nucleotides in length. In some embodiments, the fourth binding region comprises from 80 to 95 nucleotides in length. In some embodiments, the fourth binding region comprises from 85 to 95 nucleotides in length. In some embodiments, the fourth binding region comprises from 90 to 95 nucleotides in length.

In some embodiments, the fourth binding region comprises from 10 to 95 nucleotides in length. In some embodiments, the fourth binding region comprises from 15 to 90 nucleotides in length. In some embodiments, the fourth binding region comprises from 20 to 85 nucleotides in length. In some embodiments, the fourth binding region comprises from 25 to 80 nucleotides in length. In some embodiments, the fourth binding region comprises from 30 to 75 nucleotides in length. In some embodiments, the fourth binding region comprises from 35 to 70 nucleotides in length. In some embodiments, the fourth binding region comprises from 40 to 65 nucleotides in length. In some embodiments, the fourth binding region comprises from 45 to 60 nucleotides in length. In some embodiments, the fourth binding region comprises from 50 to 55 nucleotides in length.

In some embodiments, the fourth binding region comprises at least 5 nt, 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt. In some embodiments, the fourth binding region comprises 5 nt, 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt. In some embodiments, the fourth binding region comprises at least 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or 35 nt. In some embodiments, the fourth binding region comprises 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or 35 nt. In some embodiments, the fourth binding region comprises at least 5 nt. In some embodiments, the fourth binding region comprises at least 10 nt. In some embodiments, the fourth binding region comprises at least 15 nt. In some embodiments, the fourth binding region comprises at least 20 nt. In some embodiments, the fourth binding region comprises at least 25 nt. In some embodiments, the fourth binding region comprises at least 30 nt. In some embodiments, the fourth binding region comprises at least 35 nt. In some embodiments, the fourth binding region comprises at least 40 nt. In some embodiments, the fourth binding region comprises at least 45 nt. In some embodiments, the fourth binding region comprises at least 50 nt. In some embodiments, the fourth binding region comprises at least 55 nt. In some embodiments, the fourth binding region comprises at least 60 nt. In some embodiments, the fourth binding region comprises at least 65 nt. In some embodiments, the fourth binding region comprises at least 70 nt. In some embodiments, the fourth binding region comprises at least 75 nt. In some embodiments, the fourth binding region comprises at least 80 nt. In some embodiments, the fourth binding region comprises at least 85 nt. In some embodiments, the fourth binding region comprises at least 90 nt. In some embodiments, the fourth binding region comprises at least 95 nt. In some embodiments, the fourth binding region comprises at least 100 nt. In some embodiments, the fourth binding region comprises 5 nt. In some embodiments, the fourth binding region comprises 10 nt. In some embodiments, the fourth binding region comprises 15 nt. In some embodiments, the fourth binding region comprises 20 nt. In some embodiments, the fourth binding region comprises 25 nt. In some embodiments, the fourth binding region comprises 30 nt. In some embodiments, the fourth binding region comprises 35 nt. In some embodiments, the fourth binding region comprises 40 nt. In some embodiments, the fourth binding region comprises 45 nt. In some embodiments, the fourth binding region comprises 50 nt. In some embodiments, the fourth binding region comprises 55 nt. In some embodiments, the fourth binding region comprises 60 nt. In some embodiments, the fourth binding region comprises 65 nt. In some embodiments, the fourth binding region comprises 70 nt. In some embodiments, the fourth binding region comprises 75 nt. In some embodiments, the fourth binding region comprises 80 nt. In some embodiments, the fourth binding region comprises 85 nt. In some embodiments, the fourth binding region comprises 90 nt. In some embodiments, the fourth binding region comprises 95 nt. In some embodiments, the fourth binding region comprises 100 nt.

### Untranslated Regions

The circular polyribonucleotide as described herein can comprises a UTR (untranslated region). UTRs of a genomic region comprising a gene may be transcribed but not translated. A UTR can be involved in translation regulation, influence localization and stability of the polyribonucleotide, and can comprise binding sites for regulatory proteins and microRNAs. In some embodiments, the UTR comprises a ribosome binding site.

In some embodiments, the UTR comprises secondary structures, such as a hairpin loop, that regulates translation. In some embodiments, the circular polyribonucleotide comprises a UTR with one or more stretches of Adenosines and Uridines embedded within. These AU rich signatures are may increase turnover rates of the expression product.

Introduction, removal or modification of UTR AU rich elements (AREs) may be useful to modulate the stability or immunogenicity of the circular polyribonucleotide. When engineering specific polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any UTR from any gene may be incorporated into the respective flanking regions of the circular polyribonucleotide. As a non-limiting example, the UTR or a fragment thereof which may be incorporated is a UTR listed in U.S. Provisional Application Nos. US 61/775,509 and US 61/829,372, or in International Patent Application No. PCT/US2014/021522; the contents of each of which are herein incorporated by reference in its entirety. Furthermore, multiple wild-type UTRs of any known gene may be utilized. It is also within the scope of the present invention to provide artificial UTRs which are not variants of wild type genes. These UTRs or portions thereof may be placed in the same orientation as in the transcript from which they were selected or may be altered in orientation or location. Hence a 5′ or 3′ UTR may be inverted, shortened, lengthened, made chimeric with one or more other 5′ UTRs or 3′ UTRs. As used herein, the term “altered” as it relates to a UTR sequence, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In one embodiment, a double, triple or quadruple UTR, such as a 5′ or 3′ UTR, may be used. As used herein, a “double” UTR is one in which two copies of the same UTR are encoded either in series or substantially in series. For example, a double beta- globin 3′ UTR may be used as described in U.S. Pat. Publication 20100129877, the contents of which are incorporated herein by reference in its entirety.

In some embodiments, the circular polyribonucleotide comprises a 5′ UTR. The 5′ UTR can be 5′ to a binding region of the circular polyribonucletide, wherein the binding region binds to a capped polyribonucleotide. In some embodiments, a circular polyribonucleotide can comprise a poly-A region. The 5′ UTR can be 5′ of the poly-A region of the circular polyribonucleotide. In some embodiments, the circular polyribonucleotide comprises a 3′ UTR. The 3′ UTR can be 3′ to a binding region of the circular polyribonucleotide, wherein the binding region binds to a capped polyribonucleotide. In some embodiments, the circular polyribonucleotide lacks a UTR.

### PolyA Region

The circular polyribonucleotide as described herein can comprise a poly-A region. In some embodiments, a circular polyribonucleotide comprises a single poly-A region. In some embodiments, a circular polyribonucleotide comprises at least 2, 3, 4, 5, 6 or more poly-A regions. In some embodiments, a circular polyribonucleotide comprises at least 2 poly-A regions. In some embodiments, a circular polyribonucleotide comprises at least 3 poly-A regions. In some embodiments, a circular polyribonucleotide comprises at least 4 poly-A regions. In some embodiments, a circular polyribonucleotide comprises at least 5 poly-A regions. In some embodiments, a circular polyribonucleotide comprises at least 6 poly-A regions. In some embodiments, a circular polyribonucleotide comprises 2 poly-A regions. In some embodiments, a circular polyribonucleotide comprises 3 poly-A regions. In some embodiments, a circular polyribonucleotide comprises 4 poly-A regions. In some embodiments, a circular polyribonucleotide comprises 5 poly-A regions. In some embodiments, a circular polyribonucleotide comprises 6 poly-A regions.

In some embodiments, the length of a poly-A region is greater than 10 nucleotides in length. In one embodiment, the poly-A region is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A region is greater than about 10 nucleotides. In some embodiments, the poly-A region is greater than about 15 nucleotides. In some embodiments, the poly-A region is greater than about 20 nucleotides. In some embodiments, the poly-A region is greater than about 25 nucleotides. In some embodiments, the poly-A region is greater than about 30 nucleotides. In some embodiments, the poly-A region is greater than about 35 nucleotides. In some embodiments, the poly-A region is greater than about 40 nucleotides. In some embodiments, the poly-A region is greater than about 45 nucleotides. In some embodiments, the poly-A region is greater than about 50 nucleotides. In some embodiments, the poly-A region is greater than about 55 nucleotides. In some embodiments, the poly-A region is greater than about 60 nucleotides. In some embodiments, the poly-A region is greater than about 70 nucleotides. In some embodiments, the poly-A region is greater than about 80 nucleotides. In some embodiments, the poly-A region is greater than about 90 nucleotides. In some embodiments, the poly-A region is greater than about 100 nucleotides. In some embodiments, the poly-A region is greater than about 120 nucleotides. In some embodiments, the poly-A region is greater than about 140 nucleotides. In some embodiments, the poly-A region is greater than about 160 nucleotides. In some embodiments, the poly-A region is greater than about 180 nucleotides. In some embodiments, the poly-A region is greater than about 200 nucleotides. In some embodiments, the poly-A region is greater than about 250 nucleotides. In some embodiments, the poly-A region is greater than about 300 nucleotides. In some embodiments, the poly-A region is greater than about 350 nucleotides. In some embodiments, the poly-A region is greater than about 400 nucleotides. In some embodiments, the poly-A region is greater than about 450 nucleotides. In some embodiments, the poly-A region is greater than about 500 nucleotides. In some embodiments, the poly-A region is greater than about 600 nucleotides. In some embodiments, the poly-A region is greater than about 700 nucleotides. In some embodiments, the poly-A region is greater than about 800 nucleotides. In some embodiments, the poly-A region is greater than about 900 nucleotides. In some embodiments, the poly-A region is greater than about 1,000 nucleotides. In some embodiments, the poly-A region is greater than about 1,100 nucleotides. In some embodiments, the poly-A region is greater than about 1,200 nucleotides. In some embodiments, the poly-A region is greater than about 1,300 nucleotides. In some embodiments, the poly-A region is greater than about 1,400 nucleotides. In some embodiments, the poly-A region is greater than about 1,500 nucleotides. In some embodiments, the poly-A region is greater than about 1,600 nucleotides. In some embodiments, the poly-A region is greater than about 1,700 nucleotides. In some embodiments, the poly-A region is greater than about 1,800 nucleotides. In some embodiments, the poly-A region is greater than about 1,900 nucleotides. In some embodiments, the poly-A region is greater than about 2,000 nucleotides. In some embodiments, the poly-A region is greater than about 2,500 nucleotides. In some embodiments, the poly-A region is greater than about 3,000 nucleotides. In some embodiments, the poly-A region is at least about 10 nucleotides. In some embodiments, the poly-A region is at least about 15 nucleotides. In some embodiments, the poly-A region is at least about 20 nucleotides. In some embodiments, the poly-A region is at least about 25 nucleotides. In some embodiments, the poly-A region is at least about 30 nucleotides. In some embodiments, the poly-A region is at least about 35 nucleotides. In some embodiments, the poly-A region is at least about 40 nucleotides. In some embodiments, the poly-A region is at least about 45 nucleotides. In some embodiments, the poly-A region is at least about 50 nucleotides. In some embodiments, the poly-A region is at least about 55 nucleotides. In some embodiments, the poly-A region is at least about 60 nucleotides. In some embodiments, the poly-A region is at least about 70 nucleotides. In some embodiments, the poly-A region is at least about 80 nucleotides. In some embodiments, the poly-A region is at least about 90 nucleotides. In some embodiments, the poly-A region is at least about 100 nucleotides. In some embodiments, the poly-A region is at least about 120 nucleotides. In some embodiments, the poly-A region is at least about 140 nucleotides. In some embodiments, the poly-A region is at least about 160 nucleotides. In some embodiments, the poly-A region is at least about 180 nucleotides. In some embodiments, the poly-A region is at least about 200 nucleotides. In some embodiments, the poly-A region is at least about 250 nucleotides. In some embodiments, the poly-A region is at least about 300 nucleotides. In some embodiments, the poly-A region is at least about 350 nucleotides. In some embodiments, the poly-A region is at least about 400 nucleotides. In some embodiments, the poly-A region is at least about 450 nucleotides. In some embodiments, the poly-A region is at least about 500 nucleotides. In some embodiments, the poly-A region is at least about 600 nucleotides. In some embodiments, the poly-A region is at least about 700 nucleotides. In some embodiments, the poly-A region is at least about 800 nucleotides. In some embodiments, the poly-A region is at least about 900 nucleotides. In some embodiments, the poly-A region is at least about 1,000 nucleotides. In some embodiments, the poly-A region is at least about 1,100 nucleotides. In some embodiments, the poly-A region is at least about 1,200 nucleotides. In some embodiments, the poly-A region is at least about 1,300 nucleotides. In some embodiments, the poly-A region is at least about 1,400 nucleotides. In some embodiments, the poly-A region is at least about 1,500 nucleotides. In some embodiments, the poly-A region is at least about 1,600 nucleotides. In some embodiments, the poly-A region is at least about 1,700 nucleotides. In some embodiments, the poly-A region is at least about 1,800 nucleotides. In some embodiments, the poly-A region is at least about 1,900 nucleotides. In some embodiments, the poly-A region is at least about 2,000 nucleotides. In some embodiments, the poly-A region is at least about 2,500 nucleotides. In some embodiments, the poly-A region is at least about 3,000 nucleotides. In some embodiments, the poly-A region is from about 10 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000). In some embodiments, the poly-A region is from 10 to 3,000 nucleotides. In some embodiments, the poly-A region is from 30 to 50 nucleotides. In some embodiments, the poly-A region is from 30 to 100 nucleotides. In some embodiments, the poly-A region is from 30 to 250 nucleotides. In some embodiments, the poly-A region is from 30 to 500 nucleotides. In some embodiments, the poly-A region is from 30 to 750 nucleotides. In some embodiments, the poly-A region is from 30 to 1,000 nucleotides. In some embodiments, the poly-A region is from 30 to 1,500 nucleotides. In some embodiments, the poly-A region is from 30 to 2,000 nucleotides. In some embodiments, the poly-A region is from 30 to 2,500, from 50 to 100, from 50 to 250, from 50 to 500, from 50 to 750, from 50 to 1,000, from 50 to 1,500 nucleotides. In some embodiments, the poly-A region is from 50 to 2,000 nucleotides. In some embodiments, the poly-A region is from 50 to 2,500 nucleotides. In some embodiments, the poly-A region is from 50 to 3,000 nucleotides. In some embodiments, the poly-A region is from 100 to 500 nucleotides. In some embodiments, the poly-A region is from 100 to 750 nucleotides. In some embodiments, the poly-A region is from 100 to 1,000 nucleotides. In some embodiments, the poly-A region is from 100 to 1,500 nucleotides. In some embodiments, the poly-A region is from 100 to 2,000 nucleotides. In some embodiments, the poly-A region is from 100 to 2,500 nucleotides. In some embodiments, the poly-A region is from 100 to 3,000 nucleotides. In some embodiments, the poly-A region is from 500 to 750 nucleotides. In some embodiments, the poly-A region is from 500 to 1,000 nucleotides. In some embodiments, the poly-A region is from 500 to 1,500 nucleotides. In some embodiments, the poly-A region is from 500 to 2,000 nucleotides. In some embodiments, the poly-A region is from 500 to 2,500 nucleotides. In some embodiments, the poly-A region is from 500 to 3,000 nucleotides. In some embodiments, the poly-A region is from 1,000 to 1,500 nucleotides. In some embodiments, the poly-A region is from 1,000 to 2,000 nucleotides. In some embodiments, the poly-A region is from 1,000 to 2,500 nucleotides. In some embodiments, the poly-A region is from 1,000 to 3,000 nucleotides. In some embodiments, the poly-A region is from 1,500 to 2,000 nucleotides. In some embodiments, the poly-A region is from 1,500 to 2,500 nucleotides. In some embodiments, the poly-A region is from 1,500 to 3,000 nucleotides. In some embodiments, the poly-A region is from 2,000 to 3,000 nucleotides. In some embodiments, the poly-A region is from 2,000 to 2,500 nucleotides. In some embodiments, the poly-A region is from 2,500 to 3,000 nucleotides.

In one embodiment, the poly-A region is designed relative to the length of the overall circular polyribonucleotide. This design may be based on the length of the coding region, the length of a particular feature or region (such as the first or flanking regions), or based on the length of the ultimate product expressed from the circular polyribonucleotide. In this context, the poly-A sequence may be 10, 20, 30, 40, 50, 60, 70, 80, 90, or 100% greater in length than the circular polyribonucleotide or a feature thereof. The poly-A region may also be designed as a fraction of circular polyribonucleotide to which it belongs. In this context, the poly-A region may be 10, 20, 30, 40, 50, 60, 70, 80, or 90% or more of the total length of the construct or the total length of the construct minus the poly-A region. Further, engineered binding sites and conjugation of circular polyribonucleotide for Poly-A binding protein may enhance expression.

In one embodiment, the circular polyribonucleotide is designed to include a polyA-G quartet. The G-quartet is a cyclic hydrogen bonded array of four guanine nucleotides that can be formed by G-rich sequences in both DNA and RNA. In one embodiment, the G-quartet is incorporated at the end of the poly-A sequence. The resultant circular polyribonucleotide construct is assayed for stability, protein production and/or other parameters including half-life at various time points. In some embodiments, the polyA-G quartet results in protein production equivalent to at least 75% of that seen using a poly-A sequence of 120 nucleotides alone.

In some embodiments, a poly-A region is at the 3′ terminus of an expression sequence of a circular polyribonucleotide as disclosed herein. In some embodiments, a poly-A region is at a 5′ terminus of an expression sequence of a circular polyribonucleotide as disclosed herein. In some embodiments, a poly-A region is not at the 3′ terminus of an expression sequence of a circular polyribonucleotide as disclosed herein.

In some embodiments, the poly-A region is at the 5′ end of a UTR of a circular polyribonucleotide as disclosed herein. In some embodiments, the poly-A region is at the 3′ end of a UTR of a circular polyribonucleotide as disclosed herein. In some embodiments, the poly-A region is at the 5′ end of a translation initiation sequence of a circular polyribonucleotide as disclosed herein. In some embodiments, the poly-A region is at the 3′ end of an translation initiation sequence of a circular polyribonucleotide as disclosed herein. In some embodiments, the poly-A region is at the 5′ end of a IRES sequence of a circular polyribonucleotide as disclosed herein. In some embodiments, the poly-A region is at the 3′ end of a IRES sequence of a circular polyribonucleotide as disclosed herein. In some embodiments, the poly-A region is at the 5′ end of a termination element of a circular polyribonucleotide as disclosed herein. In some embodiments, the poly-A region is at the 3′ end of a termination element of a circular polyribonucleotide as disclosed herein. In some embodiments, the poly-A region is at the 5′ end of a stagger element of a circular polyribonucleotide as disclosed herein. In some embodiments, the poly-A region is at the 3′ end of a stagger element of a circular polyribonucleotide as disclosed herein. In some embodiments, the poly-A region is at the 5′ end of an encryptogen of a circular polyribonucleotide as disclosed herein. In some embodiments, the poly-A region is at the 3′ end of an encryptogen of a circular polyribonucleotide as disclosed herein. In some embodiments, the poly-A region is at the 5′ end of a binding region of a circular polyribonucleotide as disclosed herein. In some embodiments, the poly-A region is at the 3′ end of a binding region of a circular polyribonucleotide as disclosed herein. In some embodiments, the poly-A region is at the 5′ end of a first binding region of a circular polyribonucleotide as disclosed herein. In some embodiments, the poly-A region is at the 3′ end of a first binding region of a circular polyribonucleotide as disclosed herein. In some embodiments, the poly-A region is at the 5′ end of a third binding region of a circular polyribonucleotide as disclosed herein. In some embodiments, the poly-A region is at the 3′ end of a third binding region of a circular polyribonucleotide as disclosed herein. In some embodiments, the poly-A region is at the 5′ end of a spacer sequence of a circular polyribonucleotide as disclosed herein. In some embodiments, the poly-A region is at the 3′ end of a spacer sequence of a circular polyribonucleotide as disclosed herein.

In some embodiments, the circular polyribonucleotide comprises a polyA. In some embodiments, the circular polyribonucleotide lacks a polyA. In some embodiments, the circular polyribonucleotide has a modified polyA to modulate one or more characteristics of the circular polyribonucleotide. In some embodiments, the circular polyribonucleotide lacking a polyA or having modified polyA improves one or more functional characteristics, e.g., immunogenicity, half-life, expression efficiency, etc. In some embodiments, a circular polyribonucleotide comprising a polyA region has increased stability compared to a circular polyribonucleotide lacking a polyA region.

In some embodiments, a circular polyribonucleotide comprises a polyA region that functionally binds to an RNA binding protein. PolyA binding protein monomers bind to stretches of about 38 nucleotides. For example, a functional polyA region comprises a length of nucleotides that binds to at least 4 polyA binding protein, such as about 80 nucleotides or 160 nucleotides. In some embodiments, a circular polyribonucleotide lacks a polyA region that functionally binds to an RNA binding protein (e.g., lacks a functional polyA region). For example, a polyA region that is not long enough to bind to at least 4 monomers of a polyA binding protein.

### Translation Initiation Sequence

The circular polyribonucleotide as described herein can comprise a sequence encoding a polypeptide and a protein translation initiation sequence, e.g., a start codon. In some embodiments, the translation initiation sequence includes a Kozak or Shine-Dalgarno sequence. In some embodiments, the circular polyribonucleotide includes the protein translation initiation sequence, e.g., Kozak sequence, adjacent to an expression sequence. In some embodiments, the protein translation initiation sequence is a non-coding start codon. In some embodiments, the protein translation initiation sequence, e.g., Kozak sequence, is present on one or both sides of each expression sequence, leading to separation of the expression products. In some embodiments, the circular polyribonucleotide includes at least one protein translation initiation sequence adjacent to an expression sequence. In some embodiments, the protein translation initiation sequence provides conformational flexibility to the circular polyribonucleotide. In some embodiments, the protein translation initiation sequence is within a substantially single stranded region of the circular polyribonucleotide.

In some embodiments, a protein translation initiation sequence can function as a regulatory element. In some embodiments, a translation initiation sequence comprises an AUG codon. In some embodiments, a translation initiation sequence comprises any eukaryotic start codon such as AUG, CUG, GUG, UUG, ACG, AUC, AUU, AAG, AUA, or AGG. In some embodiments, a translation initiation sequence comprises a Kozak sequence.

Nucleotides flanking a codon that initiates translation, such as, but not limited to, a start codon or an alternative start codon, are known to affect the translation efficiency, the length and/or the structure of the circular polyribonucleotide. (See e.g., Matsuda and Mauro PLoS ONE, 2010 5: 11; the contents of which are herein incorporated by reference in its entirety). Masking any of the nucleotides flanking a codon that initiates translation may be used to alter the position of translation initiation, translation efficiency, length and/or structure of the circular polyribonucleotide.

The circular polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. The circular polyribonucleotide may include more than 1 start codon. The circular polyribonucleotide may include at least 2 start codons. The circular polyribonucleotide may include at least 3 start codons. The circular polyribonucleotide may include at least 4 start codons. The circular polyribonucleotide may include at least 5 start codons. The circular polyribonucleotide may include at least 6 start codons. The circular polyribonucleotide may include at least 7 start codons. The circular polyribonucleotide may include at least 8 start codons. The circular polyribonucleotide may include at least 9 start codons. The circular polyribonucleotide may include at least 10 start codons. The circular polyribonucleotide may include at least 11 start codons. The circular polyribonucleotide may include at least 12 start codons. The circular polyribonucleotide may include at least 13 start codons. The circular polyribonucleotide may include at least 14 start codons. The circular polyribonucleotide may include at least 15 start codons. The circular polyribonucleotide may include at least 16 start codons. The circular polyribonucleotide may include at least 17 start codons. The circular polyribonucleotide may include at least 18 start codons. The circular polyribonucleotide may include at least 19 start codons. The circular polyribonucleotide may include at least 20 start codons. The circular polyribonucleotide may include at least 25 start codons. The circular polyribonucleotide may include at least 30 start codons. The circular polyribonucleotide may include at least 35 start codons. The circular polyribonucleotide may include at least 40 start codons. The circular polyribonucleotide may include at least 50 start codons. The circular polyribonucleotide may include at least 60 start codons. Translation may initiate on the first start codon or may initiate downstream of the first start codon.

In some embodiments, the circular polyribonucleotide may initiate at a codon which is not the first start codon, e.g., AUG. Translation of the circular polyribonucleotide may initiate at an alternative translation initiation sequence, such as, but not limited to, ACG, AGG, AAG, CTG/CUG, GTG/GUG, ATA/AUA, ATT/AUU, TTG/UUG (see Touriol et al. Biology of the Cell 95 (2003) 169- 178 and Matsuda and Mauro PLoS ONE, 2010 5: 11; the contents of each of which are herein incorporated by reference in their entireties). In some embodiments, translation begins at an alternative protein translation initiation sequence under selective conditions, e.g., stress induced conditions. As a non-limiting example, the translation of the circular polyribonucleotide may begin at alternative protein translation initiation sequence, such as ACG. As another non-limiting example, the circular polyribonucleotide translation may begin at alternative protein translation initiation sequence, CTG/CUG. As yet another non-limiting example, the circular polyribonucleotide translation may begin at alternative protein translation initiation sequence, GTG/GUG. As yet another non-limiting example, the circular polyribonucleotide may begin translation at a repeat-associated non-AUG (RAN) sequence, such as an alternative protein translation initiation sequence that includes short stretches of repetitive RNA, e.g., CGG, GGGGCC, CAG, CTG.

In some embodiments, translation is initiated by eukaryotic initiation factor 4A (eIF4A) treatment with Rocaglates (translation is repressed by blocking 43S scanning, leading to premature, upstream translation initiation and reduced protein expression from transcripts bearing the RocA-eIF4A target sequence, see for example, www.nature.com/articles/nature17978).

### IRES

In some embodiments, the circular polyribonucleotide described herein comprises an internal ribosome entry site (IRES) element. A suitable IRES element to include in a circular polyribonucleotide comprises an RNA sequence capable of engaging an eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt. In one embodiment, the IRES element is derived from the DNA of an organism including, but not limited to, a virus, a mammal, and a Drosophila. Such viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, the IRES element is at least partially derived from a virus, for instance, it can be derived from a viral IRES element, such as ABPV_IGRpred, AEV, ALPV_IGRpred, BQCV_IGRpred, BVDV1_1-385, BVDV1_29-391, CrPV_5NCR, CrPV_IGR, crTMV_IREScp, crTMV_IRESmp75, crTMV_IRESmp228, crTMV_IREScp, crTMV_IREScp, CSFV, CVB3, DCV_IGR, EMCV-R, EoPV_5NTR, ERAV_245-961, ERBV_162-920, EV71_1-748, FeLV-Notch2, FMDV_type_C, GBV-A, GBV-B, GBV-C, gypsy_env, gypsyD5, gypsyD2, HAV_HM175, HCV_type_1a, HiPV_IGRpred, HIV-1, HoCV1_IGRpred, HRV-2, IAPV_IGRpred, idefix, KBV_IGRpred, LINE-1_ORF1_-101_to_-1, LINE-1_ORF1_-302_to_-202, LINE-1_ORF2_-138_to_-86, LINE-1_ORF1_-44_to_-1, PSIV_IGR, PV_type1_Mahoney, PV_type3_Leon, REV-A, RhPV_5NCR, RhPV_IGR, SINV1_IGRpred, SV40_661-830, TMEV, TMV_UI_IRESmp228, TRV_5NTR, TrV_IGR, or TSV_IGR. In some embodiments, the IRES element is at least partially derived from a cellular IRES, such as AML1/RUNX1, Antp-D, Antp-DE, Antp-CDE, Apaf-1, Apaf-1, AQP4, AT1R_var1, AT1R_var2, AT1R_var3, AT1R_var4, BAG1_p36delta236nt, BAG1_p36, BCL2, BiP_-222_-3, c-IAP1_285-1399, c-IAP1_1313-1462, c-jun, c-myc, Cat-1_224, CCND1, DAP5, eIF4G, eIF4GI-ext, eIF4GII, eIF4GII-long, ELG1, ELH, FGF1A, FMR1, Gtx-133-141, Gtx-1-166, Gtx-1-120, Gtx-1-196, hairless, HAP4, HIF1a, hSNM1, Hsp101, hsp70, hsp70, Hsp90, IGF2_leader2, Kv1.4_1.2, L-myc, LamB1_-335_-1, LEF1, MNT_75-267, MNT_36-160, MTG8a, MYB, MYT2_997-1152, n-MYC, NDST1, NDST2, NDST3, NDST4L, NDST4S, NRF_-653_-17, NtHSF1, ODC1, p27kip1, p53_128-269, PDGF2/c-sis, Pim-1, PITSLRE_p58, Rbm3 ,reaper, Scamper, TFIID, TIF4631, Ubx_1-966, Ubx_373-961, UNR, Ure2, UtrA, VEGF-A_-133_-1, XIAP_5-464, XIAP_305-466, or YAP1. In some embodiments, the IRES element comprises a synthetic IRES, for instance, (GAAA) 16, (PPT19)4, KMI1, KMI1, KMI2, KMI2, KMIX, X1, or X2.

In some embodiments, the circular polyribonucleotide includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the circular polyribonucleotide includes one or more IRES sequences on one or both sides of each expression sequence, leading to separation of the resulting peptide(s) and or polypeptide(s).

### Termination Element

The circular polyribonucleotide as described herein can comprise one or more expression sequences and each expression sequence may or may not have a termination element. In some embodiments, the circular polyribonucleotide includes one or more expression sequences and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide comprises a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences comprises two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation, e.g., UAA, UGA, UAG. In some embodiments, one or more termination elements in the circular polyribonucleotide are frame-shifted termination elements, such as but not limited to, off-frame or -1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell.

### Stagger Element

The circular polyribonucleotide as described herein can comprise at least one stagger element adjacent to an expression sequence. In some embodiments, a stop codon is between the binding region of the circular polyribonucleotide (e.g., second binding region) and a stagger element. In some embodiments, the circular polyribonucleotide includes a stagger element adjacent to each expression sequence. In some embodiments, the stagger element is present on one or both sides of each expression sequence, leading to separation of the expression products, e.g., peptide(s) and or polypeptide(s). In some embodiments, the stagger element is a portion of the one or more expression sequences. In some embodiments, the circular polyribonucleotide comprises one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element comprises a portion of an expression sequence of the one or more expression sequences.

In some embodiments, the circular polyribonucleotide includes a stagger element. To avoid production of a continuous expression product, e.g., peptide or polypeptide, while maintaining rolling circle translation, a stagger element may be included to induce ribosomal pausing during translation. In some embodiments, the stagger element is at 3′ end of at least one of the one or more expression sequences. The stagger element can be configured to stall a ribosome during rolling circle translation of the circular polyribonucleotide. The stagger element may include, but is not limited to a 2A-like, or CHYSEL (cis-acting hydrolase element) sequence. In some embodiments, the stagger element encodes a sequence with a C-terminal consensus sequence that is X₁X₂X₃EX₅NPGP, where X₁ is absent or G or H, X₂ is absent or D or G, X₃ is D or V or I or S or M, and X₅ is any amino acid. In some embodiments, this sequence comprises a non-conserved sequence of amino-acids with a strong alpha-helical propensity followed by the consensus sequence -D(V/I)ExNPG P, where x= any amino acid. Some nonlimiting examples of stagger elements includes GDVESNPGP, GDIEENPGP, VEPNPGP, IETNPGP, GDIESNPGP, GDVELNPGP, GDIETNPGP, GDVENPGP, GDVEENPGP, GDVEQNPGP, IESNPGP, GDIELNPGP, HDIETNPGP, HDVETNPGP, HDVEMNPGP, GDMESNPGP, GDVETNPGP, GDIEQNPGP, and DSEFNPGP.

In some embodiments, the stagger element described herein cleaves an expression product, such as between G and P of the consensus sequence described herein. As one non-limiting example, the circular polyribonucleotide includes at least one stagger element to cleave the expression product. In some embodiments, the circular polyribonucleotide includes a stagger element adjacent to at least one expression sequence. In some embodiments, the circular polyribonucleotide includes a stagger element after each expression sequence. In some embodiments, the circular polyribonucleotide includes a stagger element is present on one or both sides of each expression sequence, leading to translation of individual peptide(s) and or polypeptide(s) from each expression sequence.

In some embodiments, a stagger element comprises one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Exemplary modifications can include any modification to the sugar, the nucleobase, the internucleoside linkage (e.g. to a linking phosphate / to a phosphodiester linkage / to the phosphodiester backbone), and any combination thereof that can induce ribosomal pausing during translation. Some of the exemplary modifications provided herein are described elsewhere herein.

In some embodiments, the stagger element is present in the circular polyribonucleotide in other forms. For example, in some exemplary circular polyribonucleotides, a stagger element comprises a termination element of a first expression sequence in the circular polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular polyribonucleotide. In some cases, the first expression sequence and the expression sequence succeeding the first expression sequence are two separate expression sequences in the circular polyribonucleotide. The distance between the first stagger element and the first translation initiation sequence can enable continuous translation of the first expression sequence and its succeeding expression sequence. In some embodiments, the first stagger element comprises a termination element and separates an expression product of the first expression sequence from an expression product of its succeeding expression sequences, thereby creating discrete expression products. In some cases, the circular polyribonucleotide comprising the first stagger element upstream of the first translation initiation sequence of the succeeding sequence in the circular polyribonucleotide is continuously translated, while a corresponding circular polyribonucleotide comprising a stagger element of a second expression sequence that is upstream of a second translation initiation sequence of an expression sequence succeeding the second expression sequence is not continuously translated. In some cases, there is only one expression sequence in the circular polyribonucleotide, and the first expression sequence and its succeeding expression sequence are the same expression sequence. In some exemplary circular polyribonucleotides, a stagger element comprises a first termination element of a first expression sequence in the circular polyribonucleotide, and a nucleotide spacer sequence that separates the termination element from a downstream translation initiation sequence. In some such examples, the first stagger element is upstream of (5′ to) a first translation initiation sequence of the first expression sequence in the circular polyribonucleotide. In some cases, the distance between the first stagger element and the first protein translation initiation sequence enables continuous translation of the first expression sequence and any succeeding expression sequences. In some embodiments, the first stagger element separates one round expression product of the first expression sequence from the next round expression product of the first expression sequences, thereby creating discrete expression products. In some cases, the circular polyribonucleotide comprising the first stagger element upstream of the first protein translation initiation sequence of the first expression sequence in the circular polyribonucleotide is continuously translated, while a corresponding circular polyribonucleotide comprising a stagger element upstream of a second protein translation initiation sequence of a second expression sequence in the corresponding circular polyribonucleotide is not continuously translated. In some cases, the distance between the second stagger element and the second protein translation initiation sequence is at least 2×, 3×, 4×, 5×, 6×, 7×, 8×, 9×, or 10× greater in the corresponding circular polyribonucleotide than a distance between the first stagger element and the first protein translation initiation in the circular polyribonucleotide. In some cases, the distance between the first stagger element and the first protein translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second protein translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater than the distance between the first stagger element and the first protein translation initiation. In some embodiments, the circular polyribonucleotide comprises more than one expression sequence.

### Expression Sequences

The circular polyribonucleotide as described herein comprises at least one expression sequence that encodes a peptide or polypeptide. Such peptide may include, but is not limited to, small peptide, peptidomimetic (e.g., peptoid), amino acids, and amino acid analogs. The peptide may be linear or branched. Such peptide may have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such peptide may include, but is not limited to, a neurotransmitter, a hormone, a drug, a toxin, a viral or microbial particle, a synthetic molecule, and agonists or antagonists thereof.

The polypeptide may be linear or branched. The polypeptide may have a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

Some examples of a peptide or polypeptide include, but are not limited to, fluorescent tag or marker, antigen, peptide therapeutic, synthetic or analog peptide from naturally-bioactive peptide, agonist or antagonist peptide, anti-microbial peptide, pore-forming peptide, a bicyclic peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7): 1076-113). Such antigen binding peptides may bind a cytosolic antigen, a nuclear antigen, an intra-organellar antigen.

In some embodiments, the circular polyribonucleotide comprises one or more RNA expression sequences, each of which may encode a polypeptide. The polypeptide may be produced in substantial amounts. As such, the polypeptide may be any proteinaceous molecule that can be produced. A polypeptide can be a polypeptide that can be secreted from a cell, or localized to the cytoplasm, nucleus or membrane compartment of a cell. Some polypeptides include, but are not limited to, at least a portion of a viral envelope protein, metabolic regulatory enzymes (e.g., that regulate lipid or steroid production), an antigen, a toleragen, a cytokine, a toxin, enzymes whose absence is associated with a disease, and polypeptides that are not active in an animal until cleaved (e.g., in the gut of an animal), and a hormone.

### Therapeutic Peptides or Polypeptides

In some embodiments, the expression sequence encodes a therapeutic effector, e.g., a therapeutic peptide or polypeptide, e.g., an intracellular peptide or intracellular polypeptide, a secreted polypeptide, or a protein replacement therapeutic. In some embodiments, the expression sequence includes a sequence encoding a protein e.g., a therapeutic protein. Some examples of therapeutic proteins may include, but are not limited to, a hormone, a cytokine, an enzyme, an antibody (e.g., one or a plurality of polypeptides encoding at least a heavy chain or a light chain), a transcription factor, a receptor (e.g., a membrane receptor), a ligand, a membrane transporter, a secreted protein, a peptide, a carrier protein, a structural protein, a nuclease, or a component thereof.

The therapeutic expression sequence may be a functional variant or fragment thereof of any of the above, e.g., a protein having at least 80%, 85%, 90%, 95%, 967%, 98%, 99% identity to a protein sequence which disclosed in a table herein by reference to its UniProt ID.

In some embodiments, the circular polyribonucleotide includes an expression sequence encoding a protein e.g., a therapeutic protein. In some embodiments, therapeutic proteins that can be expressed from the circular polyribonucleotide disclosed herein have antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity. Some examples of therapeutic proteins may include, but are not limited to, an enzyme replacement protein, a protein for supplementation, a protein vaccination, antigens (e.g. tumor antigens, viral, bacterial), hormones, cytokines, antibodies, immunotherapy (e.g. cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof.

In some embodiments, exemplary proteins that can be expressed from the circular polyribonucleotide disclosed herein include human proteins, for instance, receptor binding protein, hormone, growth factor, growth factor receptor modulator, and regenerative protein (e.g., proteins implicated in proliferation and differentiation, e.g., therapeutic protein, for wound healing). In some embodiments, exemplary proteins that can be expressed from the circular polyribonucleotide disclosed herein include EGF (epithelial growth factor). In some embodiments, exemplary proteins that can be expressed from the circular polyribonucleotide disclosed herein include enzymes, for instance, oxidoreductase enzymes, metabolic enzymes, mitochondrial enzymes, oxygenases, dehydrogenases, ATP-independent enzyme, and desaturases. In some embodiments, exemplary proteins that can be expressed from the circular polyribonucleotide disclosed herein include an intracellular protein or cytosolic protein. In some embodiments, the circular polyribonucleotide expresses a NanoLuck luciferase (nLuc). In some embodiments, exemplary proteins that can be expressed from the circular polyribonucleotide disclosed herein include a secretary protein, for instance, a secretary enzyme. In some cases, the circular polyribonucleotide expresses a secretary protein that can have a short half-life therapeutic in the blood, or can be a protein with a subcellular localization signal, or protein with secretory signal peptide. In some embodiments, the circular polyribonucleotide expresses a Gaussia Luciferase (gLuc). In some cases, the circular polyribonucleotide expresses a non-human protein, for instance, a fluorescent protein, an energy-transfer acceptor, or a protein-tag like Flag, Myc, or His. In some embodiments, exemplary proteins that can be expressed from the circular polyribonucleotide include a GFP. In some embodiments, the circular polyribonucleotide expresses tagged proteins, e.g., fusion proteins or engineered proteins containing a protein tage, e.g., chitin binding protein (CBP), maltose binding protein (MBP), Fc tag, glutathione-S-transferase (GST), AviTag (GLNDIFEAQKIEWHE), Calmodulin-tag (KRRWKKNFIAVSAANRFKKISSSGAL); polyglutamate tag (EEEEEE); E-tag (GAPVPYPDPLEPR); FLAG-tag (DYKDDDDK), HA-tag (YPYDVPDYA); His-tag (HHHHHH); Myc-tag (EQKLISEEDL); NE-tag (TKENPRSNQEESYDDNES); S-tag (KETAAAKFERQHMDS); SBP-tag (MDEKTTGWRGGHVVEGLAGELEQLRARLEHHPQGQREP); Softag 1 (SLAELLNAGLGGS); Softag 3 (TQDPSRVG); Spot-tag (PDRVRAVSHWSS); Strep-tag (Strep-tag II: WSHPQFEK); TC tag (CCPGCC); Ty tag (EVHTNQDPLD); V5 tag (GKPIPNPLLGLDST); VSV-tag (YTDIEMNRLGK); or Xpress tag (DLYDDDDK).

The therapeutic expression sequence may be an antibody or antibody fragment that binds any of the above, e.g., an antibody against a protein having at least 80%, 85%, 90%, 95%, 967%, 98%, 99% identity to a protein sequence which disclosed in a table herein by reference to its UniProt ID. The term “antibody” herein is used in the broadest sense and encompasses various antibody structures, including but not limited to monoclonal antibodies, polyclonal antibodies, multispecific antibodies (e.g., bispecific antibodies), antibody fragments, and alternative scaffold binding proteins so long as they exhibit the desired antigen-binding activity. An “antibody fragment” refers to a molecule that includes at least one heavy chain or light chain and binds an antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Alternative scaffold proteins may include, e.g. Darpins, FN3 domains, Centyrins, Knottins, anticalins, nanobodies, and other single domain and multi domain proteins selected or engineered to bind a target molecule

In some embodiments, the circular polyribonucleotide expresses an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can comprise more than one expression sequence, each of which expresses a portion of an antibody, and the sum of which can constitute the antibody. In some cases, the circular polyribonucleotide comprises one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

### Exemplary Secreted Polypeptide Effectors

Exemplary secreted proteins that can be expressed are described herein, e.g., in the tables below.

### Cytokines and Cytokine Receptors

In some embodiments, an effector described herein comprises a cytokine of Table 1, or a functional variant or fragment thereof, e.g., a protein having at least 80%, 85%, 90%, 95%, 967%, 98%, 99% identity to a protein sequence disclosed in Table 1 by reference to its UniProt ID. In some embodiments, the functional variant binds to the corresponding cytokine receptor with a Kd of no more than 10%, 20%, 30%, 40%, or 50% higher or lower than the Kd of the corresponding wild-type cytokine for the same receptor under the same conditions. In some embodiments, the effector comprises a fusion protein comprising a first region (e.g., a cytokine polypeptide of Table 1 or a functional variant or fragment thereof) and a second, heterologous region. In some embodiments, the first region is a first cytokine polypeptide of Table 1. In some embodiments, the second region is a second cytokine polypeptide of Table 1, wherein the first and second cytokine polypeptides form a cytokine heterodimer with each other in a wild-type cell. In some embodiments, the polypeptide of Table 1 or functional variant thereof comprises a signal sequence, e.g., a signal sequence that is endogenous to the effector, or a heterologous signal sequence.

In some embodiments, an effector described herein comprises an antibody or fragment thereof that binds a cytokine of Table 1. In some embodiments, the antibody molecule comprises a signal sequence.

TABLE 1

Exemplary cytokines and cytokine receptors

| Cytokine | Cytokine receptor(s) | Entrez Gene ID | UniProt ID |
|---|---|---|---|
| IL-1α, IL-1β, or a heterodimer thereof | IL-1 type 1 receptor, IL-1 type 2 receptor | 3552, 3553 | P01583, P01584 |
| IL-1Ra | IL-1 type 1 receptor, IL-1 type 2 receptor | 3454, 3455 | P17181, P48551 |
| IL-2 | IL-2R | 3558 | P60568 |
| IL-3 | IL-3 receptor α + β c (CD131) | 3562 | P08700 |
| IL-4 | IL-4R type 1, IL-4R type II | 3565 | P05112 |
| IL-5 | IL-5R | 3567 | P05113 |
| IL-6 | IL-6R (sIL-6R) gp130 | 3569 | P05231 |
| IL-7 | IL-7R and sIL-7R | 3574 | P13232 |
| IL-8 | CXCR1 and CXCR2 | 3576 | P10145 |
| IL-9 | IL-9R | 3578 | P15248 |
| IL-10 | IL-10R⅟IL-10R2 complex | 3586 | P22301 |
| IL-11 | IL-11Rα 1 gp130 | 3589 | P20809 |
| IL-12 (e.g., p35, p40, or a heterodimer thereof) | IL-12Rβ1 and IL-12Rβ2 | 3593, 3592 | P29459, P29460 |
| IL-13 | IL-13Rlαl and IL-13Rlα2 | 3596 | P35225 |
| IL-14 | IL-14R | 30685 | P40222 |
| IL-15 | IL-15R | 3600 | P40933 |
| IL-16 | CD4 | 3603 | Q14005 |
| IL-17A | IL-17RA | 3605 | Q16552 |
| IL-17B | IL-17RB | 27190 | Q9UHF5 |
| IL-17C | IL-17RA to IL-17RE | 27189 | Q9P0M4 |
| IL-17D | SEF | 53342 | Q8TAD2 |
| IL-17F | IL-17RA, IL-17RC | 112744 | Q96PD4 |
| IL-18 | IL-18 receptor | 3606 | Q14116 |
| IL-19 | IL-20R⅟IL-20R2 | 29949 | Q9UHD0 |
| IL-20 | L-20R⅟IL-20R2 and IL-22R⅟ IL-20R2 | 50604 | Q9NYY1 |
| IL-21 | IL-21R | 59067 | Q9HBE4 |
| IL-22 | IL-22R | 50616 | Q9GZX6 |
| IL-23 (e.g., p19, p40, or a heterodimer thereof) | IL-23 R | 51561 | Q9NPF7 |
| IL-24 | IL-20R1/IL-20R2 and IL-22R1/IL-20R2 | 11009 | Q13007 |
| IL-25 | IL-17RA and IL-17RB | 64806 | Q9H293 |
| IL-26 | IL-10R2 chain and IL-20R1 chain | 55801 | Q9NPH9 |
| IL-27 (e.g., p28, EBI3, or a heterodimer thereof) | WSX-1 and gp130 | 246778 | Q8NEV9 |
| IL-28A, IL-28B, and IL29 | IL-28R1/IL-10R2 | 282617, 282618 | Q8IZI9, Q8IU54 |
| IL-30 | IL6R/gp130 | 246778 | Q8NEV9 |
| IL-31 | IL-31RA/OSMRβ | 386653 | Q6EBC2 |
| IL-32 | | 9235 | P24001 |
| IL-33 | ST2 | 90865 | 095760 |
| IL-34 | Colony-stimulating factor 1 receptor | 146433 | Q6ZMJ4 |
| IL-35 (e.g., p35, EBI3, or a heterodimer thereof) | IL-12Rβ2/gp 130; IL-12Rβ2/IL-12Rβ2; gpl30/gpl30 | 10148 | Q14213 |
| IL-36 | IL-36Ra | 27179 | Q9UHA7 |
| IL-37 | IL-18Rα and IL-18BP | 27178 | Q9NZH6 |
| IL-38 | IL-1R1, IL-36R | 84639 | Q8WWZ1 |
| IFN-α | IFNAR | 3454 | P17181 |
| IFN-β | IFNAR | 3454 | P17181 |
| IFN-γ | IFNGR1/IFNGR2 | 3459 | P15260 |
| TGF-β | TβR-I and TβR-II | 7046, 7048 | P36897, P37173 |
| TNF-α | TNFR1, TNFR2 | 7132, 7133 | P19438, P20333 |

### Polypeptide Hormones and Receptors

In some embodiments, an effector described herein comprises a hormone of Table 2, or a functional variant thereof, e.g., a protein having at least 80%, 85%, 90%, 95%, 967%, 98%, 99% identity to a protein sequence disclosed in Table 2 by reference to its UniProt ID. In some embodiments, the functional variant binds to the corresponding receptor with a Kd of no more than 10%, 20%, 30%, 40%, or 50% higher than the Kd of the corresponding wild-type hormone for the same receptor under the same conditions. In some embodiments, the polypeptide of Table 2 or functional variant thereof comprises a signal sequence, e.g., a signal sequence that is endogenous to the effector, or a heterologous signal sequence.

In some embodiments, an effector described herein comprises an antibody molecule (e.g., an scFv) that binds a hormone of Table 2. In some embodiments, an effector described herein comprises an antibody molecule (e.g., an scFv) that binds a hormone receptor of Table 2. In some embodiments, the antibody molecule comprises a signal sequence.

TABLE 2

Exemplary polypeptide hormones and receptors

| Hormone | Receptor | Entrez Gene ID | UniProt In |
|---|---|---|---|
| Natriuretic Peptide, e.g., Atrial Natriuretic Peptide (ANP) | NPRA, NPRB, NPRC | 4878 | P01160 |
| Brain Natriuretic Peptide (BNP) | NPRA, NPRB | 4879 | P16860 |
| C-type natriuretic peptide (CNP) | NPRB | 4880 | P23582 |
| Growth hormone (GH) | GHR | 2690 | P10912 |
| Prolactin (PRL) | PRLR | 5617 | P01236 |
| Thyroid-stimulating hormone (TSH) | TSH receptor | 7253 | P16473 |
| Adrenocorticotropic hormone (ACTH) | ACTH receptor | 5443 | P01189 |
| Follicle-stimulating hormone (FSH) | FSHR | 2492 | P23945 |
| Luteinizing hormone (LH) | LHR | 3973 | P22888 |
| Antidiuretic hormone (ADH) | Vasopressin receptors, e.g., V2; AVPR1A; AVPR1B; AVPR3; AVPR2 | 554 | P30518 |
| Oxytocin | OXTR | 5020 | P01178 |
| Calcitonin | Calcitonin receptor (CT) | 796 | P01258 |
| Parathyroid hormone (PTH) | PTHIR and PTH2R | 5741 | P01270 |
| Insulin | Insulin receptor (IR) | 3630 | P01308 |
| Glucagon | Glucagon receptor | 2641 | P01275 |
| GIP | GIPR | 2695 | P09681 |
| Fibroblast growth factor 19 (FGF 19) | FGFR4 | 9965 | 095750 |
| Fibroblast growth factor 21 (FGF2 1) | FGFR1c, 2c, 3c | 26291 | Q9NSA1 |
| Fibroblast growth factor 23 (FGF23) | FGFR1, 2, 4 | 8074 | Q9GZV9 |
| Melanocyte-stimulating hormone (alpha- MSH) | MC1R, MC4R, MC5R | | |
| Melanocyte-stimulating hormone (beta- MSH) | MC4R | | |
| Melanocyte-stimulating hormone (gamma- MSH) | MC1R, MC3R, MC4R, MC5R | | |
| Proopiomelanocortin POMC (alpha- beta-, gamma-, MSH precursor) | MC1R, MC3R, MC4R, MC5R | 5443 | P01189 |
| Glycoprotein hormones alpha chain (CGA) | | 1081 | P01215 |
| Follicle-stimulating hormone beta (FSHB) | FSHR | 2488 | P01225 |
| Leptin | LEPR | 3952 | P41159 |
| Ghrelin | GHSR | 51738 | Q9UBU3 |

### Growth Factors

In some embodiments, an effector described herein comprises a growth factor of Table 3, or a functional variant thereof, e.g., a protein having at least 80%, 85%, 90%, 95%, 967%, 98%, 99% identity to a protein sequence disclosed in Table 3 by reference to its UniProt ID. In some embodiments, the functional variant binds to the corresponding receptor with a Kd of no more than 10%, 20%, 30%, 40%, or 50% higher than the Kd of the corresponding wild-type growth factor for the same receptor under the same conditions. In some embodiments, the polypeptide of Table 3 or functional variant thereof comprises a signal sequence, e.g., a signal sequence that is endogenous to the effector, or a heterologous signal sequence.

In some embodiments, an effector described herein comprises an antibody or fragment thereof that binds a growth factor of Table 3. In some embodiments, an effector described herein comprises an antibody molecule (e.g., an scFv) that binds a growth factor receptor of Table 3. In some embodiments, the antibody molecule comprises a signal sequence.

TABLE 3

Exemplary growth factors

| PDGF family | | Entrez Gene ID | UniProt ID |
|---|---|---|---|
| PDGF (e.g., PDGF-1, PDGF-2, or a heterodimer thereof) | PDGF receptor, e.g., PDGFRα, PDGFRβ | 5156 | P16234 |
| CSF-1 | CSF1R | 1435 | P09603 |
| SCF | CD117 | 3815 | P10721 |
| VEGF family | | | |
| VEGF (e.g., isoforms VEGF 121, VEGF 165, VEGF 189, and VEGF 206) | VEGFR-1, VEGFR-2 | 2321 | P17948 |
| VEGF-B | VEGFR-1 | 2321 | P17949 |
| VEGF-C | VEGFR-2 and VEGFR -3 | 2324 | P35916 |
| PIGF | VEGFR-1 | 5281 | Q07326 |
| EGF family | | | |
| EGF | EGFR | 1950 | P01133 |
| TGF-α | EGFR | 7039 | P01135 |
| amphiregulin | EGFR | 374 | P15514 |
| HB-EGF | EGFR | 1839 | Q99075 |
| betacellulin | EGFR, ErbB-4 | 685 | P35070 |
| epiregulin | EGFR, ErbB-4 | 2069 | 014944 |
| Heregulin | EGFR, ErbB-4 | 3084 | Q02297 |
| FGF family | | | |
| FGF-1, FGF-2, FGF-3, FGF-4, FGF-5, FGF-6, FGF-7, FGF-8, FGF-9 | FGFR1, FGFR2, FGFR3, and FGFR4 | 2246, 2247, 2248, 2249, 2250, 2251, 2252, 2253, 2254 | P05230, P09038, P11487, P08620, |
| | | | P12034, P10767, P21781, P55075, P31371 |
| Insulin | IR | 3630 | P01308 |
| IGF-I | IGF-I receptor, IGF-II receptor | 3479 | P05019 |
| IGF-II | IGF-II receptor | 3481 | P01344 |
| HGF | MET receptor | 3082 | P14210 |
| MSP | RON | 4485 | P26927 |
| NGF | LNGFR, trkA | 4803 | P01138 |
| BDNF | trkB | 627 | P23560 |
| NT-3 | trkA, trkB, trkC | 4908 | P20783 |
| NT-4 | trkA, trkB | 4909 | P34130 |
| NT-5 | trkA, trkB | 4909 | P34130 |
| ANGPT1 | HPK-6/TEK | 284 | Q15389 |
| ANGPT2 | HPK-6/TEK | 285 | 015123 |
| ANGPT3 | HPK-6/TEK | 9068 | 095841 |
| ANGPT4 | HPK-6/TEK | 51378 | Q9Y264 |
| ANGPTL2 | LILRB2 & integrin α5βl | 23452 | Q9UKU9 |
| ANGPTL3 | LPL | 27329 | Q9Y5C1 |
| ANGPTL4 | | 51129 | Q9BY76 |
| ANGPTL8 | PirB | 55908 | Q6UXH0 |

### Clotting Factors

In some embodiments, an effector described herein comprises a polypeptide of Table 4, or a functional variant thereof, e.g., a protein having at least 80%, 85%, 90%, 95%, 967%, 98%, 99% identity to a protein sequence disclosed in Table 4 by reference to its UniProt ID. In some embodiments, the functional variant catalyzes the same reaction as the corresponding wild-type protein, e.g., at a rate no less than 10%, 20%, 30%, 40%, or 50% lower or higher than the wild-type protein. In some embodiments, the polypeptide of Table 4 or functional variant thereof comprises a signal sequence, e.g., a signal sequence that is endogenous to the effector, or a heterologous signal sequence.

TABLE 4

Clotting-associated factors

| Effector | Indication | Entrez Gene ID | UniProt ID |
|---|---|---|---|
| Factor I (fibrinogen) | Afibrinogenomia | 2243,2266,2244 | P02671, 02679, P02675 |
| Factor II | Factor II Deficiency | 2147 | P00734 |
| Factor IX | Hemophilia B | 2158 | P00740 |
| Factor V | Owren’s disease | 2153 | P12259 |
| Factor VIII | Hemophilia A | 2157 | P00451 |
| Factor X | Stuart-Prower Factor Deficiency | 2159 | P00742 |
| Factor XI | Hemophilia C | 2160 | P03951 |
| Factor XIII | Fibrin Stabilizing factor deficiency | 2162, 2165 | P00488, P05160 |
| vWF | von Willebrand disease | 7450 | P04275 |

### Enzyme Replacement Therapeutics

In some embodiments, an effector described herein comprises an enzyme of Table 5, or a functional variant thereof, e.g., a protein having at least 80%, 85%, 90%, 95%, 967%, 98%, 99% identity to a protein sequence disclosed in Table 5 by reference to its UniProt ID. In some embodiments, the functional variant catalyzes the same reaction as the corresponding wild-type protein, e.g., at a rate no less or no more than 10%, 20%, 30%, 40%, or 50% lower than the wild-type protein.

TABLE 5

Exemplary enzymatic effectors for enzyme deficiency

| Effector | deficiency | Entrez Gene ID | UniProt ID |
|---|---|---|---|
| 3-methylcrotonyl-CoA carboxylase | 3-methylcrotonyl-CoA carboxylase deficiency | 56922, 64087 | Q96RQ3, Q9HCC0 |
| Acetyl-CoA- glucosaminide N-acetyltransferase | Mucopolysaccharidosis MPS III (Sanfilippo’s syndrome) Type III-C | 138050 | Q68CP4 |
| ADAMTS13 | Thrombotic Thrombocytopenic Purpura | 11093 | Q76LX8 |
| adenine phosphoribosyltransfera se | Adenine phosphoribosyltransferase deficiency | 353 | P07741 |
| Adenosine deaminase | Adenosine deaminase deficiency | 100 | P00813 |
| ADP-ribose protein hydrolase | Glutamyl ribose-5-phosphate storage disease | 26119, 54936 | Q5SW96, Q9NX46 |
| alpha glucosidase | Glycogen storage disease type 2 (Pompe’s disease) | 2548 | P10253 |
| Arginase | Familial hyperarginemia | 383, 384 | P05089, P78540 |
| Arylsulfatase A | Metachromatic leukodystrophy | 410 | P15289 |
| Cathepsin K | Pycnodysostosis | 1513 | P43235 |
| Ceramidase | Farber’s disease (lipogranulomatosis) | 125981, 340485, 55331 | Q8TDN7, Q5QJU3, Q9NLTN7 |
| Cystathionine B synthase | Homocystinuria | 875 | P35520 |
| Dolichol-P-mannose synthase | Congenital disorders of N-glycosylation CDG Ie | 8813, 54344 | 060762, Q9P2X0 |
| Dolicho-P-Glc:Man9GlcNAc2-PP-dolichol glucosyltransferase | Congenital disorders of N-glycosylation CDG Ic | 84920 | Q5BKT4 |
| Dolicho-P-Man: Man5 GlcNAc2-PP-dolichol mannosyltransferase | Congenital disorders of N-glycosylation CDG Id | 10195 | Q92685 |
| Dolichyl-P-glucose:Glc- l-Man-9-GlcNAc-2-PP- dolichyl-α-3-glucosyltransferase | Congenital disorders of N-glycosylation CDG Ih | 79053 | Q9BVK2 |
| Dolichyl-P-mannose:Man-7-GlcNAc-2-PP-dolichyl- α-6-mannosyltransferase | Congenital disorders of N-glycosylation CDG Ig | 79087 | Q9BV10 |
| Factor II | Factor II Deficiency | 2147 | P00734 |
| Factor IX | Hemophilia B | 2158 | P00740 |
| Factor V | Owren’s disease | 2153 | P12259 |
| Factor VIII | Hemophilia A | 2157 | P00451 |
| Factor X | Stuart-Prower Factor Deficiency | 2159 | P00742 |
| Factor XI | Hemophilia C | 2160 | P03951 |
| Factor XIII | Fibrin Stabilizing factor deficiency | 2162, 2165 | P00488, P05160 |
| Galactosamine-6-sulfate sulfatase | Mucopolysaccharidosis MPS IV (Morquio’s syndrome) Type IV-A | 2588 | P34059 |
| Galactosylceramide β- galactosidase | Krabbe’s disease | 2581 | P54803 |
| Ganglioside β-galactosidase | GM1 gangliosidosis, generalized | 2720 | P16278 |
| Ganglioside β-galactosidase | GM2 gangliosidosis | 2720 | P16278 |
| Ganglioside β-galactosidase | Sphingolipidosis Type I | 2720 | P16278 |
| Ganglioside β- galactosidase | Sphingolipidosis Type II (juvenile type) | 2720 | P16278 |
| Ganglioside β- galactosidase | Sphingolipidosis Type III (adult type) | 2720 | P16278 |
| Glucosidase I | Congenital disorders of N-glycosylation CDG IIb | 2548 | P10253 |
| Glucosylceramide β- glucosidase | Gaucher’s disease | 2629 | P04062 |
| Heparan-S-sulfate sulfamidase | Mucopolysaccharidosis MPS III (Sanfilippo’s syndrome) Type III-A | 6448 | P51688 |
| homogentisate oxidase | Alkaptonuria | 3081 | Q93099 |
| Hyaluronidase | Mucopolysaccharidosis MPS IX (hyaluronidase deficiency) | 3373, 8692, 8372, 23553 | Q12794, Q12891, 043820, Q2M3T9 |
| Iduronate sulfate sulfatase | Mucopolysaccharidosis MPS II (Hunter’s syndrome) | 3423 | P22304 |
| Lecithin-cholesterol acyltransferase (LCAT) | Complete LCAT deficiency, Fish-eye disease, atherosclerosis, hypercholesterolemia | 3931 | 606967 |
| Lysine oxidase | Glutaric acidemia type I | 4015 | P28300 |
| Lysosomal acid lipase | Cholesteryl ester storage disease (CESD) | 3988 | P38571 |
| Lysosomal acid lipase | Lysosomal acid lipase deficiency | 3988 | P38571 |
| lysosomal acid lipase | Wolman’s disease | 3988 | P38571 |
| Lysosomal pepstatin- insensitive peptidase | Ceroid lipofuscinosis Late infantile form (CLN2, Jansky-Bielschowsky disease) | 1200 | 014773 |
| Mannose (Man) phosphate (P) isomerase | Congenital disorders of N-glycosylation CDG Ib | 4351 | P34949 |
| Mannosyl-α-1,6-glycoprotein-β-1,2-N-acetylglucosminyltransf erase | Congenital disorders of N-glycosylation CDG IIa | 4247 | Q10469 |
| Metalloproteinase-2 | Winchester syndrome | 4313 | P08253 |
| methylmalonyl-CoA mutase | Methylmalonic acidemia (vitamin b 12 non-responsive) | 4594 | P22033 |
| N-Acetyl galactosamine α-4-sulfate sulfatase (arylsulfatase B) | Mucopolysaccharidosis MPS VI (Maroteaux-Lamy syndrome) | 411 | P15848 |
| N-acetyl-D-glucosaminidase | Mucopolysaccharidosis MPS III (Sanfilippo’s syndrome) Type III-B | 4669 | P54802 |
| N-Acetyl- galactosaminidase | Schindler’s disease Type I (infantile severe form) | 4668 | P17050 |
| N-Acetyl- galactosaminidase | Schindler’s disease Type II (Kanzaki disease, adult-onset form) | 4668 | P17050 |
| N-Acetyl- galactosaminidase | Schindler’s disease Type III (intermediate form) | 4668 | P17050 |
| N-acetyl-glucosaminine-6-sulfate sulfatase | Mucopolysaccharidosis MPS III (Sanfilippo’s syndrome) Type III-D | 2799 | P15586 |
| N-acetylglucosaminyl-I-phosphotransferase | Mucolipidosis ML III (pseudo-Hurler’s polydystrophy) | 79158 | Q3T906 |
| N-Acetylglucosaminyl-1-phosphotransferase catalytic subunit | Mucolipidosis ML II (I-cell disease) | 79158 | Q3T906 |
| N-acetylglucosaminyl-1-phosphotransferase, substrate-recognition subunit | Mucolipidosis ML III (pseudo-Hurler’s polydystrophy) Type III-C | 84572 | Q9UJJ9 |
| N-Aspartylglucosaminidas e | Aspartylglucosaminuria | 175 | P20933 |
| Neuraminidase 1 (sialidase) | Sialidosis | 4758 | Q99519 |
| Palmitoyl-protein thioesterase-1 | Ceroid lipofuscinosis Adult form (CLN4, Kufs’ disease) | 5538 | P50897 |
| Palmitoyl-protein thioesterase-1 | Ceroid lipofuscinosis Infantile form (CLN1, Santavuori-Haltia disease) | 5538 | P50897 |
| Phenylalanine hydroxylase | Phenylketonuria | 5053 | P00439 |
| Phosphomannomutase-2 | Congenital disorders of N-glycosylation CDG Ia (solely neurologic and neurologic- multivisceral forms) | 5373 | 015305 |
| Porphobilinogen deaminase | Acute Intermittent Porphyria | 3145 | P08397 |
| Purine nucleoside phosphorylase | Purine nucleoside phosphorylase deficiency | 4860 | P00491 |
| pyrimidine 5′ nucleotidase | Hemolytic anemia and/or pyrimidine 5′ nucleotidase deficiency | 51251 | Q9H0P0 |
| Sphingomyelinase | Niemann-Pick disease type A | 6609 | P17405 |
| Sphingomyelinase | Niemann-Pick disease type B | 6609 | P17405 |
| Sterol 27-hydroxylase | Cerebrotendinous xanthomatosis (cholestanol lipidosis) | 1593 | Q02318 |
| Thymidine phosphorylase | Mitochondrial neurogastrointestinal encephalomyopathy (MNGIE) | 1890 | P19971 |
| Trihexosylceramide α-galactosidase | Fabry’s disease | 2717 | P06280 |
| tyrosinase, e.g., OCAl | albinism, e.g., ocular albinism | 7299 | P14679 |
| UDP-GlcNAc: dolichyl-P NAcGlc phosphotransferase | Congenital disorders of N-glycosylation CDG Ij- | 1798 | Q9H3H5 |
| UDP-N-acetylglucosamine-2-epimerase/N-acetylmannosamine kinase, sialin | Sialuria French type | 10020 | Q9Y223 |
| Uricase | Lesch-Nyhan syndrome, gout | 391051 | No protein |
| uridine diphosphate glucuronyl-transferase (e.g., UGT1A1) | Crigler-Najjar syndrome | 54658 | P22309 |
| α-1,2-Mannosyltransferase | Congenital disorders of N-glycosylation CDG 11 (608776) | 79796 | Q9H6U8 |
| α-1,2-Mannosyltransferase | Congenital disorders of N-glycosylation, type I (pre-Golgi glycosylation defects) | 79796 | Q9H6U8 |
| α-1,3-Mannosyltransferase | Congenital disorders of N-glycosylation CDG Ii | 440138 | Q2TAA5 |
| α-D-Mannosidase | α-Mannosidosis, type I (severe) or II (mild) | 10195 | Q92685 |
| α-L-Fucosidase | Fucosidosis | 4123 | Q9NTJ4 |
| α-1-Iduronidase | Mucopolysaccharidosis MPS I H/S (Hurler-Scheie syndrome) | 2517 | P04066 |
| α-1-Iduronidase | Mucopolysaccharidosis MPS I-H (Hurler’s syndrome) | 3425 | P35475 |
| α-1-Iduronidase | Mucopolysaccharidosis MPS I-S (Scheie’s syndrome) | 3425 | P35475 |
| β-1,4- Galactosyltransferase | Congenital disorders of N-glycosylation CDG lid | 3425 | P35475 |
| β-1,4- Mannosyltransferase | Congenital disorders of N-glycosylation CDG Ik | 2683 | P15291 |
| β-D-Mannosidase | β-Mannosidosis | 56052 | Q9BT22 |
| β-Galactosidase | Mucopolysaccharidosis MPS IV (Morquio’s syndrome) Type IV-B | 4126 | 000462 |
| β-Glucuronidase | Mucopolysaccharidosis MPS VII (Sly’s syndrome) | 2720 | P16278 |
| β-Hexosaminidase A | Tay-Sachs disease | 2990 | P08236 |
| β-Hexosaminidase B | Sandhoffs disease | 3073 | P06865 |

### Other Non-Enzymatic Effectors

In some embodiments, a therapeutic polypeptide described herein comprises a polypeptide of Table 6, or a functional variant thereof, .g., a protein having at least 80%, 85%, 90%, 95%, 967%, 98%, 99% identity to a protein sequence disclosed in Table 6 by reference to its UniProt ID.

TABLE 6

Exemplary non-enzymatic effectors and corresponding indications

| | | | |
|---|---|---|---|
| Effector | Indication | Entrez Gene ID | UniProt ID |
| Survival motor neuron protein (SMN) | spinal muscular atrophy | 6606 | Q16637 |
| Dystrophin | muscular dystrophy (e.g., Duchenne muscular dystrophy or Becker muscular dystrophy) | 1756 | P11532 |
| Complement protein, e.g., Complement factor C 1 | Complement Factor I deficiency | 3426 | P05156 |
| Complement factor H | Atypical hemolytic uremic syndrome | 3075 | P08603 |
| Cystinosin (lysosomal cystine transporter) | Cystinosis | 1497 | 060931 |
| Epididymal secretory protein 1 (HE 1; NPC2 protein) | Niemann-Pick disease Type C2 | 10577 | P61916 |
| GDP-fucose transporter-1 | Congenital disorders of N-glycosylation CDG | 55343 | Q96A29 |
| | IIc (Rambam-Hasharon syndrome) | | |
| GM2 activator protein | GM2 activator protein deficiency (Tay-Sachs disease AB variant, GM2A) | 2760 | Q17900 |
| Lysosomal transmembrane CLN3 protein | Ceroid lipofuscinosis Juvenile form (CLN3, Batten disease, Vogt-Spielmeyer disease) | 1207 | Q13286 |
| Lysosomal transmembrane CLN5 protein | Ceroid lipofuscinosis Variant late infantile form, Finnish type (CLN5) | 1203 | 075503 |
| Na phosphate cotransporter, sialin | Infantile sialic acid storage disorder | 26503 | Q9NRA2 |
| Na phosphate cotransporter, sialin | Sialuria Finnish type (Salla disease) | 26503 | Q9NRA2 |
| NPC1 protein | Niemann-Pick disease Type C⅟Type D | 4864 | 015118 |
| Oligomeric Golgi complex-7 | Congenital disorders of N-glycosylation CDG lie | 91949 | P83436 |
| Prosaposin | Prosaposin deficiency | 5660 | P07602 |
| Protective protein/cathepsin A (PPCA) | Galactosialidosis (Goldberg’s syndrome, combined neuraminidase and (3-galactosidase deficiency) | 5476 | P10619 |
| Protein involved in mannose-P-dolichol utilization | Congenital disorders of N-glycosylation CDG If | 9526 | 075352 |
| Saposin B | Saposin B deficiency (sulfatide activator deficiency) | 5660 | P07602 |
| Saposin C | Saposin C deficiency (Gaucher’s activator deficiency) | 5660 | P07602 |
| Sulfatase-modifying factor-1 | Mucosulfatidosis (multiple sulfatase deficiency) | 285362 | Q8NBK3 |
| Transmembrane CLN6 protein | Ceroid lipofuscinosis Variant late infantile form (CLN6) | 54982 | Q9NWW5 |
| Transmembrane CLN8 protein | Ceroid lipofuscinosis Progressive epilepsy with intellectual disability | 2055 | Q9UBY8 |
| vWF | von Willebrand disease | 7450 | P04275 |
| Factor I (fibrinogen) | Afibrinogenomia | 2243, 2244, 2266 | P02671, P02675, P02679 |
| erythropoietin (hEPO) | | | |

### Regeneration, Repair and Fibrosis Factors

Therapeutic polypeptides described herein also include growth factors, e.g., as disclosed in Table 7, or functional variants thereof, .g., a protein having at least 80%, 85%, 90%, 95%, 967%, 98%, 99% identity to a protein sequence disclosed in Table 7 by reference to its UniProt ID. Also included are antibodies or fragments thereof against such growth factors, or miRNAs that promote regeneration and repair.

TABLE 7

| | | |
|---|---|---|
| Target | Gene accession # | Protein accession # |
| VEGF-A | NG 008732 | NP_001165094 |
| NRG-1 | NG_012005 | NP_001153471 |
| FGF2 | NG_029067 | NP_001348594 |
| FGF1 | Gene ID: 2246 | NP_001341882 |
| miR-199-3p | MIMAT0000232 | n/a |
| miR-590-3p | MIMAT0004801 | n/a |
| mi-17-92 | MI0000071 | https://www.ncbi.nlm.nih.gov/pm c/articles/PMC2732113/figure/F⅟ |
| miR-222 | MI0000299 | n/a |
| miR-302-367 | MIR302A And MIR367 | https://www.ncbi.nlm.nih.gov/pm c/articles/PMC4400607/ |

### Transformation Factors

Therapeutic polypeptides described herein also include transformation factors, e.g., protein factors that transform fibroblasts into differentiated cell e.g., factors disclosed in Table 8 or functional variants thereof, .g., a protein having at least 80%, 85%, 90%, 95%, 967%, 98%, 99% identity to a protein sequence disclosed in Table 8 by reference to its UniProt ID.

TABLE 8

| | | | |
|---|---|---|---|
| Target | Indication | Gene accession # | Protein accession # |
| MESP1 | Organ Repair by transforming fibroblasts | Gene ID: 55897 | EAX02066 |
| ETS2 | Organ Repair by transforming fibroblasts | GeneID: 2114 | NP_005230 |
| HAND2 | Organ Repair by transforming fibroblasts | GeneID: 9464 | NP_068808 |
| MYOCARDIN | Organ Repair by transforming fibroblasts | GeneID: 93649 | NP_001139784 |
| ESRRA | Organ Repair by transforming fibroblasts | Gene ID: 2101 | AAH92470 |
| miR-1 | Organ Repair by transforming fibroblasts | MI0000651 | n/a |
| miR-133 | Organ Repair by transforming fibroblasts | MI000450 | n/a |
| TGFb | Organ Repair by transforming fibroblasts | GeneID: 7040 | NP_000651.3 |
| WNT | Organ Repair by transforming fibroblasts | Gene ID: 7471 | NP_005421 |
| JAK | Organ Repair by transforming fibroblasts | Gene ID: 3716 | NP_001308784 |
| NOTCH | Organ Repair by transforming fibroblasts | GeneID: 4851 | XP011517019 |

### Proteins That Stimulate Cellular Regeneration

Therapeutic polypeptides described herein also include proteins that stimulate cellular regeneration e.g., proteins disclosed in Table 9 or functional variants thereof, e.g., a protein having at least 80%, 85%, 90%, 95%, 967%, 98%, 99% identity to a protein sequence disclosed in Table 9 by reference to its UniProt ID.

TABLE 9

| | | |
|---|---|---|
| Target | Gene accession # | Protein accession # |
| MST1 | NG_016454 | NP_066278 |
| STK30 | Gene ID: 26448 | NP_036103 |
| MST2 | Gene ID: 6788 | NP_006272 |
| SAV1 | Gene ID: 60485 | NP_068590 |
| LATS1 | Gene ID: 9113 | NP_004681 |
| LATS2 | Gene ID: 26524 | NP_055387 |
| YAP1 | NG_029530 | NP_001123617 |
| CDKN2b | NG_023297 | NP_004927 |
| CDKN2a | NG_007485 | NP_478102 |

In some embodiments, the circular polyribonucleotide comprises one or more expression sequences and is configured for persistent expression in a cell of a subject in vivo. In some embodiments, the circular polyribonucleotide is configured such that expression of the one or more expression sequences in the cell at a later time point is equal to or higher than an earlier time point. In such embodiments, the expression of the one or more expression sequences can be either maintained at a relatively stable level or can increase over time. The expression of the expression sequences can be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

### Encryptogen

As described herein, the circular polyribonucleotide can further comprise an encryptogen to reduce, evade or avoid the innate immune response of a cell. In one aspect, provided herein are circular polyribonucleotide which when delivered to cells, results in a reduced immune response from the host as compared to the response triggered by a reference compound, e.g. a linear polynucleotide corresponding to the described circular polyribonucleotide or a circular polyribonucleotide lacking an encryptogen. In some embodiments, the circular polyribonucleotide has less immunogenicity than a counterpart lacking an encryptogen.

In some embodiments, an encryptogen enhances stability. There is growing body of evidence about the regulatory roles played by the UTRs in terms of stability of a nucleic acid molecule and translation. The regulatory features of a UTR may be included in the encryptogen to enhance the stability of the circular polyribonucleotide.

In some embodiments, 5′ or 3′UTRs can constitute encryptogens in a circular polyribonucleotide. For example, removal or modification of UTR AU rich elements (AREs) may be useful to modulate the stability or immunogenicity of the circular polyribonucleotide.

In some embodiments, removal of modification of AU rich elements (AREs) in expression sequence, e.g., translatable regions, can be useful to modulate the stability or immunogenicity of the circular polyribonucleotide

In some embodiments, an encryptogen comprises miRNA binding site or binding site to any other non-coding RNAs. For example, incorporation of miR-142 sites into the circular polyribonucleotide described herein may not only modulate expression in hematopoietic cells, but also reduce or abolish immune responses to a protein encoded in the circular polyribonucleotide.

In some embodiments, an encyptogen comprises one or more protein binding sites that enable a protein, e.g., an immunoprotein, to bind to the RNA sequence. By engineering protein binding sites into the circular polyribonucleotide, the circular polyribonucleotide may evade or have reduced detection by the host’s immune system, have modulated degradation, or modulated translation, by masking the circular polyribonucleotide from components of the host’s immune system. In some embodiments, the circular polyribonucleotide comprises at least one immunoprotein binding site, for example to evade immune responses, e.g., CTL responses. In some embodiments, the immunoprotein binding site is a nucleotide sequence that binds to an immunoprotein and aids in masking the circular polyribonucleotide as exogenous.

In some embodiments, an encryptogen comprises one or more modified nucleotides. Exemplary modifications can include any modification to the sugar, the nucleobase, the internucleoside linkage (e.g., to a linking phosphate / to a phosphodiester linkage / to the phosphodiester backbone), and any combination thereof that can prevent or reduce immune response against the circular polyribonucleotide. Some of the exemplary modifications provided herein are described in details below.

In some embodiments, the circular polyribonucleotide includes one or more modifications as described elsewhere herein to reduce an immune response from the host as compared to the response triggered by a reference compound, e.g., a circular polyribonucleotide lacking the modifications. In particular, the addition of one or more inosine has been shown to discriminate RNA as endogenous versus viral. See for example, Yu, Z. et al. (2015) RNA editing by ADAR1 marks dsRNA as “self. Cell Res. 25, 1283-1284, which is incorporated by reference in its entirety.

In some embodiments, the circular polyribonucleotide includes one or more expression sequences for shRNA or an RNA sequence that can be processed into siRNA, and the shRNA or siRNA targets RIG-1 and reduces expression of RIG-1. RIG-1 can sense foreign circular RNA and leads to degradation of foreign circular RNA. Therefore, a circular polynucleotide harboring sequences for RIG-1-targeting shRNA, siRNA or any other regulatory nucleic acids can reduce immunity, e.g., host cell immunity, against the circular polyribonucleotide.

In some embodiments, the circular polyribonucleotide lacks a sequence, element or structure, that aids the circular polyribonucleotide in reducing, evading or avoiding an innate immune response of a cell. In some such embodiments, the circular polyribonucleotide may lack a polyA sequence, a 5′ end, a 3′ end, phosphate group, hydroxyl group, or any combination thereof.

### Structure

In some embodiments, the circular polyribonucleotide comprises a higher order structure, e.g., a secondary or tertiary structure. In some embodiments, complementary segments of the circular polyribonucleotide fold itself into a double stranded segment, held together with hydrogen bonds between pairs, e.g., A-U and C-G. In some embodiments, helices, also known as stems, are formed intramolecularly, having a double-stranded segment connected to an end loop. In some embodiments, the circular polyribonucleotide has at least one segment with a quasi-double-stranded secondary structure. In some embodiments, a segment having a quasi-double-stranded secondary structure has at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides. In some embodiments, the circular polyribonucleotide has one or more segments (e.g., 2, 3, 4, 5, 6, or more) having a quasi-double-stranded secondary structure. In some embodiments, the segments are separated by 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides.

In some embodiments, one or more sequences of the circular polyribonucleotide include substantially single stranded vs double stranded regions. In some embodiments, the ratio of single stranded to double stranded may influence the functionality of the circular polyribonucleotide.

In some embodiments, one or more sequences of the circular polyribonucleotide that are substantially single stranded. In some embodiments, one or more sequences of the circular polyribonucleotide that are substantially single stranded may include a protein- or RNA-binding site. In some embodiments, the circular polyribonucleotide sequences that are substantially single stranded may be conformationally flexible to allow for increased interactions. In some embodiments, the sequence of the circular polyribonucleotide is purposefully engineered to include such secondary structures to bind or increase protein or nucleic acid binding.

In some embodiments, the circular polyribonucleotide sequences that are substantially double stranded. In some embodiments, one or more sequences of the circular polyribonucleotide that are substantially double stranded may include a conformational recognition site, e.g., a riboswitch or aptazyme. In some embodiments, the circular polyribonucleotide sequences that are substantially double stranded may be conformationally rigid. In some such instances, the conformationally rigid sequence may sterically hinder the circular polyribonucleotide from binding a protein or a nucleic acid. In some embodiments, the sequence of the circular polyribonucleotide is purposefully engineered to include such secondary structures to avoid or reduce protein or nucleic acid binding.

There are 16 possible base-pairings, however of these, six (AU, GU, GC, UA, UG, CG) may form actual base-pairs. The rest are called mismatches and occur at very low frequencies in helices. In some embodiments, the structure of the circular polyribonucleotide cannot easily be disrupted without impact on its function and lethal consequences, which provide a selection to maintain the secondary structure. In some embodiments, the primary structure of the stems (i.e., their nucleotide sequence) can still vary, while still maintaining helical regions. The nature of the bases is secondary to the higher structure, and substitutions are possible as long as they preserve the secondary structure. In some embodiments, the circular polyribonucleotide has a quasi-helical structure. In some embodiments, the circular polyribonucleotide has at least one segment with a quasi-helical structure. In some embodiments, a segment having a quasi-helical structure has at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides. In some embodiments, the circular polyribonucleotide has one or more segments (e.g., 2, 3, 4, 5, 6, or more) having a quasi-helical structure. In some embodiments, the segments are separated by 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides. In some embodiments, the circular polyribonucleotide includes at least one of a U-rich or A-rich sequence or a combination thereof. In some embodiments, the U-rich and/or A-rich sequences are arranged in a manner that would produce a triple quasi-helix structure. In some embodiments, the circular polyribonucleotide has a double quasi-helical structure. In some embodiments, the circular polyribonucleotide has one or more segments (e.g., 2, 3, 4, 5, 6, or more) having a double quasi-helical structure. In some embodiments, the circular polyribonucleotide includes at least one of a C-rich and/or G-rich sequence. In some embodiments, the C-rich and/or G-rich sequences are arranged in a manner that would produce triple quasi-helix structure. In some embodiments, the circular polyribonucleotide has an intramolecular triple quasi-helix structure that aids in stabilization.

In some embodiments, the circular polyribonucleotide has two quasi-helical structure (e.g., separated by a phosphodiester linkage), such that their terminal base pairs stack, and the quasi-helical structures become colinear, resulting in a “coaxially stacked” substructure.

In some embodiments, the circular polyribonucleotide comprises a tertiary structure with one or more motifs, e.g., a pseudoknot, a g-quadruplex, a helix, and coaxial stacking.

In some embodiments, the circular polyribonucleotide has at least one binding site, e.g., at least one protein binding site, at least one miRNA binding site, at least one IncRNA binding site, at least one tRNA binding site, at least one rRNA binding site, at least one snRNA binding site, at least one siRNA binding site, at least one piRNA binding site, at least one snoRNA binding site, at least one snRNA binding site, at least one exRNA binding site, at least one scaRNA binding site, at least one Y RNA binding site, at least one hnRNA binding site, and/or at least one tRNA motif.

### Regulatory Elements

In some embodiments, the circular polyribonucleotide as described herein further comprises a regulatory element, e.g., a sequence that modifies expression of an expression sequence within the circular polyribonucleotide.

A regulatory element may include a sequence that is located adjacent to an expression sequence that encodes an expression product. A regulatory element may be linked operatively to the adjacent sequence. A regulatory element may increase an amount of product expressed as compared to an amount of the expressed product when no regulatory element exists. In addition, one regulatory element can increase an amount of products expressed for multiple expression sequences attached in tandem. Hence, one regulatory element can enhance the expression of one or more expression sequences. Multiple regulatory elements are well-known to persons of ordinary skill in the art.

A regulatory element as provided herein can include a selective translation sequence. As used herein, the term “selective translation sequence” can refer to a nucleic acid sequence that selectively initiates or activates translation of an expression sequence in the circular polyribonucleotide, for instance, certain riboswtich aptazymes. A regulatory element can also include a selective degradation sequence. As used herein , the term “selective degradation sequence” can refer to a nucleic acid sequence that initiates degradation of the circular polyribonucleotide, or an expression product of the circular polyribonucleotide. Exemplary selective degradation sequence can include riboswitch aptazymes and miRNA binding sites.

In some embodiments, the regulatory element is a translation modulator. A translation modulator can modulate translation of the expression sequence in the circular polyribonucleotide. A translation modulator can be a translation enhancer or suppressor. In some embodiments, the circular polyribonucleotide includes at least one translation modulator adjacent to at least one expression sequence. In some embodiments, the circular polyribonucleotide includes a translation modulator adjacent each expression sequence. In some embodiments, the translation modulator is present on one or both sides of each expression sequence, leading to separation of the expression products, e.g., peptide(s) and or polypeptide(s).

### Regulatory Nucleic Acids

In some embodiments, the circular polyribonucleotide as described herein further comprises one or more expression sequences that encode regulatory nucleic acid, e.g., that modifies expression of an endogenous gene and/or an exogenous gene. In some embodiments, the expression sequence of a circular polyribonucleotide as provided herein can comprise a sequence that is antisense to a regulatory nucleic acid like a non-coding RNA, such as, but not limited to, tRNA, IncRNA, miRNA, rRNA, snRNA, microRNA, siRNA, piRNA, snoRNA, snRNA, exRNA, scaRNA, Y RNA, and hnRNA.

In one embodiment, the regulatory nucleic acid targets a host gene. The regulatory nucleic acids may include, but are not limited to, a nucleic acid that hybridizes to an endogenous gene (e.g., miRNA, siRNA, mRNA, IncRNA, RNA, DNA, an antisense RNA, gRNA as described herein elsewhere), nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In one embodiments, the sequence is a miRNA. In some embodiments, the regulatory nucleic acid targets a sense strand of a host gene. In some embodiments, the regulatory nucleic acid targets an antisense strand of a host gene

In some embodiments, the circular polyribonucleotide comprises a regulatory nucleic acid, such as a guide RNA (gRNA). In some embodiments, the circular polyribonucleotide comprises a guide RNA or encodes the guide RNA. A gRNA short synthetic RNA composed of a “scaffold” sequence necessary for binding to the incomplete effector moiety and a user-defined ~20 nucleotide targeting sequence for a genomic target. In practice, guide RNA sequences are generally designed to have a length of between 17 - 24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985 - 991.

The gRNA may recognize specific DNA sequences (e.g., sequences adjacent to or within a promoter, enhancer, silencer, or repressor of a gene).

In one embodiment, the gRNA is used as part of a CRISPR system for gene editing. For the purposes of gene editing, the circular polyribonucleotide may be designed to include one or multiple guide RNA sequences corresponding to a desired target DNA sequence; see, for example, Cong et al. (2013) Science, 339:819-823; Ran et al. (2013) Nature Protocols, 8:2281 - 2308. At least about 16 or 17 nucleotides of gRNA sequence are required by Cas9 for DNA cleavage to occur; for Cpf1 at least about 16 nucleotides of gRNA sequence is needed to achieve detectable DNA cleavage.

Certain regulatory nucleic acids can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

In some embodiments, the circular polyribonucleotide comprises regulatory nucleic acids that are RNA or RNA-like structures typically between about 5-500 base pairs (depending on the specific RNA structure, e.g., miRNA 5-30 bps, lncRNA 200-500 bps) and may have a nucleobase sequence identical (complementary) or nearly identical (substantially complementary) to a coding sequence in an expressed target gene within the cell.

Long non-coding RNAs (lncRNA) are defined as non-protein coding transcripts longer than 100 nucleotides. This somewhat arbitrary limit distinguishes lncRNAs from small regulatory RNAs such as microRNAs (miRNAs), short interfering RNAs (siRNAs), and other short RNAs. In general, the majority (-78%) of lncRNAs are characterized as tissue-specific. Divergent lncRNAs that are transcribed in the opposite direction to nearby protein-coding genes (comprise a significant proportion -20% of total lncRNAs in mammalian genomes) may possibly regulate the transcription of the nearby gene. In one embodiment, the circular polyribonucleotide provided herein comprises a sense strand of a lncRNA. In one embodiment, the circular polyribonucleotide provided herein comprises an antisense strand of a lncRNA.

The circular polyribonucleotide may encode a regulatory nucleic acid substantially complementary, or fully complementary, to all or a fragment of an endogenous gene or gene product (e.g., mRNA). The regulatory nucleic acids may complement sequences at the boundary between introns and exons, in between exons, or adjacent to exon, to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof. In some embodiments, the regulatory nucleic acid comprises a protein-binding site that can bind to a protein that participates in regulation of expression of an endogenous gene or an exogenous gene.

The length of the circular polyribonucleotide may encode a regulatory nucleic acid that hybridizes to a transcript of interest that is between about 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

The circular polyribonucleotide may encode a micro RNA (miRNA) molecule identical to about 5 to about 25 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

In some embodiments, the circular polyribonucleotide comprises at least one miRNA, e.g., 2, 3, 4, 5, 6, or more. In some embodiments, the circular polyribonucleotide comprises a sequence that encodes an miRNA at least about 75%, 80%, 85%, 90% 95%, 96%, 97%, 98%, 99% or 100% nucleotide sequence identity to any one of the nucleotide sequences or a sequence that is complementary to a target sequence.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA’s 5′ end (Rajewsky, Nat Genet 38 Suppl:S8-13, 2006; Lim et al., Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Lagana et al., Methods Mol. Bio., 2015, 1269:393-412).

The circular polyribonucleotide may modulate expression of RNA encoded by a gene. Because multiple genes can share some degree of sequence homology with each other, in some embodiments, the circular polyribonucleotide can be designed to target a class of genes with sufficient sequence homology. In some embodiments, the circular polyribonucleotide can contain a sequence that has complementarity to sequences that are shared amongst different gene targets or are unique for a specific gene target. In some embodiments, the circular polyribonucleotide can be designed to target conserved regions of an RNA sequence having homology between several genes thereby targeting several genes in a gene family (e.g., different gene isoforms, splice variants, mutant genes, etc.). In some embodiments, the circular polyribonucleotide can be designed to target a sequence that is unique to a specific RNA sequence of a single gene.

In some embodiments, the expression sequence has a length less than 5000bps (e.g., less than about 5000bps, 4000bps, 3000bps, 2000bps, 1000bps, 900bps, 800bps, 700bps, 600bps, 500bps, 400bps, 300bps, 200bps, 100bps, 50bps, 40bps, 30bps, 20bps, 10bps, or less). In some embodiments, the expression sequence has, independently or in addition to, a length greater than 10bps (e.g., at least about 10bps, 20bps, 30bps, 40bps, 50bps, 60bps, 70bps, 80bps, 90bps, 100bps, 200bps, 300bps, 400bps, 500bps, 600bps, 700bps, 800bps, 900bps, 1000kb, 1.1kb, 1.2kb, 1.3kb, 1.4kb, 1.5kb, 1.6kb, 1.7kb, 1.8kb, 1.9kb, 2kb, 2.1kb, 2.2kb, 2.3kb, 2.4kb, 2.5kb, 2.6kb, 2.7kb, 2.8kb, 2.9kb, 3kb, 3.1kb, 3.2kb, 3.3kb, 3.4kb, 3.5kb, 3.6kb, 3.7kb, 3.8kb, 3.9kb, 4kb, 4.1kb, 4.2kb, 4.3kb, 4.4kb, 4.5kb, 4.6kb, 4.7kb, 4.8kb, 4.9kb, 5kb or greater).

In some embodiments, the expression sequence comprises one or more of the features described herein, e.g., a sequence encoding one or more peptides or proteins, one or more regulatory element, one or more regulatory nucleic acids, e.g., one or more non-coding RNAs, other expression sequences, and any combination thereof.

### RNA-Binding

In some embodiments, the circular polyribonucleotide comprises one or more RNA binding sites. microRNAs (or miRNA) are short noncoding RNAs that bind to the 3′UTR of nucleic acid molecules and down-regulate gene expression either by reducing nucleic acid molecule stability or by inhibiting translation. The circular polyribonucleotide may comprise one or more microRNA target sequences, microRNA sequences, or microRNA seeds. Such sequences may correspond to any known microRNA, such as those taught in U.S. Publication 2005/0261218 and U.S. Publication 2005/0059005, the contents of which are incorporated herein by reference in their entirety.

A microRNA sequence comprises a “seed” region, i.e., a sequence in the region of positions 2-8 of the mature microRNA, which sequence has perfect Watson- Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K, Johnston WK, Garrett-Engele P, Lim LP, Barrel DP; Mol Cell. 2007 Jul 6;27(1):91-105; each of which is herein incorporated by reference in their entirety.

The bases of the microRNA seed are substantially complementary with the target sequence. By engineering microRNA target sequences into the circular polyribonucleotide, the circular polyribonucleotide may evade or be detected by the host’s immune system, have modulated degradation, or modulated translation, provided the microRNA in question is available. This process will reduce the hazard of off target effects upon circular polyribonucleotide delivery. Identification of microRNA, microRNA target regions, and their expression patterns and role in biology have been reported (Bonauer et al., Curr Drug Targets 2010 11 :943-949; Anand and Cheresh Curr Opin Hematol 2011 18: 171- 176; Contreras and Rao Leukemia 2012 26:404-413 (2011 Dec 20. doi: 10.1038/leu.2011.356); Barrel Cell 2009 136:215-233; Landgrafet al, Cell, 2007 129: 1401-1414; each of which is herein incorporated by reference in its entirety).

Conversely, microRNA binding sites can be engineered out of (i.e. removed from) the circular polyribonucleotide to modulate protein expression in specific tissues. Regulation of expression in multiple tissues can be accomplished through introduction or removal or one or several microRNA binding sites.

Examples of tissues where microRNA are known to regulate mRNA, and thereby protein expression, include, but are not limited to, liver (miR-122), muscle (miR- 133, miR-206, miR-208), endothelial cells (miR-17-92, miR-126), myeloid cells (miR- 142-3p, miR-142-5p, miR-16, miR-21, miR-223, miR-24, miR-27), adipose tissue (let-7, miR-30c), heart (miR-ld, miR-149), kidney (miR-192, miR-194, miR-204), and lung epithelial cells (let-7, miR-133, miR-126). MicroRNA can also regulate complex biological processes such as angiogenesis (miR-132) (Anand and Cheresh Curr Opin Hematol 2011 18: 171-176; herein incorporated by reference in its entirety). In the circular polyribonucleotide described herein, binding sites for microRNAs that are involved in such processes may be removed or introduced, in order to tailor the expression from the circular polyribonucleotide to biologically relevant cell types or to the context of relevant biological processes. A listing of MicroRNA, miR sequences and miR binding sites is listed in Table 9 of U.S. Provisional Application No. 61/753,661 filed Jan. 17, 2013, in Table 9 of U.S. Provisional Application No. 61/754,159 filed Jan. 18, 2013, and in Table 7 of U.S. Provisional Application No. 61/758,921 filed Jan. 31, 2013, each of which are herein incorporated by reference in their entireties. In some embodiments, the microRNA binding site includes, e.g. miR-7.

The circular polyribonucleotide disclosed herein can comprise a miRNA binding site that hybridize to any miRNA, such as any of those disclosed in miRNA databases such as miRBase, deepBase, miRBase, microRNA.org, miRGen 2.0; miRNAMap, PMRD, TargetScan, or VIRmiRNA. In some cases, the miRNA binding site can any site that is complementary to an miRNA whose target gene is disclosed in microRNA target gene datasese such as StarBase, StarScan, Cupid, TargetScan, TarBase, Diana-microT, miRecords, PicTar, PITA, RepTarm RNA22, miRTarBase, miRwalk, or MBSTAR.

Through an understanding of the expression patterns of microRNA in different cell types, the circular polyribonucleotide described herein can be engineered for more targeted expression in specific cell types or only under specific biological conditions. Through introduction of tissue-specific microRNA binding sites, the circular polyribonucleotide can be designed for optimal protein expression in a tissue or in the context of a biological condition. Examples of use of microRNA to drive tissue or disease-specific gene expression are listed (Getner and Naldini, Tissue Antigens. 2012, 80:393-403; herein incorporated by reference in its entirety).

In addition, microRNA seed sites may be incorporated into the circular polyribonucleotide to modulate expression in certain cells which results in a biological improvement. An example of this is incorporation of miR-142 sites. Incorporation of miR-142 sites into the circular polyribonucleotide described herein may modulate expression in hematopoietic cells, but also reduce or abolish immune responses to a protein encoded in the circular polyribonucleotide.

In some embodiments, the circular polyribonucleotide includes one or more large intergenic non-coding RNAs (lincRNA) binding sites. Large intergenic non-coding RNAs (lincRNAs) make up most of the long non-coding RNAs. LincRNAs are non-coding transcripts and, in some embodiments, are more than about 200 nucleotides long. In some embodiments, they have an exon-intron-exon structure, similar to protein-coding genes, but do not encompass open-reading frames and do not code for proteins. More than 8,000 lincRNAs have been described recently and are thought to be the largest subclass of RNAs, originating from the non-coding transcriptome in humans. Thousands of lincRNAs are known and some appear to be key regulators of diverse cellular processes. Determining the function of individual lincRNAs remains a challenge. 1incRNA expression is strikingly tissue specific compared to coding genes, and that they are typically co-expressed with their neighboring genes, albeit to a similar extent to that of pairs of neighboring protein-coding genes.

In some embodiments, the circular polyribonucleotide includes one or more lincRNAs, such as FIRRE, LINC00969, PVT1, LINC01608, JPX, LINC01572, LINC00355, Clorf132, C3orf35, RP11-734, LINC01608, CC-499B15.5, CASC15, LINC00937, RP11-191, etc., or other lincRNAs or lncRNAs such as those from known lncRNA databases.

### Protein-Binding

In some embodiments, the circular polyribonucleotide includes one or more protein binding sites that enable a protein, e.g., a ribosome, to bind to an internal site in the RNA sequence. By engineering protein binding sites, e.g., ribosome binding sites, into the circular polyribonucleotide, the circular polyribonucleotide may evade or have reduced detection by the host’s immune system, have modulated degradation, or modulated translation, by masking the circular polyribonucleotide from components of the host’s immune system.

In some embodiments, the circular polyribonucleotide comprises at least one immunoprotein binding site, for example to evade immune responses, e.g., CTL (cytotoxic T lymphocyte) responses. In some embodiments, the immunoprotein binding site is a nucleotide sequence that binds to an immunoprotein and aids in masking the circular polyribonucleotide as exogenous. In some embodiments, the immunoprotein binding site is a nucleotide sequence that binds to an immunoprotein and aids in hiding the circular polyribonucleotide as exogenous or foreign.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present invention, internal initiation (i.e., cap-independent) of translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences comprising a ribosome binding site, e.g., an initiation codon.

Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

In some embodiments, the circular polyribonucleotide encodes a protein binding sequence that binds to a protein. In some embodiments, the protein binding sequence targets or localizes the circular polyribonucleotide to a specific target. In some embodiments, the protein binding sequence specifically binds an arginine-rich region of a protein.

In some embodiments, the protein binding site includes, but is not limited to, a binding site to the protein such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO-, NOP58, NPM1, NUDT21, PCBP2, POLR2A, PRPF8, PTBP1, RBFOX2, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

### Riboswitches

In some embodiments, the circular polyribonucleotide comprises one or more riboswitches.

A riboswitch is typically considered a part of the circular polyribonucleotide that can directly bind a small target molecule, and whose binding of the target affects RNA translation, the expression product stability and activity (Tucker B J, Breaker R R (2005), Curr Opin Struct Biol 15 (3): 342-8). Thus, the circular polyribonucleotide that includes a riboswitch is directly involved in regulating its own activity, depending on the presence or absence of its target molecule. In some embodiments, a riboswitch has a region of aptamer-like affinity for a separate molecule. Thus, in the broader context of the instant invention, any aptamer included within a non-coding nucleic acid could be used for sequestration of molecules from bulk volumes. Downstream reporting of the event via “(ribo)switch” activity may be especially advantageous.

In some embodiments, the riboswitch may have an effect on gene expression including, but not limited to, transcriptional termination, inhibition of translation initiation, mRNA self-cleavage, and in eukaryotes, alteration of splicing pathways. The riboswitch may function to control gene expression through the binding or removal of a trigger molecule. Thus, subjecting a circular polyribonucleotide that includes the riboswitch to conditions that activate, deactivate or block the riboswitch to alter expression. Expression can be altered as a result of, for example, termination of transcription or blocking of ribosome binding to the RNA. Binding of a trigger molecule or an analog thereof can, depending on the nature of the riboswitch, reduce or prevent expression of the RNA molecule or promote or increase expression of the RNA molecule. Some examples of riboswitches are described herein.

In some embodiments, the riboswitch is a Cobalamin riboswitch (also B₁₂-element), which binds adenosylcobalamin (the coenzyme form of vitamin B₁₂) to regulate the biosynthesis and transport of cobalamin and similar metabolites.

In some embodiments, the riboswitch is a cyclic di-GMP riboswitches, which bind cyclic di-GMP to regulate a variety of genes. Two non-structurally related classes exist - cyclic di-GMP-1 and cyclic di-GMP-ll.

In some embodiments, the riboswitch is a FMN riboswitch (also RFN-element) which binds flavin mononucleotide (FMN) to regulate riboflavin biosynthesis and transport.

In some embodiments, the riboswitch is a glmS riboswitch, which cleaves itself when there is a sufficient concentration of glucosamine-6-phosphate.

In some embodiments, the riboswitch is a Glutamine riboswitches, which bind glutamine to regulate genes involved in glutamine and nitrogen metabolism. They also bind short peptides of unknown function. Such riboswitches fall into two classes, which are structurally related: the glnA RNA motif and Downstream-peptide motif.

In some embodiments, the riboswitch is a Glycine riboswitch, which binds glycine to regulate glycine metabolism genes. It comprises two adjacent aptamer domains in the same mRNA, and is the only known natural RNA that exhibits cooperative binding.

In some embodiments, the riboswitch is a Lysine riboswitch (also L-box), which binds lysine to regulate lysine biosynthesis, catabolism and transport.

In some embodiments, the riboswitch is a PreQ 1 riboswitch, which binds pre-queuosine to regulate genes involved in the synthesis or transport of this precursor to queuosine. Two entirely distinct classes of PreGI riboswitches are known: PreQ 1 -1 riboswitches and PreQl-11 riboswitches. The binding domain of PreQ 1 -1 riboswitches is unusually small among naturally occurring riboswitches. PreGI -II riboswitches, which are only found in certain species in the genera Streptococcus and Lactococcus, have a completely different structure, and are larger.

In some embodiments, the riboswitch is a Purine riboswitch, which binds purines to regulate purine metabolism and transport. Different forms of the purine riboswitch bind guanine (a form originally known as the G-box) or adenine. The specificity for either guanine or adenine depends completely upon Watson- Crick interactions with a single pyrimidine in the riboswitch at position Y74. In the guanine riboswitch, this residue is a cytosine (i.e. C74), in the adenine residue it is always a uracil (i.e. U74). Homologous types of purine riboswitches bind deoxyguanosine, but have more significant differences than a single nucleotide mutation.

In some embodiments, the riboswitch is a SAH riboswitch, which binds S-adenosylhomocysteine to regulate genes involved in recycling this metabolite which is produced when S-adenosylmethionine is used in methylation reactions.

In some embodiments, the riboswitch is a SAM riboswitch, which binds S-adenosyl methionine (SAM) to regulate methionine and SAM biosynthesis and transport. Three distinct SAM riboswitches are known: SAM-I (originally called S-box), SAM-II and the S_{M}K box riboswitch. SAM-I is widespread in bacteria, but SAM-II is found only in a-, β- and a few y-proteobacteria. The S_{M}K box riboswitch is found only in the order Lactobacillales. These three varieties of riboswitch have no obvious similarities in terms of sequence or structure. A fourth variety, SAM-IV, appears to have a similar ligand-binding core to that of SAM-I, but in the context of a distinct scaffold.

In some embodiments, the riboswitch is a SAM-SAH riboswitch, which binds both SAM and SAH with similar affinities. Since they are always found in a position to regulate genes encoding methionine adenosyltransferase, it was proposed that only their binding to SAM is physiologically relevant.

In some embodiments, the riboswitch is a Tetrahydrofolate riboswitch, which binds tetrahydrofolate to regulate synthesis and transport genes.

In some embodiments, the riboswitch is a theophylline binding riboswitch or a thymine pyrophosphate binding riboswitch.

In some embodiments, the riboswitch is a T. tengcongensis glmS catalytic riboswitch, which senses glucosamine-6 phosphate (Klein and Ferre-D′Amare 2006).

In some embodiments, the riboswitch is a TPP riboswitch (also THI-box), which binds thiamine pyrophosphate (TPP) to regulate thiamine biosynthesis and transport, as well as transport of similar metabolites. It is the only riboswitch found so far in eukaryotes.

In some embodiments, the riboswitch is a Moco riboswitch, which binds molybdenum cofactor, to regulate genes involved in biosynthesis and transport of this coenzyme, as well as enzymes that use it or its derivatives as a cofactor.

In some embodiments, the riboswitch is a Adenine sensing add-A riboswitch, found in the 5′ UTR of the adenine deaminase encoding gene of Vibrio vulnificus.

### Aptazyme

In some embodiments, the circular polyribonucleotide comprises an aptazyme. Aptazyme is a switch for conditional expression in which an aptamer region is used as an allosteric control element and coupled to a region of catalytic RNA (a “ribozyme” as described below). In some embodiments, the aptazyme is active in cell type specific translation. In some embodiments, the aptazyme is active under cell state specific translation, e.g., virally infected cells or in the presence of viral nucleic acids or viral proteins.

A ribozyme (from ribonucleic acid enzyme, also called RNA enzyme or catalytic RNA) is a RNA molecule that catalyzes a chemical reaction. Many natural ribozymes catalyze either the hydrolysis of one of their own phosphodiester bonds, or the hydrolysis of bonds in other RNAs, but they have also been found to catalyze the aminotransferase activity of the ribosome. More recently it has been shown that catalytic RNAs can be “evolved” by in vitro methods [1. Agresti J J, Kelly B T, Jaschke A, Griffiths A D: Selection of ribozymes that catalyse multiple-turnover Diels-Alder cycloadditions by using in vitro compartmentalization. Proc Natl Acad Sci USA 2005, 102: 16170-16175; 2. Sooter L J, Riedel T, Davidson E A, Levy M, Cox J C, Ellington A D: Toward automated nucleic acid enzyme selection. Biological Chemistry 2001, 382(9): 1327-1334.]. Winkler et al. have shown [Winkler W C, Nahvi A, Roth A, Collins J A, Breaker R R: Control of gene expression by a natural metabolite-responsive ribozyme. Nature 2004, 428:281-286.] that, similar to riboswitch activity discussed above, ribozymes and their reaction products can regulate gene expression. In the context of the instant invention, it may be particularly advantageous to place a catalytic RNA or ribozyme within a larger non-coding RNA such that the ribozyme is present at many copies within the cell for the purposes of chemical transformation of a molecule from a bulk volume. Furthermore, encoding both aptamers and ribozymes in the same non-coding RNA may be particularly advantageous.

Some nonlimiting examples of ribozymes include hammerhead ribozyme, VL ribozyme, leadzyme, hairpin ribozyme.

In some embodiments, the aptazyme is a ribozyme that can cleave RNA sequences and which can be regulated as a result of binding ligand/modulator. The ribozyme may also be a self-cleaving ribozyme. As such, they combine the properties of ribozymes and aptamers. Aptazymes offer advantages over conventional aptamers due to their potential for activity in trans, the fact that they act catalytically to inactivate expression and that inactivation, due to cleavage of their own or heterologous transcript, is irreversible.

In some embodiments, the aptazyme is included in an untranslated region of the circular polyribonucleotide and in the absence of ligand/modulator is inactive, allowing expression of the transgene. Expression can be turned off (or down-regulated) by addition of the ligand. It should be noted that aptazymes which are downregulated in response to the presence of a particular modulator can be used in control systems where upregulation of gene expression in response to modulator is desired.

Aptazymes may also permit development of systems for self-regulation of circular polyribonucleotide expression. For example, the protein product of the circular polyribonucleotide is the rate determining enzyme in the synthesis of a particular small molecule could be modified to include an aptazyme selected to have increased catalytic activity in the presence of that molecule, thereby providing an autoregulatory feedback loop for its synthesis. Alternatively, the aptazyme activity can be selected to be sensitive to accumulation of the protein product from the circular polyribonucleotide, or any other cellular macromolecule.

In some embodiments, the circular polyribonucleotide may include an aptamer sequence. Some nonlimiting examples include an RNA aptamer binding lysozyme, a Toggle-25t which is an RNA aptamer that includes 2′fluoropyrimidine nucleotides bind thrombins with high specificity and affinity, RNATat that binds human immunodeficiency virus trans-acting responsive element (HIV TAR), RNA aptamer-binding hemin, RNA aptamer-binding interferon y, RNA aptamer binding vascular endothelial growth factor (VEGF), RNA aptamer binding prostate specific antigen (PSA), RNA aptamer binding dopamine, and RNA aptamer binding the non-classical oncogene, heat shock factor 1 (HSF1). Replication element

The circular polyribonucleotide as described herein can further encode a sequence and/or motifs useful for replication. Replication of a circular polyribonucleotide may occur by generating a complement circular polyribonucleotide. In some embodiments, the circular polyribonucleotide includes a motif to initiate transcription, where transcription is driven by either endogenous cellular machinery (DNA-dependent RNA polymerase) or an RNA-depended RNA polymerase encoded by the circular polyribonucleotide. The product of rolling-circle transcriptional event may be cut by a ribozyme to generate either complementary or propagated circular polyribonucleotide at unit length. The ribozymes may be encoded by the circular polyribonucleotide, its complement, or by an RNA sequence in trans. In some embodiments, the encoded ribozymes may include a sequence or motif that regulates (inhibits or promotes) activity of the ribozyme to control circular RNA propagation. In some embodiments, unit-length sequences may be ligated into a circular form by a cellular RNA ligase. In some embodiments, the circular polyribonucleotide includes a replication element that aids in self amplification. Examples of such replication elements include, but are not limited to, HDV replication domains described elsewhere herein, RNA promotor of Potato Spindle Tuber Viroid (see for example Kolonko 2005 Virology), and replication competent circular RNA sense and/or antisense ribozymes such as antigenomic 5′-

CGGGUCGGCAUGGCAUCUCCACCUCCUCGCGGUCCGACCUGGGCAUCCGA
AGGAGGACGCACGUCCACUCGGAUGGCUAAGGGAGAGCCA-3′ or genomic 5′-
UGGCCGGCAUGGUCCCAGCCUCCUCGCUGGCGCCGGCUGGGCAACAUUCC
GAGGGGACCGUCCCCUCGGUAAUGGCGAAUGGGACCCA-3′ .

In some embodiments, the circular polyribonucleotide includes at least one stagger element as described herein to aid in replication. A stagger element within the circular polyribonucleotide can cleave long transcripts replicated from the circular polyribonucleotide to a specific length that could subsequently circularize to form a complement to the circular polyribonucleotide.

In another embodiment, the circular polyribonucleotide includes at least one ribozyme sequence to cleave long transcripts replicated from the circular polyribonucleotide to a specific length, where another encoded ribozyme cuts the transcripts at the ribozyme sequence. Circularization forms a complement to the circular polyribonucleotide.

In some embodiments, the circular polyribonucleotide is substantially resistant to degradation, e.g., by exonucleases.

In some embodiments, the circular polyribonucleotide replicates within a cell. In some embodiments, the circular polyribonucleotide replicates within in a cell at a rate of between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween. In some embodiments, the circular polyribonucleotide is replicated within a cell and is passed to daughter cells. In some embodiments, a cell passes at least one circular polyribonucleotide to daughter cells with an efficiency of at least 25%, 50%, 60%, 70%, 80%, 85%, 90%, 95%, or 99%. In some embodiments, cell undergoing meiosis passes the circular polyribonucleotide to daughter cells with an efficiency of at least 25%, 50%, 60%, 70%, 80%, 85%, 90%, 95%, or 99%. In some embodiments, a cell undergoing mitosis passes the circular polyribonucleotide to daughter cells with an efficiency of at least 25%, 50%, 60%, 70%, 80%, 85%, 90%, 95%, or 99%.

In some embodiments, the circular polyribonucleotide replicates within the host cell. In one embodiment, the circular polyribonucleotide is capable of replicating in a mammalian cell, e.g., human cell.

While in some embodiments the circular polyribonucleotide replicates in the host cell, the circular polyribonucleotide does not integrate into the genome of the host, e.g., with the host’s chromosomes. In some embodiments, the circular polyribonucleotide has a negligible recombination frequency, e.g., with the host’s chromosomes. In some embodiments, the circular polyribonucleotide has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host’s chromosomes.

### Other Sequences

In some embodiments, the circular polyribonucleotide as described herein further includes another nucleic acid sequence. In some embodiments, the circular polyribonucleotide may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In one embodiment, the circular polyribonucleotide includes an siRNA to target a different loci of the same gene expression product as the circular polyribonucleotide. In one embodiment, the circular polyribonucleotide includes an siRNA to target a different gene expression product as the circular polyribonucleotide.

In some embodiments, the circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent that is comparable to or similar to in the absence of exonuclease. In some embodiments, the circular polyribonucleotide lacks degradation by exonucleases. In some embodiments, the circular polyribonucleotide has reduced degradation when exposed to exonuclease. In some embodiments, the circular polyribonucleotide lacks binding to a cap-binding protein In some embodiments, the circular polyribonucleotide lacks a 5′ cap.

In some embodiments, the circular polyribonucleotide lacks a 5′-UTR and is competent for protein express from its one or more expression sequences. In some embodiments, the circular polyribonucleotide lacks a 3′-UTR and is competent for protein express from its one or more expression sequences. In some embodiments, the circular polyribonucleotide lacks a poly-A sequence and is competent for protein express from its one or more expression sequences. In some embodiments, the circular polyribonucleotide lacks a termination element and is competent for protein express from its one or more expression sequences. In some embodiments, the circular polyribonucleotide lacks an internal ribosomal entry site and is competent for protein express from its one or more expression sequences. In some embodiments, the circular polyribonucleotide lacks a cap and is competent for protein express from its one or more expression sequences. In some embodiments, the circular polyribonucleotide lacks a 5′-UTR, a 3′-UTR, and an IRES, and is competent for protein express from its one or more expression sequences. In some embodiments, the circular polyribonucleotide comprises one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (siRNA, lncRNAs, shRNA), and a sequence that encodes a therapeutic mRNA or protein.

The other sequence may have a length from about 2 to about 10000 nts, about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

As a result of its circularization, the circular polyribonucleotide may include certain characteristics that distinguish it from linear RNA. For example, the circular polyribonucleotide is less susceptible to degradation by exonuclease as compared to linear RNA. As such, the circular polyribonucleotide is more stable than a linear RNA, especially when incubated in the presence of an exonuclease. The increased stability of the circular polyribonucleotide compared with linear RNA makes circular polyribonucleotide more useful as a cell transforming reagent to produce polypeptides and can be stored more easily and for longer than linear RNA. The stability of the circular polyribonucleotide treated with exonuclease can be tested using methods standard in art which determine whether RNA degradation has occurred (e.g., by gel electrophoresis).

Moreover, unlike linear RNA, the circular polyribonucleotide is less susceptible to dephosphorylation when the circular polyribonucleotide is incubated with phosphatase, such as calf intestine phosphatase.

### Nucleotide Spacer Sequences

In some embodiments, the circular polyribonucleotide as described herein further comprises a spacer sequence.

In some embodiments, the circular polyribonucleotide comprises at least one spacer sequence. In some embodiments, the circular polyribonucleotide comprises 1, 2, 3, 4, 5, 6, 7 or more spacer sequences.

In some embodiments, the circular polyribonucleotide comprises a ratio of spacer sequence to non-spacer sequence of the circular polyribonucleotide, e.g., expression sequences, of about 0.05: 1, about 0.06: 1, about 0.07: 1,about 0.08: 1, about 0.09: 1, about 0.1:1, about 0.12: 1, about 0.125: 1, about 0.15: 1, about 0.175:1, about 0.2:1, about 0.225: 1, about 0.25: 1, about 0.3:1, about 0.35: 1, about 0.4:1, about 0.45: 1, about 0.5:1, about 0.55: 1, about 0.6:1, about 0.65: 1, about 0.7:1, about 0.75: 1, about 0.8:1, about 0.85: 1, about 0.9:1, about 0.95: 1, about 0.98: 1, about 1:1, about 1.02:1, about 1.05: 1, about 1.1:1, about 1.15: 1, about 1.2: 1, about 1.25: 1, about 1.3:1, about 1.35: 1, about 1.4:1, about 1.45: 1, about 1.5:1, about 1.55: 1, about 1.6:1, about 1.65: 1, about 1.7:1, about 1.75: 1, about 1.8:1, about 1.85: 1, about 1.9:1, about 1.95: 1, about 1.975: 1, about 1.98: 1, or about 2:1.

In some embodiments, the spacer sequence comprises a ratio of spacer sequence to a downstream (e.g., 3′ of the spacer sequence) non-spacer element of the circular polyribonucleotide of about 0.5:1, about 0.06:1, about 0.07: 1,about 0.08: 1, about 0.09:1, about 0.1:1, about 0.12: 1, about 0.125:1, about 0.15: 1, about 0.175: 1, about 0.2:1, about 0.225: 1, about 0.25: 1, about 0.3:1, about 0.35: 1, about 0.4:1, about 0.45:1, about 0.5:1, about 0.55: 1, about 0.6:1, about 0.65: 1, about 0.7:1, about 0.75: 1, about 0.8:1, about 0.85:1, about 0.9:1, about 0.95: 1, about 0.98: 1, about 1:1, about 1.02: 1, about 1.05: 1, about 1.1:1, about 1.15:1, about 1.2:1, about 1.3:1, about 1.4:1, about 1.5:1, about 1.6:1, about 1.7:1, about 1.8:1, about 1.9:1, about 1.95: 1, about 1.975:1, about 1.98: 1, about 2.1: 1, about 2.2: 1, about 2.3: 1, about 2.4: 1, about 2.5:1, about 2.6:1, about 2.7:1, about 2.8:1, about 2.9:1, about 3: 1, about 3.1:1, about 3.2:1, about 3.3:1, about 3.4:1, about 3.5:1, about 3.6:1, about 3.7:1, about 3.8:1, about 3.85:1,about 3.9:1, about 3.95: 1, about 3.98: 1, or about 4:1. In some embodiments, the spacer sequence comprises a ratio of spacer sequence to an upstream (e.g., 5′ of the spacer sequence) non-spacer element of the circular polyribonucleotide of about 0.5:1, about 0.06: 1, about 0.07: 1,about 0.08: 1, about 0.09: 1, about 0.1: 1, about 0.12:1, about 0.125:1, about 0.15:1, about 0.175:1, about 0.2:1, about 0.225:1, about 0.25:1, about 0.3:1, about 0.35:1, about 0.4:1, about 0.45:1, about 0.5:1, about 0.55:1, about 0.6:1, about 0.65:1, about 0.7:1, about 0.75:1, about 0.8:1, about 0.85:1, about 0.9:1, about 0.95:1, about 0.98:1, about 1:1, about 1.02: 1, about 1.05:1, about 1.1:1, about 1.15:1, about 1.2:1, about 1.3:1, about 1.4:1, about 1.5:1, about 1.6:1, about 1.7:1, about 1.8:1, about 1.9:1, about 1.95:1, about 1.975:1, about 1.98:1, about 2.1:1, about 2.2:1, about 2.3:1, about 2.4:1, about 2.5:1, about 2.6:1, about 2.7:1, about 2.8:1, about 2.9:1, about 3:1, about 3.1:1, about 3.2:1, about 3.3:1, about 3.4:1, about 3.5:1, about 3.6:1, about 3.7:1, about 3.8:1, about 3.85: 1,about 3.9:1, about 3.95:1, about 3.98:1, or about 4:1.

In some embodiments, the spacer sequence comprises a sequence of at least 3 ribonucleotides, at least 4 ribonucleotides, at least 5 ribonucleotides, at least about 8 ribonucleotides, at least about 10 ribonucleotides, at least about 12 ribonucleotides, at least about 15 ribonucleotides, at least about 20 ribonucleotides, at least about 25 ribonucleotides, at least about 30 ribonucleotides, at least about 40 ribonucleotides, at least about 50 ribonucleotides, at least about 60 ribonucleotides, at least about 70 ribonucleotides, at least about 80 ribonucleotides, at least about 90 ribonucleotides, at least about 100 ribonucleotides, at least about 120 ribonucleotides, at least about 150 ribonucleotides, at least about 200 ribonucleotides, at least about 250 ribonucleotides, at least about 300 ribonucleotides, at least about 400 ribonucleotides, at least about 500 ribonucleotides, at least about 600 ribonucleotides, at least about 700 ribonucleotides, at least about 800 ribonucleotides, at least about 900 ribonucleotides, or at least about 100 ribonucleotides.

In some embodiments, the spacer sequence may be a nucleic acid sequence or molecule having low GC content, for example less than 65%, 60%, 55%, 50%, 55%, 50%, 45%, 40%, 39%, 38%, 37%, 36%, 35%, 34%, 33%, 32%, 31%, 30%, 29%, 28%, 27%, 26%, 25%, 24%, 23%, 22%, 20%, 19%, 18%, 17%, 16%, 15%, 14%, 13%, 12%, 11%, 10%, 9%, 8%, 7%, 6%, 5%, 4%, 3%, 2% or 1%, across the full length of the spacer, or across at least 50%, 60%, 70%, 80%, 90%, 91%, 92%, 93%, 94%, 95%, 96%, 97%, 98% or 99% contiguous nucleic acid residues of the spacer. In some embodiments, the spacer sequence may comprise at least 95%, 90%, 85%, 80%, 75%, 70%, 65%, 60%, 55%, 50%, 55%, 50%, 45%, 40%, 35%, 30%, 20% or any percentage therebetween of adenine ribonucleotides. In some embodiments, the spacer sequence comprises at least 5 or more adenine ribonucleotides in a row. In some embodiments, the spacer sequence comprises at least 6 adenine ribonucleotides in a row, at least 7 adenine ribonucleotides in a row, at least 8 ribonucleotides, at least about 10 adenine ribonucleotides in a row, at least about 12 adenine ribonucleotides in a row, at least about 15 adenine ribonucleotides in a row, at least about 20 adenine ribonucleotides in a row, at least about 25 adenine ribonucleotides in a row, at least about 30 adenine ribonucleotides in a row, at least about 40 adenine ribonucleotides in a row, at least about 50 adenine ribonucleotides in a row, at least about 60 adenine ribonucleotides in a row, at least about 70 adenine ribonucleotides in a row, at least about 80 adenine ribonucleotides in a row, at least about 90 adenine ribonucleotides in a row, at least about 95 adenine ribonucleotides in a row, at least about 100 adenine ribonucleotides in a row, at least about 150 adenine ribonucleotides in a row, at least about 200 adenine ribonucleotides in a row, at least about 250 adenine ribonucleotides in a row, at least about 300 adenine ribonucleotides in a row, at least about 350 adenine ribonucleotides in a row, at least about 400 adenine ribonucleotides in a row, at least about 450 adenine ribonucleotides in a row, at least about 500 adenine ribonucleotides in a row, at least about 550 adenine ribonucleotides in a row, at least about 600 adenine ribonucleotides in a row, at least about 700 adenine ribonucleotides in a row, at least about 800 adenine ribonucleotides in a row, at least about 900 adenine ribonucleotides in a row, or at least about 1000 adenine ribonucleotides in a row.

In some embodiments, the spacer sequence is situated between one or more elements. In some embodiments, the spacer sequence provides conformational flexibility between the elements. In some embodiments, the conformational flexibility is due to the spacer sequence being substantially free of a secondary structure. In some embodiments, the spacer sequence is substantially free of a secondary structure, such as less than 40kcal/mol, less than -39, -38, -37, -36, - 35, -34, -33, -32, -31, -30, -29, -28, -27, -26, -25, -24, -23, -22, -20, -19, -18, -17, -16, -15, -14, -13, - 12, -11, -10, -9, -8, -7, -6, -5, -4, -3, -2 or -1 kcal/mol. The spacer may include a nucleic acid, such as DNA or RNA.

In some embodiments, the spacer sequence may encode an RNA sequence, and preferably a protein or peptide sequence, including a secretion signal peptide.

In some embodiments, the spacer sequence may be non-coding. Where the spacer is a non-coding sequence, a translation initiation sequence may be provided in the coding sequence of an adjacent sequence. In some embodiments, it is envisaged that the first nucleic acid residue of the coding sequence may be the A residue of a translation initiation sequence, such as AUG. Where the spacer encodes an RNA or protein or peptide sequence, a translation initiation sequence may be provided in the spacer sequence.

In some embodiments, the spacer is operably linked to another sequence described herein.

### Non-Nucleic Acid Linkers

The circular polyribonucleotide described herein may further comprise a non-nucleic acid linker. In some embodiments, the circular polyribonucleotide described herein has a non-nucleic acid linker between one or more of the sequences or elements described herein. In one embodiment, one or more sequences or elements described herein are linked with the linker. The non-nucleic acid linker may be a chemical bond, e.g., one or more covalent bonds or non-covalent bonds. In some embodiments, the non-nucleic acid linker is a peptide or protein linker. Such a linker may be between 2-30 amino acids, or longer. The linker includes flexible, rigid or cleavable linkers described herein.

The most commonly used flexible linkers have sequences consisting primarily of stretches of Gly and Ser residues (“GS” linker). Flexible linkers may be useful for joining domains that require a certain degree of movement or interaction and may include small, non-polar (e.g., Gly) or polar (e.g., Ser or Thr) amino acids. Incorporation of Ser or Thr can also maintain the stability of the linker in aqueous solutions by forming hydrogen bonds with the water molecules, and therefore reduce unfavorable interactions between the linker and the protein moieties.

Rigid linkers are useful to keep a fixed distance between domains and to maintain their independent functions. Rigid linkers may also be useful when a spatial separation of the domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains in vivo. In some embodiments, linkers may be cleaved under specific conditions, such as the presence of reducing reagents or proteases. In vivo cleavable linkers may utilize the reversible nature of a disulfide bond. One example includes a thrombin-sensitive sequence (e.g., PRS) between the two Cys residues. In vitro thrombin treatment of CPRSC results in the cleavage of the thrombin-sensitive sequence, while the reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under pathological conditions (e.g. cancer or inflammation), in specific cells or tissues, or constrained within certain cellular compartments. The specificity of many proteases offers slower cleavage of the linker in constrained compartments.

Examples of linking molecules include a hydrophobic linker, such as a negatively charged sulfonate group; lipids, such as a poly (--CHz--) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more polypeptides. Non-covalent linkers are also included, such as hydrophobic lipid globules to which the polypeptide is linked, for example through a hydrophobic region of the polypeptide or a hydrophobic extension of the polypeptide, such as a series of residues rich in leucine, isoleucine, valine, or perhaps also alanine, phenylalanine, or even tyrosine, methionine, glycine or other hydrophobic residue. The polypeptide may be linked using charge-based chemistry, such that a positively charged moiety of the polypeptide is linked to a negative charge of another polypeptide or nucleic acid.

### Stability/Half-Life

In some embodiments, the circular polyribonucleotide provided herein has increase half-life over a reference, e.g., a linear polyribonucleotide having the same nucleotide sequence but is not circularized (linear counterpart). In some embodiments, the circular polyribonucleotide is substantially resistant to degradation, e.g., exonuclease. In some embodiments, the circular polyribonucleotide is resistant to self-degradation. In some embodiments, the circular polyribonucleotide lacks an enzymatic cleavage site, e.g., a dicer cleavage site. In some embodiments, the circular polyribonucleotide has a half-life at least about 5%, at least about 10%, at least about 20%, at least about 30%, at least about 40%, at least about 50%, at least about 60%, at least about 70%, at least about 80%, at least about 90%, at least about 100%, at least about 120%, at least about 140%, at least about 150%, at least about 160%, at least about 180%, at least about 200%, at least about 300%, at least about 400%, at least about 500%, at least about 600%, at least about 700% at least about 800%, at least about 900%, at least about 1000% or at least about 10000%, longer than a reference, e.g., a linear counterpart.

In some embodiments, the circular polyribonucleotide persists in a cell during cell division. In some embodiments, the circular polyribonucleotide persists in daughter cells after mitosis. In some embodiments, the circular polyribonucleotide is replicated within a cell and is passed to daughter cells. In some embodiments, the circular polyribonucleotide comprises a replication element that mediates self-replication of the circular polyribonucleotide. In some embodiments, the replication element mediates transcription of the circular polyribonucleotide into a linear polyribonucleotide that is complementary to the circular polyribonucleotide (linear complementary). In some embodiments, the linear complementary polyribonucleotide can be circularized in vivo in cells into a complementary circular polyribonucleotide. In some embodiments, the complementary polyribonucleotide can further self-replicate into another circular polyribonucleotide, which has the same or similar nucleotide sequence as the starting circular polyribonucleotide. One exemplary self-replication element includes HDV replication domain (as described by Beeharry et al, Virol, 2014, 450-451: 165-173). In some embodiments, a cell passes at least one circular polyribonucleotide to daughter cells with an efficiency of at least 25%, 50%, 60%, 70%, 80%, 85%, 90%, 95%, or 99%. In some embodiments, cell undergoing meiosis passes the circular polyribonucleotide to daughter cells with an efficiency of at least 25%, 50%, 60%, 70%, 80%, 85%, 90%, 95%, or 99%. In some embodiments, a cell undergoing mitosis passes the circular polyribonucleotide to daughter cells with an efficiency of at least 25%, 50%, 60%, 70%, 80%, 85%, 90%, 95%, or 99%.

### Methods of Production

The circular polyribonucleotides as described herein can be produced as follows from a linear version of the circular polyribonucleotide as described herein. In some embodiments, the circular polyribonucleotide includes a deoxyribonucleic acid sequence that is non-naturally occurring and can be produced using recombinant technology (methods described in detail below; e.g., derived in vitro using a DNA plasmid) or chemical synthesis.

It is within the scope of the invention that a DNA molecule used to produce an RNA circle can comprise a DNA sequence of a naturally-occurring original nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

The circular polyribonucleotide may be prepared according to any available technique including, but not limited to chemical synthesis and enzymatic synthesis. In some embodiments, a linear primary construct or linear mRNA may be cyclized, or concatemerized to create a circular polyribonucleotide described herein. The mechanism of cyclization or concatemerization may occur through methods such as, but not limited to, chemical, enzymatic, splint ligation), or ribozyme catalyzed methods. The newly formed 5 ′-/3 ′-linkage may be an intramolecular linkage or an intermolecular linkage.

Methods of making the circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Various methods of synthesizing circular polyribonucleotides are also described in the art (see, e.g., U.S. Pat. No. 6210931, U.S. Pat. No. 5773244, U.S. Pat. No. 5766903, U.S. Pat. No. 5712128, U.S. Pat. No. 5426180, U.S. Publication No. 20100137407, International Publication No. WO 1992001813 and International Publication No. W02010084371; the contents of each of which are herein incorporated by reference in their entireties).

In some embodiments, the circular polyribonucleotides may be cleaned up after production to remove production impurities, e.g., free ribonucleic acids, linear or nicked RNA, DNA, proteins, etc. In some embodiments, the circular polyribonucleotides may be purified by any known method commonly used in the art. Examples of nonlimiting purification methods include, column chromatography, gel excision, size exclusion, etc.

### Circularization

The circular polyribonucleotides as described herein can be circularized as follows from a linear version of the circular polyribonucleotide as described herein. In one embodiment, a linear circular polyribonucleotide may be cyclized, or concatemerized. In some embodiments, the linear circular polyribonucleotide may be cyclized in vitro prior to formulation and/or delivery. In some embodiments, the linear circular polyribonucleotide may be cyclized within a cell.

### Extracellular Circularization

In some embodiments, the linear circular polyribonucleotide is cyclized, or concatemerized using a chemical method to form a circular polyribonucleotide. In some chemical methods, the 5′-end and the 3′-end of the nucleic acid (e.g., a linear circular polyribonucleotide) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In one embodiment, a DNA or RNA ligase may be used to enzymatically link a 5′-phosphorylated nucleic acid molecule (e.g., a linear circular polyribonucleotide) to the 3′-hydroxyl group of a nucleic acid (e.g., a linear nucleic acid) forming a new phosphorodiester linkage. In an example reaction, a linear circular polyribonucleotide is incubated at 37° C. for 1 hour with 1-10 units of T4 RNA ligase (New England Biolabs, Ipswich, MA) according to the manufacturer’s protocol. The ligation reaction may occur in the presence of a linear nucleic acid capable of base-pairing with both the 5′- and 3′- region in juxtaposition to assist the enzymatic ligation reaction. In one embodiment, the ligation is splint ligation. For example, a splint ligase, like SplintR® ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In one embodiment, a DNA or RNA ligase may be used in the synthesis of the circular polynucleotides. As a non-limiting example, the ligase may be a circ ligase or circular ligase.

In one embodiment, either the 5′-or 3′-end of the linear circular polyribonucleotide can encode a ligase ribozyme sequence such that during in vitro transcription, the resultant linear circular polyribonucleotide includes an active ribozyme sequence capable of ligating the 5′-end of the linear circular polyribonucleotide to the 3′-end of the linear circular polyribonucleotide. The ligase ribozyme may be derived from the Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In one embodiment, a linear circular polyribonucleotide may be cyclized or concatermerized by using at least one non-nucleic acid moiety. In one aspect, the at least one non-nucleic acid moiety may react with regions or features near the 5′ terminus and/or near the 3′ terminus of the linear circular polyribonucleotide in order to cyclize or concatermerize the linear circular polyribonucleotide. In another aspect, the at least one non-nucleic acid moiety may be located in or linked to or near the 5′ terminus and/or the 3′ terminus of the linear circular polyribonucleotide. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In one embodiment, a linear circular polyribonucleotide may be cyclized or concatermerized due to a non-nucleic acid moiety that causes an attraction between atoms, molecular surfaces at, near or linked to the 5′ and 3′ ends of the linear circular polyribonucleotide. As a non-limiting example, one or more linear circular polyribonucleotides may be cyclized or concatermized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In one embodiment, the linear circular polyribonucleotide may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′ terminus may associate with each other causing a linear circular polyribonucleotide to cyclize or concatemerize. In another aspect, the peptides covalently linked to the ribozyme RNA near the 5′ terminus and the 3′ terminus may cause the linear primary construct or linear mRNA to cyclize or concatemerize after being subjected to ligated using various methods known in the art such as, but not limited to, protein ligation. Non-limiting examples of ribozymes for use in the linear primary constructs or linear RNA of the present invention or a non-exhaustive listing of methods to incorporate and/or covalently link peptides are described in U.S. Pat. Application No. US20030082768, the contents of which is here in incorporated by reference in its entirety.

In some embodiments, the linear circular polyribonucleotide may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear circular polyribonucleotide into a 5′ monophosphate may occur by a two-step reaction comprising: (a) contacting the 5′ nucleotide of the linear circular polyribonucleotide with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%.

### Splicing Element

In some embodiment, the circular polyribonucleotide includes at least one splicing element. In a circular polyribonucleotide as provided herein, a splicing element can be a complete splicing element that can mediate splicing of the circular polyribonucleotide. Alternatively, the spicing element can also be a residual splicing element from a completed splicing event. For instance, in some cases, a splicing element of a linear polyribonucleotide can mediate a splicing event that results in circularization of the linear polyribonucleotide, thereby the resultant circular polyribonucleotide comprises a residual splicing element from such splicing-mediated circularization event. In some cases, the residual splicing element is not able to mediate any splicing. In other cases, the residual splicing element can still mediate splicing under certain circumstances. In some embodiments, the splicing element is adjacent to at least one expression sequence. In some embodiments, the circular polyribonucleotide includes a splicing element adjacent each expression sequence. In some embodiments, the splicing element is on one or both sides of each expression sequence, leading to separation of the expression products, e.g., peptide(s) and or polypeptide(s).

In some embodiments, the circular polyribonucleotide includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, Alu elements in flanking introns, and motifs found in (suptable4 enriched motifs) cis-sequence elements proximal to backsplice events such as sequences in the 200 bp preceding (upstream of) or following (downstream from) a backsplice site with flanking exons. In some embodiments, the circular polyribonucleotide includes at least one repetitive nucleotide sequence described elsewhere herein as an internal splicing element. In such embodiments, the repetitive nucleotide sequence may include repeated sequences from the Alu family of introns. In some embodiments, a splicing-related ribosome binding protein can regulate circular polyribonucleotide biogenesis (e.g. the Muscleblind and Quaking (QKI) splicing factors).

In some embodiments, the circular polyribonucleotide may include canonical splice sites that flank head-to-tail junctions of the circular polyribonucleotide.

In some embodiments, the circular polyribonucleotide may include a bulge-helix-bulge motif, comprising a 4-base pair stem flanked by two 3-nucleotide bulges. Cleavage occurs at a site in the bulge region, generating characteristic fragments with terminal 5′-hydroxyl group and 2′, 3′-cyclic phosphate. Circularization proceeds by nucleophilic attack of the 5′-OH group onto the 2′, 3′-cyclic phosphate of the same molecule forming a 3′, 5′-phosphodiester bridge.

In some embodiments, the circular polyribonucleotide may include a multimeric repeating RNA sequence that harbors a HPR element. The HPR comprises a 2′,3′-cyclic phosphate and a 5′-OH termini. The HPR element self-processes the 5′- and 3′-ends of the linear circular polyribonucleotide, thereby ligating the ends together.

In some embodiments, the circular polyribonucleotide may include a sequence that mediates self-ligation. In one embodiment, the circular polyribonucleotide may include a HDV sequence (e.g., HDV replication domain conserved sequence,

GGCUCAUCUCGACAAGAGGCGGCAGUCCUCAGUACUCUUACUCUUUUCUG
UAAAGAGGAGACUGCUGGACUCGCCGCCCAAGUUCGAGCAUGAGCC
or
GGCUAGAGGCGGCAGUCCUCAGUACUCUUACUCUUUUCUGUAAAGAGGAG
ACUGCUGGACUCGCCGCCCGAGCC)

to self-ligate. In one embodiment, the circular polyribonucleotide may include loop E sequence (e.g., in PSTVd) to self-ligate. In another embodiment, the circular polyribonucleotide may include a self-circularizing intron, e.g., a 5′ and 3′ slice junction, or a self-circularizing catalytic intron such as a Group 1, Group II or Group III Introns. Nonlimiting examples of group I intron self-splicing sequences may include self-splicing permuted intron-exon sequences derived from T4 bacteriophage gene td, and the intervening sequence (IVS) rRNA of Tetrahymena.

### Other Circularization Methods

In some embodiments, linear circular polyribonucleotides may include complementary sequences, including either repetitive or nonrepetitive nucleic acid sequences within individual introns or across flanking introns. Repetitive nucleic acid sequence are sequences that occur within a segment of the circular polyribonucleotide. In some embodiments, the circular polyribonucleotide includes a repetitive nucleic acid sequence. In some embodiments, the repetitive nucleotide sequence includes poly CA or poly UG sequences. In some embodiments, the circular polyribonucleotide includes at least one repetitive nucleic acid sequence that hybridizes to a complementary repetitive nucleic acid sequence in another segment of the circular polyribonucleotide, with the hybridized segment forming an internal double strand. In some embodiments, repetitive nucleic acid sequences and complementary repetitive nucleic acid sequences from two separate circular polyribonucleotides hybridize to generate a single circularized polyribonucleotide, with the hybridized segments forming internal double strands. In some embodiments, the complementary sequences are found at the 5′ and 3′ ends of the linear circular polyribonucleotides. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the circular polyribonucleotide. Such methods may include, but are not limited to click chemistry (e.g., alkyne and azide based methods, or clickable bases), olefin metathesis, phosphoramidate ligation, hemiaminal-imine crosslinking, base modification, and any combination thereof.

In some embodiments, enzymatic methods of circularization may be used to generate the circular polyribonucleotide. In some embodiments, a ligation enzyme, e.g., DNA or RNA ligase, may be used to generate a template of the circular polyribonuclease or complement, a complementary strand of the circular polyribonuclease, or the circular polyribonuclease.

Circularization of the circular polyribonucleotide may be accomplished by methods known in the art, for example, those described in “RNA circularization strategies in vivo and in vitro” by Petkovic and Muller from Nucleic Acids Res, 2015, 43(4): 2454-2465, and “In vitro circularization of RNA” by Muller and Appel, from RNA Biol, 2017, 14(8): 1018-1027.

The circular polyribonucleotide may encode a sequence and/or motifs useful for replication. Exemplary replication elements include binding sites for RNA polymerase. Other types of replication elements are described in paragraphs [0280] - [0286] of WO2019/118919, which is hereby incorporated by reference in its entirety. In some embodiments, the circular polyribonucleotide as disclosed herein lacks a replication element, e.g., lacks an RNA-dependent RNA polymerase binding site.

In some embodiments, the circular polyribonucleotide lacks a poly-A sequence and a replication element.

### Translation Efficiency

In some embodiments, the translation efficiency of a circular polyribonucleotide as provided herein is greater than a reference, e.g., a linear counterpart, a linear expression sequence, or a linear circular polyribonucleotide. In some embodiments, a circular polyribonucleotide as provided herein has the translation efficiency that is at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 450%, 500%, 600%, 70%, 800%, 900%, 1000%, 2000%, 5000%, 10000%, 100000%, or more greater than that of a reference. In some embodiments, a circular polyribonucleotide has a translation efficiency 10% greater than that of a linear counterpart. In some embodiments, a circular polyribonucleotide has a translation efficiency 300% greater than that of a linear counterpart.

In some embodiments, the circular polyribonucleotide produces stoichiometric ratios of expression products. Rolling circle translation continuously produces expression products at substantially equivalent ratios. In some embodiments, the circular polyribonucleotide has a stoichiometric translation efficiency, such that expression products are produced at substantially equivalent ratios. In some embodiments, the circular polyribonucleotide has a stoichiometric translation efficiency of multiple expression products, e.g., products from 2, 3, 4, 5, 6, 7, 8, 9, 10, 11, 12, or more expression sequences.

### Rolling Circle Translation

In some embodiments, once translation of the circular polyribonucleotide is initiated, the ribosome bound to the circular polyribonucleotide does not disengage from the circular polyribonucleotide before finishing at least one round of translation of the circular polyribonucleotide. In some embodiments, the circular polyribonucleotide as described herein is competent for rolling circle translation. In some embodiments, during rolling circle translation, once translation of the circular polyribonucleotide is initiated, the ribosome bound to the circular polyribonucleotide does not disengage from the circular polyribonucleotide before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 10⁵ rounds, or at least 10⁶ rounds of translation of the circular polyribonucleotide.

In some embodiments, the rolling circle translation of the circular polyribonucleotide leads to generation of polypeptide product that is translated from more than one round of translation of the circular polyribonucleotide (“continuous” expression product). In some embodiments, the circular polyribonucleotide comprises a stagger element, and rolling circle translation of the circular polyribonucleotide leads to generation of polypeptide product that is generated from a single round of translation or less than a single round of translation of the circular polyribonucleotide (“discrete” expression product). In some embodiments, the circular polyribonucleotide is configured such that at least 10%, 20%, 30%, 40%, 50%, at least 60%, at least 70%, at least 80%, at least 90%, at least 95%, at least 96%, at least 97%, at least 98%, at least 99%, or 100% of total polypeptides (molar/molar) generated during the rolling circle translation of the circular polyribonucleotide are discrete polypeptides. In some embodiments, the amount ratio of the discrete products over the total polypeptides is tested in an in vitro translation system. In some embodiments, the in vitro translation system used for the test of amount ratio comprises rabbit reticulocyte lysate. In some embodiments, the amount ratio is tested in an in vivo translation system, such as a eukaryotic cell or a prokaryotic cell, a cultured cell or a cell in an organism.

### Modifications

In some aspects, the disclosure provides compositions and methods that comprise modified capped polyribonucleotides and modified circular polyribonucleotides. The term “modified nucleotide” refers to any nucleotide analog or derivative that has one or more chemical modifications to the chemical composition of an unmodified natural ribonucleotide, such as a natural unmodified nucleotide adenosine (A), uridine (U), guaninie (G), cytidine (C) as shown by the chemical formulae in Table 10, and monophosphate. The chemical modifications of the modified ribonucleotide can be modifications to any one or more functional groups of the ribonucleotide, such as, the sugar the nucleobase, or the internucleoside linkage (e.g. to a linking phosphate / to a phosphodiester linkage / to the phosphodiester backbone).

TABLE 10

Unmodified Natural Ribonucleosides

| | | |
|---|---|---|
| Ribonucleoside | IUPAC name | Chemical Formula |
| Adenosine | (2R,3R,4S,5R)-2-(6-amino-9H-purin-9-yl)-5-(hydroxymethyl)oxolane-3,4-diol | 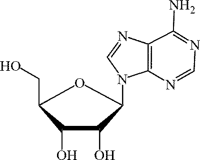 |
| | | C10H13N5O4 |
| Uridine | 1-[(3R,4S,5R)-3,4-dihydroxy-5-(hydroxymethyl)oxolan-2-yl]pyrimidine-2,4-dione | 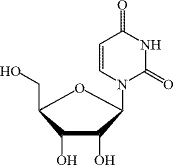 |
| | | C₉H₁₂N₂O₆ |
| Guanine | 2-amino-9H-purin-6(1H)-one | 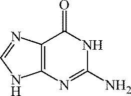 [SMILES: Nc1nc2[nH]cnc2c(=O)[nH]1] |
| | | C₅H₅N₅O |
| Cytidine | 4-amino-1-[(2R,3R,4S,5R)-3,4-dihydroxy-5-(hydroxymethyl)oxolan-2-yl]pyrimidin-2(1H)-one | 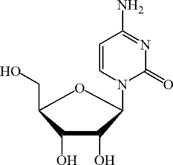 |
| | | C₉H₁₃N₃O₅ |

The polyribonucleotide of the capped polyribonucleotide as described herein can comprise one or more substitutions, insertions and/or additions, deletions, and covalent modifications with respect to reference sequences, in particular, the parent polyribonucleotide, are included within the scope of this invention. The circular polyribonucleotide as described herein can comprise one or more substitutions, insertions and/or additions, deletions, and covalent modifications with respect to reference sequences, in particular, the parent polyribonucleotide, are included within the scope of this invention.

In some embodiments, the polyribonucleotide of the capped polyribonucleotide or the circular polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The one or more post-transcriptional modifications can be any post-transcriptional modification, such as any of the more than one hundred different nucleoside modifications that have been identified in RNA ( Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyluridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-azacytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methylguanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The polyribonucleotide of the capped polyribonucleotide or circular polyribonucleotide may include any useful modification, such as to the sugar, the nucleobase, or the internucleoside linkage (e.g., to a linking phosphate / to a phosphodiester linkage / to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the polyribonucleotide of the capped polyribonucleotide or circular polyribonucleotide includes at least one N(6)methyladenosine (m6A) modification to increase translation efficiency. In some embodiments, the N(6)methyladenosine (m6A) modification can reduce immunogeneicity of the circular polyribonucleotide.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the ribonucleotides of the circular polyribonucleotide may enhance immune evasion. The circular polyribonucleotide may be synthesized and/or modified by methods well established in the art, such as those described in “Current protocols in nucleic acid chemistry,” Beaucage, S.L. et al. (Eds.), John Wiley & Sons, Inc., New York, NY, USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases may also include 5- methylcytidine and pseudouridine. In some embodiments, base modifications may modulate expression, immune response, stability, subcellular localization, to name a few functional effects, of the circular polyribonucleotide. In some embodiments, the modification includes a bi-orthogonal nucleotides, e.g., an unnatural base. See for example, Kimoto et al, Chem Commun (Camb), 2017, 53:12309, DOI: 10.1039/c7cc06661a, which is hereby incorporated by reference.

In some embodiments, sugar modifications (e.g., at the 2′ position or 4′ position) or replacement of the sugar one or more ribonucleotides of the polyribonucleotide of the capped polyribonucleotide or circular polyribonucleotide may, as well as backbone modifications, include modification or replacement of the phosphodiester linkages. Specific examples of polyribonucleotide of the capped polyribonucleotide or circular polyribonucleotide include, but are not limited to polyribonucleotide of the capped polyribonucleotide or circular polyribonucleotide including modified backbones or no natural internucleoside linkages such as internucleoside modifications, including modification or replacement of the phosphodiester linkages. Polyribonucleotide of the capped polyribonucleotide or circular polyribonucleotides having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, the polyribonucleotide of the capped polyribonucleotide or circular polyribonucleotide will include ribonucleotides with a phosphorus atom in its internucleoside backbone.

Modified polyribonucleotide of the capped polyribonucleotide or modified circular polyribonucleotide backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the circular polyribonucleotide may be negatively or positively charged.

The modified nucleotides, which may be incorporated into the polyribonucleotide of the capped polyribonucleotide or circular polyribonucleotide, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene -phosphonates).

The a-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the circular polyribonucleotide is expected to reduce the innate immune response through weaker binding/activation of cellular innate immune molecules.

In specific embodiments, a modified nucleoside includes an alpha-thio- nucleoside (e.g., 5′-0-(1-thiophosphate)-adenosine, 5′-0-(1-thiophosphate)-cytidine (a- thio-cytidine), 5′-0-(1-thiophosphate)-guanosine, 5′-0-(1-thiophosphate)-uridine, or 5′-0- (1 -thiophosphate)-pseudouridine).

Other internucleoside linkages that may be employed according to the present invention, including internucleoside linkages which do not contain a phosphorous atom, are described herein.

In some embodiments, the polyribonucleotide of the capped polyribonucleotide or circular polyribonucleotide may include one or more cytotoxic nucleosides. For example, cytotoxic nucleosides may be incorporated into circular polyribonucleotide, such as bifunctional modification. Cytotoxic nucleoside may include, but are not limited to, adenosine arabinoside, 5-azacytidine, 4′-thio- aracytidine, cyclopentenylcytosine, cladribine, clofarabine, cytarabine, cytosine arabinoside, 1-(2-C-cyano-2-deoxy-beta-D-arabino- pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(lH,3H)-dione), troxacitabine, tezacitabine, 2′- deoxy-2′-methylidenecytidine (DMDC), and 6-mercaptopurine. Additional examples include fludarabine phosphate, N4-behenoyl-1-beta-D-arabinofuranosylcytosine, N4-octadecyl- 1 -beta-D-arabinofuranosylcytosine, N4- palmitoyl-1-(2-C-cyano-2-deoxy-beta-D-arabino-pentofuranosyl) cytosine, and P-4055 (cytarabine 5′-elaidic acid ester).

The polyribonucleotide of the capped polyribonucleotide or circular polyribonucleotide may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the polyribonucleotide of the capped polyribonucleotide or circular polyribonucleotide, or in a given predetermined sequence region thereof. In some embodiments, the polyribonucleotide of the capped polyribonucleotide or circular polyribonucleotide includes a pseudouridine. In some embodiments, the polyribonucleotide of the capped polyribonucleotide or circular polyribonucleotide includes an inosine, which may aid in the immune system characterizing the polyribonucleotide of the capped polyribonucleotide or circular polyribonucleotide as endogenous versus viral RNAs. The incorporation of inosine may also mediate improved RNA stability/reduced degradation. See for example, Yu, Z. et al. (2015) RNA editing by ADAR1 marks dsRNA as “self. Cell Res. 25, 1283-1284, which is incorporated by reference in its entirety.

In some embodiments, all nucleotides in the polyribonucleotide of the capped polyribonucleotide or circular polyribonucleotide (or in a given sequence region thereof) are modified. In some embodiments, the modification may include an m6A, which may augment expression; an inosine, which may attenuate an immune response; pseudouridine, which may increase RNA stability, or translational readthrough (stagger element), an m5C, which may increase stability; and a 2,2,7-trimethylguanosine, which aids subcellular translocation (e.g., nuclear localization).

Different sugar modifications, nucleotide modifications, and/or internucleoside linkages (e.g., backbone structures) may exist at various positions in the circular polyribonucleotide. One of ordinary skill in the art will appreciate that the nucleotide analogs or other modification(s) may be located at any position(s) of the polyribonucleotide of the capped polyribonucleotide or circular polyribonucleotide, such that the function of the polyribonucleotide of the capped polyribonucleotide or circular polyribonucleotide is not substantially decreased. A modification may also be a non-coding region modification. The polyribonucleotide of the capped polyribonucleotide or circular polyribonucleotide may include from about 1% to about 100% modified nucleotides (either in relation to overall nucleotide content, or in relation to one or more types of nucleotide, i.e. any one or more of A, G, U or C) or any intervening percentage. For example, the polyribonucleotide of the capped polyribonucleotide comprisesfrom 1% to 20% modified nucleotides. The polyribonucleotide of the capped polyribonucleotide comprises from 1% to 25% modified nucleotides. The polyribonucleotide of the capped polyribonucleotide comprises from 1% to 50% modified nucleotides. The polyribonucleotide of the capped polyribonucleotide comprises from 1% to 60% modified nucleotides. The polyribonucleotide of the capped polyribonucleotide comprises from 1% to 70% modified nucleotides. The polyribonucleotide of the capped polyribonucleotide comprises from 1% to 80% modified nucleotides. The polyribonucleotide of the capped polyribonucleotide comprises from 1% to 90% modified nucleotides. The polyribonucleotide of the capped polyribonucleotide comprises from 1 % to 95% modified nucleotides. The polyribonucleotide of the capped polyribonucleotide comprises from 10% to 20% modified nucleotides. The polyribonucleotide of the capped polyribonucleotide comprises from 10% to 25% modified nucleotides. The polyribonucleotide of the capped polyribonucleotide comprises from 10% to 50% modified nucleotides. The polyribonucleotide of the capped polyribonucleotide comprises from 10% to 60% modified nucleotides. The polyribonucleotide of the capped polyribonucleotide comprises from 10% to 70% modified nucleotides. The polyribonucleotide of the capped polyribonucleotide comprises from 10% to 80% modified nucleotides. The polyribonucleotide of the capped polyribonucleotide comprises from 10% to 90% modified nucleotides. The polyribonucleotide of the capped polyribonucleotide comprises from 10% to 95% modified nucleotides. The polyribonucleotide of the capped polyribonucleotide comprises from 10% to 100% modified nucleotides. The polyribonucleotide of the capped polyribonucleotide comprises from 20% to 25% modified nucleotides. The polyribonucleotide of the capped polyribonucleotide comprises from 20% to 50% modified nucleotides. The polyribonucleotide of the capped polyribonucleotide comprises from 20% to 60% modified nucleotides. The polyribonucleotide of the capped polyribonucleotide comprises from 20% to 70% modified nucleotides. The polyribonucleotide of the capped polyribonucleotide comprises from 20% to 80% modified nucleotides. The polyribonucleotide of the capped polyribonucleotide comprises from 20% to 90% modified nucleotides. The polyribonucleotide of the capped polyribonucleotide comprises from 20% to 95% modified nucleotides. The polyribonucleotide of the capped polyribonucleotide comprises from 20% to 100% modified nucleotides. The polyribonucleotide of the capped polyribonucleotide comprises from 50% to 60% modified nucleotides. The polyribonucleotide of the capped polyribonucleotide comprises from 50% to 70% modified nucleotides. The polyribonucleotide of the capped polyribonucleotide comprises from 50% to 80% modified nucleotides. The polyribonucleotide of the capped polyribonucleotide comprises from 50% to 90% modified nucleotides. The polyribonucleotide of the capped polyribonucleotide comprises from 50% to 95% modified nucleotides. The polyribonucleotide of the capped polyribonucleotide comprises from 50% to 100% modified nucleotides. The polyribonucleotide of the capped polyribonucleotide comprises from 70% to 80% modified nucleotides. The polyribonucleotide of the capped polyribonucleotide comprises from 70% to 90% modified nucleotides. The polyribonucleotide of the capped polyribonucleotide comprises from 70% to 95% modified nucleotides. The polyribonucleotide of the capped polyribonucleotide comprises from 70% to 100% modified nucleotides. The polyribonucleotide of the capped polyribonucleotide comprises from 80% to 90% modified nucleotides. The polyribonucleotide of the capped polyribonucleotide comprises from 80% to 95% modified nucleotides. The polyribonucleotide of the capped polyribonucleotide comprises from 80% to 100% modified nucleotides. The polyribonucleotide of the capped polyribonucleotide comprises from 90% to 95% modified nucleotides. The polyribonucleotide of the capped polyribonucleotide comprises from 90% to 100% modified nucleotides. The polyribonucleotide of the capped polyribonucleotide comprises from 95% to 100% modified nucleotides. For example, the circular polyribonucleotide comprises from 1% to 20% modified nucleotides. The circular polyribonucleotide comprises from 1% to 25% modified nucleotides. The circular polyribonucleotide comprises from 1% to 50% modified nucleotides. The circular polyribonucleotide comprises from 1% to 60% modified nucleotides. The circular polyribonucleotide comprises from 1% to 70% modified nucleotides. The circular polyribonucleotide comprises from 1% to 80% modified nucleotides. The circular polyribonucleotide comprises from 1% to 90% modified nucleotides. The circular polyribonucleotide comprises from 1% to 95% modified nucleotides. The circular polyribonucleotide comprises from 10% to 20% modified nucleotides. The circular polyribonucleotide comprises from 10% to 25% modified nucleotides. The circular polyribonucleotide comprises from 10% to 50% modified nucleotides. The circular polyribonucleotide comprises from 10% to 60% modified nucleotides. The circular polyribonucleotide comprises from 10% to 70% modified nucleotides. The circular polyribonucleotide comprises from 10% to 80% modified nucleotides. The circular polyribonucleotide comprises from 10% to 90% modified nucleotides. The circular polyribonucleotide comprises from 10% to 95% modified nucleotides. The circular polyribonucleotide comprises from 10% to 100% modified nucleotides. The circular polyribonucleotide comprises from 20% to 25% modified nucleotides. The circular polyribonucleotide comprises from 20% to 50% modified nucleotides. The circular polyribonucleotide comprises from 20% to 60% modified nucleotides. The circular polyribonucleotide comprises from 20% to 70% modified nucleotides. The circular polyribonucleotide comprises from 20% to 80% modified nucleotides. The circular polyribonucleotide comprises from 20% to 90% modified nucleotides. The circular polyribonucleotide comprises from 20% to 95% modified nucleotides. The circular polyribonucleotide comprises from 20% to 100% modified nucleotides. circular polyribonucleotide comprises from 50% to 60% modified nucleotides. The circular polyribonucleotide comprises from 50% to 70% modified nucleotides. The circular polyribonucleotide comprises from 50% to 80% modified nucleotides. The circular polyribonucleotide comprises from 50% to 90% modified nucleotides. The circular polyribonucleotide comprises from 50% to 95% modified nucleotides. The circular polyribonucleotide comprises from 50% to 100% modified nucleotides. The circular polyribonucleotide comprises from 70% to 80% modified nucleotides. The circular polyribonucleotide comprises from 70% to 90% modified nucleotides. The circular polyribonucleotide comprises from 70% to 95% modified nucleotides. The circular polyribonucleotide comprises from 70% to 100% modified nucleotides. The circular polyribonucleotide comprises from 80% to 90% modified nucleotides. The circular polyribonucleotide comprises from 80% to 95% modified nucleotides. The circular polyribonucleotide comprises from 80% to 100% modified nucleotides. The circular polyribonucleotide comprises from 90% to 95% modified nucleotides. The circular polyribonucleotide comprises from 90% to 100% modified nucleotides. The circular polyribonucleotide comprises from 95% to 100% modified nucleotides.

### Complex

The present invention includes a method of producing a complex comprising binding a first binding region of a capped polyribonucleotide as described herein to a second binding region of a circular polyribonucleotide as described herein, thereby producing the complex. Furthermore, the present invention includes a composition comprising this complex, wherein the composition comprises a capped polyribonucleotide as described herein and the circular polyribonucleotide as described herein, wherein a first binding region of the capped polyribonucleotide is bound to the second binding region of the circular polyribonucleotide.

The present invention further includes a method of producing a complex comprising binding a first binding region of a first capped polyribonucleotide as described herein to a second binding region of a circular polyribonucleotide as described herein and binding a third binding region of a second capped polyribonucleotide as described herein to a fourth binding region of the circular polyribonucleotide, thereby producing the complex. Furthermore, the present invention includes a composition comprising this complex, wherein the composition comprises a first capped polyribonucleotide as described herein, a second capped polyribonucleotide as described herein, and the circular polyribonucleotide as described herein, wherein a first binding region of the first capped polyribonucleotide is bound to the second binding region of the circular polyribonucleotide and the third binding region of the second capped polyribonucleotide is bound to the fourth binding region of the circular polyribonucleotide.

The present invention further includes a method of producing a complex comprising binding a plurality of binding regions of a plurality of capped polyribonucleotides as described herein to a plurality of binding regions of a circular polyribonucleotide as described, thereby producing the complex. Furthermore, the present invention includes a composition comprising this complex, wherein the composition comprises a plurality of capped polynucleotides as described herein, and the circular polyribonucleotide as described herein, wherein a plurality of binding regions of the plurality of capped polynucleotides are bound to a plurality of binding regions of the circular polyribonucleotide.

In some embodiments, the production of the complex of a capped polyribonucleotide bound to a circular polyribonucleotide takes place in vitro. For instance, the first binding region of a capped polyribonucleotide is bound to the second binding region of a circular polyribonucleotide in vitro and then, the complex is administered to a cell, tissue, or a subject in need thereof. In some embodiments, the first binding region of a capped polyribonucleotide is bound to the second binding region of a circular polyribonucleotide in vitro and then, the complex is administered to a cell. In some embodiments, the first binding region of a capped polyribonucleotide is bound to the second binding region of a circular polyribonucleotide in vitro and then, the complex is administered to a tissue. In some embodiments, the first binding region of a capped polyribonucleotide is bound to the second binding region of a circular polyribonucleotide in vitro and then, the complex is administered to a subject in need thereof. In some embodiments, the production of the complex of a capped polyribonucleotide and a circular polyribonucleotide takes place in vivo. For example, a capped polyribonucleotide and a circular polyribonucleotide are administered to a cell, tissue, or to a subject in need thereof, and then, the first binding region of the capped polyribonucleotide is bound to the second binding region of a circular polyribonucleotide in vivo. For instance, the first binding region of a first capped polyribonucleotide is bound to the second binding region of a circular polyribonucleotide and the third binding region of a second capped polyribonucleotide is bound to the fourth binding region of the circular polyribonucleotide in vitro and then, the complex is administered to a cell, tissue, or a subject in need thereof. In some embodiments the first binding region of a first capped polyribonucleotide is bound to the second binding region of a circular polyribonucleotide and the third binding region of a second capped polyribonucleotide is bound to the fourth binding region of the circular polyribonucleotide in vitro and then, the complex is administered to a cell. In some embodiments, the first binding region of a first capped polyribonucleotide is bound to the second binding region of a circular polyribonucleotide and the third binding region of a second capped polyribonucleotide is bound to the fourth binding region of the circular polyribonucleotide in vitro and then, the complex is administered to a tissue. In some embodiments, the first binding region of a first capped polyribonucleotide is bound to the second binding region of a circular polyribonucleotide and the third binding region of a second capped polyribonucleotide is bound to the fourth binding region of the circular polyribonucleotide in vitro and then, the complex is administered to a subject in need thereof. In some embodiments, the production of the complex of a capped polyribonucleotide and a circular polyribonucleotide takes place in vivo. For example, a first capped polyribonucleotide, a second capped polyribonucleotide, and a circular polyribonucleotide are administered to a cell, tissue, or to a subject in need thereof, and then, the first binding region of the first capped polyribonucleotide is bound to the second binding region of a circular polyribonucleotide and the third binding region of the second capped polyribonucleotide is bound to the fourth binding region of the circular polyribonucleotide in vivo.

### Pharmaceutical Compositions

In some aspects, the invention described herein comprises pharmaceutical compositions comprising a capped polyribonucleotide as described herein and a circular polyribonucleotide as described herein. In some other aspects, the invention described herein comprises pharmaceutical compositions comprising a polyribonucleotide comprising a 5′ modified guanosine cap, and a circular polyribonucleotide. In some other aspects, the invention described herein comprises pharmaceutical compositions comprising a complex, wherein the complex comprises a capped polyribonucleotide as described herein and the circular polyribonucleotide as described herein, wherein a first binding region of the capped polyribonucleotide is bound to the second binding region of the circular polyribonucleotide to form the complex.

In some embodiments, the pharmaceutical composition further comprises a pharmaceutically acceptable excipient. A pharmaceutically acceptable excipient can be a non-carrier excipient. A non-carrier excipient serves as a vehicle or medium for a composition, such as a circular polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Pharmaceutical compositions described herein can be used in therapeutic and veterinary. In some embodiments, pharmaceutical compositions (e.g., comprising a circular polyribonucleotide and the capped polyribonucleotide as described herein) provided herein are suitable for administration to a subject, wherein the subject is a non-human animal, for example, suitable for veterinary use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, any animals, such as humans and/or other primates; mammals, including commercially relevant mammals, e.g., pet and live-stock animals, such as cattle, pigs, horses, sheep, goats, cats, dogs, mice, and/or rats; and/or birds, including commercially relevant birds such as parrots, poultry, chickens, ducks, geese, hens or roosters and/or turkeys; zoo animals, e.g., a feline; non-mammal animals, e.g., reptiles, fish, amphibians, etc..

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In some embodiments, the pharmaceutical compositions comprise molecules that contains the circular polyribonucleotide-binding moiety and the ribosome-binding moiety. In some embodiments, the circular polyribonucleotide-binding moiety and the ribosome-binding moiety are directly or indirectly linked or conjugated. In some embodiments, the circular polyribonucleotide-binding moiety and the ribosome-binding moiety are independently, for example, a polynucleotide, a polyribonucleotide, a polypeptide or protein, e.g., an antibody and a ribosome-binding protein, a small molecule, a carbohydrate, or a lipid. In some embodiments, the circular polyribonucleotide-binding moiety that is, for example, a polynucleotide, a polyribonucleotide, a polypeptide or protein, e.g., an antibody and a ribosome-binding protein, a small molecule, a carbohydrate, or a lipid, binds to the circular polyribonucleotide.

### Methods of Expression

The present invention includes a method for protein expression, comprising translating at least a region of the circular polyribonucleotide as provided herein using a capped polyribonucleotide as described herein. In some embodiments, the capped polyribonucleotide as described herein drives expression of the expression sequence in the circular polyribonucleotide by recruiting a ribosome. In some embodiments, the capped polyribonucleotide as described herein drives expression of the expression sequence in the circular polyribonucleotide when the capped polyribonucleotide is bound to the circular polyribonucleotide. In some embodiments, one or more capped polyribonucleotides as described herein drives expression of the expression sequence in the circular polyribonucleotide when the capped polyribonucleotides are bound to the circular polyribonucleotide.

In some embodiments, the administration of the circular polyribonucleotide is conducted using any delivery method described herein. In some embodiments, the circular polyribonucleotide is administered to the subject via intravenous injection. In some embodiments, the administration of the circular polyribonucleotide includes, but is not limited to, prenatal administration, neonatal administration, postnatal administration, oral, by injection (e.g., intravenous, intraarterial, intraperitoneal, intradermal, subcutaneous and intramuscular), by ophthalmic administration and by intranasal administration.

In some embodiments, the methods for protein expression comprise modification, folding, or other post-translation modification of the translation product. In some embodiments, the methods for protein expression comprise post-translation modification in vivo, e.g., via cellular machinery.

In some embodiments, the cell is a eukaryotic cell. In some embodiments, the cell is a mammalian cell. In some embodiments, the cell is a human cell. In some embodiments, the cell is an animal cell. In some embodiments, the cell is an immune cell. In some embodiments, the tissue is a connective tissue, a muscle tissue, a nervous tissue, or an epithelial tissue. In some embodiments, the tissue is an organ (e.g., liver, lung, spleen, kidney, etc.). In some embodiments, the subject is a mammal. In some embodiments, the subject is a human. In some embodiments, the subject is a pet. In some embodiments, the subject is a live-stock animal.

### Expression

In some aspects, the invention described herein comprises a method of expressing one or more expression sequences from a circular polyribonucleotide in a cell, tissue, or subject, comprising binding a first binding region of a capped polyribonucleotide as provided herein to a second binding region of a circular polyribonucleotide as provided herein to produce a complex, wherein the circular polyribonucleotide comprises the one or more expression sequences; and delivering the complex to the cell; wherein the complex affects expression of the one or more expression sequences of the circular polyribonucleotide in the cell. The complex can affect expression by increasing translation when the capped polyribonucleotide is bound to the circular polyribonucleotide compared to translation when from the circular polyribonucleotide in the absence of the capped polyribonucleotide.

In some other aspects, the invention described herein comprises a method of expressing one or more expression sequences from a circular polyribonucleotide in a cell, comprising delivering a capped polyribonucleotide as provided herein to the cell; and delivering a circular polyribonucleotide as provided herein comprising the one or more expression sequences to the cell; wherein the first binding region of a capped polyribonucleotide binds to the second binding region of a circular polyribonucleotide to produce a complex that affects expression of the one or more expression sequences of the circular polyribonucleotide in the cell. The complex can affect expression by increasing translation when the capped polyribonucleotide is bound to the circular polyribonucleotide compared to translation when from the circular polyribonucleotide in the absence of the capped polyribonucleotide.

In some aspects, the invention described herein comprises a method of expressing one or more expression sequences from a circular polyribonucleotide in a cell, tissue, or subject, comprising binding a first binding region of a first capped polyribonucleotide as provided herein to a second binding region of a circular polyribonucleotide as provided herein and a third binding region of a second capped polyribonucleotide as provided herein to a fourth binding region of the circular polyribonucleotide to produce a complex, wherein the circular polyribonucleotide comprises the one or more expression sequences; and delivering the complex to the cell; wherein the complex affects expression of the one or more expression sequences of the circular polyribonucleotide in the cell. The complex can affect expression by increasing translation when the first capped polyribonucleotide and the second capped polyribonucleotide are bound to the circular polyribonucleotide compared to translation when from the circular polyribonucleotide in the absence of the first capped polyribonucleotide. The complex can affect expression by increasing translation when the first capped polyribonucleotide and the second capped polyribonucleotide are bound to the circular polyribonucleotide compared to translation when from the circular polyribonucleotide in the absence of the second capped polyribonucleotide. The complex can affect expression by increasing translation when the first capped polyribonucleotide and the second capped polyribonucleotide are bound to the circular polyribonucleotide compared to translation when from the circular polyribonucleotide in the absence of the first capped polyribonucleotide and the second capped polyribonucleotide.

In some other aspects, the invention described herein comprises a method of expressing one or more expression sequences from a circular polyribonucleotide in a cell, comprising delivering a first capped polyribonucleotide and a second capped polyribonucleotide as provided herein to the cell; and delivering a circular polyribonucleotide as provided herein comprising the one or more expression sequences to the cell; wherein the first binding region of a first capped polyribonucleotide binds to the second binding region of a circular polyribonucleotide and the third binding region of a second capped polyribonucleotide binds to the fourth binding region of the circular polyribonucleotide to produce a complex that affects expression of the one or more expression sequences of the circular polyribonucleotide in the cell. he complex can affect expression by increasing translation when the first capped polyribonucleotide and the second capped polyribonucleotied are bound to the circular polyribonucleotide compared to translation when from the circular polyribonucleotide in the absence of the first capped polyribonucleotide. The complex can affect expression by increasing translation when the first capped polyribonucleotide and the second capped polyribonucleotied are bound to the circular polyribonucleotide compared to translation when from the circular polyribonucleotide in the absence of the second capped polyribonucleotide. The complex can affect expression by increasing translation when the first capped polyribonucleotide and the second capped polyribonucleotied are bound to the circular polyribonucleotide compared to translation when from the circular polyribonucleotide in the absence of the first capped polyribonucleotide and the second capped polyribonucleotide.

In some embodiments, the methods for protein expression comprises translation of at least 10%, at least 20%, at least 30%, at least 40%, at least 50%, at least 60%, at least 70%, at least 80%, at least 90%, or at least 95% of the total length of the circular polyribonucleotide into polypeptides. In some embodiments, the methods for protein expression comprises translation of the circular polyribonucleotide into polypeptides of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids. In some embodiments, the methods for protein expression comprises translation of the circular polyribonucleotide into polypeptides of at least 5 amino acids. In some embodiments, the methods for protein expression comprises translation of the circular polyribonucleotide into polypeptides of at least 10 amino acids. In some embodiments, the methods for protein expression comprises translation of the circular polyribonucleotide into polypeptides of at least 15 amino acids. In some embodiments, the methods for protein expression comprises translation of the circular polyribonucleotide into polypeptides of at least 20 amino acids. In some embodiments, the methods for protein expression comprises translation of the circular polyribonucleotide into polypeptides of at least 50 amino acids. In some embodiments, the methods for protein expression comprises translation of the circular polyribonucleotide into polypeptides of at least 100 amino acids. In some embodiments, the methods for protein expression comprises translation of the circular polyribonucleotide into polypeptides of at least 150 amino acids. In some embodiments, the methods for protein expression comprises translation of the circular polyribonucleotide into polypeptides of at least 200 amino acids. In some embodiments, the methods for protein expression comprises translation of the circular polyribonucleotide into polypeptides of at least 250 amino acids. In some embodiments, the methods for protein expression comprises translation of the circular polyribonucleotide into polypeptides of at least 300 amino acids. In some embodiments, the methods for protein expression comprises translation of the circular polyribonucleotide into polypeptides of at least 400 amino acids. In some embodiments, the methods for protein expression comprises translation of the circular polyribonucleotide into polypeptides of at least 500 amino acids. In some embodiments, the methods for protein expression comprises translation of the circular polyribonucleotide into polypeptides of at least 600 amino acids. In some embodiments, the methods for protein expression comprises translation of the circular polyribonucleotide into polypeptides of at least 700 amino acids. In some embodiments, the methods for protein expression comprises translation of the circular polyribonucleotide into polypeptides of at least 800 amino acids. In some embodiments, the methods for protein expression comprises translation of the circular polyribonucleotide into polypeptides of at least 900 amino acids. In some embodiments, the methods for protein expression comprises translation of the circular polyribonucleotide into polypeptides of at least 1000 amino acids. In some embodiments, the methods for protein expression comprises translation of the circular polyribonucleotide into polypeptides of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, or about 1000 amino acids. In some embodiments, the methods for protein expression comprises translation of the circular polyribonucleotide into polypeptides of about 5 amino acids. In some embodiments, the methods for protein expression comprises translation of the circular polyribonucleotide into polypeptides of about 10 amino acids. In some embodiments, the methods for protein expression comprises translation of the circular polyribonucleotide into polypeptides of about 15 amino acids. In some embodiments, the methods for protein expression comprises translation of the circular polyribonucleotide into polypeptides of about 20 amino acids. In some embodiments, the methods for protein expression comprises translation of the circular polyribonucleotide into polypeptides of about 50 amino acids. In some embodiments, the methods for protein expression comprises translation of the circular polyribonucleotide into polypeptides of about 100 amino acids. In some embodiments, the methods for protein expression comprises translation of the circular polyribonucleotide into polypeptides of about 150 amino acids. In some embodiments, the methods for protein expression comprises translation of the circular polyribonucleotide into polypeptides of about 200 amino acids. In some embodiments, the methods for protein expression comprises translation of the circular polyribonucleotide into polypeptides of about 250 amino acids. In some embodiments, the methods for protein expression comprises translation of the circular polyribonucleotide into polypeptides of about 300 amino acids. In some embodiments, the methods for protein expression comprises translation of the circular polyribonucleotide into polypeptides of about 400 amino acids. In some embodiments, the methods for protein expression comprises translation of the circular polyribonucleotide into polypeptides of about 500 amino acids. In some embodiments, the methods for protein expression comprises translation of the circular polyribonucleotide into polypeptides of about 600 amino acids. In some embodiments, the methods for protein expression comprises translation of the circular polyribonucleotide into polypeptides of about 700 amino acids. In some embodiments, the methods for protein expression comprises translation of the circular polyribonucleotide into polypeptides of about 800 amino acids. In some embodiments, the methods for protein expression comprises translation of the circular polyribonucleotide into polypeptides of about 900 amino acids. In some embodiments, the methods for protein expression comprises translation of the circular polyribonucleotide into polypeptides of about 1000 amino acids. In some embodiments, the methods comprise translation of the circular polyribonucleotide into continuous polypeptides as provided herein, discrete polypeptides as provided herein, or both.

In some embodiments, the translation of the at least a region of the circular polyribonucleotide takes place in vitro, such as rabbit reticulocyte lysate. In some embodiments, the translation of the at least a region of the circular polyribonucleotide takes place in vivo, for instance, after transfection of a eukaryotic cell, or transformation of a prokaryotic cell such as a bacteria.

In some aspects, the present disclosure provides methods of in vivo expression of one or more expression sequences in a subject, comprising: administering a capped polyribonucleotide and a circular polyribonucleotide to a cell of the subject wherein the circular polyribonucleotide comprises the one or more expression sequences; and expressing the one or more expression sequences from the circular polyribonucleotide in the cell.

### Increased in Vitro Expression

In some aspects, the invention as provided herein comprises a method of in vitro expression of one or more expression sequences, comprising: providing a complex comprising a circular polyribonucleotide as provided herein comprising the one or more expression sequences, and a capped polyribonucleotide as provided herein, wherein the first binding region of the capped polyribonucleotide is bound to the second binding region of the circular polyribonucleotide; and administering the complex to a cell in vitro, wherein expression of the one or more expression sequences from the complex in the cell is higher than expression from the circular polyribonucleotide alone (e.g., a composition lacking the capped polynucleotide).

In some other aspects, the invention as provided herein comprises a method of in vitro expression of one or more expression sequences, comprising: administering to a cell in vitro a circular polyribonucleotide as provided herein comprising the one or more expression sequences, and a capped polyribonucleotide as provided herein to a cell, wherein the first binding region of the capped polyribonucleotide is bound to the second binding region of the circular polyribonucleotide to form a complex in the cell and the expression of the one or more expression sequences from the complex in the cell is higher than expression from the circular polyribonucleotide alone (e.g., lacking the capped polyribonucleotide).

In some aspects, the invention as provided herein comprises a method of in vitro expression of one or more expression sequences, comprising: providing a complex comprising a circular polyribonucleotide as provided herein comprising the one or more expression sequences, a first capped polyribonucleotide as provided herein, wherein the first binding region of the first capped polyribonucleotide is bound to the second binding region of the circular polyribonucleotide, and a second capped polyribonucleotide as provided herein, wherein the third binding region of the second capped polyribonucleotide is bound to the fourth binding region of the circular polyribonucleotide; and administering the complex to a cell in vitro, wherein expression of the one or more expression sequences from the complex in the cell is higher than expression from the circular polyribonucleotide alone (e.g., a composition lacking the capped polynucleotide). In some aspects, the invention as provided herein comprises a method of in vitro expression of one or more expression sequences, comprising: providing a complex comprising a circular polyribonucleotide as provided herein comprising the one or more expression sequences, a first capped polyribonucleotide as provided herein, wherein the first binding region of the first capped polyribonucleotide is bound to the second binding region of the circular polyribonucleotide, and a second capped polyribonucleotide as provided herein, wherein the third binding region of the second capped polyribonucleotide is bound to the fourth binding region of the circular polyribonucleotide; and administering the complex to a cell in vitro, wherein expression of the one or more expression sequences from the complex in the cell is higher than expression from the circular polyribonucleotide bound to the first capped polyribonucleotide. In some aspects, the invention as provided herein comprises a method of in vitro expression of one or more expression sequences, comprising: providing a complex comprising a circular polyribonucleotide as provided herein comprising the one or more expression sequences, a first capped polyribonucleotide as provided herein, wherein the first binding region of the first capped polyribonucleotide is bound to the second binding region of the circular polyribonucleotide, and a second capped polyribonucleotide as provided herein, wherein the third binding region of the second capped polyribonucleotide is bound to the fourth binding region of the circular polyribonucleotide; and administering the complex to a cell in vitro, wherein expression of the one or more expression sequences from the complex in the cell is higher than expression from the circular polyribonucleotide bound to the second capped polyribonucleotide.

In some other aspects, the invention as provided herein comprises a method of in vitro expression of one or more expression sequences, comprising: administering to a cell in vitro a circular polyribonucleotide as provided herein comprising the one or more expression sequences, a first capped polyribonucleotide as provided herein to a cell, wherein the first binding region of the first capped polyribonucleotide is bound to the second binding region of the circular polyribonucleotide, and a second capped polyribonucleotide as provided herein to a cell, wherein the third binding region of the second capped polyribonucleotide is bound to the fourth binding region of the circular polyribonucleotide to form a complex in the cell and the expression of the one or more expression sequences from the complex in the cell is higher than expression from the circular polyribonucleotide alone (e.g., lacking the capped polyribonucleotide). In some other aspects, the invention as provided herein comprises a method of in vitro expression of one or more expression sequences, comprising: administering to a cell in vitro a circular polyribonucleotide as provided herein comprising the one or more expression sequences, a first capped polyribonucleotide as provided herein to a cell, wherein the first binding region of the first capped polyribonucleotide is bound to the second binding region of the circular polyribonucleotide, and a second capped polyribonucleotide as provided herein to a cell, wherein the third binding region of the second capped polyribonucleotide is bound to the fourth binding region of the circular polyribonucleotide to form a complex in the cell and the expression of the one or more expression sequences from the complex in the cell is higher than expression from the circular polyribonucleotide bound to the first capped polyribonucleotide. In some other aspects, the invention as provided herein comprises a method of in vitro expression of one or more expression sequences, comprising: administering to a cell in vitro a circular polyribonucleotide as provided herein comprising the one or more expression sequences, a first capped polyribonucleotide as provided herein to a cell, wherein the first binding region of the first capped polyribonucleotide is bound to the second binding region of the circular polyribonucleotide, and a second capped polyribonucleotide as provided herein to a cell, wherein the third binding region of the second capped polyribonucleotide is bound to the fourth binding region of the circular polyribonucleotide to form a complex in the cell and the expression of the one or more expression sequences from the complex in the cell is higher than expression from the circular polyribonucleotide bound to the second capped polyribonucleotide.

In some embodiments, the expression of the one or more expression sequences from the complex in the cell is at least 10%, 20%, 30%, 40%, 50%, 60%, 70%, 80%, 90%, 100%, 200%, 300%, 400%, 500%, 600%, 700%, 800%, 900%, 1000%, 5000%, 10000%, or more higher than the expression from the circular polyribonucleotide alone (e.g., lacking the capped polyribonucleotide). In some embodiments, the expression of the one or more expression sequences from the complex in the cell is at least 10% higher than the expression from the circular polyribonucleotide alone. In some embodiments, the expression of the one or more expression sequences from the complex in the cell is at least 20% higher than the expression from the circular polyribonucleotide alone. In some embodiments, the expression of the one or more expression sequences from the complex in the cell is at least 30% higher than the expression from the circular polyribonucleotide alone. In some embodiments, the expression of the one or more expression sequences from the complex in the cell is at least 40% higher than the expression from the circular polyribonucleotide alone. In some embodiments, the expression of the one or more expression sequences from the complex in the cell is at least 50% higher than the expression from the circular polyribonucleotide alone. In some embodiments, the expression of the one or more expression sequences from the complex in the cell is at least 60% higher than the expression from the circular polyribonucleotide alone. In some embodiments, the expression of the one or more expression sequences from the complex in the cell is at least 70% higher than the expression from the circular polyribonucleotide alone. In some embodiments, the expression of the one or more expression sequences from the complex in the cell is at least 80% higher than the expression from the circular polyribonucleotide alone. In some embodiments, the expression of the one or more expression sequences from the complex in the cell is at least 90% higher than the expression from the circular polyribonucleotide alone. In some embodiments, the expression of the one or more expression sequences from the complex in the cell is at least 100% higher than the expression from the circular polyribonucleotide alone. In some embodiments, the expression of the one or more expression sequences from the complex in the cell is at least 200% higher than the expression from the circular polyribonucleotide alone. In some embodiments, the expression of the one or more expression sequences from the complex in the cell is at least 300% higher than the expression from the circular polyribonucleotide alone. In some embodiments, the expression of the one or more expression sequences from the complex in the cell is at least 400% higher than the expression from the circular polyribonucleotide alone. In some embodiments, the expression of the one or more expression sequences from the complex in the cell is at least 500% higher than the expression from the circular polyribonucleotide alone. In some embodiments, the expression of the one or more expression sequences from the complex in the cell is at least 600% higher than the expression from the circular polyribonucleotide alone. In some embodiments, the expression of the one or more expression sequences from the complex in the cell is at least 700% higher than the expression from the circular polyribonucleotide alone. In some embodiments, the expression of the one or more expression sequences from the complex in the cell is at least 800% higher than the expression from the circular polyribonucleotide alone. In some embodiments, the expression of the one or more expression sequences from the complex in the cell is at least 900% higher than the expression from the circular polyribonucleotide alone. In some embodiments, the expression of the one or more expression sequences from the complex in the cell is at least 1000% higher than the expression from the circular polyribonucleotide alone. In some embodiments, the expression of the one or more expression sequences from the complex in the cell is at least 5000% higher than the expression from the circular polyribonucleotide alone. In some embodiments, the expression of the one or more expression sequences from the complex in the cell is at least 10000% higher than the expression from the circular polyribonucleotide alone. In some embodiments, the expression of the one or more expression sequences from the complex in the cell is 10%, 20%, 30%, 40%, 50%, 60%, 70%, 80%, 90%, 100%, 200%, 300%, 400%, 500%, 600%, 700%, 800%, 900%, 1000%, 5000%, 10000%, or more higher than the expression from the circular polyribonucleotide alone (e.g., lacking the capped polyribonucleotide). In some embodiments, the expression of the one or more expression sequences from the complex in the cell is 10% higher than the expression from the circular polyribonucleotide alone. In some embodiments, the expression of the one or more expression sequences from the complex in the cell is 20% higher than the expression from the circular polyribonucleotide alone. In some embodiments, the expression of the one or more expression sequences from the complex in the cell is 30% higher than the expression from the circular polyribonucleotide alone. In some embodiments, the expression of the one or more expression sequences from the complex in the cell is 40% higher than the expression from the circular polyribonucleotide alone. In some embodiments, the expression of the one or more expression sequences from the complex in the cell is 50% higher than the expression from the circular polyribonucleotide alone. In some embodiments, the expression of the one or more expression sequences from the complex in the cell is 60% higher than the expression from the circular polyribonucleotide alone. In some embodiments, the expression of the one or more expression sequences from the complex in the cell is 70% higher than the expression from the circular polyribonucleotide alone. In some embodiments, the expression of the one or more expression sequences from the complex in the cell is 80% higher than the expression from the circular polyribonucleotide alone. In some embodiments, the expression of the one or more expression sequences from the complex in the cell is 90% higher than the expression from the circular polyribonucleotide alone. In some embodiments, the expression of the one or more expression sequences from the complex in the cell is 100% higher than the expression from the circular polyribonucleotide alone. In some embodiments, the expression of the one or more expression sequences from the complex in the cell is 200% higher than the expression from the circular polyribonucleotide alone. In some embodiments, the expression of the one or more expression sequences from the complex in the cell is 300% higher than the expression from the circular polyribonucleotide alone. In some embodiments, the expression of the one or more expression sequences from the complex in the cell is 400% higher than the expression from the circular polyribonucleotide alone. In some embodiments, the expression of the one or more expression sequences from the complex in the cell is 500% higher than the expression from the circular polyribonucleotide alone. In some embodiments, the expression of the one or more expression sequences from the complex in the cell is 600% higher than the expression from the circular polyribonucleotide alone. In some embodiments, the expression of the one or more expression sequences from the complex in the cell is 700% higher than the expression from the circular polyribonucleotide alone. In some embodiments, the expression of the one or more expression sequences from the complex in the cell is 800% higher than the expression from the circular polyribonucleotide alone. In some embodiments, the expression of the one or more expression sequences from the complex in the cell is 900% higher than the expression from the circular polyribonucleotide alone. In some embodiments, the expression of the one or more expression sequences from the complex in the cell is 1000% higher than the expression from the circular polyribonucleotide alone. In some embodiments, the expression of the one or more expression sequences from the complex in the cell is 5000% higher than the expression from the circular polyribonucleotide alone. In some embodiments, the expression of the one or more expression sequences from the complex in the cell is 10000% higher than the expression from the circular polyribonucleotide alone. In some embodiments, the expression of the one or more expression sequences from the complex in the cell is higher than the expression from the circular polyribonucleotide alone (e.g., lacking a capped polynucleotide) at least by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 5000, 10000 fold or more. In some embodiments, the expression of the one or more expression sequences from the complex in the cell is higher than the expression from the circular polyribonucleotide alone (e.g., lacking a capped polynucleotide) at least by 2 fold. In some embodiments, the expression of the one or more expression sequences from the complex in the cell is higher than the expression from the circular polyribonucleotide alone (e.g., lacking a capped polynucleotide) at least by 3 fold. In some embodiments, the expression of the one or more expression sequences from the complex in the cell is higher than the expression from the circular polyribonucleotide alone (e.g., lacking a capped polynucleotide) at least by 4 fold. In some embodiments, the expression of the one or more expression sequences from the complex in the cell is higher than the expression from the circular polyribonucleotide alone (e.g., lacking a capped polynucleotide) at least by 5 fold. In some embodiments, the expression of the one or more expression sequences from the complex in the cell is higher than the expression from the circular polyribonucleotide alone (e.g., lacking a capped polynucleotide) at least by 6 fold. In some embodiments, the expression of the one or more expression sequences from the complex in the cell is higher than the expression from the circular polyribonucleotide alone (e.g., lacking a capped polynucleotide) at least by 7 fold. In some embodiments, the expression of the one or more expression sequences from the complex in the cell is higher than the expression from the circular polyribonucleotide alone (e.g., lacking a capped polynucleotide) at least by 8 fold. In some embodiments, the expression of the one or more expression sequences from the complex in the cell is higher than the expression from the circular polyribonucleotide alone (e.g., lacking a capped polynucleotide) at least by 9 fold. In some embodiments, the expression of the one or more expression sequences from the complex in the cell is higher than the expression from the circular polyribonucleotide alone (e.g., lacking a capped polynucleotide) at least by 10 fold. In some embodiments, the expression of the one or more expression sequences from the complex in the cell is higher than the expression from the circular polyribonucleotide alone (e.g., lacking a capped polynucleotide) at least by 15 fold. In some embodiments, the expression of the one or more expression sequences from the complex in the cell is higher than the expression from the circular polyribonucleotide alone (e.g., lacking a capped polynucleotide) at least by 20 fold. In some embodiments, the expression of the one or more expression sequences from the complex in the cell is higher than the expression from the circular polyribonucleotide alone (e.g., lacking a capped polynucleotide) at least by 25 fold. In some embodiments, the expression of the one or more expression sequences from the complex in the cell is higher than the expression from the circular polyribonucleotide alone (e.g., lacking a capped polynucleotide) at least by 30 fold. In some embodiments, the expression of the one or more expression sequences from the complex in the cell is higher than the expression from the circular polyribonucleotide alone (e.g., lacking a capped polynucleotide) at least by 35 fold. In some embodiments, the expression of the one or more expression sequences from the complex in the cell is higher than the expression from the circular polyribonucleotide alone (e.g., lacking a capped polynucleotide) at least by 40 fold. In some embodiments, the expression of the one or more expression sequences from the complex in the cell is higher than the expression from the circular polyribonucleotide alone (e.g., lacking a capped polynucleotide) at least by 45 fold. In some embodiments, the expression of the one or more expression sequences from the complex in the cell is higher than the expression from the circular polyribonucleotide alone (e.g., lacking a capped polynucleotide) at least by 50 fold. In some embodiments, the expression of the one or more expression sequences from the complex in the cell is higher than the expression from the circular polyribonucleotide alone (e.g., lacking a capped polynucleotide) at least by 55 fold. In some embodiments, the expression of the one or more expression sequences from the complex in the cell is higher than the expression from the circular polyribonucleotide alone (e.g., lacking a capped polynucleotide) at least by 60 fold. In some embodiments, the expression of the one or more expression sequences from the complex in the cell is higher than the expression from the circular polyribonucleotide alone (e.g., lacking a capped polynucleotide) at least by 65 fold. In some embodiments, the expression of the one or more expression sequences from the complex in the cell is higher than the expression from the circular polyribonucleotide alone (e.g., lacking a capped polynucleotide) at least by 70 fold. In some embodiments, the expression of the one or more expression sequences from the complex in the cell is higher than the expression from the circular polyribonucleotide alone (e.g., lacking a capped polynucleotide) at least by 75 fold. In some embodiments, the expression of the one or more expression sequences from the complex in the cell is higher than the expression from the circular polyribonucleotide alone (e.g., lacking a capped polynucleotide) at least by 80 fold. In some embodiments, the expression of the one or more expression sequences from the complex in the cell is higher than the expression from the circular polyribonucleotide alone (e.g., lacking a capped polynucleotide) at least by 85 fold. In some embodiments, the expression of the one or more expression sequences from the complex in the cell is higher than the expression from the circular polyribonucleotide alone (e.g., lacking a capped polynucleotide) at least by 90 fold. In some embodiments, the expression of the one or more expression sequences from the complex in the cell is higher than the expression from the circular polyribonucleotide alone (e.g., lacking a capped polynucleotide) at least by 95 fold. In some embodiments, the expression of the one or more expression sequences from the complex in the cell is higher than the expression from the circular polyribonucleotide alone (e.g., lacking a capped polynucleotide) at least by 100 fold. In some embodiments, the expression of the one or more expression sequences from the complex in the cell is higher than the expression from the circular polyribonucleotide alone (e.g., lacking a capped polynucleotide) at least by 200 fold. In some embodiments, the expression of the one or more expression sequences from the complex in the cell is higher than the expression from the circular polyribonucleotide alone (e.g., lacking a capped polynucleotide) at least by 300 fold. In some embodiments, the expression of the one or more expression sequences from the complex in the cell is higher than the expression from the circular polyribonucleotide alone (e.g., lacking a capped polynucleotide) at least by 400 fold. In some embodiments, the expression of the one or more expression sequences from the complex in the cell is higher than the expression from the circular polyribonucleotide alone (e.g., lacking a capped polynucleotide) at least by 500 fold. In some embodiments, the expression of the one or more expression sequences from the complex in the cell is higher than the expression from the circular polyribonucleotide alone (e.g., lacking a capped polynucleotide) at least by 600 fold. In some embodiments, the expression of the one or more expression sequences from the complex in the cell is higher than the expression from the circular polyribonucleotide alone (e.g., lacking a capped polynucleotide) at least by 700 fold. In some embodiments, the expression of the one or more expression sequences from the complex in the cell is higher than the expression from the circular polyribonucleotide alone (e.g., lacking a capped polynucleotide) at least by 800 fold. In some embodiments, the expression of the one or more expression sequences from the complex in the cell is higher than the expression from the circular polyribonucleotide alone (e.g., lacking a capped polynucleotide) at least by 900 fold. In some embodiments, the expression of the one or more expression sequences from the complex in the cell is higher than the expression from the circular polyribonucleotide alone (e.g., lacking a capped polynucleotide) at least by 1000 fold. In some embodiments, the expression of the one or more expression sequences from the complex in the cell is higher than the expression from the circular polyribonucleotide alone (e.g., lacking a capped polynucleotide) at least by 5000 fold. In some embodiments, the expression of the one or more expression sequences from the complex in the cell is higher than the expression from the circular polyribonucleotide alone (e.g., lacking a capped polynucleotide) at least by 10000 fold. In some embodiments, the expression of the one or more expression sequences from the complex in the cell is higher than the expression from the circular polyribonucleotide alone (e.g., lacking a capped polynucleotide) by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 50000, 10000 fold or more. In some embodiments, the expression of the one or more expression sequences from the complex in the cell is higher than the expression from the circular polyribonucleotide alone (e.g., lacking a capped polynucleotide) by 2 fold. In some embodiments, the expression of the one or more expression sequences from the complex in the cell is higher than the expression from the circular polyribonucleotide alone (e.g., lacking a capped polynucleotide) by 3 fold. In some embodiments, the expression of the one or more expression sequences from the complex in the cell is higher than the expression from the circular polyribonucleotide alone (e.g., lacking a capped polynucleotide) by 4 fold. In some embodiments, the expression of the one or more expression sequences from the complex in the cell is higher than the expression from the circular polyribonucleotide alone (e.g., lacking a capped polynucleotide) by 5 fold. In some embodiments, the expression of the one or more expression sequences from the complex in the cell is higher than the expression from the circular polyribonucleotide alone (e.g., lacking a capped polynucleotide) by 6 fold. In some embodiments, the expression of the one or more expression sequences from the complex in the cell is higher than the expression from the circular polyribonucleotide alone (e.g., lacking a capped polynucleotide) by 7 fold. In some embodiments, the expression of the one or more expression sequences from the complex in the cell is higher than the expression from the circular polyribonucleotide alone (e.g., lacking a capped polynucleotide) by 8 fold. In some embodiments, the expression of the one or more expression sequences from the complex in the cell is higher than the expression from the circular polyribonucleotide alone (e.g., lacking a capped polynucleotide) by 9 fold. In some embodiments, the expression of the one or more expression sequences from the complex in the cell is higher than the expression from the circular polyribonucleotide alone (e.g., lacking a capped polynucleotide) by 10 fold. In some embodiments, the expression of the one or more expression sequences from the complex in the cell is higher than the expression from the circular polyribonucleotide alone (e.g., lacking a capped polynucleotide) by 15 fold. In some embodiments, the expression of the one or more expression sequences from the complex in the cell is higher than the expression from the circular polyribonucleotide alone (e.g., lacking a capped polynucleotide) by 20 fold. In some embodiments, the expression of the one or more expression sequences from the complex in the cell is higher than the expression from the circular polyribonucleotide alone (e.g., lacking a capped polynucleotide) by 25 fold. In some embodiments, the expression of the one or more expression sequences from the complex in the cell is higher than the expression from the circular polyribonucleotide alone (e.g., lacking a capped polynucleotide) by 30 fold. In some embodiments, the expression of the one or more expression sequences from the complex in the cell is higher than the expression from the circular polyribonucleotide alone (e.g., lacking a capped polynucleotide) by 35 fold. In some embodiments, the expression of the one or more expression sequences from the complex in the cell is higher than the expression from the circular polyribonucleotide alone (e.g., lacking a capped polynucleotide) by 40 fold. In some embodiments, the expression of the one or more expression sequences from the complex in the cell is higher than the expression from the circular polyribonucleotide alone (e.g., lacking a capped polynucleotide) by 45 fold. In some embodiments, the expression of the one or more expression sequences from the complex in the cell is higher than the expression from the circular polyribonucleotide alone (e.g., lacking a capped polynucleotide) by 50 fold. In some embodiments, the expression of the one or more expression sequences from the complex in the cell is higher than the expression from the circular polyribonucleotide alone (e.g., lacking a capped polynucleotide) by 55 fold. In some embodiments, the expression of the one or more expression sequences from the complex in the cell is higher than the expression from the circular polyribonucleotide alone (e.g., lacking a capped polynucleotide) by 60 fold. In some embodiments, the expression of the one or more expression sequences from the complex in the cell is higher than the expression from the circular polyribonucleotide alone (e.g., lacking a capped polynucleotide) by 65 fold. In some embodiments, the expression of the one or more expression sequences from the complex in the cell is higher than the expression from the circular polyribonucleotide alone (e.g., lacking a capped polynucleotide) by 70 fold. In some embodiments, the expression of the one or more expression sequences from the complex in the cell is higher than the expression from the circular polyribonucleotide alone (e.g., lacking a capped polynucleotide) by 75 fold. In some embodiments, the expression of the one or more expression sequences from the complex in the cell is higher than the expression from the circular polyribonucleotide alone (e.g., lacking a capped polynucleotide) by 80 fold. In some embodiments, the expression of the one or more expression sequences from the complex in the cell is higher than the expression from the circular polyribonucleotide alone (e.g., lacking a capped polynucleotide) by 85 fold. In some embodiments, the expression of the one or more expression sequences from the complex in the cell is higher than the expression from the circular polyribonucleotide alone (e.g., lacking a capped polynucleotide) by 90 fold. In some embodiments, the expression of the one or more expression sequences from the complex in the cell is higher than the expression from the circular polyribonucleotide alone (e.g., lacking a capped polynucleotide) by 95 fold. In some embodiments, the expression of the one or more expression sequences from the complex in the cell is higher than the expression from the circular polyribonucleotide alone (e.g., lacking a capped polynucleotide) by 100 fold. In some embodiments, the expression of the one or more expression sequences from the complex in the cell is higher than the expression from the circular polyribonucleotide alone (e.g., lacking a capped polynucleotide) by 200 fold. In some embodiments, the expression of the one or more expression sequences from the complex in the cell is higher than the expression from the circular polyribonucleotide alone (e.g., lacking a capped polynucleotide) by 300 fold. In some embodiments, the expression of the one or more expression sequences from the complex in the cell is higher than the expression from the circular polyribonucleotide alone (e.g., lacking a capped polynucleotide) by 400 fold. In some embodiments, the expression of the one or more expression sequences from the complex in the cell is higher than the expression from the circular polyribonucleotide alone (e.g., lacking a capped polynucleotide) by 500 fold. In some embodiments, the expression of the one or more expression sequences from the complex in the cell is higher than the expression from the circular polyribonucleotide alone (e.g., lacking a capped polynucleotide) by 600 fold. In some embodiments, the expression of the one or more expression sequences from the complex in the cell is higher than the expression from the circular polyribonucleotide alone (e.g., lacking a capped polynucleotide) by 700 fold. In some embodiments, the expression of the one or more expression sequences from the complex in the cell is higher than the expression from the circular polyribonucleotide alone (e.g., lacking a capped polynucleotide) by 800 fold. In some embodiments, the expression of the one or more expression sequences from the complex in the cell is higher than the expression from the circular polyribonucleotide alone (e.g., lacking a capped polynucleotide) by 900 fold. In some embodiments, the expression of the one or more expression sequences from the complex in the cell is higher than the expression from the circular polyribonucleotide alone (e.g., lacking a capped polynucleotide) by 1000 fold. In some embodiments, the expression of the one or more expression sequences from the complex in the cell is higher than the expression from the circular polyribonucleotide alone (e.g., lacking a capped polynucleotide) by 5000 fold. In some embodiments, the expression of the one or more expression sequences from the complex in the cell is higher than the expression from the circular polyribonucleotide alone (e.g., lacking a capped polynucleotide) by 10000 fold.

In some other aspects, the invention as provided herein comprises a method of in vitro expression of one or more expression sequences, comprising: administering to a cell in vitro a circular polyribonucleotide as provided herein comprising the one or more expression sequences, and a capped polyribonucleotide as provided herein to a cell, wherein the first binding region of the capped polyribonucleotide is bound to the second binding region of the circular polyribonucleotide to form a complex in the cell and the expression of the one or more expression sequences from the complex in the cell is greater than expression from the circular polyribonucleotide alone (e.g., lacking the capped polyribonucleotide).

In some embodiments, the expression of the one or more expression sequences from the complex in the cell is at least 10%, 20%, 30%, 40%, 50%, 60%, 70%, 80%, 90%, 100%, 200%, 300%, 400%, 500%, 600%, 700%, 800%, 900%, 1000%, 5000%, 10000%, or more greater than the expression from the circular polyribonucleotide alone (e.g., lacking a capped polynucleotide). In some embodiments, the expression of the one or more expression sequences from the complex in the cell is at least 10% greater than the expression from the circular polyribonucleotide alone (e.g., lacking a capped polynucleotide). In some embodiments, the expression of the one or more expression sequences from the complex in the cell is at least 20% greater than the expression from the circular polyribonucleotide alone (e.g., lacking a capped polynucleotide). In some embodiments, the expression of the one or more expression sequences from the complex in the cell is at least 30% greater than the expression from the circular polyribonucleotide alone (e.g., lacking a capped polynucleotide). In some embodiments, the expression of the one or more expression sequences from the complex in the cell is at least 40% greater than the expression from the circular polyribonucleotide alone (e.g., lacking a capped polynucleotide). In some embodiments, the expression of the one or more expression sequences from the complex in the cell is at least 50% greater than the expression from the circular polyribonucleotide alone (e.g., lacking a capped polynucleotide). In some embodiments, the expression of the one or more expression sequences from the complex in the cell is at least 60% greater than the expression from the circular polyribonucleotide alone (e.g., lacking a capped polynucleotide). In some embodiments, the expression of the one or more expression sequences from the complex in the cell is at least 70% greater than the expression from the circular polyribonucleotide alone (e.g., lacking a capped polynucleotide). In some embodiments, the expression of the one or more expression sequences from the complex in the cell is at least 80% greater than the expression from the circular polyribonucleotide alone (e.g., lacking a capped polynucleotide). In some embodiments, the expression of the one or more expression sequences from the complex in the cell is at least 90% greater than the expression from the circular polyribonucleotide alone (e.g., lacking a capped polynucleotide). In some embodiments, the expression of the one or more expression sequences from the complex in the cell is at least 100% greater than the expression from the circular polyribonucleotide alone (e.g., lacking a capped polynucleotide). In some embodiments, the expression of the one or more expression sequences from the complex in the cell is at least 200% greater than the expression from the circular polyribonucleotide alone (e.g., lacking a capped polynucleotide). In some embodiments, the expression of the one or more expression sequences from the complex in the cell is at least 300% greater than the expression from the circular polyribonucleotide alone (e.g., lacking a capped polynucleotide). In some embodiments, the expression of the one or more expression sequences from the complex in the cell is at least 400% greater than the expression from the circular polyribonucleotide alone (e.g., lacking a capped polynucleotide). In some embodiments, the expression of the one or more expression sequences from the complex in the cell is at least 500% greater than the expression from the circular polyribonucleotide alone (e.g., lacking a capped polynucleotide). In some embodiments, the expression of the one or more expression sequences from the complex in the cell is at least 600% greater than the expression from the circular polyribonucleotide alone (e.g., lacking a capped polynucleotide). In some embodiments, the expression of the one or more expression sequences from the complex in the cell is at least 700% greater than the expression from the circular polyribonucleotide alone (e.g., lacking a capped polynucleotide). In some embodiments, the expression of the one or more expression sequences from the complex in the cell is at least 800% greater than the expression from the circular polyribonucleotide alone (e.g., lacking a capped polynucleotide). In some embodiments, the expression of the one or more expression sequences from the complex in the cell is at least 900% greater than the expression from the circular polyribonucleotide alone (e.g., lacking a capped polynucleotide). In some embodiments, the expression of the one or more expression sequences from the complex in the cell is at least 1000% greater than the expression from the circular polyribonucleotide alone (e.g., lacking a capped polynucleotide). In some embodiments, the expression of the one or more expression sequences from the complex in the cell is at least 5000% greater than the expression from the circular polyribonucleotide alone (e.g., lacking a capped polynucleotide). In some embodiments, the expression of the one or more expression sequences from the complex in the cell is at least 10000% greater than the expression from the circular polyribonucleotide alone (e.g., lacking a capped polynucleotide). In some embodiments, the expression of the one or more expression sequences from the complex in the cell is 10%, 20%, 30%, 40%, 50%, 60%, 70%, 80%, 90%, 100%, 200%, 300%, 400%, 500%, 600%, 700%, 800%, 900%, 1000%, 5000%, 10000%, or more greater than the expression from the circular polyribonucleotide alone (e.g., lacking a capped polynucleotide). In some embodiments, the expression of the one or more expression sequences from the complex in the cell is 10% greater than the expression from the circular polyribonucleotide alone (e.g., lacking a capped polynucleotide). In some embodiments, the expression of the one or more expression sequences from the complex in the cell is 20% greater than the expression from the circular polyribonucleotide alone (e.g., lacking a capped polynucleotide). In some embodiments, the expression of the one or more expression sequences from the complex in the cell is 30% greater than the expression from the circular polyribonucleotide alone (e.g., lacking a capped polynucleotide). In some embodiments, the expression of the one or more expression sequences from the complex in the cell is 40% greater than the expression from the circular polyribonucleotide alone (e.g., lacking a capped polynucleotide). In some embodiments, the expression of the one or more expression sequences from the complex in the cell is 50% greater than the expression from the circular polyribonucleotide alone (e.g., lacking a capped polynucleotide). In some embodiments, the expression of the one or more expression sequences from the complex in the cell is 60% greater than the expression from the circular polyribonucleotide alone (e.g., lacking a capped polynucleotide). In some embodiments, the expression of the one or more expression sequences from the complex in the cell is 70% greater than the expression from the circular polyribonucleotide alone (e.g., lacking a capped polynucleotide). In some embodiments, the expression of the one or more expression sequences from the complex in the cell is 80% greater than the expression from the circular polyribonucleotide alone (e.g., lacking a capped polynucleotide). In some embodiments, the expression of the one or more expression sequences from the complex in the cell is 90% greater than the expression from the circular polyribonucleotide alone (e.g., lacking a capped polynucleotide). In some embodiments, the expression of the one or more expression sequences from the complex in the cell is 100% greater than the expression from the circular polyribonucleotide alone (e.g., lacking a capped polynucleotide). In some embodiments, the expression of the one or more expression sequences from the complex in the cell is 200% greater than the expression from the circular polyribonucleotide alone (e.g., lacking a capped polynucleotide). In some embodiments, the expression of the one or more expression sequences from the complex in the cell is 300% greater than the expression from the circular polyribonucleotide alone (e.g., lacking a capped polynucleotide). In some embodiments, the expression of the one or more expression sequences from the complex in the cell is 400% greater than the expression from the circular polyribonucleotide alone (e.g., lacking a capped polynucleotide). In some embodiments, the expression of the one or more expression sequences from the complex in the cell is 500% greater than the expression from the circular polyribonucleotide alone (e.g., lacking a capped polynucleotide). In some embodiments, the expression of the one or more expression sequences from the complex in the cell is 600% greater than the expression from the circular polyribonucleotide alone (e.g., lacking a capped polynucleotide). In some embodiments, the expression of the one or more expression sequences from the complex in the cell is 700% greater than the expression from the circular polyribonucleotide alone (e.g., lacking a capped polynucleotide). In some embodiments, the expression of the one or more expression sequences from the complex in the cell is 800% greater than the expression from the circular polyribonucleotide alone (e.g., lacking a capped polynucleotide). In some embodiments, the expression of the one or more expression sequences from the complex in the cell is 900% greater than the expression from the circular polyribonucleotide alone (e.g., lacking a capped polynucleotide). In some embodiments, the expression of the one or more expression sequences from the complex in the cell is 1000% greater than the expression from the circular polyribonucleotide alone (e.g., lacking a capped polynucleotide). In some embodiments, the expression of the one or more expression sequences from the complex in the cell is 5000% greater than the expression from the circular polyribonucleotide alone (e.g., lacking a capped polynucleotide). In some embodiments, the expression of the one or more expression sequences from the complex in the cell is 10000% greater than the expression from the circular polyribonucleotide alone (e.g., lacking a capped polynucleotide). In some embodiments, the expression of the one or more expression sequences from the complex in the cell is greater than the expression from the circular polyribonucleotide alone (e.g., lacking a capped polynucleotide) at least by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 5000, 10000 fold or more. In some embodiments, the expression of the one or more expression sequences from the complex in the cell is greater than the expression from the circular polyribonucleotide alone (e.g., lacking a capped polynucleotide) at least by 2 fold. In some embodiments, the expression of the one or more expression sequences from the complex in the cell is greater than the expression from the circular polyribonucleotide alone (e.g., lacking a capped polynucleotide) at least by 3 fold. In some embodiments, the expression of the one or more expression sequences from the complex in the cell is greater than the expression from the circular polyribonucleotide alone (e.g., lacking a capped polynucleotide) at least by 4 fold. In some embodiments, the expression of the one or more expression sequences from the complex in the cell is greater than the expression from the circular polyribonucleotide alone (e.g., lacking a capped polynucleotide) at least by 5 fold. In some embodiments, the expression of the one or more expression sequences from the complex in the cell is greater than the expression from the circular polyribonucleotide alone (e.g., lacking a capped polynucleotide) at least by 6 fold. In some embodiments, the expression of the one or more expression sequences from the complex in the cell is greater than the expression from the circular polyribonucleotide alone (e.g., lacking a capped polynucleotide) at least by 7 fold. In some embodiments, the expression of the one or more expression sequences from the complex in the cell is greater than the expression from the circular polyribonucleotide alone (e.g., lacking a capped polynucleotide) at least by 8 fold. In some embodiments, the expression of the one or more expression sequences from the complex in the cell is greater than the expression from the circular polyribonucleotide alone (e.g., lacking a capped polynucleotide) at least by 9 fold. In some embodiments, the expression of the one or more expression sequences from the complex in the cell is greater than the expression from the circular polyribonucleotide alone (e.g., lacking a capped polynucleotide) at least by 10 fold. In some embodiments, the expression of the one or more expression sequences from the complex in the cell is greater than the expression from the circular polyribonucleotide alone (e.g., lacking a capped polynucleotide) at least by 15 fold. In some embodiments, the expression of the one or more expression sequences from the complex in the cell is greater than the expression from the circular polyribonucleotide alone (e.g., lacking a capped polynucleotide) at least by 20 fold. In some embodiments, the expression of the one or more expression sequences from the complex in the cell is greater than the expression from the circular polyribonucleotide alone (e.g., lacking a capped polynucleotide) at least by 25 fold. In some embodiments, the expression of the one or more expression sequences from the complex in the cell is greater than the expression from the circular polyribonucleotide alone (e.g., lacking a capped polynucleotide) at least by 30 fold. In some embodiments, the expression of the one or more expression sequences from the complex in the cell is greater than the expression from the circular polyribonucleotide alone (e.g., lacking a capped polynucleotide) at least by 35 fold. In some embodiments, the expression of the one or more expression sequences from the complex in the cell is greater than the expression from the circular polyribonucleotide alone (e.g., lacking a capped polynucleotide) at least by 40 fold. In some embodiments, the expression of the one or more expression sequences from the complex in the cell is greater than the expression from the circular polyribonucleotide alone (e.g., lacking a capped polynucleotide) at least by 45 fold. In some embodiments, the expression of the one or more expression sequences from the complex in the cell is greater than the expression from the circular polyribonucleotide alone (e.g., lacking a capped polynucleotide) at least by 50 fold. In some embodiments, the expression of the one or more expression sequences from the complex in the cell is greater than the expression from the circular polyribonucleotide alone (e.g., lacking a capped polynucleotide) at least by 55 fold. In some embodiments, the expression of the one or more expression sequences from the complex in the cell is greater than the expression from the circular polyribonucleotide alone (e.g., lacking a capped polynucleotide) at least by 60 fold. In some embodiments, the expression of the one or more expression sequences from the complex in the cell is greater than the expression from the circular polyribonucleotide alone (e.g., lacking a capped polynucleotide) at least by 65 fold. In some embodiments, the expression of the one or more expression sequences from the complex in the cell is greater than the expression from the circular polyribonucleotide alone (e.g., lacking a capped polynucleotide) at least by 70 fold. In some embodiments, the expression of the one or more expression sequences from the complex in the cell is greater than the expression from the circular polyribonucleotide alone (e.g., lacking a capped polynucleotide) at least by 75 fold. In some embodiments, the expression of the one or more expression sequences from the complex in the cell is greater than the expression from the circular polyribonucleotide alone (e.g., lacking a capped polynucleotide) at least by 80 fold. In some embodiments, the expression of the one or more expression sequences from the complex in the cell is greater than the expression from the circular polyribonucleotide alone (e.g., lacking a capped polynucleotide) at least by 85 fold. In some embodiments, the expression of the one or more expression sequences from the complex in the cell is greater than the expression from the circular polyribonucleotide alone (e.g., lacking a capped polynucleotide) at least by 90 fold. In some embodiments, the expression of the one or more expression sequences from the complex in the cell is greater than the expression from the circular polyribonucleotide alone (e.g., lacking a capped polynucleotide) at least by 95 fold. In some embodiments, the expression of the one or more expression sequences from the complex in the cell is greater than the expression from the circular polyribonucleotide alone (e.g., lacking a capped polynucleotide) at least by 100 fold. In some embodiments, the expression of the one or more expression sequences from the complex in the cell is greater than the expression from the circular polyribonucleotide alone (e.g., lacking a capped polynucleotide) at least by 200 fold. In some embodiments, the expression of the one or more expression sequences from the complex in the cell is greater than the expression from the circular polyribonucleotide alone (e.g., lacking a capped polynucleotide) at least by 300 fold. In some embodiments, the expression of the one or more expression sequences from the complex in the cell is greater than the expression from the circular polyribonucleotide alone (e.g., lacking a capped polynucleotide) at least by 400 fold. In some embodiments, the expression of the one or more expression sequences from the complex in the cell is greater than the expression from the circular polyribonucleotide alone (e.g., lacking a capped polynucleotide) at least by 500 fold. In some embodiments, the expression of the one or more expression sequences from the complex in the cell is greater than the expression from the circular polyribonucleotide alone (e.g., lacking a capped polynucleotide) at least by 600 fold. In some embodiments, the expression of the one or more expression sequences from the complex in the cell is greater than the expression from the circular polyribonucleotide alone (e.g., lacking a capped polynucleotide) at least by 700 fold. In some embodiments, the expression of the one or more expression sequences from the complex in the cell is greater than the expression from the circular polyribonucleotide alone (e.g., lacking a capped polynucleotide) at least by 800 fold. In some embodiments, the expression of the one or more expression sequences from the complex in the cell is greater than the expression from the circular polyribonucleotide alone (e.g., lacking a capped polynucleotide) at least by 900 fold. In some embodiments, the expression of the one or more expression sequences from the complex in the cell is greater than the expression from the circular polyribonucleotide alone (e.g., lacking a capped polynucleotide) at least by 1000 fold. In some embodiments, the expression of the one or more expression sequences from the complex in the cell is greater than the expression from the circular polyribonucleotide alone (e.g., lacking a capped polynucleotide) at least by 5000 fold. In some embodiments, the expression of the one or more expression sequences from the complex in the cell is greater than the expression from the circular polyribonucleotide alone (e.g., lacking a capped polynucleotide) at least by 10000 fold. In some embodiments, the expression of the one or more expression sequences from the complex in the cell is greater than the expression from the circular polyribonucleotide alone (e.g., lacking a capped polynucleotide) by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 50000, 10000 fold or more. In some embodiments, the expression of the one or more expression sequences from the complex in the cell is greater than the expression from the circular polyribonucleotide alone (e.g., lacking a capped polynucleotide) by 2 fold. In some embodiments, the expression of the one or more expression sequences from the complex in the cell is greater than the expression from the circular polyribonucleotide alone (e.g., lacking a capped polynucleotide) by 3 fold. In some embodiments, the expression of the one or more expression sequences from the complex in the cell is greater than the expression from the circular polyribonucleotide alone (e.g., lacking a capped polynucleotide) by 4 fold. In some embodiments, the expression of the one or more expression sequences from the complex in the cell is greater than the expression from the circular polyribonucleotide alone (e.g., lacking a capped polynucleotide) by 5 fold. In some embodiments, the expression of the one or more expression sequences from the complex in the cell is greater than the expression from the circular polyribonucleotide alone (e.g., lacking a capped polynucleotide) by 6 fold. In some embodiments, the expression of the one or more expression sequences from the complex in the cell is greater than the expression from the circular polyribonucleotide alone (e.g., lacking a capped polynucleotide) by 7 fold. In some embodiments, the expression of the one or more expression sequences from the complex in the cell is greater than the expression from the circular polyribonucleotide alone (e.g., lacking a capped polynucleotide) by 8 fold. In some embodiments, the expression of the one or more expression sequences from the complex in the cell is greater than the expression from the circular polyribonucleotide alone (e.g., lacking a capped polynucleotide) by 9 fold. In some embodiments, the expression of the one or more expression sequences from the complex in the cell is greater than the expression from the circular polyribonucleotide alone (e.g., lacking a capped polynucleotide) by 10 fold. In some embodiments, the expression of the one or more expression sequences from the complex in the cell is greater than the expression from the circular polyribonucleotide alone (e.g., lacking a capped polynucleotide) by 15 fold. In some embodiments, the expression of the one or more expression sequences from the complex in the cell is greater than the expression from the circular polyribonucleotide alone (e.g., lacking a capped polynucleotide) by 20 fold. In some embodiments, the expression of the one or more expression sequences from the complex in the cell is greater than the expression from the circular polyribonucleotide alone (e.g., lacking a capped polynucleotide) by 25 fold. In some embodiments, the expression of the one or more expression sequences from the complex in the cell is greater than the expression from the circular polyribonucleotide alone (e.g., lacking a capped polynucleotide) by 30 fold. In some embodiments, the expression of the one or more expression sequences from the complex in the cell is greater than the expression from the circular polyribonucleotide alone (e.g., lacking a capped polynucleotide) by 35 fold. In some embodiments, the expression of the one or more expression sequences from the complex in the cell is greater than the expression from the circular polyribonucleotide alone (e.g., lacking a capped polynucleotide) by 40 fold. In some embodiments, the expression of the one or more expression sequences from the complex in the cell is greater than the expression from the circular polyribonucleotide alone (e.g., lacking a capped polynucleotide) by 45 fold. In some embodiments, the expression of the one or more expression sequences from the complex in the cell is greater than the expression from the circular polyribonucleotide alone (e.g., lacking a capped polynucleotide) by 50 fold. In some embodiments, the expression of the one or more expression sequences from the complex in the cell is greater than the expression from the circular polyribonucleotide alone (e.g., lacking a capped polynucleotide) by 55 fold. In some embodiments, the expression of the one or more expression sequences from the complex in the cell is greater than the expression from the circular polyribonucleotide alone (e.g., lacking a capped polynucleotide) by 60 fold. In some embodiments, the expression of the one or more expression sequences from the complex in the cell is greater than the expression from the circular polyribonucleotide alone (e.g., lacking a capped polynucleotide) by 65 fold. In some embodiments, the expression of the one or more expression sequences from the complex in the cell is greater than the expression from the circular polyribonucleotide alone (e.g., lacking a capped polynucleotide) by 70 fold. In some embodiments, the expression of the one or more expression sequences from the complex in the cell is greater than the expression from the circular polyribonucleotide alone (e.g., lacking a capped polynucleotide) by 75 fold. In some embodiments, the expression of the one or more expression sequences from the complex in the cell is greater than the expression from the circular polyribonucleotide alone (e.g., lacking a capped polynucleotide) by 80 fold. In some embodiments, the expression of the one or more expression sequences from the complex in the cell is greater than the expression from the circular polyribonucleotide alone (e.g., lacking a capped polynucleotide) by 85 fold. In some embodiments, the expression of the one or more expression sequences from the complex in the cell is greater than the expression from the circular polyribonucleotide alone (e.g., lacking a capped polynucleotide) by 90 fold. In some embodiments, the expression of the one or more expression sequences from the complex in the cell is greater than the expression from the circular polyribonucleotide alone (e.g., lacking a capped polynucleotide) by 95 fold. In some embodiments, the expression of the one or more expression sequences from the complex in the cell is greater than the expression from the circular polyribonucleotide alone (e.g., lacking a capped polynucleotide) by 100 fold. In some embodiments, the expression of the one or more expression sequences from the complex in the cell is greater than the expression from the circular polyribonucleotide alone (e.g., lacking a capped polynucleotide) by 200 fold. In some embodiments, the expression of the one or more expression sequences from the complex in the cell is greater than the expression from the circular polyribonucleotide alone (e.g., lacking a capped polynucleotide) by 300 fold. In some embodiments, the expression of the one or more expression sequences from the complex in the cell is greater than the expression from the circular polyribonucleotide alone (e.g., lacking a capped polynucleotide) by 400 fold. In some embodiments, the expression of the one or more expression sequences from the complex in the cell is greater than the expression from the circular polyribonucleotide alone (e.g., lacking a capped polynucleotide) by 500 fold. In some embodiments, the expression of the one or more expression sequences from the complex in the cell is greater than the expression from the circular polyribonucleotide alone (e.g., lacking a capped polynucleotide) by 600 fold. In some embodiments, the expression of the one or more expression sequences from the complex in the cell is greater than the expression from the circular polyribonucleotide alone (e.g., lacking a capped polynucleotide) by 700 fold. In some embodiments, the expression of the one or more expression sequences from the complex in the cell is greater than the expression from the circular polyribonucleotide alone (e.g., lacking a capped polynucleotide) by 800 fold. In some embodiments, the expression of the one or more expression sequences from the complex in the cell is greater than the expression from the circular polyribonucleotide alone (e.g., lacking a capped polynucleotide) by 900 fold. In some embodiments, the expression of the one or more expression sequences from the complex in the cell is greater than the expression from the circular polyribonucleotide alone (e.g., lacking a capped polynucleotide) by 1000 fold. In some embodiments, the expression of the one or more expression sequences from the complex in the cell is greater than the expression from the circular polyribonucleotide alone (e.g., lacking a capped polynucleotide) by 50000 fold. In some embodiments, the expression of the one or more expression sequences from the complex in the cell is greater than the expression from the circular polyribonucleotide alone (e.g., lacking a capped polynucleotide) by 10000 fold.

In some other aspects, the invention as provided herein comprises a method of in vitro expression of one or more expression sequences, comprising: administering to a cell in vitro a circular polyribonucleotide as provided herein comprising the one or more expression sequences, and a capped polyribonucleotide as provided herein to a cell, wherein the first binding region of the capped polyribonucleotide is bound to the second binding region of the circular polyribonucleotide to form a complex in the cell and the expression of the one or more expression sequences from the complex in the cell is increased compared to expression from the circular polyribonucleotide alone.

In some embodiments, the expression of the one or more expression sequences from the complex in the cell is increased by at least 10%, 20%, 30%, 40%, 50%, 60%, 70%, 80%, 90%, 100%, 200%, 300%, 400%, 500%, 600%, 700%, 800%, 900%, 1000%, 5000%, 10000%, or more compared to the expression from the circular polyribonucleotide alone (e.g., lacking a capped polynucleotide). In some embodiments, the expression of the one or more expression sequences from the complex in the cell is increased by at least 10% compared to the expression from the circular polyribonucleotide alone (e.g., lacking a capped polynucleotide). In some embodiments, the expression of the one or more expression sequences from the complex in the cell is increased by at least 20% compared to the expression from the circular polyribonucleotide alone (e.g., lacking a capped polynucleotide). In some embodiments, the expression of the one or more expression sequences from the complex in the cell is increased by at least 30% compared to the expression from the circular polyribonucleotide alone (e.g., lacking a capped polynucleotide). In some embodiments, the expression of the one or more expression sequences from the complex in the cell is increased by at least 40% compared to the expression from the circular polyribonucleotide alone (e.g., lacking a capped polynucleotide). In some embodiments, the expression of the one or more expression sequences from the complex in the cell is increased by at least 50% compared to the expression from the circular polyribonucleotide alone (e.g., lacking a capped polynucleotide). In some embodiments, the expression of the one or more expression sequences from the complex in the cell is increased by at least 60% compared to the expression from the circular polyribonucleotide alone (e.g., lacking a capped polynucleotide). In some embodiments, the expression of the one or more expression sequences from the complex in the cell is increased by at least 70% compared to the expression from the circular polyribonucleotide alone (e.g., lacking a capped polynucleotide). In some embodiments, the expression of the one or more expression sequences from the complex in the cell is increased by at least 80% compared to the expression from the circular polyribonucleotide alone (e.g., lacking a capped polynucleotide). In some embodiments, the expression of the one or more expression sequences from the complex in the cell is increased by at least 90% compared to the expression from the circular polyribonucleotide alone (e.g., lacking a capped polynucleotide). In some embodiments, the expression of the one or more expression sequences from the complex in the cell is increased by at least 100% compared to the expression from the circular polyribonucleotide alone (e.g., lacking a capped polynucleotide). In some embodiments, the expression of the one or more expression sequences from the complex in the cell is increased by at least 200% compared to the expression from the circular polyribonucleotide alone (e.g., lacking a capped polynucleotide). In some embodiments, the expression of the one or more expression sequences from the complex in the cell is increased by at least 300% compared to the expression from the circular polyribonucleotide alone (e.g., lacking a capped polynucleotide). In some embodiments, the expression of the one or more expression sequences from the complex in the cell is increased by at least 400% compared to the expression from the circular polyribonucleotide alone (e.g., lacking a capped polynucleotide). In some embodiments, the expression of the one or more expression sequences from the complex in the cell is increased by at least 500% compared to the expression from the circular polyribonucleotide alone (e.g., lacking a capped polynucleotide). In some embodiments, the expression of the one or more expression sequences from the complex in the cell is increased by at least 600% compared to the expression from the circular polyribonucleotide alone (e.g., lacking a capped polynucleotide). In some embodiments, the expression of the one or more expression sequences from the complex in the cell is increased by at least 700% compared to the expression from the circular polyribonucleotide alone (e.g., lacking a capped polynucleotide). In some embodiments, the expression of the one or more expression sequences from the complex in the cell is increased by at least 800% compared to the expression from the circular polyribonucleotide alone (e.g., lacking a capped polynucleotide). In some embodiments, the expression of the one or more expression sequences from the complex in the cell is increased by at least 900% compared to the expression from the circular polyribonucleotide alone (e.g., lacking a capped polynucleotide). In some embodiments, the expression of the one or more expression sequences from the complex in the cell is increased by at least 1000% compared to the expression from the circular polyribonucleotide alone (e.g., lacking a capped polynucleotide). In some embodiments, the expression of the one or more expression sequences from the complex in the cell is increased by at least 5000% compared to the expression from the circular polyribonucleotide alone (e.g., lacking a capped polynucleotide). In some embodiments, the expression of the one or more expression sequences from the complex in the cell is increased by at least 10000% compared to the expression from the circular polyribonucleotide alone (e.g., lacking a capped polynucleotide). In some embodiments, the expression of the one or more expression sequences from the complex in the cell is increased by 10%, 20%, 30%, 40%, 50%, 60%, 70%, 80%, 90%, 100%, 200%, 300%, 400%, 500%, 600%, 700%, 800%, 900%, 1000%, 5000%, 10000%, or more compared to the expression from the circular polyribonucleotide alone (e.g., lacking a capped polynucleotide). In some embodiments, the expression of the one or more expression sequences from the complex in the cell is increased by 10% compared to the expression from the circular polyribonucleotide alone (e.g., lacking a capped polynucleotide). In some embodiments, the expression of the one or more expression sequences from the complex in the cell is increased by 20% compared to the expression from the circular polyribonucleotide alone (e.g., lacking a capped polynucleotide). In some embodiments, the expression of the one or more expression sequences from the complex in the cell is increased by 30% compared to the expression from the circular polyribonucleotide alone (e.g., lacking a capped polynucleotide). In some embodiments, the expression of the one or more expression sequences from the complex in the cell is increased by 40% compared to the expression from the circular polyribonucleotide alone (e.g., lacking a capped polynucleotide). In some embodiments, the expression of the one or more expression sequences from the complex in the cell is increased by 50% compared to the expression from the circular polyribonucleotide alone (e.g., lacking a capped polynucleotide). In some embodiments, the expression of the one or more expression sequences from the complex in the cell is increased by 60% compared to the expression from the circular polyribonucleotide alone (e.g., lacking a capped polynucleotide). In some embodiments, the expression of the one or more expression sequences from the complex in the cell is increased by 70% compared to the expression from the circular polyribonucleotide alone (e.g., lacking a capped polynucleotide). In some embodiments, the expression of the one or more expression sequences from the complex in the cell is increased by 80% compared to the expression from the circular polyribonucleotide alone (e.g., lacking a capped polynucleotide). In some embodiments, the expression of the one or more expression sequences from the complex in the cell is increased by 90% compared to the expression from the circular polyribonucleotide alone (e.g., lacking a capped polynucleotide). In some embodiments, the expression of the one or more expression sequences from the complex in the cell is increased by 100% compared to the expression from the circular polyribonucleotide alone (e.g., lacking a capped polynucleotide). In some embodiments, the expression of the one or more expression sequences from the complex in the cell is increased by 200% compared to the expression from the circular polyribonucleotide alone (e.g., lacking a capped polynucleotide). In some embodiments, the expression of the one or more expression sequences from the complex in the cell is increased by 300% compared to the expression from the circular polyribonucleotide alone (e.g., lacking a capped polynucleotide). In some embodiments, the expression of the one or more expression sequences from the complex in the cell is increased by 400% compared to the expression from the circular polyribonucleotide alone (e.g., lacking a capped polynucleotide). In some embodiments, the expression of the one or more expression sequences from the complex in the cell is increased by 500% compared to the expression from the circular polyribonucleotide alone (e.g., lacking a capped polynucleotide). In some embodiments, the expression of the one or more expression sequences from the complex in the cell is increased by 600% compared to the expression from the circular polyribonucleotide alone (e.g., lacking a capped polynucleotide). In some embodiments, the expression of the one or more expression sequences from the complex in the cell is increased by 700% compared to the expression from the circular polyribonucleotide alone (e.g., lacking a capped polynucleotide). In some embodiments, the expression of the one or more expression sequences from the complex in the cell is increased by 800% compared to the expression from the circular polyribonucleotide alone (e.g., lacking a capped polynucleotide). In some embodiments, the expression of the one or more expression sequences from the complex in the cell is increased by 900% compared to the expression from the circular polyribonucleotide alone (e.g., lacking a capped polynucleotide). In some embodiments, the expression of the one or more expression sequences from the complex in the cell is increased by 1000% compared to the expression from the circular polyribonucleotide alone (e.g., lacking a capped polynucleotide). In some embodiments, the expression of the one or more expression sequences from the complex in the cell is increased by 5000% compared to the expression from the circular polyribonucleotide alone (e.g., lacking a capped polynucleotide). In some embodiments, the expression of the one or more expression sequences from the complex in the cell is increased by 10000% compared to the expression from the circular polyribonucleotide alone (e.g., lacking a capped polynucleotide). In some embodiments, the expression of the one or more expression sequences from the complex in the cell is increased compared to the expression from the circular polyribonucleotide alone (e.g., lacking a capped polynucleotide) at least by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 5000, 10000 fold or more. In some embodiments, the expression of the one or more expression sequences from the complex in the cell is increased compared to the expression from the circular polyribonucleotide alone (e.g., lacking a capped polynucleotide) at least by 2 fold. In some embodiments, the expression of the one or more expression sequences from the complex in the cell is increased compared to the expression from the circular polyribonucleotide alone (e.g., lacking a capped polynucleotide) at least by 3 fold. In some embodiments, the expression of the one or more expression sequences from the complex in the cell is increased compared to the expression from the circular polyribonucleotide alone (e.g., lacking a capped polynucleotide) at least by 4 fold. In some embodiments, the expression of the one or more expression sequences from the complex in the cell is increased compared to the expression from the circular polyribonucleotide alone (e.g., lacking a capped polynucleotide) at least by 5 fold. In some embodiments, the expression of the one or more expression sequences from the complex in the cell is increased compared to the expression from the circular polyribonucleotide alone (e.g., lacking a capped polynucleotide) at least by 6 fold. In some embodiments, the expression of the one or more expression sequences from the complex in the cell is increased compared to the expression from the circular polyribonucleotide alone (e.g., lacking a capped polynucleotide) at least by 7 fold. In some embodiments, the expression of the one or more expression sequences from the complex in the cell is increased compared to the expression from the circular polyribonucleotide alone (e.g., lacking a capped polynucleotide) at least by 8 fold. In some embodiments, the expression of the one or more expression sequences from the complex in the cell is increased compared to the expression from the circular polyribonucleotide alone (e.g., lacking a capped polynucleotide) at least by 9 fold. In some embodiments, the expression of the one or more expression sequences from the complex in the cell is increased compared to the expression from the circular polyribonucleotide alone (e.g., lacking a capped polynucleotide) at least by 10 fold. In some embodiments, the expression of the one or more expression sequences from the complex in the cell is increased compared to the expression from the circular polyribonucleotide alone (e.g., lacking a capped polynucleotide) at least by 15 fold. In some embodiments, the expression of the one or more expression sequences from the complex in the cell is increased compared to the expression from the circular polyribonucleotide alone (e.g., lacking a capped polynucleotide) at least by 20 fold. In some embodiments, the expression of the one or more expression sequences from the complex in the cell is increased compared to the expression from the circular polyribonucleotide alone (e.g., lacking a capped polynucleotide) at least by 25 fold. In some embodiments, the expression of the one or more expression sequences from the complex in the cell is increased compared to the expression from the circular polyribonucleotide alone (e.g., lacking a capped polynucleotide) at least by 30 fold. In some embodiments, the expression of the one or more expression sequences from the complex in the cell is increased compared to the expression from the circular polyribonucleotide alone (e.g., lacking a capped polynucleotide) at least by 35 fold. In some embodiments, the expression of the one or more expression sequences from the complex in the cell is increased compared to the expression from the circular polyribonucleotide alone (e.g., lacking a capped polynucleotide) at least by 40 fold. In some embodiments, the expression of the one or more expression sequences from the complex in the cell is increased compared to the expression from the circular polyribonucleotide alone (e.g., lacking a capped polynucleotide) at least by 45 fold. In some embodiments, the expression of the one or more expression sequences from the complex in the cell is increased compared to the expression from the circular polyribonucleotide alone (e.g., lacking a capped polynucleotide) at least by 50 fold. In some embodiments, the expression of the one or more expression sequences from the complex in the cell is increased compared to the expression from the circular polyribonucleotide alone (e.g., lacking a capped polynucleotide) at least by 55 fold. In some embodiments, the expression of the one or more expression sequences from the complex in the cell is increased compared to the expression from the circular polyribonucleotide alone (e.g., lacking a capped polynucleotide) at least by 60 fold. In some embodiments, the expression of the one or more expression sequences from the complex in the cell is increased compared to the expression from the circular polyribonucleotide alone (e.g., lacking a capped polynucleotide) at least by 65 fold. In some embodiments, the expression of the one or more expression sequences from the complex in the cell is increased compared to the expression from the circular polyribonucleotide alone (e.g., lacking a capped polynucleotide) at least by 70 fold. In some embodiments, the expression of the one or more expression sequences from the complex in the cell is increased compared to the expression from the circular polyribonucleotide alone (e.g., lacking a capped polynucleotide) at least by 75 fold. In some embodiments, the expression of the one or more expression sequences from the complex in the cell is increased compared to the expression from the circular polyribonucleotide alone (e.g., lacking a capped polynucleotide) at least by 80 fold. In some embodiments, the expression of the one or more expression sequences from the complex in the cell is increased compared to the expression from the circular polyribonucleotide alone (e.g., lacking a capped polynucleotide) at least by 85 fold. In some embodiments, the expression of the one or more expression sequences from the complex in the cell is increased compared to the expression from the circular polyribonucleotide alone (e.g., lacking a capped polynucleotide) at least by 90 fold. In some embodiments, the expression of the one or more expression sequences from the complex in the cell is increased compared to the expression from the circular polyribonucleotide alone (e.g., lacking a capped polynucleotide) at least by 95 fold. In some embodiments, the expression of the one or more expression sequences from the complex in the cell is increased compared to the expression from the circular polyribonucleotide alone (e.g., lacking a capped polynucleotide) at least by 100 fold. In some embodiments, the expression of the one or more expression sequences from the complex in the cell is increased compared to the expression from the circular polyribonucleotide alone (e.g., lacking a capped polynucleotide) at least by 200 fold. In some embodiments, the expression of the one or more expression sequences from the complex in the cell is increased compared to the expression from the circular polyribonucleotide alone (e.g., lacking a capped polynucleotide) at least by 300 fold. In some embodiments, the expression of the one or more expression sequences from the complex in the cell is increased compared to the expression from the circular polyribonucleotide alone (e.g., lacking a capped polynucleotide) at least by 400 fold. In some embodiments, the expression of the one or more expression sequences from the complex in the cell is increased compared to the expression from the circular polyribonucleotide alone (e.g., lacking a capped polynucleotide) at least by 500 fold. In some embodiments, the expression of the one or more expression sequences from the complex in the cell is increased compared to the expression from the circular polyribonucleotide alone (e.g., lacking a capped polynucleotide) at least by 600 fold. In some embodiments, the expression of the one or more expression sequences from the complex in the cell is increased compared to the expression from the circular polyribonucleotide alone (e.g., lacking a capped polynucleotide) at least by 700 fold. In some embodiments, the expression of the one or more expression sequences from the complex in the cell is increased compared to the expression from the circular polyribonucleotide alone (e.g., lacking a capped polynucleotide) at least by 800 fold. In some embodiments, the expression of the one or more expression sequences from the complex in the cell is increased compared to the expression from the circular polyribonucleotide alone (e.g., lacking a capped polynucleotide) at least by 900 fold. In some embodiments, the expression of the one or more expression sequences from the complex in the cell is increased compared to the expression from the circular polyribonucleotide alone (e.g., lacking a capped polynucleotide) at least by 1000 fold. In some embodiments, the expression of the one or more expression sequences from the complex in the cell is increased compared to the expression from the circular polyribonucleotide alone (e.g., lacking a capped polynucleotide) at least by 5000 fold. In some embodiments, the expression of the one or more expression sequences from the complex in the cell is increased compared to the expression from the circular polyribonucleotide alone (e.g., lacking a capped polynucleotide) at least by 10000 fold. In some embodiments, the expression of the one or more expression sequences from the complex in the cell is increased compared to the expression from the circular polyribonucleotide alone (e.g., lacking a capped polynucleotide) by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 50000, 10000 fold or more. In some embodiments, the expression of the one or more expression sequences from the complex in the cell is increased compared to the expression from the circular polyribonucleotide alone (e.g., lacking a capped polynucleotide) by 2 fold. In some embodiments, the expression of the one or more expression sequences from the complex in the cell is increased compared to the expression from the circular polyribonucleotide alone (e.g., lacking a capped polynucleotide) by 3 fold. In some embodiments, the expression of the one or more expression sequences from the complex in the cell is increased compared to the expression from the circular polyribonucleotide alone (e.g., lacking a capped polynucleotide) by 4 fold. In some embodiments, the expression of the one or more expression sequences from the complex in the cell is increased compared to the expression from the circular polyribonucleotide alone (e.g., lacking a capped polynucleotide) by 5 fold. In some embodiments, the expression of the one or more expression sequences from the complex in the cell is increased compared to the expression from the circular polyribonucleotide alone (e.g., lacking a capped polynucleotide) by 6 fold. In some embodiments, the expression of the one or more expression sequences from the complex in the cell is increased compared to the expression from the circular polyribonucleotide alone (e.g., lacking a capped polynucleotide) by 7 fold. In some embodiments, the expression of the one or more expression sequences from the complex in the cell is increased compared to the expression from the circular polyribonucleotide alone (e.g., lacking a capped polynucleotide) by 8 fold. In some embodiments, the expression of the one or more expression sequences from the complex in the cell is increased compared to the expression from the circular polyribonucleotide alone (e.g., lacking a capped polynucleotide) by 9 fold. In some embodiments, the expression of the one or more expression sequences from the complex in the cell is increased compared to the expression from the circular polyribonucleotide alone (e.g., lacking a capped polynucleotide) by 10 fold. In some embodiments, the expression of the one or more expression sequences from the complex in the cell is increased compared to the expression from the circular polyribonucleotide alone (e.g., lacking a capped polynucleotide) by 15 fold. In some embodiments, the expression of the one or more expression sequences from the complex in the cell is increased compared to the expression from the circular polyribonucleotide alone (e.g., lacking a capped polynucleotide) by 20 fold. In some embodiments, the expression of the one or more expression sequences from the complex in the cell is increased compared to the expression from the circular polyribonucleotide alone (e.g., lacking a capped polynucleotide) by 25 fold. In some embodiments, the expression of the one or more expression sequences from the complex in the cell is increased compared to the expression from the circular polyribonucleotide alone (e.g., lacking a capped polynucleotide) by 30 fold. In some embodiments, the expression of the one or more expression sequences from the complex in the cell is increased compared to the expression from the circular polyribonucleotide alone (e.g., lacking a capped polynucleotide) by 35 fold. In some embodiments, the expression of the one or more expression sequences from the complex in the cell is increased compared to the expression from the circular polyribonucleotide alone (e.g., lacking a capped polynucleotide) by 40 fold. In some embodiments, the expression of the one or more expression sequences from the complex in the cell is increased compared to the expression from the circular polyribonucleotide alone (e.g., lacking a capped polynucleotide) by 45 fold. In some embodiments, the expression of the one or more expression sequences from the complex in the cell is increased compared to the expression from the circular polyribonucleotide alone (e.g., lacking a capped polynucleotide) by 50 fold. In some embodiments, the expression of the one or more expression sequences from the complex in the cell is increased compared to the expression from the circular polyribonucleotide alone (e.g., lacking a capped polynucleotide) by 55 fold. In some embodiments, the expression of the one or more expression sequences from the complex in the cell is increased compared to the expression from the circular polyribonucleotide alone (e.g., lacking a capped polynucleotide) by 60 fold. In some embodiments, the expression of the one or more expression sequences from the complex in the cell is increased compared to the expression from the circular polyribonucleotide alone (e.g., lacking a capped polynucleotide) by 65 fold. In some embodiments, the expression of the one or more expression sequences from the complex in the cell is increased compared to the expression from the circular polyribonucleotide alone (e.g., lacking a capped polynucleotide) by 70 fold. In some embodiments, the expression of the one or more expression sequences from the complex in the cell is increased compared to the expression from the circular polyribonucleotide alone (e.g., lacking a capped polynucleotide) by 75 fold. In some embodiments, the expression of the one or more expression sequences from the complex in the cell is increased compared to the expression from the circular polyribonucleotide alone (e.g., lacking a capped polynucleotide) by 80 fold. In some embodiments, the expression of the one or more expression sequences from the complex in the cell is increased compared to the expression from the circular polyribonucleotide alone (e.g., lacking a capped polynucleotide) by 85 fold. In some embodiments, the expression of the one or more expression sequences from the complex in the cell is increased compared to the expression from the circular polyribonucleotide alone (e.g., lacking a capped polynucleotide) by 90 fold. In some embodiments, the expression of the one or more expression sequences from the complex in the cell is increased compared to the expression from the circular polyribonucleotide alone (e.g., lacking a capped polynucleotide) by 95 fold. In some embodiments, the expression of the one or more expression sequences from the complex in the cell is increased compared to the expression from the circular polyribonucleotide alone (e.g., lacking a capped polynucleotide) by 100 fold. In some embodiments, the expression of the one or more expression sequences from the complex in the cell is increased compared to the expression from the circular polyribonucleotide alone (e.g., lacking a capped polynucleotide) by 200 fold. In some embodiments, the expression of the one or more expression sequences from the complex in the cell is increased compared to the expression from the circular polyribonucleotide alone (e.g., lacking a capped polynucleotide) by 300 fold. In some embodiments, the expression of the one or more expression sequences from the complex in the cell is increased compared to the expression from the circular polyribonucleotide alone (e.g., lacking a capped polynucleotide) by 400 fold. In some embodiments, the expression of the one or more expression sequences from the complex in the cell is increased compared to the expression from the circular polyribonucleotide alone (e.g., lacking a capped polynucleotide) by 500 fold. In some embodiments, the expression of the one or more expression sequences from the complex in the cell is increased compared to the expression from the circular polyribonucleotide alone (e.g., lacking a capped polynucleotide) by 600 fold. In some embodiments, the expression of the one or more expression sequences from the complex in the cell is increased compared to the expression from the circular polyribonucleotide alone (e.g., lacking a capped polynucleotide) by 700 fold. In some embodiments, the expression of the one or more expression sequences from the complex in the cell is increased compared to the expression from the circular polyribonucleotide alone (e.g., lacking a capped polynucleotide) by 800 fold. In some embodiments, the expression of the one or more expression sequences from the complex in the cell is increased compared to the expression from the circular polyribonucleotide alone (e.g., lacking a capped polynucleotide) by 900 fold. In some embodiments, the expression of the one or more expression sequences from the complex in the cell is increased compared to the expression from the circular polyribonucleotide alone (e.g., lacking a capped polynucleotide) by 1000 fold. In some embodiments, the expression of the one or more expression sequences from the complex in the cell is increased compared to the expression from the circular polyribonucleotide alone (e.g., lacking a capped polynucleotide) by 50000 fold. In some embodiments, the expression of the one or more expression sequences from the complex in the cell is increased compared to the expression from the circular polyribonucleotide alone (e.g., lacking a capped polynucleotide) by 10000 fold.

### Increased in Vivo Expression

In some aspects, the invention as provided herein comprises a method of in vivo expression of one or more expression sequences, comprising: providing a complex comprising a circular polyribonucleotide as provided herein comprising the one or more expression sequences, and a capped polyribonucleotide as provided herein, wherein the first binding region of the capped polyribonucleotide is bound to the second binding region of the circular polyribonucleotide; and administering the complex to a cell in vivo, wherein expression of the one or more expression sequences from the complex in the cell is higher than expression from the circular polyribonucleotide alone (e.g., lacking a capped polynucleotide).

In some other aspects, the invention as provided herein a method of in vivo expression of one or more expression sequences, comprising: administering to a cell in vivo a circular polyribonucleotide as provided herein comprising the one or more expression sequences, and a capped polyribonucleotide as provided herein to a cell, wherein the first binding region of the capped polyribonucleotide is bound to the second binding region of the circular polyribonucleotide to form a complex in the cell and the expression of the one or more expression sequences from the complex in the cell is higher than expression from the circular polyribonucleotide alone (e.g., lacking a capped polynucleotide).

In some aspects, the invention as provided herein comprises a method of in vivo expression of one or more expression sequences, comprising: providing a complex comprising a circular polyribonucleotide as provided herein comprising the one or more expression sequences, a first capped polyribonucleotide as provided herein, wherein the first binding region of the first capped polyribonucleotide is bound to the second binding region of the circular polyribonucleotide, and a second capped polyribonucleotide as provided herein, wherein the third binding region of the second capped polyribonucleotide is bound to the fourth binding region of the circular polyribonucleotide; and administering the complex to a cell in vivo, wherein expression of the one or more expression sequences from the complex in the cell is higher than expression from the circular polyribonucleotide alone (e.g., lacking a capped polynucleotide). In some aspects, the invention as provided herein comprises a method of in vivo expression of one or more expression sequences, comprising: providing a complex comprising a circular polyribonucleotide as provided herein comprising the one or more expression sequences, a first capped polyribonucleotide as provided herein, wherein the first binding region of the first capped polyribonucleotide is bound to the second binding region of the circular polyribonucleotide, and a second capped polyribonucleotide as provided herein, wherein the third binding region of the second capped polyribonucleotide is bound to the fourth binding region of the circular polyribonucleotide; and administering the complex to a cell in vivo, wherein expression of the one or more expression sequences from the complex in the cell is higher than expression from the circular polyribonucleotide bound to the first capped polyribonucleotide. In some aspects, the invention as provided herein comprises a method of in vivo expression of one or more expression sequences, comprising: providing a complex comprising a circular polyribonucleotide as provided herein comprising the one or more expression sequences, a first capped polyribonucleotide as provided herein, wherein the first binding region of the first capped polyribonucleotide is bound to the second binding region of the circular polyribonucleotide, and a second capped polyribonucleotide as provided herein, wherein the third binding region of the second capped polyribonucleotide is bound to the fourth binding region of the circular polyribonucleotide; and administering the complex to a cell in vivo, wherein expression of the one or more expression sequences from the complex in the cell is higher than expression from the circular polyribonucleotide bound to the second capped polyribonucleotide.

In some other aspects, the invention as provided herein a method of in vivo expression of one or more expression sequences, comprising: administering to a cell in vivo a circular polyribonucleotide as provided herein comprising the one or more expression sequences, a first capped polyribonucleotide as provided herein to a cell, wherein the first binding region of the first capped polyribonucleotide is bound to the second binding region of the circular polyribonucleotide, and a second capped polyribonucleotide as provided herein to a cell, wherein the third binding region of the second capped polyribonucleotide is bound to the fourth binding region of the circular polyribonucleotide to form a complex in the cell and the expression of the one or more expression sequences from the complex in the cell is higher than expression from the circular polyribonucleotide alone (e.g., lacking a capped polynucleotide). In some other aspects, the invention as provided herein a method of in vivo expression of one or more expression sequences, comprising: administering to a cell in vivo a circular polyribonucleotide as provided herein comprising the one or more expression sequences, a first capped polyribonucleotide as provided herein to a cell, wherein the first binding region of the first capped polyribonucleotide is bound to the second binding region of the circular polyribonucleotide, and a second capped polyribonucleotide as provided herein to a cell, wherein the third binding region of the second capped polyribonucleotide is bound to the fourth binding region of the circular polyribonucleotide to form a complex in the cell and the expression of the one or more expression sequences from the complex in the cell is higher than expression from the circular polyribonucleotide bound to the first capped polyribonucleotide. In some other aspects, the invention as provided herein a method of in vivo expression of one or more expression sequences, comprising: administering to a cell in vivo a circular polyribonucleotide as provided herein comprising the one or more expression sequences, a first capped polyribonucleotide as provided herein to a cell, wherein the first binding region of the first capped polyribonucleotide is bound to the second binding region of the circular polyribonucleotide, and a second capped polyribonucleotide as provided herein to a cell, wherein the third binding region of the second capped polyribonucleotide is bound to the fourth binding region of the circular polyribonucleotide to form a complex in the cell and the expression of the one or more expression sequences from the complex in the cell is higher than expression from the circular polyribonucleotide bound to the second capped polyribonucleotide.

In some embodiments, the expression of the one or more expression sequences from the complex in the cell is at least 10%, 20%, 30%, 40%, 50%, 60%, 70%, 80%, 90%, 100%, 200%, 300%, 400%, 500%, 600%, 700%, 800%, 900%, 1000%, 5000%, 10000% or more higher than the expression from the circular polyribonucleotide alone (e.g., lacking a capped polynucleotide). In some embodiments, the expression of the one or more expression sequences from the complex in the cell is at least 10% higher than the expression from the circular polyribonucleotide alone (e.g., lacking a capped polynucleotide). In some embodiments, the expression of the one or more expression sequences from the complex in the cell is at least 20% higher than the expression from the circular polyribonucleotide alone (e.g., lacking a capped polynucleotide). In some embodiments, the expression of the one or more expression sequences from the complex in the cell is at least 30% higher than the expression from the circular polyribonucleotide alone (e.g., lacking a capped polynucleotide). In some embodiments, the expression of the one or more expression sequences from the complex in the cell is at least 40% higher than the expression from the circular polyribonucleotide alone (e.g., lacking a capped polynucleotide). In some embodiments, the expression of the one or more expression sequences from the complex in the cell is at least 50% higher than the expression from the circular polyribonucleotide alone (e.g., lacking a capped polynucleotide). In some embodiments, the expression of the one or more expression sequences from the complex in the cell is at least 60% higher than the expression from the circular polyribonucleotide alone (e.g., lacking a capped polynucleotide). In some embodiments, the expression of the one or more expression sequences from the complex in the cell is at least 70% higher than the expression from the circular polyribonucleotide alone (e.g., lacking a capped polynucleotide). In some embodiments, the expression of the one or more expression sequences from the complex in the cell is at least 80% higher than the expression from the circular polyribonucleotide alone (e.g., lacking a capped polynucleotide). In some embodiments, the expression of the one or more expression sequences from the complex in the cell is at least 90% higher than the expression from the circular polyribonucleotide alone (e.g., lacking a capped polynucleotide). In some embodiments, the expression of the one or more expression sequences from the complex in the cell is at least 100% higher than the expression from the circular polyribonucleotide alone (e.g., lacking a capped polynucleotide). In some embodiments, the expression of the one or more expression sequences from the complex in the cell is at least 200% higher than the expression from the circular polyribonucleotide alone (e.g., lacking a capped polynucleotide). In some embodiments, the expression of the one or more expression sequences from the complex in the cell is at least 300% higher than the expression from the circular polyribonucleotide alone (e.g., lacking a capped polynucleotide). In some embodiments, the expression of the one or more expression sequences from the complex in the cell is at least 400% higher than the expression from the circular polyribonucleotide alone (e.g., lacking a capped polynucleotide). In some embodiments, the expression of the one or more expression sequences from the complex in the cell is at least 500% higher than the expression from the circular polyribonucleotide alone (e.g., lacking a capped polynucleotide). In some embodiments, the expression of the one or more expression sequences from the complex in the cell is at least 600% higher than the expression from the circular polyribonucleotide alone (e.g., lacking a capped polynucleotide). In some embodiments, the expression of the one or more expression sequences from the complex in the cell is at least 700% higher than the expression from the circular polyribonucleotide alone (e.g., lacking a capped polynucleotide). In some embodiments, the expression of the one or more expression sequences from the complex in the cell is at least 800% higher than the expression from the circular polyribonucleotide alone (e.g., lacking a capped polynucleotide). In some embodiments, the expression of the one or more expression sequences from the complex in the cell is at least 900% higher than the expression from the circular polyribonucleotide alone (e.g., lacking a capped polynucleotide). In some embodiments, the expression of the one or more expression sequences from the complex in the cell is at least 1000% higher than the expression from the circular polyribonucleotide alone (e.g., lacking a capped polynucleotide). In some embodiments, the expression of the one or more expression sequences from the complex in the cell is at least 5000% higher than the expression from the circular polyribonucleotide alone (e.g., lacking a capped polynucleotide). In some embodiments, the expression of the one or more expression sequences from the complex in the cell is at least 10000% higher than the expression from the circular polyribonucleotide alone (e.g., lacking a capped polynucleotide). In some embodiments, the expression of the one or more expression sequences from the complex in the cell is 10%, 20%, 30%, 40%, 50%, 60%, 70%, 80%, 90%, 100%, 200%, 300%, 400%, 500%, 600%, 700%, 800%, 900%, 1000%, 5000%, 10000% or more higher than the expression from the circular polyribonucleotide alone (e.g., lacking a capped polynucleotide). In some embodiments, the expression of the one or more expression sequences from the complex in the cell is 10% higher than the expression from the circular polyribonucleotide alone (e.g., lacking a capped polynucleotide). In some embodiments, the expression of the one or more expression sequences from the complex in the cell is 20% higher than the expression from the circular polyribonucleotide alone (e.g., lacking a capped polynucleotide). In some embodiments, the expression of the one or more expression sequences from the complex in the cell is 30% higher than the expression from the circular polyribonucleotide alone (e.g., lacking a capped polynucleotide). In some embodiments, the expression of the one or more expression sequences from the complex in the cell is 40% higher than the expression from the circular polyribonucleotide alone (e.g., lacking a capped polynucleotide). In some embodiments, the expression of the one or more expression sequences from the complex in the cell is 50% higher than the expression from the circular polyribonucleotide alone (e.g., lacking a capped polynucleotide). In some embodiments, the expression of the one or more expression sequences from the complex in the cell is 60% higher than the expression from the circular polyribonucleotide alone (e.g., lacking a capped polynucleotide). In some embodiments, the expression of the one or more expression sequences from the complex in the cell is 70% higher than the expression from the circular polyribonucleotide alone (e.g., lacking a capped polynucleotide). In some embodiments, the expression of the one or more expression sequences from the complex in the cell is 80% higher than the expression from the circular polyribonucleotide alone (e.g., lacking a capped polynucleotide). In some embodiments, the expression of the one or more expression sequences from the complex in the cell is 90% higher than the expression from the circular polyribonucleotide alone (e.g., lacking a capped polynucleotide). In some embodiments, the expression of the one or more expression sequences from the complex in the cell is 100% higher than the expression from the circular polyribonucleotide alone (e.g., lacking a capped polynucleotide). In some embodiments, the expression of the one or more expression sequences from the complex in the cell is 200% higher than the expression from the circular polyribonucleotide alone (e.g., lacking a capped polynucleotide). In some embodiments, the expression of the one or more expression sequences from the complex in the cell is 300% higher than the expression from the circular polyribonucleotide alone (e.g., lacking a capped polynucleotide). In some embodiments, the expression of the one or more expression sequences from the complex in the cell is 400% higher than the expression from the circular polyribonucleotide alone (e.g., lacking a capped polynucleotide). In some embodiments, the expression of the one or more expression sequences from the complex in the cell is 500% higher than the expression from the circular polyribonucleotide alone (e.g., lacking a capped polynucleotide). In some embodiments, the expression of the one or more expression sequences from the complex in the cell is 600% higher than the expression from the circular polyribonucleotide alone (e.g., lacking a capped polynucleotide). In some embodiments, the expression of the one or more expression sequences from the complex in the cell is 700% higher than the expression from the circular polyribonucleotide alone (e.g., lacking a capped polynucleotide). In some embodiments, the expression of the one or more expression sequences from the complex in the cell is 800% higher than the expression from the circular polyribonucleotide alone (e.g., lacking a capped polynucleotide). In some embodiments, the expression of the one or more expression sequences from the complex in the cell is 900% higher than the expression from the circular polyribonucleotide alone (e.g., lacking a capped polynucleotide). In some embodiments, the expression of the one or more expression sequences from the complex in the cell is 1000% higher than the expression from the circular polyribonucleotide alone (e.g., lacking a capped polynucleotide). In some embodiments, the expression of the one or more expression sequences from the complex in the cell is 5000% higher than the expression from the circular polyribonucleotide alone (e.g., lacking a capped polynucleotide). In some embodiments, the expression of the one or more expression sequences from the complex in the cell is 10000% higher than the expression from the circular polyribonucleotide alone (e.g., lacking a capped polynucleotide). In some embodiments, the expression of the one or more expression sequences from the complex in the cell is higher than the expression from the circular polyribonucleotide alone at least by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 5000, 10000 fold or more. In some embodiments, the expression of the one or more expression sequences from the complex in the cell is higher than the expression from the circular polyribonucleotide alone at least by 2 fold. In some embodiments, the expression of the one or more expression sequences from the complex in the cell is higher than the expression from the circular polyribonucleotide alone at least by 3 fold. In some embodiments, the expression of the one or more expression sequences from the complex in the cell is higher than the expression from the circular polyribonucleotide alone at least by 4 fold. In some embodiments, the expression of the one or more expression sequences from the complex in the cell is higher than the expression from the circular polyribonucleotide alone at least by 5 fold. In some embodiments, the expression of the one or more expression sequences from the complex in the cell is higher than the expression from the circular polyribonucleotide alone at least by 6 fold. In some embodiments, the expression of the one or more expression sequences from the complex in the cell is higher than the expression from the circular polyribonucleotide alone at least by 7 fold. In some embodiments, the expression of the one or more expression sequences from the complex in the cell is higher than the expression from the circular polyribonucleotide alone at least by 8 fold. In some embodiments, the expression of the one or more expression sequences from the complex in the cell is higher than the expression from the circular polyribonucleotide alone at least by 9 fold. In some embodiments, the expression of the one or more expression sequences from the complex in the cell is higher than the expression from the circular polyribonucleotide alone at least by 10 fold. In some embodiments, the expression of the one or more expression sequences from the complex in the cell is higher than the expression from the circular polyribonucleotide alone at least by 15 fold. In some embodiments, the expression of the one or more expression sequences from the complex in the cell is higher than the expression from the circular polyribonucleotide alone at least by 20 fold. In some embodiments, the expression of the one or more expression sequences from the complex in the cell is higher than the expression from the circular polyribonucleotide alone at least by 25 fold. In some embodiments, the expression of the one or more expression sequences from the complex in the cell is higher than the expression from the circular polyribonucleotide alone at least by 30 fold. In some embodiments, the expression of the one or more expression sequences from the complex in the cell is higher than the expression from the circular polyribonucleotide alone at least by 35 fold. In some embodiments, the expression of the one or more expression sequences from the complex in the cell is higher than the expression from the circular polyribonucleotide alone at least by 40 fold. In some embodiments, the expression of the one or more expression sequences from the complex in the cell is higher than the expression from the circular polyribonucleotide alone at least by 45 fold. In some embodiments, the expression of the one or more expression sequences from the complex in the cell is higher than the expression from the circular polyribonucleotide alone at least by 50 fold. In some embodiments, the expression of the one or more expression sequences from the complex in the cell is higher than the expression from the circular polyribonucleotide alone at least by 55 fold. In some embodiments, the expression of the one or more expression sequences from the complex in the cell is higher than the expression from the circular polyribonucleotide alone at least by 60 fold. In some embodiments, the expression of the one or more expression sequences from the complex in the cell is higher than the expression from the circular polyribonucleotide alone at least by 65 fold. In some embodiments, the expression of the one or more expression sequences from the complex in the cell is higher than the expression from the circular polyribonucleotide alone at least by 70 fold. In some embodiments, the expression of the one or more expression sequences from the complex in the cell is higher than the expression from the circular polyribonucleotide alone at least by 75 fold. In some embodiments, the expression of the one or more expression sequences from the complex in the cell is higher than the expression from the circular polyribonucleotide alone at least by 80 fold. In some embodiments, the expression of the one or more expression sequences from the complex in the cell is higher than the expression from the circular polyribonucleotide alone at least by 85 fold. In some embodiments, the expression of the one or more expression sequences from the complex in the cell is higher than the expression from the circular polyribonucleotide alone at least by 90 fold. In some embodiments, the expression of the one or more expression sequences from the complex in the cell is higher than the expression from the circular polyribonucleotide alone at least by 95 fold. In some embodiments, the expression of the one or more expression sequences from the complex in the cell is higher than the expression from the circular polyribonucleotide alone at least by 100 fold. In some embodiments, the expression of the one or more expression sequences from the complex in the cell is higher than the expression from the circular polyribonucleotide alone at least by 200 fold. In some embodiments, the expression of the one or more expression sequences from the complex in the cell is higher than the expression from the circular polyribonucleotide alone at least by 300 fold. In some embodiments, the expression of the one or more expression sequences from the complex in the cell is higher than the expression from the circular polyribonucleotide alone at least by 400 fold. In some embodiments, the expression of the one or more expression sequences from the complex in the cell is higher than the expression from the circular polyribonucleotide alone at least by 500 fold. In some embodiments, the expression of the one or more expression sequences from the complex in the cell is higher than the expression from the circular polyribonucleotide alone at least by 600 fold. In some embodiments, the expression of the one or more expression sequences from the complex in the cell is higher than the expression from the circular polyribonucleotide alone at least by 700 fold. In some embodiments, the expression of the one or more expression sequences from the complex in the cell is higher than the expression from the circular polyribonucleotide alone at least by 800 fold. In some embodiments, the expression of the one or more expression sequences from the complex in the cell is higher than the expression from the circular polyribonucleotide alone at least by 900 fold. In some embodiments, the expression of the one or more expression sequences from the complex in the cell is higher than the expression from the circular polyribonucleotide alone at least by 1000 fold. In some embodiments, the expression of the one or more expression sequences from the complex in the cell is higher than the expression from the circular polyribonucleotide alone at least by 5000 fold. In some embodiments, the expression of the one or more expression sequences from the complex in the cell is higher than the expression from the circular polyribonucleotide alone at least by 10000 fold. In some embodiments, the expression of the one or more expression sequences from the complex in the cell is higher than the expression from the circular polyribonucleotide alone (e.g., lacking a capped polynucleotide) by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 5000, 10000 fold or more. In some embodiments, the expression of the one or more expression sequences from the complex in the cell is higher than the expression from the circular polyribonucleotide alone (e.g., lacking a capped polynucleotide) by 2 fold. In some embodiments, the expression of the one or more expression sequences from the complex in the cell is higher than the expression from the circular polyribonucleotide alone (e.g., lacking a capped polynucleotide) by 3 fold. In some embodiments, the expression of the one or more expression sequences from the complex in the cell is higher than the expression from the circular polyribonucleotide alone (e.g., lacking a capped polynucleotide) by 4 fold. In some embodiments, the expression of the one or more expression sequences from the complex in the cell is higher than the expression from the circular polyribonucleotide alone (e.g., lacking a capped polynucleotide) by 5 fold. In some embodiments, the expression of the one or more expression sequences from the complex in the cell is higher than the expression from the circular polyribonucleotide alone (e.g., lacking a capped polynucleotide) by 6 fold. In some embodiments, the expression of the one or more expression sequences from the complex in the cell is higher than the expression from the circular polyribonucleotide alone (e.g., lacking a capped polynucleotide) by 7 fold. In some embodiments, the expression of the one or more expression sequences from the complex in the cell is higher than the expression from the circular polyribonucleotide alone (e.g., lacking a capped polynucleotide) by 8 fold. In some embodiments, the expression of the one or more expression sequences from the complex in the cell is higher than the expression from the circular polyribonucleotide alone (e.g., lacking a capped polynucleotide) by 9 fold. In some embodiments, the expression of the one or more expression sequences from the complex in the cell is higher than the expression from the circular polyribonucleotide alone (e.g., lacking a capped polynucleotide) by 10 fold. In some embodiments, the expression of the one or more expression sequences from the complex in the cell is higher than the expression from the circular polyribonucleotide alone (e.g., lacking a capped polynucleotide) by 15 fold. In some embodiments, the expression of the one or more expression sequences from the complex in the cell is higher than the expression from the circular polyribonucleotide alone (e.g., lacking a capped polynucleotide) by 20 fold. In some embodiments, the expression of the one or more expression sequences from the complex in the cell is higher than the expression from the circular polyribonucleotide alone (e.g., lacking a capped polynucleotide) by 25 fold. In some embodiments, the expression of the one or more expression sequences from the complex in the cell is higher than the expression from the circular polyribonucleotide alone (e.g., lacking a capped polynucleotide) by 30 fold. In some embodiments, the expression of the one or more expression sequences from the complex in the cell is higher than the expression from the circular polyribonucleotide alone (e.g., lacking a capped polynucleotide) by 35 fold. In some embodiments, the expression of the one or more expression sequences from the complex in the cell is higher than the expression from the circular polyribonucleotide alone (e.g., lacking a capped polynucleotide) by 40 fold. In some embodiments, the expression of the one or more expression sequences from the complex in the cell is higher than the expression from the circular polyribonucleotide alone (e.g., lacking a capped polynucleotide) by 45 fold. In some embodiments, the expression of the one or more expression sequences from the complex in the cell is higher than the expression from the circular polyribonucleotide alone (e.g., lacking a capped polynucleotide) by 50 fold. In some embodiments, the expression of the one or more expression sequences from the complex in the cell is higher than the expression from the circular polyribonucleotide alone (e.g., lacking a capped polynucleotide) by 55 fold. In some embodiments, the expression of the one or more expression sequences from the complex in the cell is higher than the expression from the circular polyribonucleotide alone (e.g., lacking a capped polynucleotide) by 60 fold. In some embodiments, the expression of the one or more expression sequences from the complex in the cell is higher than the expression from the circular polyribonucleotide alone (e.g., lacking a capped polynucleotide) by 65 fold. In some embodiments, the expression of the one or more expression sequences from the complex in the cell is higher than the expression from the circular polyribonucleotide alone (e.g., lacking a capped polynucleotide) by 70 fold. In some embodiments, the expression of the one or more expression sequences from the complex in the cell is higher than the expression from the circular polyribonucleotide alone (e.g., lacking a capped polynucleotide) by 75 fold. In some embodiments, the expression of the one or more expression sequences from the complex in the cell is higher than the expression from the circular polyribonucleotide alone (e.g., lacking a capped polynucleotide) by 80 fold. In some embodiments, the expression of the one or more expression sequences from the complex in the cell is higher than the expression from the circular polyribonucleotide alone (e.g., lacking a capped polynucleotide) by 85 fold. In some embodiments, the expression of the one or more expression sequences from the complex in the cell is higher than the expression from the circular polyribonucleotide alone (e.g., lacking a capped polynucleotide) by 90 fold. In some embodiments, the expression of the one or more expression sequences from the complex in the cell is higher than the expression from the circular polyribonucleotide alone (e.g., lacking a capped polynucleotide) by 95 fold. In some embodiments, the expression of the one or more expression sequences from the complex in the cell is higher than the expression from the circular polyribonucleotide alone (e.g., lacking a capped polynucleotide) by 100 fold. In some embodiments, the expression of the one or more expression sequences from the complex in the cell is higher than the expression from the circular polyribonucleotide alone (e.g., lacking a capped polynucleotide) by 200 fold. In some embodiments, the expression of the one or more expression sequences from the complex in the cell is higher than the expression from the circular polyribonucleotide alone (e.g., lacking a capped polynucleotide) by 300 fold. In some embodiments, the expression of the one or more expression sequences from the complex in the cell is higher than the expression from the circular polyribonucleotide alone (e.g., lacking a capped polynucleotide) by 400 fold. In some embodiments, the expression of the one or more expression sequences from the complex in the cell is higher than the expression from the circular polyribonucleotide alone (e.g., lacking a capped polynucleotide) by 500 fold. In some embodiments, the expression of the one or more expression sequences from the complex in the cell is higher than the expression from the circular polyribonucleotide alone (e.g., lacking a capped polynucleotide) by 600 fold. In some embodiments, the expression of the one or more expression sequences from the complex in the cell is higher than the expression from the circular polyribonucleotide alone (e.g., lacking a capped polynucleotide) by 700 fold. In some embodiments, the expression of the one or more expression sequences from the complex in the cell is higher than the expression from the circular polyribonucleotide alone (e.g., lacking a capped polynucleotide) by 800 fold. In some embodiments, the expression of the one or more expression sequences from the complex in the cell is higher than the expression from the circular polyribonucleotide alone (e.g., lacking a capped polynucleotide) by 900 fold. In some embodiments, the expression of the one or more expression sequences from the complex in the cell is higher than the expression from the circular polyribonucleotide alone (e.g., lacking a capped polynucleotide) by 1000 fold. In some embodiments, the expression of the one or more expression sequences from the complex in the cell is higher than the expression from the circular polyribonucleotide alone (e.g., lacking a capped polynucleotide) by 5000 fold. In some embodiments, the expression of the one or more expression sequences from the complex in the cell is higher than the expression from the circular polyribonucleotide alone (e.g., lacking a capped polynucleotide) by 10000 fold.

In some other aspects, the invention as provided herein a method of in vivo expression of one or more expression sequences, comprising: administering to a cell in vivo a circular polyribonucleotide as provided herein comprising the one or more expression sequences, and a capped polyribonucleotide as provided herein to a cell, wherein the first binding region of the capped polyribonucleotide is bound to the second binding region of the circular polyribonucleotide to form a complex in the cell and the expression of the one or more expression sequences from the complex in the cell is greater than expression from the circular polyribonucleotide alone (e.g., lacking a capped polynucleotide).

In some embodiments, the expression of the one or more expression sequences from the complex in the cell is at least 10%, 20%, 30%, 40%, 50%, 60%, 70%, 80%, 90%, 100%, 200%, 300%, 400%, 500%, 600%, 700%, 800%, 900%, 1000%, 5000%, 10000% or more greater than the expression from the circular polyribonucleotide alone (e.g., lacking a capped polynucleotide). In some embodiments, the expression of the one or more expression sequences from the complex in the cell is at least 10% greater than the expression from the circular polyribonucleotide alone (e.g., lacking a capped polynucleotide). In some embodiments, the expression of the one or more expression sequences from the complex in the cell is at least 20% greater than the expression from the circular polyribonucleotide alone (e.g., lacking a capped polynucleotide). In some embodiments, the expression of the one or more expression sequences from the complex in the cell is at least 30% greater than the expression from the circular polyribonucleotide alone (e.g., lacking a capped polynucleotide). In some embodiments, the expression of the one or more expression sequences from the complex in the cell is at least 40% greater than the expression from the circular polyribonucleotide alone (e.g., lacking a capped polynucleotide). In some embodiments, the expression of the one or more expression sequences from the complex in the cell is at least 50% greater than the expression from the circular polyribonucleotide alone (e.g., lacking a capped polynucleotide). In some embodiments, the expression of the one or more expression sequences from the complex in the cell is at least 60% greater than the expression from the circular polyribonucleotide alone (e.g., lacking a capped polynucleotide). In some embodiments, the expression of the one or more expression sequences from the complex in the cell is at least 70% greater than the expression from the circular polyribonucleotide alone (e.g., lacking a capped polynucleotide). In some embodiments, the expression of the one or more expression sequences from the complex in the cell is at least 80% greater than the expression from the circular polyribonucleotide alone (e.g., lacking a capped polynucleotide). In some embodiments, the expression of the one or more expression sequences from the complex in the cell is at least 90% greater than the expression from the circular polyribonucleotide alone (e.g., lacking a capped polynucleotide). In some embodiments, the expression of the one or more expression sequences from the complex in the cell is at least 100% greater than the expression from the circular polyribonucleotide alone (e.g., lacking a capped polynucleotide). In some embodiments, the expression of the one or more expression sequences from the complex in the cell is at least 200% greater than the expression from the circular polyribonucleotide alone (e.g., lacking a capped polynucleotide). In some embodiments, the expression of the one or more expression sequences from the complex in the cell is at least 300% greater than the expression from the circular polyribonucleotide alone (e.g., lacking a capped polynucleotide). In some embodiments, the expression of the one or more expression sequences from the complex in the cell is at least 400% greater than the expression from the circular polyribonucleotide alone (e.g., lacking a capped polynucleotide). In some embodiments, the expression of the one or more expression sequences from the complex in the cell is at least 500% greater than the expression from the circular polyribonucleotide alone (e.g., lacking a capped polynucleotide). In some embodiments, the expression of the one or more expression sequences from the complex in the cell is at least 600% greater than the expression from the circular polyribonucleotide alone (e.g., lacking a capped polynucleotide). In some embodiments, the expression of the one or more expression sequences from the complex in the cell is at least 700% greater than the expression from the circular polyribonucleotide alone (e.g., lacking a capped polynucleotide). In some embodiments, the expression of the one or more expression sequences from the complex in the cell is at least 800% greater than the expression from the circular polyribonucleotide alone (e.g., lacking a capped polynucleotide). In some embodiments, the expression of the one or more expression sequences from the complex in the cell is at least 900% greater than the expression from the circular polyribonucleotide alone (e.g., lacking a capped polynucleotide). In some embodiments, the expression of the one or more expression sequences from the complex in the cell is at least 1000% greater than the expression from the circular polyribonucleotide alone (e.g., lacking a capped polynucleotide). In some embodiments, the expression of the one or more expression sequences from the complex in the cell is at least 5000% greater than the expression from the circular polyribonucleotide alone (e.g., lacking a capped polynucleotide). In some embodiments, the expression of the one or more expression sequences from the complex in the cell is at least 10000% greater than the expression from the circular polyribonucleotide alone (e.g., lacking a capped polynucleotide). In some embodiments, the expression of the one or more expression sequences from the complex in the cell is 10%, 20%, 30%, 40%, 50%, 60%, 70%, 80%, 90%, 100%, 200%, 300%, 400%, 500%, 600%, 700%, 800%, 900%, 1000%, 5000%, 10000% or more greater than the expression from the circular polyribonucleotide alone (e.g., lacking a capped polynucleotide). In some embodiments, the expression of the one or more expression sequences from the complex in the cell is 10% greater than the expression from the circular polyribonucleotide alone (e.g., lacking a capped polynucleotide). In some embodiments, the expression of the one or more expression sequences from the complex in the cell is 20% greater than the expression from the circular polyribonucleotide alone (e.g., lacking a capped polynucleotide). In some embodiments, the expression of the one or more expression sequences from the complex in the cell is 30% greater than the expression from the circular polyribonucleotide alone (e.g., lacking a capped polynucleotide). In some embodiments, the expression of the one or more expression sequences from the complex in the cell is 40% greater than the expression from the circular polyribonucleotide alone (e.g., lacking a capped polynucleotide). In some embodiments, the expression of the one or more expression sequences from the complex in the cell is 50% greater than the expression from the circular polyribonucleotide alone (e.g., lacking a capped polynucleotide). In some embodiments, the expression of the one or more expression sequences from the complex in the cell is 60% greater than the expression from the circular polyribonucleotide alone (e.g., lacking a capped polynucleotide). In some embodiments, the expression of the one or more expression sequences from the complex in the cell is 70% greater than the expression from the circular polyribonucleotide alone (e.g., lacking a capped polynucleotide). In some embodiments, the expression of the one or more expression sequences from the complex in the cell is 80% greater than the expression from the circular polyribonucleotide alone (e.g., lacking a capped polynucleotide). In some embodiments, the expression of the one or more expression sequences from the complex in the cell is 90% greater than the expression from the circular polyribonucleotide alone (e.g., lacking a capped polynucleotide). In some embodiments, the expression of the one or more expression sequences from the complex in the cell is 100% greater than the expression from the circular polyribonucleotide alone (e.g., lacking a capped polynucleotide). In some embodiments, the expression of the one or more expression sequences from the complex in the cell is 200% greater than the expression from the circular polyribonucleotide alone (e.g., lacking a capped polynucleotide). In some embodiments, the expression of the one or more expression sequences from the complex in the cell is 300% greater than the expression from the circular polyribonucleotide alone (e.g., lacking a capped polynucleotide). In some embodiments, the expression of the one or more expression sequences from the complex in the cell is 400% greater than the expression from the circular polyribonucleotide alone (e.g., lacking a capped polynucleotide). In some embodiments, the expression of the one or more expression sequences from the complex in the cell is 500% greater than the expression from the circular polyribonucleotide alone (e.g., lacking a capped polynucleotide). In some embodiments, the expression of the one or more expression sequences from the complex in the cell is 600% greater than the expression from the circular polyribonucleotide alone (e.g., lacking a capped polynucleotide). In some embodiments, the expression of the one or more expression sequences from the complex in the cell is 700% greater than the expression from the circular polyribonucleotide alone (e.g., lacking a capped polynucleotide). In some embodiments, the expression of the one or more expression sequences from the complex in the cell is 800% greater than the expression from the circular polyribonucleotide alone (e.g., lacking a capped polynucleotide). In some embodiments, the expression of the one or more expression sequences from the complex in the cell is 900% greater than the expression from the circular polyribonucleotide alone (e.g., lacking a capped polynucleotide). In some embodiments, the expression of the one or more expression sequences from the complex in the cell is 1000% greater than the expression from the circular polyribonucleotide alone (e.g., lacking a capped polynucleotide). In some embodiments, the expression of the one or more expression sequences from the complex in the cell is 5000% greater than the expression from the circular polyribonucleotide alone (e.g., lacking a capped polynucleotide). In some embodiments, the expression of the one or more expression sequences from the complex in the cell is 10000% greater than the expression from the circular polyribonucleotide alone (e.g., lacking a capped polynucleotide). In some embodiments, the expression of the one or more expression sequences from the complex in the cell is higher than the expression from the circular polyribonucleotide alone (e.g., lacking a capped polynucleotide) at least by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 5000, 10000 fold or more. In some embodiments, the expression of the one or more expression sequences from the complex in the cell is higher than the expression from the circular polyribonucleotide alone (e.g., lacking a capped polynucleotide) at least by 2 fold. In some embodiments, the expression of the one or more expression sequences from the complex in the cell is higher than the expression from the circular polyribonucleotide alone (e.g., lacking a capped polynucleotide) at least by 3 fold. In some embodiments, the expression of the one or more expression sequences from the complex in the cell is higher than the expression from the circular polyribonucleotide alone (e.g., lacking a capped polynucleotide) at least by 4 fold. In some embodiments, the expression of the one or more expression sequences from the complex in the cell is higher than the expression from the circular polyribonucleotide alone (e.g., lacking a capped polynucleotide) at least by 5 fold. In some embodiments, the expression of the one or more expression sequences from the complex in the cell is higher than the expression from the circular polyribonucleotide alone (e.g., lacking a capped polynucleotide) at least by 6 fold. In some embodiments, the expression of the one or more expression sequences from the complex in the cell is higher than the expression from the circular polyribonucleotide alone (e.g., lacking a capped polynucleotide) at least by 7 fold. In some embodiments, the expression of the one or more expression sequences from the complex in the cell is higher than the expression from the circular polyribonucleotide alone (e.g., lacking a capped polynucleotide) at least by 8 fold. In some embodiments, the expression of the one or more expression sequences from the complex in the cell is higher than the expression from the circular polyribonucleotide alone (e.g., lacking a capped polynucleotide) at least by 9 fold. In some embodiments, the expression of the one or more expression sequences from the complex in the cell is higher than the expression from the circular polyribonucleotide alone (e.g., lacking a capped polynucleotide) at least by 10 fold. In some embodiments, the expression of the one or more expression sequences from the complex in the cell is higher than the expression from the circular polyribonucleotide alone (e.g., lacking a capped polynucleotide) at least by 15 fold. In some embodiments, the expression of the one or more expression sequences from the complex in the cell is higher than the expression from the circular polyribonucleotide alone (e.g., lacking a capped polynucleotide) at least by 20 fold. In some embodiments, the expression of the one or more expression sequences from the complex in the cell is higher than the expression from the circular polyribonucleotide alone (e.g., lacking a capped polynucleotide) at least by 25 fold. In some embodiments, the expression of the one or more expression sequences from the complex in the cell is higher than the expression from the circular polyribonucleotide alone (e.g., lacking a capped polynucleotide) at least by 30 fold. In some embodiments, the expression of the one or more expression sequences from the complex in the cell is higher than the expression from the circular polyribonucleotide alone (e.g., lacking a capped polynucleotide) at least by 35 fold. In some embodiments, the expression of the one or more expression sequences from the complex in the cell is higher than the expression from the circular polyribonucleotide alone (e.g., lacking a capped polynucleotide) at least by 40 fold. In some embodiments, the expression of the one or more expression sequences from the complex in the cell is higher than the expression from the circular polyribonucleotide alone (e.g., lacking a capped polynucleotide) at least by 45 fold. In some embodiments, the expression of the one or more expression sequences from the complex in the cell is higher than the expression from the circular polyribonucleotide alone (e.g., lacking a capped polynucleotide) at least by 50 fold. In some embodiments, the expression of the one or more expression sequences from the complex in the cell is higher than the expression from the circular polyribonucleotide alone (e.g., lacking a capped polynucleotide) at least by 55 fold. In some embodiments, the expression of the one or more expression sequences from the complex in the cell is higher than the expression from the circular polyribonucleotide alone (e.g., lacking a capped polynucleotide) at least by 60 fold. In some embodiments, the expression of the one or more expression sequences from the complex in the cell is higher than the expression from the circular polyribonucleotide alone (e.g., lacking a capped polynucleotide) at least by 65 fold. In some embodiments, the expression of the one or more expression sequences from the complex in the cell is higher than the expression from the circular polyribonucleotide alone (e.g., lacking a capped polynucleotide) at least by 70 fold. In some embodiments, the expression of the one or more expression sequences from the complex in the cell is higher than the expression from the circular polyribonucleotide alone (e.g., lacking a capped polynucleotide) at least by 75 fold. In some embodiments, the expression of the one or more expression sequences from the complex in the cell is higher than the expression from the circular polyribonucleotide alone (e.g., lacking a capped polynucleotide) at least by 80 fold. In some embodiments, the expression of the one or more expression sequences from the complex in the cell is higher than the expression from the circular polyribonucleotide alone (e.g., lacking a capped polynucleotide) at least by 85 fold. In some embodiments, the expression of the one or more expression sequences from the complex in the cell is higher than the expression from the circular polyribonucleotide alone (e.g., lacking a capped polynucleotide) at least by 90 fold. In some embodiments, the expression of the one or more expression sequences from the complex in the cell is higher than the expression from the circular polyribonucleotide alone (e.g., lacking a capped polynucleotide) at least by 95 fold. In some embodiments, the expression of the one or more expression sequences from the complex in the cell is higher than the expression from the circular polyribonucleotide alone (e.g., lacking a capped polynucleotide) at least by 100 fold. In some embodiments, the expression of the one or more expression sequences from the complex in the cell is higher than the expression from the circular polyribonucleotide alone (e.g., lacking a capped polynucleotide) at least by 200 fold. In some embodiments, the expression of the one or more expression sequences from the complex in the cell is higher than the expression from the circular polyribonucleotide alone (e.g., lacking a capped polynucleotide) at least by 300 fold. In some embodiments, the expression of the one or more expression sequences from the complex in the cell is higher than the expression from the circular polyribonucleotide alone (e.g., lacking a capped polynucleotide) at least by 400 fold. In some embodiments, the expression of the one or more expression sequences from the complex in the cell is higher than the expression from the circular polyribonucleotide alone (e.g., lacking a capped polynucleotide) at least by 500 fold. In some embodiments, the expression of the one or more expression sequences from the complex in the cell is higher than the expression from the circular polyribonucleotide alone (e.g., lacking a capped polynucleotide) at least by 600 fold. In some embodiments, the expression of the one or more expression sequences from the complex in the cell is higher than the expression from the circular polyribonucleotide alone (e.g., lacking a capped polynucleotide) at least by 700 fold. In some embodiments, the expression of the one or more expression sequences from the complex in the cell is higher than the expression from the circular polyribonucleotide alone (e.g., lacking a capped polynucleotide) at least by 800 fold. In some embodiments, the expression of the one or more expression sequences from the complex in the cell is higher than the expression from the circular polyribonucleotide alone (e.g., lacking a capped polynucleotide) at least by 900 fold. In some embodiments, the expression of the one or more expression sequences from the complex in the cell is higher than the expression from the circular polyribonucleotide alone (e.g., lacking a capped polynucleotide) at least by 1000 fold. In some embodiments, the expression of the one or more expression sequences from the complex in the cell is higher than the expression from the circular polyribonucleotide alone (e.g., lacking a capped polynucleotide) at least by 5000 fold. In some embodiments, the expression of the one or more expression sequences from the complex in the cell is higher than the expression from the circular polyribonucleotide alone (e.g., lacking a capped polynucleotide) at least by 10000 fold. In some embodiments, the expression of the one or more expression sequences from the complex in the cell is greater than the expression from the circular polyribonucleotide alone (e.g., lacking a capped polynucleotide) by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 5000, 10000 fold or more. In some embodiments, the expression of the one or more expression sequences from the complex in the cell is greater than the expression from the circular polyribonucleotide alone (e.g., lacking a capped polynucleotide) by 2 fold. In some embodiments, the expression of the one or more expression sequences from the complex in the cell is greater than the expression from the circular polyribonucleotide alone (e.g., lacking a capped polynucleotide) by 3 fold. In some embodiments, the expression of the one or more expression sequences from the complex in the cell is greater than the expression from the circular polyribonucleotide alone (e.g., lacking a capped polynucleotide) by 4 fold. In some embodiments, the expression of the one or more expression sequences from the complex in the cell is greater than the expression from the circular polyribonucleotide alone (e.g., lacking a capped polynucleotide) by 5 fold. In some embodiments, the expression of the one or more expression sequences from the complex in the cell is greater than the expression from the circular polyribonucleotide alone (e.g., lacking a capped polynucleotide) by 6 fold. In some embodiments, the expression of the one or more expression sequences from the complex in the cell is greater than the expression from the circular polyribonucleotide alone (e.g., lacking a capped polynucleotide) by 7 fold. In some embodiments, the expression of the one or more expression sequences from the complex in the cell is greater than the expression from the circular polyribonucleotide alone (e.g., lacking a capped polynucleotide) by 8 fold. In some embodiments, the expression of the one or more expression sequences from the complex in the cell is greater than the expression from the circular polyribonucleotide alone (e.g., lacking a capped polynucleotide) by 9 fold. In some embodiments, the expression of the one or more expression sequences from the complex in the cell is greater than the expression from the circular polyribonucleotide alone (e.g., lacking a capped polynucleotide) by 10 fold. In some embodiments, the expression of the one or more expression sequences from the complex in the cell is greater than the expression from the circular polyribonucleotide alone (e.g., lacking a capped polynucleotide) by 15 fold. In some embodiments, the expression of the one or more expression sequences from the complex in the cell is greater than the expression from the circular polyribonucleotide alone (e.g., lacking a capped polynucleotide) by 20 fold. In some embodiments, the expression of the one or more expression sequences from the complex in the cell is greater than the expression from the circular polyribonucleotide alone (e.g., lacking a capped polynucleotide) by 25 fold. In some embodiments, the expression of the one or more expression sequences from the complex in the cell is greater than the expression from the circular polyribonucleotide alone (e.g., lacking a capped polynucleotide) by 30 fold. In some embodiments, the expression of the one or more expression sequences from the complex in the cell is greater than the expression from the circular polyribonucleotide alone (e.g., lacking a capped polynucleotide) by 35 fold. In some embodiments, the expression of the one or more expression sequences from the complex in the cell is greater than the expression from the circular polyribonucleotide alone (e.g., lacking a capped polynucleotide) by 40 fold. In some embodiments, the expression of the one or more expression sequences from the complex in the cell is greater than the expression from the circular polyribonucleotide alone (e.g., lacking a capped polynucleotide) by 45 fold. In some embodiments, the expression of the one or more expression sequences from the complex in the cell is greater than the expression from the circular polyribonucleotide alone (e.g., lacking a capped polynucleotide) by 50 fold. In some embodiments, the expression of the one or more expression sequences from the complex in the cell is greater than the expression from the circular polyribonucleotide alone (e.g., lacking a capped polynucleotide) by 55 fold. In some embodiments, the expression of the one or more expression sequences from the complex in the cell is greater than the expression from the circular polyribonucleotide alone (e.g., lacking a capped polynucleotide) by 60 fold. In some embodiments, the expression of the one or more expression sequences from the complex in the cell is greater than the expression from the circular polyribonucleotide alone (e.g., lacking a capped polynucleotide) by 65 fold. In some embodiments, the expression of the one or more expression sequences from the complex in the cell is greater than the expression from the circular polyribonucleotide alone (e.g., lacking a capped polynucleotide) by 70 fold. In some embodiments, the expression of the one or more expression sequences from the complex in the cell is greater than the expression from the circular polyribonucleotide alone (e.g., lacking a capped polynucleotide) by 75 fold. In some embodiments, the expression of the one or more expression sequences from the complex in the cell is greater than the expression from the circular polyribonucleotide alone (e.g., lacking a capped polynucleotide) by 80 fold. In some embodiments, the expression of the one or more expression sequences from the complex in the cell is greater than the expression from the circular polyribonucleotide alone (e.g., lacking a capped polynucleotide) by 85 fold. In some embodiments, the expression of the one or more expression sequences from the complex in the cell is greater than the expression from the circular polyribonucleotide alone (e.g., lacking a capped polynucleotide) by 90 fold. In some embodiments, the expression of the one or more expression sequences from the complex in the cell is greater than the expression from the circular polyribonucleotide alone (e.g., lacking a capped polynucleotide) by 95 fold. In some embodiments, the expression of the one or more expression sequences from the complex in the cell is greater than the expression from the circular polyribonucleotide alone (e.g., lacking a capped polynucleotide) by 100 fold. In some embodiments, the expression of the one or more expression sequences from the complex in the cell is greater than the expression from the circular polyribonucleotide alone (e.g., lacking a capped polynucleotide) by 200 fold. In some embodiments, the expression of the one or more expression sequences from the complex in the cell is greater than the expression from the circular polyribonucleotide alone (e.g., lacking a capped polynucleotide) by 300 fold. In some embodiments, the expression of the one or more expression sequences from the complex in the cell is greater than the expression from the circular polyribonucleotide alone (e.g., lacking a capped polynucleotide) by 400 fold. In some embodiments, the expression of the one or more expression sequences from the complex in the cell is greater than the expression from the circular polyribonucleotide alone (e.g., lacking a capped polynucleotide) by 500 fold. In some embodiments, the expression of the one or more expression sequences from the complex in the cell is greater than the expression from the circular polyribonucleotide alone (e.g., lacking a capped polynucleotide) by 600 fold. In some embodiments, the expression of the one or more expression sequences from the complex in the cell is greater than the expression from the circular polyribonucleotide alone (e.g., lacking a capped polynucleotide) by 700 fold. In some embodiments, the expression of the one or more expression sequences from the complex in the cell is greater than the expression from the circular polyribonucleotide alone (e.g., lacking a capped polynucleotide) by 800 fold. In some embodiments, the expression of the one or more expression sequences from the complex in the cell is greater than the expression from the circular polyribonucleotide alone (e.g., lacking a capped polynucleotide) by 900 fold. In some embodiments, the expression of the one or more expression sequences from the complex in the cell is greater than the expression from the circular polyribonucleotide alone (e.g., lacking a capped polynucleotide) by 1000 fold. In some embodiments, the expression of the one or more expression sequences from the complex in the cell is greater than the expression from the circular polyribonucleotide alone (e.g., lacking a capped polynucleotide) by 5000 fold. In some embodiments, the expression of the one or more expression sequences from the complex in the cell is greater than the expression from the circular polyribonucleotide alone (e.g., lacking a capped polynucleotide) by 10000 fold.

In some other aspects, the invention as provided herein a method of in vivo expression of one or more expression sequences, comprising: administering to a cell in vivo a circular polyribonucleotide as provided herein comprising the one or more expression sequences, and a capped polyribonucleotide as provided herein to a cell, wherein the first binding region of the capped polyribonucleotide is bound to the second binding region of the circular polyribonucleotide to form a complex in the cell and the expression of the one or more expression sequences from the complex in the cell is increased compared to expression from the circular polyribonucleotide alone (e.g., lacking a capped polynucleotide). In some embodiments, increased expression results in greater overall protein production.

In some embodiments, the expression of the one or more expression sequences from the complex in the cell is increased by at least 10%, 20%, 30%, 40%, 50%, 60%, 70%, 80%, 90%, 100%, 200%, 300%, 400%, 500%, 600%, 700%, 800%, 900%, 1000%, 5000%, 10000% or more compared to the expression from the circular polyribonucleotide alone (e.g., lacking a capped polynucleotide). In some embodiments, the expression of the one or more expression sequences from the complex in the cell is increased by at least 10% compared to the expression from the circular polyribonucleotide alone (e.g., lacking a capped polynucleotide). In some embodiments, the expression of the one or more expression sequences from the complex in the cell is increased by at least 20% compared to the expression from the circular polyribonucleotide alone (e.g., lacking a capped polynucleotide). In some embodiments, the expression of the one or more expression sequences from the complex in the cell is increased by at least 30% compared to the expression from the circular polyribonucleotide alone (e.g., lacking a capped polynucleotide). In some embodiments, the expression of the one or more expression sequences from the complex in the cell is increased by at least 40% compared to the expression from the circular polyribonucleotide alone (e.g., lacking a capped polynucleotide). In some embodiments, the expression of the one or more expression sequences from the complex in the cell is increased by at least 50% compared to the expression from the circular polyribonucleotide alone (e.g., lacking a capped polynucleotide). In some embodiments, the expression of the one or more expression sequences from the complex in the cell is increased by at least 60% compared to the expression from the circular polyribonucleotide alone (e.g., lacking a capped polynucleotide). In some embodiments, the expression of the one or more expression sequences from the complex in the cell is increased by at least 70% compared to the expression from the circular polyribonucleotide alone (e.g., lacking a capped polynucleotide). In some embodiments, the expression of the one or more expression sequences from the complex in the cell is increased by at least 80% compared to the expression from the circular polyribonucleotide alone (e.g., lacking a capped polynucleotide). In some embodiments, the expression of the one or more expression sequences from the complex in the cell is increased by at least 90% compared to the expression from the circular polyribonucleotide alone (e.g., lacking a capped polynucleotide). In some embodiments, the expression of the one or more expression sequences from the complex in the cell is increased by at least 100% compared to the expression from the circular polyribonucleotide alone (e.g., lacking a capped polynucleotide). In some embodiments, the expression of the one or more expression sequences from the complex in the cell is increased by at least 200% compared to the expression from the circular polyribonucleotide alone (e.g., lacking a capped polynucleotide). In some embodiments, the expression of the one or more expression sequences from the complex in the cell is increased by at least 300% compared to the expression from the circular polyribonucleotide alone (e.g., lacking a capped polynucleotide). In some embodiments, the expression of the one or more expression sequences from the complex in the cell is increased by at least 400% compared to the expression from the circular polyribonucleotide alone (e.g., lacking a capped polynucleotide). In some embodiments, the expression of the one or more expression sequences from the complex in the cell is increased by at least 500% compared to the expression from the circular polyribonucleotide alone (e.g., lacking a capped polynucleotide). In some embodiments, the expression of the one or more expression sequences from the complex in the cell is increased by at least 600% compared to the expression from the circular polyribonucleotide alone (e.g., lacking a capped polynucleotide). In some embodiments, the expression of the one or more expression sequences from the complex in the cell is increased by at least 700% compared to the expression from the circular polyribonucleotide alone (e.g., lacking a capped polynucleotide). In some embodiments, the expression of the one or more expression sequences from the complex in the cell is increased by at least 800% compared to the expression from the circular polyribonucleotide alone (e.g., lacking a capped polynucleotide). In some embodiments, the expression of the one or more expression sequences from the complex in the cell is increased by at least 900% compared to the expression from the circular polyribonucleotide alone (e.g., lacking a capped polynucleotide). In some embodiments, the expression of the one or more expression sequences from the complex in the cell is increased by at least 1000% compared to the expression from the circular polyribonucleotide alone (e.g., lacking a capped polynucleotide). In some embodiments, the expression of the one or more expression sequences from the complex in the cell is increased by at least 5000% compared to the expression from the circular polyribonucleotide alone (e.g., lacking a capped polynucleotide). In some embodiments, the expression of the one or more expression sequences from the complex in the cell is increased by at least 10000% compared to the expression from the circular polyribonucleotide alone (e.g., lacking a capped polynucleotide). In some embodiments, the expression of the one or more expression sequences from the complex in the cell is increased by 10%, 20%, 30%, 40%, 50%, 60%, 70%, 80%, 90%, 100%, 200%, 300%, 400%, 500%, 600%, 700%, 800%, 900%, 1000%, 5000%, 10000% or more compared to the expression from the circular polyribonucleotide alone (e.g., lacking a capped polynucleotide). In some embodiments, the expression of the one or more expression sequences from the complex in the cell is increased by 10% compared to the expression from the circular polyribonucleotide alone (e.g., lacking a capped polynucleotide). In some embodiments, the expression of the one or more expression sequences from the complex in the cell is increased by 20% compared to the expression from the circular polyribonucleotide alone (e.g., lacking a capped polynucleotide). In some embodiments, the expression of the one or more expression sequences from the complex in the cell is increased by 30% compared to the expression from the circular polyribonucleotide alone (e.g., lacking a capped polynucleotide). In some embodiments, the expression of the one or more expression sequences from the complex in the cell is increased by 40% compared to the expression from the circular polyribonucleotide alone (e.g., lacking a capped polynucleotide). In some embodiments, the expression of the one or more expression sequences from the complex in the cell is increased by 50% compared to the expression from the circular polyribonucleotide alone (e.g., lacking a capped polynucleotide). In some embodiments, the expression of the one or more expression sequences from the complex in the cell is increased by 60% compared to the expression from the circular polyribonucleotide alone (e.g., lacking a capped polynucleotide). In some embodiments, the expression of the one or more expression sequences from the complex in the cell is increased by 70% compared to the expression from the circular polyribonucleotide alone (e.g., lacking a capped polynucleotide). In some embodiments, the expression of the one or more expression sequences from the complex in the cell is increased by 80% compared to the expression from the circular polyribonucleotide alone (e.g., lacking a capped polynucleotide). In some embodiments, the expression of the one or more expression sequences from the complex in the cell is increased by 90% compared to the expression from the circular polyribonucleotide alone (e.g., lacking a capped polynucleotide). In some embodiments, the expression of the one or more expression sequences from the complex in the cell is increased by 100% compared to the expression from the circular polyribonucleotide alone (e.g., lacking a capped polynucleotide). In some embodiments, the expression of the one or more expression sequences from the complex in the cell is increased by 200% compared to the expression from the circular polyribonucleotide alone (e.g., lacking a capped polynucleotide). In some embodiments, the expression of the one or more expression sequences from the complex in the cell is increased by 300% compared to the expression from the circular polyribonucleotide alone (e.g., lacking a capped polynucleotide). In some embodiments, the expression of the one or more expression sequences from the complex in the cell is increased by 400% compared to the expression from the circular polyribonucleotide alone (e.g., lacking a capped polynucleotide). In some embodiments, the expression of the one or more expression sequences from the complex in the cell is increased by 500% compared to the expression from the circular polyribonucleotide alone (e.g., lacking a capped polynucleotide). In some embodiments, the expression of the one or more expression sequences from the complex in the cell is increased by 600% compared to the expression from the circular polyribonucleotide alone (e.g., lacking a capped polynucleotide). In some embodiments, the expression of the one or more expression sequences from the complex in the cell is increased by 700% compared to the expression from the circular polyribonucleotide alone (e.g., lacking a capped polynucleotide). In some embodiments, the expression of the one or more expression sequences from the complex in the cell is increased by 800% compared to the expression from the circular polyribonucleotide alone (e.g., lacking a capped polynucleotide). In some embodiments, the expression of the one or more expression sequences from the complex in the cell is increased by 900% compared to the expression from the circular polyribonucleotide alone (e.g., lacking a capped polynucleotide). In some embodiments, the expression of the one or more expression sequences from the complex in the cell is increased by 1000% compared to the expression from the circular polyribonucleotide alone (e.g., lacking a capped polynucleotide). In some embodiments, the expression of the one or more expression sequences from the complex in the cell is increased by 5000% compared to the expression from the circular polyribonucleotide alone (e.g., lacking a capped polynucleotide). In some embodiments, the expression of the one or more expression sequences from the complex in the cell is increased by 10000% compared to the expression from the circular polyribonucleotide alone (e.g., lacking a capped polynucleotide). In some embodiments, the expression of the one or more expression sequences from the complex in the cell is increased compared to the expression from the circular polyribonucleotide alone (e.g., lacking a capped polynucleotide) at least by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 5000, 10000 fold or more. In some embodiments, the expression of the one or more expression sequences from the complex in the cell is increased compared to the expression from the circular polyribonucleotide alone (e.g., lacking a capped polynucleotide) at least by 2 fold. In some embodiments, the expression of the one or more expression sequences from the complex in the cell is increased compared to the expression from the circular polyribonucleotide alone (e.g., lacking a capped polynucleotide) at least by 3 fold. In some embodiments, the expression of the one or more expression sequences from the complex in the cell is increased compared to the expression from the circular polyribonucleotide alone (e.g., lacking a capped polynucleotide) at least by 4 fold. In some embodiments, the expression of the one or more expression sequences from the complex in the cell is increased compared to the expression from the circular polyribonucleotide alone (e.g., lacking a capped polynucleotide) at least by 5 fold. In some embodiments, the expression of the one or more expression sequences from the complex in the cell is increased compared to the expression from the circular polyribonucleotide alone (e.g., lacking a capped polynucleotide) at least by 6 fold. In some embodiments, the expression of the one or more expression sequences from the complex in the cell is increased compared to the expression from the circular polyribonucleotide alone (e.g., lacking a capped polynucleotide) at least by 7 fold. In some embodiments, the expression of the one or more expression sequences from the complex in the cell is increased compared to the expression from the circular polyribonucleotide alone (e.g., lacking a capped polynucleotide) at least by 8 fold. In some embodiments, the expression of the one or more expression sequences from the complex in the cell is increased compared to the expression from the circular polyribonucleotide alone (e.g., lacking a capped polynucleotide) at least by 9 fold. In some embodiments, the expression of the one or more expression sequences from the complex in the cell is increased compared to the expression from the circular polyribonucleotide alone (e.g., lacking a capped polynucleotide) at least by 10 fold. In some embodiments, the expression of the one or more expression sequences from the complex in the cell is increased compared to the expression from the circular polyribonucleotide alone (e.g., lacking a capped polynucleotide) at least by 15 fold. In some embodiments, the expression of the one or more expression sequences from the complex in the cell is increased compared to the expression from the circular polyribonucleotide alone (e.g., lacking a capped polynucleotide) at least by 20 fold. In some embodiments, the expression of the one or more expression sequences from the complex in the cell is increased compared to the expression from the circular polyribonucleotide alone (e.g., lacking a capped polynucleotide) at least by 25 fold. In some embodiments, the expression of the one or more expression sequences from the complex in the cell is increased compared to the expression from the circular polyribonucleotide alone (e.g., lacking a capped polynucleotide) at least by 30 fold. In some embodiments, the expression of the one or more expression sequences from the complex in the cell is increased compared to the expression from the circular polyribonucleotide alone (e.g., lacking a capped polynucleotide) at least by 35 fold. In some embodiments, the expression of the one or more expression sequences from the complex in the cell is increased compared to the expression from the circular polyribonucleotide alone (e.g., lacking a capped polynucleotide) at least by 40 fold. In some embodiments, the expression of the one or more expression sequences from the complex in the cell is increased compared to the expression from the circular polyribonucleotide alone (e.g., lacking a capped polynucleotide) at least by 45 fold. In some embodiments, the expression of the one or more expression sequences from the complex in the cell is increased compared to the expression from the circular polyribonucleotide alone (e.g., lacking a capped polynucleotide) at least by 50 fold. In some embodiments, the expression of the one or more expression sequences from the complex in the cell is increased compared to the expression from the circular polyribonucleotide alone (e.g., lacking a capped polynucleotide) at least by, 55 fold. In some embodiments, the expression of the one or more expression sequences from the complex in the cell is increased compared to the expression from the circular polyribonucleotide alone (e.g., lacking a capped polynucleotide) at least by 60 fold. In some embodiments, the expression of the one or more expression sequences from the complex in the cell is increased compared to the expression from the circular polyribonucleotide alone (e.g., lacking a capped polynucleotide) at least by 65 fold. In some embodiments, the expression of the one or more expression sequences from the complex in the cell is increased compared to the expression from the circular polyribonucleotide alone (e.g., lacking a capped polynucleotide) at least by 70 fold. In some embodiments, the expression of the one or more expression sequences from the complex in the cell is increased compared to the expression from the circular polyribonucleotide alone (e.g., lacking a capped polynucleotide) at least by 75 fold. In some embodiments, the expression of the one or more expression sequences from the complex in the cell is increased compared to the expression from the circular polyribonucleotide alone (e.g., lacking a capped polynucleotide) at least by 80 fold. In some embodiments, the expression of the one or more expression sequences from the complex in the cell is increased compared to the expression from the circular polyribonucleotide alone (e.g., lacking a capped polynucleotide) at least by 85 fold. In some embodiments, the expression of the one or more expression sequences from the complex in the cell is increased compared to the expression from the circular polyribonucleotide alone (e.g., lacking a capped polynucleotide) at least by 90 fold. In some embodiments, the expression of the one or more expression sequences from the complex in the cell is increased compared to the expression from the circular polyribonucleotide alone (e.g., lacking a capped polynucleotide) at least by 95 fold. In some embodiments, the expression of the one or more expression sequences from the complex in the cell is increased compared to the expression from the circular polyribonucleotide alone (e.g., lacking a capped polynucleotide) at least by 100 fold. In some embodiments, the expression of the one or more expression sequences from the complex in the cell is increased compared to the expression from the circular polyribonucleotide alone (e.g., lacking a capped polynucleotide) at least by 200 fold. In some embodiments, the expression of the one or more expression sequences from the complex in the cell is increased compared to the expression from the circular polyribonucleotide alone (e.g., lacking a capped polynucleotide) at least by 300 fold. In some embodiments, the expression of the one or more expression sequences from the complex in the cell is increased compared to the expression from the circular polyribonucleotide alone (e.g., lacking a capped polynucleotide) at least by 400 fold. In some embodiments, the expression of the one or more expression sequences from the complex in the cell is increased compared to the expression from the circular polyribonucleotide alone (e.g., lacking a capped polynucleotide) at least by 500 fold. In some embodiments, the expression of the one or more expression sequences from the complex in the cell is increased compared to the expression from the circular polyribonucleotide alone (e.g., lacking a capped polynucleotide) at least by 600 fold. In some embodiments, the expression of the one or more expression sequences from the complex in the cell is increased compared to the expression from the circular polyribonucleotide alone (e.g., lacking a capped polynucleotide) at least by 700 fold. In some embodiments, the expression of the one or more expression sequences from the complex in the cell is increased compared to the expression from the circular polyribonucleotide alone (e.g., lacking a capped polynucleotide) at least by 800 fold. In some embodiments, the expression of the one or more expression sequences from the complex in the cell is increased compared to the expression from the circular polyribonucleotide alone (e.g., lacking a capped polynucleotide) at least by 900 fold. In some embodiments, the expression of the one or more expression sequences from the complex in the cell is increased compared to the expression from the circular polyribonucleotide alone (e.g., lacking a capped polynucleotide) at least by 1000 fold. In some embodiments, the expression of the one or more expression sequences from the complex in the cell is increased compared to the expression from the circular polyribonucleotide alone (e.g., lacking a capped polynucleotide) at least by 5000 fold. In some embodiments, the expression of the one or more expression sequences from the complex in the cell is increased compared to the expression from the circular polyribonucleotide alone (e.g., lacking a capped polynucleotide) at least by 10000 fold. In some embodiments, the expression of the one or more expression sequences from the complex in the cell is increased compared to the expression from the circular polyribonucleotide alone (e.g., lacking a capped polynucleotide) by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 5000, 10000 fold or more. In some embodiments, the expression of the one or more expression sequences from the complex in the cell is increased compared to the expression from the circular polyribonucleotide alone (e.g., lacking a capped polynucleotide) by 2 fold. In some embodiments, the expression of the one or more expression sequences from the complex in the cell is increased compared to the expression from the circular polyribonucleotide alone (e.g., lacking a capped polynucleotide) by 3 fold. In some embodiments, the expression of the one or more expression sequences from the complex in the cell is increased compared to the expression from the circular polyribonucleotide alone (e.g., lacking a capped polynucleotide) by 4 fold. In some embodiments, the expression of the one or more expression sequences from the complex in the cell is increased compared to the expression from the circular polyribonucleotide alone (e.g., lacking a capped polynucleotide) by 5 fold. In some embodiments, the expression of the one or more expression sequences from the complex in the cell is increased compared to the expression from the circular polyribonucleotide alone (e.g., lacking a capped polynucleotide) by 6 fold. In some embodiments, the expression of the one or more expression sequences from the complex in the cell is increased compared to the expression from the circular polyribonucleotide alone (e.g., lacking a capped polynucleotide) by 7 fold. In some embodiments, the expression of the one or more expression sequences from the complex in the cell is increased compared to the expression from the circular polyribonucleotide alone (e.g., lacking a capped polynucleotide) by 8 fold. In some embodiments, the expression of the one or more expression sequences from the complex in the cell is increased compared to the expression from the circular polyribonucleotide alone (e.g., lacking a capped polynucleotide) by 9 fold. In some embodiments, the expression of the one or more expression sequences from the complex in the cell is increased compared to the expression from the circular polyribonucleotide alone (e.g., lacking a capped polynucleotide) by 10 fold. In some embodiments, the expression of the one or more expression sequences from the complex in the cell is increased compared to the expression from the circular polyribonucleotide alone (e.g., lacking a capped polynucleotide) by 15 fold. In some embodiments, the expression of the one or more expression sequences from the complex in the cell is increased compared to the expression from the circular polyribonucleotide alone (e.g., lacking a capped polynucleotide) by 20 fold. In some embodiments, the expression of the one or more expression sequences from the complex in the cell is increased compared to the expression from the circular polyribonucleotide alone (e.g., lacking a capped polynucleotide) by 25 fold. In some embodiments, the expression of the one or more expression sequences from the complex in the cell is increased compared to the expression from the circular polyribonucleotide alone (e.g., lacking a capped polynucleotide) by 30 fold. In some embodiments, the expression of the one or more expression sequences from the complex in the cell is increased compared to the expression from the circular polyribonucleotide alone (e.g., lacking a capped polynucleotide) by 35 fold. In some embodiments, the expression of the one or more expression sequences from the complex in the cell is increased compared to the expression from the circular polyribonucleotide alone (e.g., lacking a capped polynucleotide) by 40 fold. In some embodiments, the expression of the one or more expression sequences from the complex in the cell is increased compared to the expression from the circular polyribonucleotide alone (e.g., lacking a capped polynucleotide) by 45 fold. In some embodiments, the expression of the one or more expression sequences from the complex in the cell is increased compared to the expression from the circular polyribonucleotide alone (e.g., lacking a capped polynucleotide) by 50 fold. In some embodiments, the expression of the one or more expression sequences from the complex in the cell is increased compared to the expression from the circular polyribonucleotide alone (e.g., lacking a capped polynucleotide) by 55 fold. In some embodiments, the expression of the one or more expression sequences from the complex in the cell is increased compared to the expression from the circular polyribonucleotide alone (e.g., lacking a capped polynucleotide) by 60 fold. In some embodiments, the expression of the one or more expression sequences from the complex in the cell is increased compared to the expression from the circular polyribonucleotide alone (e.g., lacking a capped polynucleotide) by 65 fold. In some embodiments, the expression of the one or more expression sequences from the complex in the cell is increased compared to the expression from the circular polyribonucleotide alone (e.g., lacking a capped polynucleotide) by 70 fold. In some embodiments, the expression of the one or more expression sequences from the complex in the cell is increased compared to the expression from the circular polyribonucleotide alone (e.g., lacking a capped polynucleotide) by 75 fold. In some embodiments, the expression of the one or more expression sequences from the complex in the cell is increased compared to the expression from the circular polyribonucleotide alone (e.g., lacking a capped polynucleotide) by 80 fold. In some embodiments, the expression of the one or more expression sequences from the complex in the cell is increased compared to the expression from the circular polyribonucleotide alone (e.g., lacking a capped polynucleotide) by 85 fold. In some embodiments, the expression of the one or more expression sequences from the complex in the cell is increased compared to the expression from the circular polyribonucleotide alone (e.g., lacking a capped polynucleotide) by 90 fold. In some embodiments, the expression of the one or more expression sequences from the complex in the cell is increased compared to the expression from the circular polyribonucleotide alone (e.g., lacking a capped polynucleotide) by 95 fold. In some embodiments, the expression of the one or more expression sequences from the complex in the cell is increased compared to the expression from the circular polyribonucleotide alone (e.g., lacking a capped polynucleotide) by 100 fold. In some embodiments, the expression of the one or more expression sequences from the complex in the cell is increased compared to the expression from the circular polyribonucleotide alone (e.g., lacking a capped polynucleotide) by 200 fold. In some embodiments, the expression of the one or more expression sequences from the complex in the cell is increased compared to the expression from the circular polyribonucleotide alone (e.g., lacking a capped polynucleotide) by 300 fold. In some embodiments, the expression of the one or more expression sequences from the complex in the cell is increased compared to the expression from the circular polyribonucleotide alone (e.g., lacking a capped polynucleotide) by 400 fold. In some embodiments, the expression of the one or more expression sequences from the complex in the cell is increased compared to the expression from the circular polyribonucleotide alone (e.g., lacking a capped polynucleotide) by 500 fold. In some embodiments, the expression of the one or more expression sequences from the complex in the cell is increased compared to the expression from the circular polyribonucleotide alone (e.g., lacking a capped polynucleotide) by 600 fold. In some embodiments, the expression of the one or more expression sequences from the complex in the cell is increased compared to the expression from the circular polyribonucleotide alone (e.g., lacking a capped polynucleotide) by 700 fold. In some embodiments, the expression of the one or more expression sequences from the complex in the cell is increased compared to the expression from the circular polyribonucleotide alone (e.g., lacking a capped polynucleotide) by 800 fold. In some embodiments, the expression of the one or more expression sequences from the complex in the cell is increased compared to the expression from the circular polyribonucleotide alone (e.g., lacking a capped polynucleotide) by 900 fold. In some embodiments, the expression of the one or more expression sequences from the complex in the cell is increased compared to the expression from the circular polyribonucleotide alone (e.g., lacking a capped polynucleotide) by 1000 fold. In some embodiments, the expression of the one or more expression sequences from the complex in the cell is increased compared to the expression from the circular polyribonucleotide alone (e.g., lacking a capped polynucleotide) by 5000 fold. In some embodiments, the expression of the one or more expression sequences from the complex in the cell is increased compared to the expression from the circular polyribonucleotide alone (e.g., lacking a capped polynucleotide) by 10000 fold.

In some embodiments, increased expression from the complex in the cell results in increased protein production compared to protein production from the circular polyribonucleotide alone (e.g., lacking a capped polynucleotide). In some embodiments, the protein production from the complex in the cell is increased by at least 10%, 20%, 30%, 40%, 50%, 60%, 70%, 80%, 90%, 100%, 200%, 300%, 400%, 500%, 600%, 700%, 800%, 900%, 1000%, 5000%, 10000% or more compared to the protein production from the circular polyribonucleotide alone (e.g., lacking a capped polynucleotide). In some embodiments, the protein production from the complex in the cell is increased by at least 10% compared to the protein production from the circular polyribonucleotide alone (e.g., lacking a capped polynucleotide). In some embodiments, the protein production from the complex in the cell is increased by at least 20% compared to the protein production from the circular polyribonucleotide alone (e.g., lacking a capped polynucleotide). In some embodiments, the protein production from the complex in the cell is increased by at least 30% compared to the protein production from the circular polyribonucleotide alone (e.g., lacking a capped polynucleotide). In some embodiments, the protein production from the complex in the cell is increased by at least 40% compared to the protein production from the circular polyribonucleotide alone (e.g., lacking a capped polynucleotide). In some embodiments, the protein production from the complex in the cell is increased by at least 50% compared to the protein production from the circular polyribonucleotide alone (e.g., lacking a capped polynucleotide). In some embodiments, the protein production from the complex in the cell is increased by at least 60% compared to the protein production from the circular polyribonucleotide alone (e.g., lacking a capped polynucleotide). In some embodiments, the protein production from the complex in the cell is increased by at least 70% compared to the protein production from the circular polyribonucleotide alone (e.g., lacking a capped polynucleotide). In some embodiments, the protein production from the complex in the cell is increased by at least 80% compared to the protein production from the circular polyribonucleotide alone (e.g., lacking a capped polynucleotide). In some embodiments, the protein production from the complex in the cell is increased by at least 90% compared to the protein production from the circular polyribonucleotide alone (e.g., lacking a capped polynucleotide). In some embodiments, the protein production from the complex in the cell is increased by at least 100% compared to the protein production from the circular polyribonucleotide alone (e.g., lacking a capped polynucleotide). In some embodiments, the protein production from the complex in the cell is increased by at least 200% compared to the protein production from the circular polyribonucleotide alone (e.g., lacking a capped polynucleotide). In some embodiments, the protein production from the complex in the cell is increased by at least 300% compared to the protein production from the circular polyribonucleotide alone (e.g., lacking a capped polynucleotide). In some embodiments, the protein production from the complex in the cell is increased by at least 400% compared to the protein production from the circular polyribonucleotide alone (e.g., lacking a capped polynucleotide). In some embodiments, the protein production from the complex in the cell is increased by at least 500% compared to the protein production from the circular polyribonucleotide alone (e.g., lacking a capped polynucleotide). In some embodiments, the protein production from the complex in the cell is increased by at least 600% compared to the protein production from the circular polyribonucleotide alone (e.g., lacking a capped polynucleotide). In some embodiments, the protein production from the complex in the cell is increased by at least 700% compared to the protein production from the circular polyribonucleotide alone (e.g., lacking a capped polynucleotide). In some embodiments, the protein production from the complex in the cell is increased by at least 800% compared to the protein production from the circular polyribonucleotide alone (e.g., lacking a capped polynucleotide). In some embodiments, the protein production from the complex in the cell is increased by at least 900% compared to the protein production from the circular polyribonucleotide alone (e.g., lacking a capped polynucleotide). In some embodiments, the protein production from the complex in the cell is increased by at least 1000% compared to the protein production from the circular polyribonucleotide alone (e.g., lacking a capped polynucleotide). In some embodiments, the protein production from the complex in the cell is increased by at least 5000% compared to the protein production from the circular polyribonucleotide alone (e.g., lacking a capped polynucleotide). In some embodiments, the protein production from the complex in the cell is increased by at least 10000% compared to the protein production from the circular polyribonucleotide alone (e.g., lacking a capped polynucleotide). In some embodiments, the protein production from the complex in the cell is increased by 10%, 20%, 30%, 40%, 50%, 60%, 70%, 80%, 90%, 100%, 200%, 300%, 400%, 500%, 600%, 700%, 800%, 900%, 1000%, 5000%, 10000% or more compared to the protein production from the circular polyribonucleotide alone (e.g., lacking a capped polynucleotide). In some embodiments, the protein production from the complex in the cell is increased by 10% compared to the protein production from the circular polyribonucleotide alone (e.g., lacking a capped polynucleotide). In some embodiments, the protein production from the complex in the cell is increased by 20% compared to the protein production from the circular polyribonucleotide alone (e.g., lacking a capped polynucleotide). In some embodiments, the protein production from the complex in the cell is increased by 30% compared to the protein production from the circular polyribonucleotide alone (e.g., lacking a capped polynucleotide). In some embodiments, the protein production from the complex in the cell is increased by 40% compared to the protein production from the circular polyribonucleotide alone (e.g., lacking a capped polynucleotide). In some embodiments, the protein production from the complex in the cell is increased by 50% compared to the protein production from the circular polyribonucleotide alone (e.g., lacking a capped polynucleotide). In some embodiments, the protein production from the complex in the cell is increased by 60% compared to the protein production from the circular polyribonucleotide alone (e.g., lacking a capped polynucleotide). In some embodiments, the protein production from the complex in the cell is increased by 70% compared to the protein production from the circular polyribonucleotide alone (e.g., lacking a capped polynucleotide). In some embodiments, the protein production from the complex in the cell is increased by 80% compared to the protein production from the circular polyribonucleotide alone (e.g., lacking a capped polynucleotide). In some embodiments, the protein production from the complex in the cell is increased by 90% compared to the protein production from the circular polyribonucleotide alone (e.g., lacking a capped polynucleotide). In some embodiments, the protein production from the complex in the cell is increased by 100% compared to the protein production from the circular polyribonucleotide alone (e.g., lacking a capped polynucleotide). In some embodiments, the protein production from the complex in the cell is increased by 200% compared to the protein production from the circular polyribonucleotide alone (e.g., lacking a capped polynucleotide). In some embodiments, the protein production from the complex in the cell is increased by 300% compared to the protein production from the circular polyribonucleotide alone (e.g., lacking a capped polynucleotide). In some embodiments, the protein production from the complex in the cell is increased by 400% compared to the protein production from the circular polyribonucleotide alone (e.g., lacking a capped polynucleotide). In some embodiments, the protein production from the complex in the cell is increased by 500% compared to the protein production from the circular polyribonucleotide alone (e.g., lacking a capped polynucleotide). In some embodiments, the protein production from the complex in the cell is increased by 600% compared to the protein production from the circular polyribonucleotide alone (e.g., lacking a capped polynucleotide). In some embodiments, the protein production from the complex in the cell is increased by 700% compared to the protein production from the circular polyribonucleotide alone (e.g., lacking a capped polynucleotide). In some embodiments, the protein production from the complex in the cell is increased by 800% compared to the protein production from the circular polyribonucleotide alone (e.g., lacking a capped polynucleotide). In some embodiments, the protein production from the complex in the cell is increased by 900% compared to the protein production from the circular polyribonucleotide alone (e.g., lacking a capped polynucleotide). In some embodiments, the protein production from the complex in the cell is increased by 1000% compared to the protein production from the circular polyribonucleotide alone (e.g., lacking a capped polynucleotide). In some embodiments, the protein production from the complex in the cell is increased by 5000% compared to the protein production from the circular polyribonucleotide alone (e.g., lacking a capped polynucleotide). In some embodiments, the protein production from the complex in the cell is increased by 10000% compared to the protein production from the circular polyribonucleotide alone (e.g., lacking a capped polynucleotide). In some embodiments, the protein production from the complex in the cell is increased compared to the protein production from the circular polyribonucleotide alone (e.g., lacking a capped polynucleotide) at least by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 5000, 10000 fold or more. In some embodiments, the protein production from the complex in the cell is increased compared to the protein production from the circular polyribonucleotide alone (e.g., lacking a capped polynucleotide) at least by 2 fold. In some embodiments, the protein production from the complex in the cell is increased compared to the protein production from the circular polyribonucleotide alone (e.g., lacking a capped polynucleotide) at least by 3 fold. In some embodiments, the protein production from the complex in the cell is increased compared to the protein production from the circular polyribonucleotide alone (e.g., lacking a capped polynucleotide) at least by 4 fold. In some embodiments, the protein production from the complex in the cell is increased compared to the protein production from the circular polyribonucleotide alone (e.g., lacking a capped polynucleotide) at least by 5 fold. In some embodiments, the protein production from the complex in the cell is increased compared to the protein production from the circular polyribonucleotide alone (e.g., lacking a capped polynucleotide) at least by 6 fold. In some embodiments, the protein production from the complex in the cell is increased compared to the protein production from the circular polyribonucleotide alone (e.g., lacking a capped polynucleotide) at least by 7 fold. In some embodiments, the protein production from the complex in the cell is increased compared to the protein production from the circular polyribonucleotide alone (e.g., lacking a capped polynucleotide) at least by 8 fold. In some embodiments, the protein production from the complex in the cell is increased compared to the protein production from the circular polyribonucleotide alone (e.g., lacking a capped polynucleotide) at least by 9 fold. In some embodiments, the protein production from the complex in the cell is increased compared to the protein production from the circular polyribonucleotide alone (e.g., lacking a capped polynucleotide) at least by 10 fold. In some embodiments, the protein production from the complex in the cell is increased compared to the protein production from the circular polyribonucleotide alone (e.g., lacking a capped polynucleotide) at least by 15 fold. In some embodiments, the protein production from the complex in the cell is increased compared to the protein production from the circular polyribonucleotide alone (e.g., lacking a capped polynucleotide) at least by 20 fold. In some embodiments, the protein production from the complex in the cell is increased compared to the protein production from the circular polyribonucleotide alone (e.g., lacking a capped polynucleotide) at least by 25 fold. In some embodiments, the protein production from the complex in the cell is increased compared to the protein production from the circular polyribonucleotide alone (e.g., lacking a capped polynucleotide) at least by 30 fold. In some embodiments, the protein production from the complex in the cell is increased compared to the protein production from the circular polyribonucleotide alone (e.g., lacking a capped polynucleotide) at least by 35 fold. In some embodiments, the protein production from the complex in the cell is increased compared to the protein production from the circular polyribonucleotide alone (e.g., lacking a capped polynucleotide) at least by 40 fold. In some embodiments, the protein production from the complex in the cell is increased compared to the protein production from the circular polyribonucleotide alone (e.g., lacking a capped polynucleotide) at least by 45 fold. In some embodiments, the protein production from the complex in the cell is increased compared to the protein production from the circular polyribonucleotide alone (e.g., lacking a capped polynucleotide) at least by 50 fold. In some embodiments, the protein production from the complex in the cell is increased compared to the protein production from the circular polyribonucleotide alone (e.g., lacking a capped polynucleotide) at least by 55 fold. In some embodiments, the protein production from the complex in the cell is increased compared to the protein production from the circular polyribonucleotide alone (e.g., lacking a capped polynucleotide) at least by 60 fold. In some embodiments, the protein production from the complex in the cell is increased compared to the protein production from the circular polyribonucleotide alone (e.g., lacking a capped polynucleotide) at least by 65 fold. In some embodiments, the protein production from the complex in the cell is increased compared to the protein production from the circular polyribonucleotide alone (e.g., lacking a capped polynucleotide) at least by 70 fold. In some embodiments, the protein production from the complex in the cell is increased compared to the protein production from the circular polyribonucleotide alone (e.g., lacking a capped polynucleotide) at least by 75 fold. In some embodiments, the protein production from the complex in the cell is increased compared to the protein production from the circular polyribonucleotide alone (e.g., lacking a capped polynucleotide) at least by 80 fold. In some embodiments, the protein production from the complex in the cell is increased compared to the protein production from the circular polyribonucleotide alone (e.g., lacking a capped polynucleotide) at least by 85 fold. In some embodiments, the protein production from the complex in the cell is increased compared to the protein production from the circular polyribonucleotide alone (e.g., lacking a capped polynucleotide) at least by 90 fold. In some embodiments, the protein production from the complex in the cell is increased compared to the protein production from the circular polyribonucleotide alone (e.g., lacking a capped polynucleotide) at least by 95 fold. In some embodiments, the protein production from the complex in the cell is increased compared to the protein production from the circular polyribonucleotide alone (e.g., lacking a capped polynucleotide) at least by 100 fold. In some embodiments, the protein production from the complex in the cell is increased compared to the protein production from the circular polyribonucleotide alone (e.g., lacking a capped polynucleotide) at least by 200 fold. In some embodiments, the protein production from the complex in the cell is increased compared to the protein production from the circular polyribonucleotide alone (e.g., lacking a capped polynucleotide) at least by 300 fold. In some embodiments, the protein production from the complex in the cell is increased compared to the protein production from the circular polyribonucleotide alone (e.g., lacking a capped polynucleotide) at least by 400 fold. In some embodiments, the protein production from the complex in the cell is increased compared to the protein production from the circular polyribonucleotide alone (e.g., lacking a capped polynucleotide) at least by 500 fold. In some embodiments, the protein production from the complex in the cell is increased compared to the protein production from the circular polyribonucleotide alone (e.g., lacking a capped polynucleotide) at least by 600 fold. In some embodiments, the protein production from the complex in the cell is increased compared to the protein production from the circular polyribonucleotide alone (e.g., lacking a capped polynucleotide) at least by 700 fold. In some embodiments, the protein production from the complex in the cell is increased compared to the protein production from the circular polyribonucleotide alone (e.g., lacking a capped polynucleotide) at least by 800 fold. In some embodiments, the protein production from the complex in the cell is increased compared to the protein production from the circular polyribonucleotide alone (e.g., lacking a capped polynucleotide) at least by 900 fold. In some embodiments, the protein production from the complex in the cell is increased compared to the protein production from the circular polyribonucleotide alone (e.g., lacking a capped polynucleotide) at least by 1000 fold. In some embodiments, the protein production from the complex in the cell is increased compared to the protein production from the circular polyribonucleotide alone (e.g., lacking a capped polynucleotide) at least by 5000 fold. In some embodiments, the protein production from the complex in the cell is increased compared to the protein production from the circular polyribonucleotide alone (e.g., lacking a capped polynucleotide) at least by 10000 fold. In some embodiments, the protein production from the complex in the cell is increased compared to the protein production from the circular polyribonucleotide alone (e.g., lacking a capped polynucleotide) by 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 5000, 10000 fold or more. In some embodiments, the protein production from the complex in the cell is increased compared to the protein production from the circular polyribonucleotide alone (e.g., lacking a capped polynucleotide) by 2 fold. In some embodiments, the protein production from the complex in the cell is increased compared to the protein production from the circular polyribonucleotide alone (e.g., lacking a capped polynucleotide) by 3 fold. In some embodiments, the protein production from the complex in the cell is increased compared to the protein production from the circular polyribonucleotide alone (e.g., lacking a capped polynucleotide) by 4 fold. In some embodiments, the protein production from the complex in the cell is increased compared to the protein production from the circular polyribonucleotide alone (e.g., lacking a capped polynucleotide) by 5 fold. In some embodiments, the protein production from the complex in the cell is increased compared to the protein production from the circular polyribonucleotide alone (e.g., lacking a capped polynucleotide) by 6 fold. In some embodiments, the protein production from the complex in the cell is increased compared to the protein production from the circular polyribonucleotide alone (e.g., lacking a capped polynucleotide) by 7 fold. In some embodiments, the protein production from the complex in the cell is increased compared to the protein production from the circular polyribonucleotide alone (e.g., lacking a capped polynucleotide) by 8 fold. In some embodiments, the protein production from the complex in the cell is increased compared to the protein production from the circular polyribonucleotide alone (e.g., lacking a capped polynucleotide) by 9 fold. In some embodiments, the protein production from the complex in the cell is increased compared to the protein production from the circular polyribonucleotide alone (e.g., lacking a capped polynucleotide) by 10 fold. In some embodiments, the protein production from the complex in the cell is increased compared to the protein production from the circular polyribonucleotide alone (e.g., lacking a capped polynucleotide) by 15 fold. In some embodiments, the protein production from the complex in the cell is increased compared to the protein production from the circular polyribonucleotide alone (e.g., lacking a capped polynucleotide) by 20 fold. In some embodiments, the protein production from the complex in the cell is increased compared to the protein production from the circular polyribonucleotide alone (e.g., lacking a capped polynucleotide) by 25 fold. In some embodiments, the protein production from the complex in the cell is increased compared to the protein production from the circular polyribonucleotide alone (e.g., lacking a capped polynucleotide) by 30 fold. In some embodiments, the protein production from the complex in the cell is increased compared to the protein production from the circular polyribonucleotide alone (e.g., lacking a capped polynucleotide) by 35 fold. In some embodiments, the protein production from the complex in the cell is increased compared to the protein production from the circular polyribonucleotide alone (e.g., lacking a capped polynucleotide) by 40 fold. In some embodiments, the protein production from the complex in the cell is increased compared to the protein production from the circular polyribonucleotide alone (e.g., lacking a capped polynucleotide) by 45 fold. In some embodiments, the protein production from the complex in the cell is increased compared to the protein production from the circular polyribonucleotide alone (e.g., lacking a capped polynucleotide) by 50 fold. In some embodiments, the protein production from the complex in the cell is increased compared to the protein production from the circular polyribonucleotide alone (e.g., lacking a capped polynucleotide) by 55 fold. In some embodiments, the protein production from the complex in the cell is increased compared to the protein production from the circular polyribonucleotide alone (e.g., lacking a capped polynucleotide) by 60 fold. In some embodiments, the protein production from the complex in the cell is increased compared to the protein production from the circular polyribonucleotide alone (e.g., lacking a capped polynucleotide) by 65 fold. In some embodiments, the protein production from the complex in the cell is increased compared to the protein production from the circular polyribonucleotide alone (e.g., lacking a capped polynucleotide) by 70 fold. In some embodiments, the protein production from the complex in the cell is increased compared to the protein production from the circular polyribonucleotide alone (e.g., lacking a capped polynucleotide) by 75 fold. In some embodiments, the protein production from the complex in the cell is increased compared to the protein production from the circular polyribonucleotide alone (e.g., lacking a capped polynucleotide) by 80 fold. In some embodiments, the protein production from the complex in the cell is increased compared to the protein production from the circular polyribonucleotide alone (e.g., lacking a capped polynucleotide) by 85 fold. In some embodiments, the protein production from the complex in the cell is increased compared to the protein production from the circular polyribonucleotide alone (e.g., lacking a capped polynucleotide) by 90 fold. In some embodiments, the protein production from the complex in the cell is increased compared to the protein production from the circular polyribonucleotide alone (e.g., lacking a capped polynucleotide) by 95 fold. In some embodiments, the protein production from the complex in the cell is increased compared to the protein production from the circular polyribonucleotide alone (e.g., lacking a capped polynucleotide) by 100 fold. In some embodiments, the protein production from the complex in the cell is increased compared to the protein production from the circular polyribonucleotide alone (e.g., lacking a capped polynucleotide) by 200 fold. In some embodiments, the protein production from the complex in the cell is increased compared to the protein production from the circular polyribonucleotide alone (e.g., lacking a capped polynucleotide) by 300 fold. In some embodiments, the protein production from the complex in the cell is increased compared to the protein production from the circular polyribonucleotide alone (e.g., lacking a capped polynucleotide) by 400 fold. In some embodiments, the protein production from the complex in the cell is increased compared to the protein production from the circular polyribonucleotide alone (e.g., lacking a capped polynucleotide) by 500 fold. In some embodiments, the protein production from the complex in the cell is increased compared to the protein production from the circular polyribonucleotide alone (e.g., lacking a capped polynucleotide) by 600 fold. In some embodiments, the protein production from the complex in the cell is increased compared to the protein production from the circular polyribonucleotide alone (e.g., lacking a capped polynucleotide) by 700 fold. In some embodiments, the protein production from the complex in the cell is increased compared to the protein production from the circular polyribonucleotide alone (e.g., lacking a capped polynucleotide) by 800 fold. In some embodiments, the protein production from the complex in the cell is increased compared to the protein production from the circular polyribonucleotide alone (e.g., lacking a capped polynucleotide) by 900 fold. In some embodiments, the protein production from the complex in the cell is increased compared to the protein production from the circular polyribonucleotide alone (e.g., lacking a capped polynucleotide) by 1000 fold. In some embodiments, the protein production from the complex in the cell is increased compared to the protein production from the circular polyribonucleotide alone (e.g., lacking a capped polynucleotide) by 5000 fold. In some embodiments, the protein production from the complex in the cell is increased compared to the protein production from the circular polyribonucleotide alone (e.g., lacking a capped polynucleotide) by 10000 fold. In some embodiments, the protein production from the complex in the cell is increased compared to the protein production from the circular polyribonucleotide alone (e.g., lacking a capped polynucleotide) over an interval of time after administration. An interval of time can be at least 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs 20 hrs, 21 hrs, 22 hrs, 23 hrs or more longer than after administering. An interval of time can be at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more longer than after administering. An interval of time can be at least 1 hr after administering. An interval of time can be at least 2 hrs after administering. An interval of time can be at least 3 hrs after administering. An interval of time can be at least 4 hrs after administering. An interval of time can be at least 5 hrs after administering. An interval of time can be at least 6 hrs after administering. An interval of time can be at least 7 hrs after administering. An interval of time can be at least 8 hrs after administering. An interval of time can be at least 9 hrs after administering. An interval of time can be at least 10 hrs after administering. An interval of time can be at least 11 hrs after administering. An interval of time can be at least 12 hrs after administering. An interval of time can be at least 13 hrs after administering. An interval of time can be at least 14 hrs after administering. An interval of time can be at least 15 hrs after administering. An interval of time can be at least 16 hrs after administering. An interval of time can be at least 17 hrs after administering. An interval of time can be at least 18 hrs after administering. An interval of time can be at least 19 hrs after administering. An interval of time can be at least 20 hrs after administering. An interval of time can be at least 21 hrs after administering. An interval of time can be at least 22 hrs after administering. An interval of time can be at least 23 hrs or more longer than after administering. An interval of time can be at least 1 day after administering. An interval of time can be at least 2 days after administering. An interval of time can be at least 3 days after administering. An interval of time can be at least 4 days after administering. An interval of time can be at least 5 days after administering. An interval of time can be at least 6 days after administering. An interval of time can be at least 7 days after administering. An interval of time can be at least 8 days after administering. An interval of time can be at least 9 days after administering. An interval of time can be at least 10 days after administering. An interval of time can be at least 11 days after administering. An interval of time can be at least 12 days after administering. An interval of time can be at least 13 days after administering. An interval of time can be at least 14 days after administering. An interval of time can be at least 15 days after administering. An interval of time can be at least 16 days after administering. An interval of time can be at least 17 days after administering. An interval of time can be at least 18 days after administering. An interval of time can be at least 19 days after administering. An interval of time can be at least 20 days after administering. An interval of time can be at least 21 days after administering. An interval of time can be at least 22 days after administering. An interval of time can be at least 23 days after administering. An interval of time can be at least 24 days after administering. An interval of time can be at least 25 days after administering. An interval of time can be at least 26 days after administering. An interval of time can be at least 27 days after administering. An interval of time can be at least 28 days after administering. An interval of time can be at least 29 days after administering. An interval of time can be at least 30 days than after administering.

### Prolonged Expression

In some aspects, the invention as provided herein comprises a method of expression of one or more expression sequences in a subject, comprising: providing a complex comprising: a circular polyribonucleotide as provided herein comprising the one or more expression sequences, and a capped polyribonucleotide as provided herein, wherein the first binding region of the capped polyribonucleotide is bound to the second binding region of the circular polyribonucleotide; and administering the complex to a cell of the subject, wherein expression of the one or more expression sequences from the complex in the subject is longer than after administering a linear counterpart of the circular polyribonucleotide alone (e.g., lacking a capped polynucleotide).

In some other aspects, the invention as provided herein comprises a method of expression of one or more expression sequences in a subject, comprising: administering to the subject a circular polyribonucleotide as provided herein comprising the one or more expression sequences, and a capped polyribonucleotide as provided herein, wherein the first binding region of the capped polyribonucleotide is bound to the second binding region of the circular polyribonucleotide to form a complex in the subject and expression of the one or more expression sequences from the complex in a cell of the subject is longer than after administering a linear counterpart of the circular polyribonucleotide alone (e.g., lacking a capped polynucleotide).

In some embodiments, the expression of the one or more expression sequences from the complex in the subject is for at least 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs or more longer than after administering a linear counterpart of the circular polyribonucleotide alone (e.g., lacking a capped polynucleotide). In some embodiments, the expression of the one or more expression sequences from the complex in the subject is for at least 1 hr longer than after administering a linear counterpart of the circular polyribonucleotide alone (e.g., lacking a capped polynucleotide). In some embodiments, the expression of the one or more expression sequences from the complex in the subject is for at least 2 hrs longer than after administering a linear counterpart of the circular polyribonucleotide alone (e.g., lacking a capped polynucleotide). In some embodiments, the expression of the one or more expression sequences from the complex in the subject is for at least 3 hrs longer than after administering a linear counterpart of the circular polyribonucleotide alone (e.g., lacking a capped polynucleotide). In some embodiments, the expression of the one or more expression sequences from the complex in the subject is for at least 4 hrs longer than after administering a linear counterpart of the circular polyribonucleotide alone (e.g., lacking a capped polynucleotide). In some embodiments, the expression of the one or more expression sequences from the complex in the subject is for at least 5 hrs longer than after administering a linear counterpart of the circular polyribonucleotide alone (e.g., lacking a capped polynucleotide). In some embodiments, the expression of the one or more expression sequences from the complex in the subject is for at least 6 hrs longer than after administering a linear counterpart of the circular polyribonucleotide alone (e.g., lacking a capped polynucleotide). In some embodiments, the expression of the one or more expression sequences from the complex in the subject is for at least 7 hrs longer than after administering a linear counterpart of the circular polyribonucleotide alone (e.g., lacking a capped polynucleotide). In some embodiments, the expression of the one or more expression sequences from the complex in the subject is for at least 8 hrs longer than after administering a linear counterpart of the circular polyribonucleotide alone (e.g., lacking a capped polynucleotide). In some embodiments, the expression of the one or more expression sequences from the complex in the subject is for at least 9 hrs longer than after administering a linear counterpart of the circular polyribonucleotide alone (e.g., lacking a capped polynucleotide). In some embodiments, the expression of the one or more expression sequences from the complex in the subject is for at least 10 hrs longer than after administering a linear counterpart of the circular polyribonucleotide alone (e.g., lacking a capped polynucleotide). In some embodiments, the expression of the one or more expression sequences from the complex in the subject is for at least 11 hrs longer than after administering a linear counterpart of the circular polyribonucleotide alone (e.g., lacking a capped polynucleotide). In some embodiments, the expression of the one or more expression sequences from the complex in the subject is for at least 12 hrs longer than after administering a linear counterpart of the circular polyribonucleotide alone (e.g., lacking a capped polynucleotide). In some embodiments, the expression of the one or more expression sequences from the complex in the subject is for at least 13 hrs longer than after administering a linear counterpart of the circular polyribonucleotide alone (e.g., lacking a capped polynucleotide). In some embodiments, the expression of the one or more expression sequences from the complex in the subject is for at least 14 hrs longer than after administering a linear counterpart of the circular polyribonucleotide alone (e.g., lacking a capped polynucleotide). In some embodiments, the expression of the one or more expression sequences from the complex in the subject is for at least 15 hrs longer than after administering a linear counterpart of the circular polyribonucleotide alone (e.g., lacking a capped polynucleotide). In some embodiments, the expression of the one or more expression sequences from the complex in the subject is for at least 16 hrs longer than after administering a linear counterpart of the circular polyribonucleotide alone (e.g., lacking a capped polynucleotide). In some embodiments, the expression of the one or more expression sequences from the complex in the subject is for at least 17 hrs longer than after administering a linear counterpart of the circular polyribonucleotide alone (e.g., lacking a capped polynucleotide). In some embodiments, the expression of the one or more expression sequences from the complex in the subject is for at least 18 hrs longer than after administering a linear counterpart of the circular polyribonucleotide alone (e.g., lacking a capped polynucleotide). In some embodiments, the expression of the one or more expression sequences from the complex in the subject is for at least 19 hrs longer than after administering a linear counterpart of the circular polyribonucleotide alone (e.g., lacking a capped polynucleotide). In some embodiments, the expression of the one or more expression sequences from the complex in the subject is for at least 20 hrs longer than after administering a linear counterpart of the circular polyribonucleotide alone (e.g., lacking a capped polynucleotide). In some embodiments, the expression of the one or more expression sequences from the complex in the subject is for at least 21 hrs longer than after administering a linear counterpart of the circular polyribonucleotide alone (e.g., lacking a capped polynucleotide). In some embodiments, the expression of the one or more expression sequences from the complex in the subject is for at least 22 hrs longer than after administering a linear counterpart of the circular polyribonucleotide alone (e.g., lacking a capped polynucleotide). In some embodiments, the expression of the one or more expression sequences from the complex in the subject is for at least 23 hrs longer than after administering a linear counterpart of the circular polyribonucleotide alone (e.g., lacking a capped polynucleotide). In some embodiments, the expression of the one or more expression sequences from the complex in the subject is for 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, or more longer than after administering a linear counterpart of the circular polyribonucleotide alone (e.g., lacking a capped polynucleotide). In some embodiments, the expression of the one or more expression sequences from the complex in the subject is for 1 hr longer than after administering a linear counterpart of the circular polyribonucleotide alone (e.g., lacking a capped polynucleotide). In some embodiments, the expression of the one or more expression sequences from the complex in the subject is for 2 hrs longer than after administering a linear counterpart of the circular polyribonucleotide alone (e.g., lacking a capped polynucleotide). In some embodiments, the expression of the one or more expression sequences from the complex in the subject is for 3 hrs longer than after administering a linear counterpart of the circular polyribonucleotide alone (e.g., lacking a capped polynucleotide). In some embodiments, the expression of the one or more expression sequences from the complex in the subject is for 4 hrs longer than after administering a linear counterpart of the circular polyribonucleotide alone (e.g., lacking a capped polynucleotide). In some embodiments, the expression of the one or more expression sequences from the complex in the subject is for 5 hrs longer than after administering a linear counterpart of the circular polyribonucleotide alone (e.g., lacking a capped polynucleotide). In some embodiments, the expression of the one or more expression sequences from the complex in the subject is for 6 hrs longer than after administering a linear counterpart of the circular polyribonucleotide alone (e.g., lacking a capped polynucleotide). In some embodiments, the expression of the one or more expression sequences from the complex in the subject is for 7 hrs longer than after administering a linear counterpart of the circular polyribonucleotide alone (e.g., lacking a capped polynucleotide). In some embodiments, the expression of the one or more expression sequences from the complex in the subject is for 8 hrs longer than after administering a linear counterpart of the circular polyribonucleotide alone (e.g., lacking a capped polynucleotide). In some embodiments, the expression of the one or more expression sequences from the complex in the subject is for 9 hrs longer than after administering a linear counterpart of the circular polyribonucleotide alone (e.g., lacking a capped polynucleotide). In some embodiments, the expression of the one or more expression sequences from the complex in the subject is for 10 hrs longer than after administering a linear counterpart of the circular polyribonucleotide alone (e.g., lacking a capped polynucleotide). In some embodiments, the expression of the one or more expression sequences from the complex in the subject is for 11 hrs longer than after administering a linear counterpart of the circular polyribonucleotide alone (e.g., lacking a capped polynucleotide). In some embodiments, the expression of the one or more expression sequences from the complex in the subject is for 12 hrs longer than after administering a linear counterpart of the circular polyribonucleotide alone (e.g., lacking a capped polynucleotide). In some embodiments, the expression of the one or more expression sequences from the complex in the subject is for 13 hrs longer than after administering a linear counterpart of the circular polyribonucleotide alone (e.g., lacking a capped polynucleotide). In some embodiments, the expression of the one or more expression sequences from the complex in the subject is for 14 hrs longer than after administering a linear counterpart of the circular polyribonucleotide alone (e.g., lacking a capped polynucleotide). In some embodiments, the expression of the one or more expression sequences from the complex in the subject is for 15 hrs longer than after administering a linear counterpart of the circular polyribonucleotide alone (e.g., lacking a capped polynucleotide). In some embodiments, the expression of the one or more expression sequences from the complex in the subject is for 16 hrs longer than after administering a linear counterpart of the circular polyribonucleotide alone (e.g., lacking a capped polynucleotide). In some embodiments, the expression of the one or more expression sequences from the complex in the subject is for 17 hrs longer than after administering a linear counterpart of the circular polyribonucleotide alone (e.g., lacking a capped polynucleotide). In some embodiments, the expression of the one or more expression sequences from the complex in the subject is for 18 hrs longer than after administering a linear counterpart of the circular polyribonucleotide alone (e.g., lacking a capped polynucleotide). In some embodiments, the expression of the one or more expression sequences from the complex in the subject is for 19 hrs longer than after administering a linear counterpart of the circular polyribonucleotide alone (e.g., lacking a capped polynucleotide). In some embodiments, the expression of the one or more expression sequences from the complex in the subject is for 20 hrs longer than after administering a linear counterpart of the circular polyribonucleotide alone (e.g., lacking a capped polynucleotide). In some embodiments, the expression of the one or more expression sequences from the complex in the subject is for 21 hrs longer than after administering a linear counterpart of the circular polyribonucleotide alone (e.g., lacking a capped polynucleotide). In some embodiments, the expression of the one or more expression sequences from the complex in the subject is for 22 hrs longer than after administering a linear counterpart of the circular polyribonucleotide alone (e.g., lacking a capped polynucleotide). In some embodiments, the expression of the one or more expression sequences from the complex in the subject is for 23 hrs longer than after administering a linear counterpart of the circular polyribonucleotide alone (e.g., lacking a capped polynucleotide). In some embodiments, the expression of the one or more expression sequences from the complex in the subject is for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more longer than after administering a linear counterpart of the circular polyribonucleotide alone (e.g., lacking a capped polynucleotide). In some embodiments, the expression of the one or more expression sequences from the complex in the subject is for at least 1 day longer than after administering a linear counterpart of the circular polyribonucleotide alone (e.g., lacking a capped polynucleotide). In some embodiments, the expression of the one or more expression sequences from the complex in the subject is for at least 2 days longer than after administering a linear counterpart of the circular polyribonucleotide alone (e.g., lacking a capped polynucleotide). In some embodiments, the expression of the one or more expression sequences from the complex in the subject is for at least 3 days longer than after administering a linear counterpart of the circular polyribonucleotide alone (e.g., lacking a capped polynucleotide). In some embodiments, the expression of the one or more expression sequences from the complex in the subject is for at least 4 days longer than after administering a linear counterpart of the circular polyribonucleotide alone (e.g., lacking a capped polynucleotide). In some embodiments, the expression of the one or more expression sequences from the complex in the subject is for at least 5 days longer than after administering a linear counterpart of the circular polyribonucleotide alone (e.g., lacking a capped polynucleotide). In some embodiments, the expression of the one or more expression sequences from the complex in the subject is for at least 6 days longer than after administering a linear counterpart of the circular polyribonucleotide alone (e.g., lacking a capped polynucleotide). In some embodiments, the expression of the one or more expression sequences from the complex in the subject is for at least 7 days longer than after administering a linear counterpart of the circular polyribonucleotide alone (e.g., lacking a capped polynucleotide). In some embodiments, the expression of the one or more expression sequences from the complex in the subject is for at least 8 days longer than after administering a linear counterpart of the circular polyribonucleotide alone (e.g., lacking a capped polynucleotide). In some embodiments, the expression of the one or more expression sequences from the complex in the subject is for at least 9 days longer than after administering a linear counterpart of the circular polyribonucleotide alone (e.g., lacking a capped polynucleotide). In some embodiments, the expression of the one or more expression sequences from the complex in the subject is for at least 10 days longer than after administering a linear counterpart of the circular polyribonucleotide alone (e.g., lacking a capped polynucleotide). In some embodiments, the expression of the one or more expression sequences from the complex in the subject is for at least 11 days longer than after administering a linear counterpart of the circular polyribonucleotide alone (e.g., lacking a capped polynucleotide). In some embodiments, the expression of the one or more expression sequences from the complex in the subject is for at least 12 days longer than after administering a linear counterpart of the circular polyribonucleotide alone (e.g., lacking a capped polynucleotide). In some embodiments, the expression of the one or more expression sequences from the complex in the subject is for at least 13 days longer than after administering a linear counterpart of the circular polyribonucleotide alone (e.g., lacking a capped polynucleotide). In some embodiments, the expression of the one or more expression sequences from the complex in the subject is for at least 14 days longer than after administering a linear counterpart of the circular polyribonucleotide alone (e.g., lacking a capped polynucleotide). In some embodiments, the expression of the one or more expression sequences from the complex in the subject is for at least 15 days longer than after administering a linear counterpart of the circular polyribonucleotide alone (e.g., lacking a capped polynucleotide). In some embodiments, the expression of the one or more expression sequences from the complex in the subject is for at least 16 days longer than after administering a linear counterpart of the circular polyribonucleotide alone (e.g., lacking a capped polynucleotide). In some embodiments, the expression of the one or more expression sequences from the complex in the subject is for at least 17 days longer than after administering a linear counterpart of the circular polyribonucleotide alone (e.g., lacking a capped polynucleotide). In some embodiments, the expression of the one or more expression sequences from the complex in the subject is for at least 18 days longer than after administering a linear counterpart of the circular polyribonucleotide alone (e.g., lacking a capped polynucleotide). In some embodiments, the expression of the one or more expression sequences from the complex in the subject is for at least 19 days longer than after administering a linear counterpart of the circular polyribonucleotide alone (e.g., lacking a capped polynucleotide). In some embodiments, the expression of the one or more expression sequences from the complex in the subject is for at least 20 days longer than after administering a linear counterpart of the circular polyribonucleotide alone (e.g., lacking a capped polynucleotide). In some embodiments, the expression of the one or more expression sequences from the complex in the subject is for at least 21 days longer than after administering a linear counterpart of the circular polyribonucleotide alone (e.g., lacking a capped polynucleotide). In some embodiments, the expression of the one or more expression sequences from the complex in the subject is for at least 22 days longer than after administering a linear counterpart of the circular polyribonucleotide alone (e.g., lacking a capped polynucleotide). In some embodiments, the expression of the one or more expression sequences from the complex in the subject is for at least 23 days longer than after administering a linear counterpart of the circular polyribonucleotide alone (e.g., lacking a capped polynucleotide). In some embodiments, the expression of the one or more expression sequences from the complex in the subject is for at least 24 days longer than after administering a linear counterpart of the circular polyribonucleotide alone (e.g., lacking a capped polynucleotide). In some embodiments, the expression of the one or more expression sequences from the complex in the subject is for at least 25 days longer than after administering a linear counterpart of the circular polyribonucleotide alone (e.g., lacking a capped polynucleotide). In some embodiments, the expression of the one or more expression sequences from the complex in the subject is for at least 26 days longer than after administering a linear counterpart of the circular polyribonucleotide alone (e.g., lacking a capped polynucleotide). In some embodiments, the expression of the one or more expression sequences from the complex in the subject is for at least 27 days longer than after administering a linear counterpart of the circular polyribonucleotide alone (e.g., lacking a capped polynucleotide). In some embodiments, the expression of the one or more expression sequences from the complex in the subject is for at least 28 days longer than after administering a linear counterpart of the circular polyribonucleotide alone (e.g., lacking a capped polynucleotide). In some embodiments, the expression of the one or more expression sequences from the complex in the subject is for at least 29 days longer than after administering a linear counterpart of the circular polyribonucleotide alone (e.g., lacking a capped polynucleotide). In some embodiments, the expression of the one or more expression sequences from the complex in the subject is for at least 30 days longer than after administering a linear counterpart of the circular polyribonucleotide alone (e.g., lacking a capped polynucleotide). In some embodiments, the expression of the one or more expression sequences from the complex in the subject is for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more longer than after administering a linear counterpart of the circular polyribonucleotide alone (e.g., lacking a capped polynucleotide). In some embodiments, the expression of the one or more expression sequences from the complex in the subject is for 1 day longer than after administering a linear counterpart of the circular polyribonucleotide alone (e.g., lacking a capped polynucleotide). In some embodiments, the expression of the one or more expression sequences from the complex in the subject is for 2 days longer than after administering a linear counterpart of the circular polyribonucleotide alone (e.g., lacking a capped polynucleotide). In some embodiments, the expression of the one or more expression sequences from the complex in the subject is for 3 days longer than after administering a linear counterpart of the circular polyribonucleotide alone (e.g., lacking a capped polynucleotide). In some embodiments, the expression of the one or more expression sequences from the complex in the subject is for 4 days longer than after administering a linear counterpart of the circular polyribonucleotide alone (e.g., lacking a capped polynucleotide). In some embodiments, the expression of the one or more expression sequences from the complex in the subject is for 5 days longer than after administering a linear counterpart of the circular polyribonucleotide alone (e.g., lacking a capped polynucleotide). In some embodiments, the expression of the one or more expression sequences from the complex in the subject is for 6 days longer than after administering a linear counterpart of the circular polyribonucleotide alone (e.g., lacking a capped polynucleotide). In some embodiments, the expression of the one or more expression sequences from the complex in the subject is for 7 days longer than after administering a linear counterpart of the circular polyribonucleotide alone (e.g., lacking a capped polynucleotide). In some embodiments, the expression of the one or more expression sequences from the complex in the subject is for 8 days longer than after administering a linear counterpart of the circular polyribonucleotide alone (e.g., lacking a capped polynucleotide). In some embodiments, the expression of the one or more expression sequences from the complex in the subject is for 9 day longer than after administering a linear counterpart of the circular polyribonucleotide alone (e.g., lacking a capped polynucleotide). In some embodiments, the expression of the one or more expression sequences from the complex in the subject is for 10 days longer than after administering a linear counterpart of the circular polyribonucleotide alone (e.g., lacking a capped polynucleotide). In some embodiments, the expression of the one or more expression sequences from the complex in the subject is for 11 days longer than after administering a linear counterpart of the circular polyribonucleotide alone (e.g., lacking a capped polynucleotide). In some embodiments, the expression of the one or more expression sequences from the complex in the subject is for 12 days longer than after administering a linear counterpart of the circular polyribonucleotide alone (e.g., lacking a capped polynucleotide). In some embodiments, the expression of the one or more expression sequences from the complex in the subject is for 13 days longer than after administering a linear counterpart of the circular polyribonucleotide alone (e.g., lacking a capped polynucleotide). In some embodiments, the expression of the one or more expression sequences from the complex in the subject is for 14 days longer than after administering a linear counterpart of the circular polyribonucleotide alone (e.g., lacking a capped polynucleotide). In some embodiments, the expression of the one or more expression sequences from the complex in the subject is for 15 days longer than after administering a linear counterpart of the circular polyribonucleotide alone (e.g., lacking a capped polynucleotide). In some embodiments, the expression of the one or more expression sequences from the complex in the subject is for 16 days longer than after administering a linear counterpart of the circular polyribonucleotide alone (e.g., lacking a capped polynucleotide). In some embodiments, the expression of the one or more expression sequences from the complex in the subject is for 17 days longer than after administering a linear counterpart of the circular polyribonucleotide alone (e.g., lacking a capped polynucleotide). In some embodiments, the expression of the one or more expression sequences from the complex in the subject is for 18 days longer than after administering a linear counterpart of the circular polyribonucleotide alone (e.g., lacking a capped polynucleotide). In some embodiments, the expression of the one or more expression sequences from the complex in the subject is for 19 days longer than after administering a linear counterpart of the circular polyribonucleotide alone (e.g., lacking a capped polynucleotide). In some embodiments, the expression of the one or more expression sequences from the complex in the subject is for 20 days longer than after administering a linear counterpart of the circular polyribonucleotide alone (e.g., lacking a capped polynucleotide). In some embodiments, the expression of the one or more expression sequences from the complex in the subject is for 21 days longer than after administering a linear counterpart of the circular polyribonucleotide alone (e.g., lacking a capped polynucleotide). In some embodiments, the expression of the one or more expression sequences from the complex in the subject is for 22 days longer than after administering a linear counterpart of the circular polyribonucleotide alone (e.g., lacking a capped polynucleotide). In some embodiments, the expression of the one or more expression sequences from the complex in the subject is for 23 days longer than after administering a linear counterpart of the circular polyribonucleotide alone (e.g., lacking a capped polynucleotide). In some embodiments, the expression of the one or more expression sequences from the complex in the subject is for 24 days longer than after administering a linear counterpart of the circular polyribonucleotide alone (e.g., lacking a capped polynucleotide). In some embodiments, the expression of the one or more expression sequences from the complex in the subject is for 25 days longer than after administering a linear counterpart of the circular polyribonucleotide alone (e.g., lacking a capped polynucleotide). In some embodiments, the expression of the one or more expression sequences from the complex in the subject is for 26 days longer than after administering a linear counterpart of the circular polyribonucleotide alone (e.g., lacking a capped polynucleotide). In some embodiments, the expression of the one or more expression sequences from the complex in the subject is for 27 days longer than after administering a linear counterpart of the circular polyribonucleotide alone (e.g., lacking a capped polynucleotide). In some embodiments, the expression of the one or more expression sequences from the complex in the subject is for 28 days longer than after administering a linear counterpart of the circular polyribonucleotide alone (e.g., lacking a capped polynucleotide). In some embodiments, the expression of the one or more expression sequences from the complex in the subject is for 29 days longer than after administering a linear counterpart of the circular polyribonucleotide alone (e.g., lacking a capped polynucleotide). In some embodiments, the expression of the one or more expression sequences from the complex in the subject is for 30 days longer than after administering a linear counterpart of the circular polyribonucleotide alone (e.g., lacking a capped polynucleotide). In some embodiments, the expression of the one or more expression sequences from the complex in the subject is for at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, or more longer than after administering a circular polyribonucleotide alone (e.g., lacking a capped polynucleotide). In some embodiments, the expression of the one or more expression sequences from the complex in the subject is for at least 1 month longer than after administering a circular polyribonucleotide alone (e.g., lacking a capped polynucleotide). In some embodiments, the expression of the one or more expression sequences from the complex in the subject is for at least 2 months longer than after administering a circular polyribonucleotide alone (e.g., lacking a capped polynucleotide). In some embodiments, the expression of the one or more expression sequences from the complex in the subject is for at least 3 months longer than after administering a circular polyribonucleotide alone (e.g., lacking a capped polynucleotide). In some embodiments, the expression of the one or more expression sequences from the complex in the subject is for at least 4 months longer than after administering a circular polyribonucleotide alone (e.g., lacking a capped polynucleotide). In some embodiments, the expression of the one or more expression sequences from the complex in the subject is for at least 5 months longer than after administering a circular polyribonucleotide alone (e.g., lacking a capped polynucleotide). In some embodiments, the expression of the one or more expression sequences from the complex in the subject is for at least 6 months longer than after administering a circular polyribonucleotide alone (e.g., lacking a capped polynucleotide). In some embodiments, the expression of the one or more expression sequences from the complex in the subject is for at least 7 months longer than after administering a circular polyribonucleotide alone (e.g., lacking a capped polynucleotide). In some embodiments, the expression of the one or more expression sequences from the complex in the subject is for at least 8 months longer than after administering a circular polyribonucleotide alone (e.g., lacking a capped polynucleotide). In some embodiments, the expression of the one or more expression sequences from the complex in the subject is for at least 9 months longer than after administering a circular polyribonucleotide alone (e.g., lacking a capped polynucleotide). In some embodiments, the expression of the one or more expression sequences from the complex in the subject is for at least 10 months longer than after administering a circular polyribonucleotide alone (e.g., lacking a capped polynucleotide). In some embodiments, the expression of the one or more expression sequences from the complex in the subject is for at least 11 months longer than after administering a circular polyribonucleotide alone (e.g., lacking a capped polynucleotide). In some embodiments, the expression of the one or more expression sequences from the complex in the subject is for at least 12 months longer than after administering a circular polyribonucleotide alone (e.g., lacking a capped polynucleotide). In some embodiments, the expression of the one or more expression sequences from the complex in the subject is for at least 13 months longer than after administering a circular polyribonucleotide alone (e.g., lacking a capped polynucleotide). In some embodiments, the expression of the one or more expression sequences from the complex in the subject is for at least 14 months longer than after administering a circular polyribonucleotide alone (e.g., lacking a capped polynucleotide). In some embodiments, the expression of the one or more expression sequences from the complex in the subject is for at least 15 months longer than after administering a circular polyribonucleotide alone (e.g., lacking a capped polynucleotide). In some embodiments, the expression of the one or more expression sequences from the complex in the subject is for at least 16 months longer than after administering a circular polyribonucleotide alone (e.g., lacking a capped polynucleotide). In some embodiments, the expression of the one or more expression sequences from the complex in the subject is for at least 17 months longer than after administering a circular polyribonucleotide alone (e.g., lacking a capped polynucleotide). In some embodiments, the expression of the one or more expression sequences from the complex in the subject is for at least 18 months longer than after administering a circular polyribonucleotide alone (e.g., lacking a capped polynucleotide). In some embodiments, the expression of the one or more expression sequences from the complex in the subject is for at least 19 months longer than after administering a circular polyribonucleotide alone (e.g., lacking a capped polynucleotide). In some embodiments, the expression of the one or more expression sequences from the complex in the subject is for at least 20 months longer than after administering a circular polyribonucleotide alone (e.g., lacking a capped polynucleotide). In some embodiments, the expression of the one or more expression sequences from the complex in the subject is for at least 21 months longer than after administering a circular polyribonucleotide alone (e.g., lacking a capped polynucleotide). In some embodiments, the expression of the one or more expression sequences from the complex in the subject is for at least 22 months longer than after administering a circular polyribonucleotide alone (e.g., lacking a capped polynucleotide). In some embodiments, the expression of the one or more expression sequences from the complex in the subject is for at least 23 months longer than after administering a circular polyribonucleotide alone (e.g., lacking a capped polynucleotide). In some embodiments, the expression of the one or more expression sequences from the complex in the subject is for at least 24 months longer than after administering a circular polyribonucleotide alone (e.g., lacking a capped polynucleotide). In some embodiments, the expression of the one or more expression sequences from the complex in the subject is for 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months 23 months, 24 months, or more longer than after administering a linear counterpart of the circular polyribonucleotide alone (e.g., lacking a capped polynucleotide). In some embodiments, the expression of the one or more expression sequences from the complex in the subject is for 1 month longer than after administering a linear counterpart of the circular polyribonucleotide alone (e.g., lacking a capped polynucleotide). In some embodiments, the expression of the one or more expression sequences from the complex in the subject is for 2 months longer than after administering a linear counterpart of the circular polyribonucleotide alone (e.g., lacking a capped polynucleotide). In some embodiments, the expression of the one or more expression sequences from the complex in the subject is for 3 months longer than after administering a linear counterpart of the circular polyribonucleotide alone (e.g., lacking a capped polynucleotide). In some embodiments, the expression of the one or more expression sequences from the complex in the subject is for 4 months longer than after administering a linear counterpart of the circular polyribonucleotide alone (e.g., lacking a capped polynucleotide). In some embodiments, the expression of the one or more expression sequences from the complex in the subject is for 5 months longer than after administering a linear counterpart of the circular polyribonucleotide alone (e.g., lacking a capped polynucleotide). In some embodiments, the expression of the one or more expression sequences from the complex in the subject is for 6 months longer than after administering a linear counterpart of the circular polyribonucleotide alone (e.g., lacking a capped polynucleotide). In some embodiments, the expression of the one or more expression sequences from the complex in the subject is for 7 months longer than after administering a linear counterpart of the circular polyribonucleotide alone (e.g., lacking a capped polynucleotide). In some embodiments, the expression of the one or more expression sequences from the complex in the subject is for 8 months longer than after administering a linear counterpart of the circular polyribonucleotide alone (e.g., lacking a capped polynucleotide). In some embodiments, the expression of the one or more expression sequences from the complex in the subject is for 9 months longer than after administering a linear counterpart of the circular polyribonucleotide alone (e.g., lacking a capped polynucleotide). In some embodiments, the expression of the one or more expression sequences from the complex in the subject is for 10 months longer than after administering a linear counterpart of the circular polyribonucleotide alone (e.g., lacking a capped polynucleotide). In some embodiments, the expression of the one or more expression sequences from the complex in the subject is for 11 months longer than after administering a linear counterpart of the circular polyribonucleotide alone (e.g., lacking a capped polynucleotide). In some embodiments, the expression of the one or more expression sequences from the complex in the subject is for 12 months longer than after administering a linear counterpart of the circular polyribonucleotide alone (e.g., lacking a capped polynucleotide). In some embodiments, the expression of the one or more expression sequences from the complex in the subject is for 13 months longer than after administering a linear counterpart of the circular polyribonucleotide alone (e.g., lacking a capped polynucleotide). In some embodiments, the expression of the one or more expression sequences from the complex in the subject is for 14 months longer than after administering a linear counterpart of the circular polyribonucleotide alone (e.g., lacking a capped polynucleotide). In some embodiments, the expression of the one or more expression sequences from the complex in the subject is for 15 months longer than after administering a linear counterpart of the circular polyribonucleotide alone (e.g., lacking a capped polynucleotide). In some embodiments, the expression of the one or more expression sequences from the complex in the subject is for 16 months longer than after administering a linear counterpart of the circular polyribonucleotide alone (e.g., lacking a capped polynucleotide). In some embodiments, the expression of the one or more expression sequences from the complex in the subject is for 17 months longer than after administering a linear counterpart of the circular polyribonucleotide alone (e.g., lacking a capped polynucleotide). In some embodiments, the expression of the one or more expression sequences from the complex in the subject is for 18 months longer than after administering a linear counterpart of the circular polyribonucleotide alone (e.g., lacking a capped polynucleotide). In some embodiments, the expression of the one or more expression sequences from the complex in the subject is for 19 months longer than after administering a linear counterpart of the circular polyribonucleotide alone (e.g., lacking a capped polynucleotide). In some embodiments, the expression of the one or more expression sequences from the complex in the subject is for 20 months longer than after administering a linear counterpart of the circular polyribonucleotide alone (e.g., lacking a capped polynucleotide). In some embodiments, the expression of the one or more expression sequences from the complex in the subject is for 21 months longer than after administering a linear counterpart of the circular polyribonucleotide alone (e.g., lacking a capped polynucleotide). In some embodiments, the expression of the one or more expression sequences from the complex in the subject is for 22 months longer than after administering a linear counterpart of the circular polyribonucleotide alone (e.g., lacking a capped polynucleotide). In some embodiments, the expression of the one or more expression sequences from the complex in the subject is for 23 months longer than after administering a linear counterpart of the circular polyribonucleotide alone (e.g., lacking a capped polynucleotide). In some embodiments, the expression of the one or more expression sequences from the complex in the subject is for 24 months longer than after administering a linear counterpart of the circular polyribonucleotide alone (e.g., lacking a capped polynucleotide).

In some aspects, the invention as provided herein comprises a method of expression of one or more expression sequences in a subject, comprising: providing a complex comprising: a circular polyribonucleotide as provided herein comprising the one or more expression sequences, and a capped polyribonucleotide as provided herein, wherein the first binding region of the capped polyribonucleotide is bound to the second binding region of the circular polyribonucleotide; and administering the complex to a cell of the subject, wherein expression of the one or more expression sequences from the complex in the subject is longer than after administering a circular polyribonucleotide alone (e.g., lacking a capped polynucleotide).

In some other aspects, the invention as provided herein comprises a method of expression of one or more expression sequences in a subject, comprising: administering to the subject a circular polyribonucleotide as provided herein comprising the one or more expression sequences, and a capped polyribonucleotide as provided herein, wherein the first binding region of the capped polyribonucleotide is bound to the second binding region of the circular polyribonucleotide to form a complex in the subject and expression of the one or more expression sequences from the complex in a cell of the subject is longer than after administering a circular polyribonucleotide alone (e.g., lacking a capped polynucleotide).

In some embodiments, the expression of the one or more expression sequences from the complex in the subject is for at least 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs or more longer than after administering a circular polyribonucleotide alone (e.g., lacking a capped polynucleotide). In some embodiments, the expression of the one or more expression sequences from the complex in the subject is for at least 1 hr longer than after administering a circular polyribonucleotide alone (e.g., lacking a capped polynucleotide). In some embodiments, the expression of the one or more expression sequences from the complex in the subject is for at least 2 hrs longer than after administering a circular polyribonucleotide alone (e.g., lacking a capped polynucleotide). In some embodiments, the expression of the one or more expression sequences from the complex in the subject is for at least 3 hrs longer than after administering a circular polyribonucleotide alone (e.g., lacking a capped polynucleotide). In some embodiments, the expression of the one or more expression sequences from the complex in the subject is for at least 4 hrs longer than after administering a circular polyribonucleotide alone (e.g., lacking a capped polynucleotide). In some embodiments, the expression of the one or more expression sequences from the complex in the subject is for at least 5 hrs longer than after administering a circular polyribonucleotide alone (e.g., lacking a capped polynucleotide). In some embodiments, the expression of the one or more expression sequences from the complex in the subject is for at least 6 hrs longer than after administering a circular polyribonucleotide alone (e.g., lacking a capped polynucleotide). In some embodiments, the expression of the one or more expression sequences from the complex in the subject is for at least 7 hrs longer than after administering a circular polyribonucleotide alone (e.g., lacking a capped polynucleotide). In some embodiments, the expression of the one or more expression sequences from the complex in the subject is for at least 8 hrs longer than after administering a circular polyribonucleotide alone (e.g., lacking a capped polynucleotide). In some embodiments, the expression of the one or more expression sequences from the complex in the subject is for at least 9 hrs longer than after administering a circular polyribonucleotide alone (e.g., lacking a capped polynucleotide). In some embodiments, the expression of the one or more expression sequences from the complex in the subject is for at least 10 hrs longer than after administering a circular polyribonucleotide alone (e.g., lacking a capped polynucleotide). In some embodiments, the expression of the one or more expression sequences from the complex in the subject is for at least 11 hrs longer than after administering a circular polyribonucleotide alone (e.g., lacking a capped polynucleotide). In some embodiments, the expression of the one or more expression sequences from the complex in the subject is for at least 12 hrs longer than after administering a circular polyribonucleotide alone (e.g., lacking a capped polynucleotide). In some embodiments, the expression of the one or more expression sequences from the complex in the subject is for at least 13 hrs longer than after administering a circular polyribonucleotide alone (e.g., lacking a capped polynucleotide). In some embodiments, the expression of the one or more expression sequences from the complex in the subject is for at least 14 hrs longer than after administering a circular polyribonucleotide alone (e.g., lacking a capped polynucleotide). In some embodiments, the expression of the one or more expression sequences from the complex in the subject is for at least 15 hrs longer than after administering a circular polyribonucleotide alone (e.g., lacking a capped polynucleotide). In some embodiments, the expression of the one or more expression sequences from the complex in the subject is for at least 16 hrs longer than after administering a circular polyribonucleotide alone (e.g., lacking a capped polynucleotide). In some embodiments, the expression of the one or more expression sequences from the complex in the subject is for at least 17 hrs longer than after administering a circular polyribonucleotide alone (e.g., lacking a capped polynucleotide). In some embodiments, the expression of the one or more expression sequences from the complex in the subject is for at least 18 hrs longer than after administering a circular polyribonucleotide alone (e.g., lacking a capped polynucleotide). In some embodiments, the expression of the one or more expression sequences from the complex in the subject is for at least 19 hrs longer than after administering a circular polyribonucleotide alone (e.g., lacking a capped polynucleotide). In some embodiments, the expression of the one or more expression sequences from the complex in the subject is for at least 20 hrs longer than after administering a circular polyribonucleotide alone (e.g., lacking a capped polynucleotide). In some embodiments, the expression of the one or more expression sequences from the complex in the subject is for at least 21 hrs longer than after administering a circular polyribonucleotide alone (e.g., lacking a capped polynucleotide). In some embodiments, the expression of the one or more expression sequences from the complex in the subject is for at least 22 hrs longer than after administering a circular polyribonucleotide alone (e.g., lacking a capped polynucleotide). In some embodiments, the expression of the one or more expression sequences from the complex in the subject is for at least 23 hrs longer than after administering a circular polyribonucleotide alone (e.g., lacking a capped polynucleotide). In some embodiments, the expression of the one or more expression sequences from the complex in the subject is for 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, or more longer than after administering a circular polyribonucleotide alone (e.g., lacking a capped polynucleotide). In some embodiments, the expression of the one or more expression sequences from the complex in the subject is for 1 hr longer than after administering a circular polyribonucleotide alone (e.g., lacking a capped polynucleotide). In some embodiments, the expression of the one or more expression sequences from the complex in the subject is for 2 hrs longer than after administering a circular polyribonucleotide alone (e.g., lacking a capped polynucleotide). In some embodiments, the expression of the one or more expression sequences from the complex in the subject is for 3 hrs longer than after administering a circular polyribonucleotide alone (e.g., lacking a capped polynucleotide). In some embodiments, the expression of the one or more expression sequences from the complex in the subject is for 4 hrs longer than after administering a circular polyribonucleotide alone (e.g., lacking a capped polynucleotide). In some embodiments, the expression of the one or more expression sequences from the complex in the subject is for 5 hrs longer than after administering a circular polyribonucleotide alone (e.g., lacking a capped polynucleotide). In some embodiments, the expression of the one or more expression sequences from the complex in the subject is for 6 hrs longer than after administering a circular polyribonucleotide alone (e.g., lacking a capped polynucleotide). In some embodiments, the expression of the one or more expression sequences from the complex in the subject is for 7 hrs longer than after administering a circular polyribonucleotide alone (e.g., lacking a capped polynucleotide). In some embodiments, the expression of the one or more expression sequences from the complex in the subject is for 8 hrs longer than after administering a circular polyribonucleotide alone (e.g., lacking a capped polynucleotide). In some embodiments, the expression of the one or more expression sequences from the complex in the subject is for 9 hrs longer than after administering a circular polyribonucleotide alone (e.g., lacking a capped polynucleotide). In some embodiments, the expression of the one or more expression sequences from the complex in the subject is for 10 hrs longer than after administering a circular polyribonucleotide alone (e.g., lacking a capped polynucleotide). In some embodiments, the expression of the one or more expression sequences from the complex in the subject is for 11 hrs longer than after administering a circular polyribonucleotide alone (e.g., lacking a capped polynucleotide). In some embodiments, the expression of the one or more expression sequences from the complex in the subject is for 12 hrs longer than after administering a circular polyribonucleotide alone (e.g., lacking a capped polynucleotide). In some embodiments, the expression of the one or more expression sequences from the complex in the subject is for, 13 hrs longer than after administering a circular polyribonucleotide alone (e.g., lacking a capped polynucleotide). In some embodiments, the expression of the one or more expression sequences from the complex in the subject is for 14 hrs longer than after administering a circular polyribonucleotide alone (e.g., lacking a capped polynucleotide). In some embodiments, the expression of the one or more expression sequences from the complex in the subject is for 15 hrs longer than after administering a circular polyribonucleotide alone (e.g., lacking a capped polynucleotide). In some embodiments, the expression of the one or more expression sequences from the complex in the subject is for 16 hrs longer than after administering a circular polyribonucleotide alone (e.g., lacking a capped polynucleotide). In some embodiments, the expression of the one or more expression sequences from the complex in the subject is for 17 hrs longer than after administering a circular polyribonucleotide alone (e.g., lacking a capped polynucleotide). In some embodiments, the expression of the one or more expression sequences from the complex in the subject is for 18 hrs longer than after administering a circular polyribonucleotide alone (e.g., lacking a capped polynucleotide). In some embodiments, the expression of the one or more expression sequences from the complex in the subject is for 19 hrs longer than after administering a circular polyribonucleotide alone (e.g., lacking a capped polynucleotide). In some embodiments, the expression of the one or more expression sequences from the complex in the subject is for 20 hrs longer than after administering a circular polyribonucleotide alone (e.g., lacking a capped polynucleotide). In some embodiments, the expression of the one or more expression sequences from the complex in the subject is for 21 hrs longer than after administering a circular polyribonucleotide alone (e.g., lacking a capped polynucleotide). In some embodiments, the expression of the one or more expression sequences from the complex in the subject is for 22 hrs longer than after administering a circular polyribonucleotide alone (e.g., lacking a capped polynucleotide). In some embodiments, the expression of the one or more expression sequences from the complex in the subject is for 23 hrs longer than after administering a circular polyribonucleotide alone (e.g., lacking a capped polynucleotide). In some embodiments, the expression of the one or more expression sequences from the complex in the subject is for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more longer than after administering a circular polyribonucleotide alone (e.g., lacking a capped polynucleotide). In some embodiments, the expression of the one or more expression sequences from the complex in the subject is for at least 1 day longer than after administering a circular polyribonucleotide alone (e.g., lacking a capped polynucleotide). In some embodiments, the expression of the one or more expression sequences from the complex in the subject is for at least 2 days longer than after administering a circular polyribonucleotide alone (e.g., lacking a capped polynucleotide). In some embodiments, the expression of the one or more expression sequences from the complex in the subject is for at least 3 days longer than after administering a circular polyribonucleotide alone (e.g., lacking a capped polynucleotide). In some embodiments, the expression of the one or more expression sequences from the complex in the subject is for at least 4 days longer than after administering a circular polyribonucleotide alone (e.g., lacking a capped polynucleotide). In some embodiments, the expression of the one or more expression sequences from the complex in the subject is for at least 5 days longer than after administering a circular polyribonucleotide alone (e.g., lacking a capped polynucleotide). In some embodiments, the expression of the one or more expression sequences from the complex in the subject is for at least 6 days longer than after administering a circular polyribonucleotide alone (e.g., lacking a capped polynucleotide). In some embodiments, the expression of the one or more expression sequences from the complex in the subject is for at least 7 days longer than after administering a circular polyribonucleotide alone (e.g., lacking a capped polynucleotide). In some embodiments, the expression of the one or more expression sequences from the complex in the subject is for at least 8 days longer than after administering a circular polyribonucleotide alone (e.g., lacking a capped polynucleotide). In some embodiments, the expression of the one or more expression sequences from the complex in the subject is for at least 9 days longer than after administering a circular polyribonucleotide alone (e.g., lacking a capped polynucleotide). In some embodiments, the expression of the one or more expression sequences from the complex in the subject is for at least 10 days longer than after administering a circular polyribonucleotide alone (e.g., lacking a capped polynucleotide). In some embodiments, the expression of the one or more expression sequences from the complex in the subject is for at least 11 days longer than after administering a circular polyribonucleotide alone (e.g., lacking a capped polynucleotide). In some embodiments, the expression of the one or more expression sequences from the complex in the subject is for at least 12 days longer than after administering a circular polyribonucleotide alone (e.g., lacking a capped polynucleotide). In some embodiments, the expression of the one or more expression sequences from the complex in the subject is for at least 13 days longer than after administering a circular polyribonucleotide alone (e.g., lacking a capped polynucleotide). In some embodiments, the expression of the one or more expression sequences from the complex in the subject is for at least 14 days longer than after administering a circular polyribonucleotide alone (e.g., lacking a capped polynucleotide). In some embodiments, the expression of the one or more expression sequences from the complex in the subject is for at least 15 days longer than after administering a circular polyribonucleotide alone (e.g., lacking a capped polynucleotide). In some embodiments, the expression of the one or more expression sequences from the complex in the subject is for at least 16 days longer than after administering a circular polyribonucleotide alone (e.g., lacking a capped polynucleotide). In some embodiments, the expression of the one or more expression sequences from the complex in the subject is for at least 17 days longer than after administering a circular polyribonucleotide alone (e.g., lacking a capped polynucleotide). In some embodiments, the expression of the one or more expression sequences from the complex in the subject is for at least 18 days longer than after administering a circular polyribonucleotide alone (e.g., lacking a capped polynucleotide). In some embodiments, the expression of the one or more expression sequences from the complex in the subject is for at least 19 days longer than after administering a circular polyribonucleotide alone (e.g., lacking a capped polynucleotide). In some embodiments, the expression of the one or more expression sequences from the complex in the subject is for at least 20 days longer than after administering a circular polyribonucleotide alone (e.g., lacking a capped polynucleotide). In some embodiments, the expression of the one or more expression sequences from the complex in the subject is for at least 21 days longer than after administering a circular polyribonucleotide alone (e.g., lacking a capped polynucleotide). In some embodiments, the expression of the one or more expression sequences from the complex in the subject is for at least 22 days longer than after administering a circular polyribonucleotide alone (e.g., lacking a capped polynucleotide). In some embodiments, the expression of the one or more expression sequences from the complex in the subject is for at least 23 days longer than after administering a circular polyribonucleotide alone (e.g., lacking a capped polynucleotide). In some embodiments, the expression of the one or more expression sequences from the complex in the subject is for at least 24 days longer than after administering a circular polyribonucleotide alone (e.g., lacking a capped polynucleotide). In some embodiments, the expression of the one or more expression sequences from the complex in the subject is for at least 25 days longer than after administering a circular polyribonucleotide alone (e.g., lacking a capped polynucleotide). In some embodiments, the expression of the one or more expression sequences from the complex in the subject is for at least 26 days longer than after administering a circular polyribonucleotide alone (e.g., lacking a capped polynucleotide). In some embodiments, the expression of the one or more expression sequences from the complex in the subject is for at least 27 days longer than after administering a circular polyribonucleotide alone (e.g., lacking a capped polynucleotide). In some embodiments, the expression of the one or more expression sequences from the complex in the subject is for at least 28 days longer than after administering a circular polyribonucleotide alone (e.g., lacking a capped polynucleotide). In some embodiments, the expression of the one or more expression sequences from the complex in the subject is for at least 29 days longer than after administering a circular polyribonucleotide alone (e.g., lacking a capped polynucleotide). In some embodiments, the expression of the one or more expression sequences from the complex in the subject is for at least 30 days longer than after administering a circular polyribonucleotide alone (e.g., lacking a capped polynucleotide). In some embodiments, the expression of the one or more expression sequences from the complex in the subject is for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more longer than after administering a circular polyribonucleotide alone (e.g., lacking a capped polynucleotide). In some embodiments, the expression of the one or more expression sequences from the complex in the subject is for 1 day longer than after administering a circular polyribonucleotide alone (e.g., lacking a capped polynucleotide). In some embodiments, the expression of the one or more expression sequences from the complex in the subject is for 2 days longer than after administering a circular polyribonucleotide alone (e.g., lacking a capped polynucleotide). In some embodiments, the expression of the one or more expression sequences from the complex in the subject is for 3 days longer than after administering a circular polyribonucleotide alone (e.g., lacking a capped polynucleotide). In some embodiments, the expression of the one or more expression sequences from the complex in the subject is for 4 days longer than after administering a circular polyribonucleotide alone (e.g., lacking a capped polynucleotide). In some embodiments, the expression of the one or more expression sequences from the complex in the subject is for 5 days longer than after administering a circular polyribonucleotide alone (e.g., lacking a capped polynucleotide). In some embodiments, the expression of the one or more expression sequences from the complex in the subject is for 6 days longer than after administering a circular polyribonucleotide alone (e.g., lacking a capped polynucleotide). In some embodiments, the expression of the one or more expression sequences from the complex in the subject is for 7 days longer than after administering a circular polyribonucleotide alone (e.g., lacking a capped polynucleotide). In some embodiments, the expression of the one or more expression sequences from the complex in the subject is for 8 days longer than after administering a circular polyribonucleotide alone (e.g., lacking a capped polynucleotide). In some embodiments, the expression of the one or more expression sequences from the complex in the subject is for 9 days longer than after administering a circular polyribonucleotide alone (e.g., lacking a capped polynucleotide). In some embodiments, the expression of the one or more expression sequences from the complex in the subject is for 10 days longer than after administering a circular polyribonucleotide alone (e.g., lacking a capped polynucleotide). In some embodiments, the expression of the one or more expression sequences from the complex in the subject is for 11 days longer than after administering a circular polyribonucleotide alone (e.g., lacking a capped polynucleotide). In some embodiments, the expression of the one or more expression sequences from the complex in the subject is for 12 days longer than after administering a circular polyribonucleotide alone (e.g., lacking a capped polynucleotide). In some embodiments, the expression of the one or more expression sequences from the complex in the subject is for 13 days longer than after administering a circular polyribonucleotide alone (e.g., lacking a capped polynucleotide). In some embodiments, the expression of the one or more expression sequences from the complex in the subject is for 14 days longer than after administering a circular polyribonucleotide alone (e.g., lacking a capped polynucleotide). In some embodiments, the expression of the one or more expression sequences from the complex in the subject is for 15 days, longer than after administering a circular polyribonucleotide alone (e.g., lacking a capped polynucleotide). In some embodiments, the expression of the one or more expression sequences from the complex in the subject is for 16 days longer than after administering a circular polyribonucleotide alone (e.g., lacking a capped polynucleotide). In some embodiments, the expression of the one or more expression sequences from the complex in the subject is for 17 days longer than after administering a circular polyribonucleotide alone (e.g., lacking a capped polynucleotide). In some embodiments, the expression of the one or more expression sequences from the complex in the subject is for 18 days longer than after administering a circular polyribonucleotide alone (e.g., lacking a capped polynucleotide). In some embodiments, the expression of the one or more expression sequences from the complex in the subject is for 19 days longer than after administering a circular polyribonucleotide alone (e.g., lacking a capped polynucleotide). In some embodiments, the expression of the one or more expression sequences from the complex in the subject is for 20 days longer than after administering a circular polyribonucleotide alone (e.g., lacking a capped polynucleotide). In some embodiments, the expression of the one or more expression sequences from the complex in the subject is for 21 days longer than after administering a circular polyribonucleotide alone (e.g., lacking a capped polynucleotide). In some embodiments, the expression of the one or more expression sequences from the complex in the subject is for 22 days longer than after administering a circular polyribonucleotide alone (e.g., lacking a capped polynucleotide). In some embodiments, the expression of the one or more expression sequences from the complex in the subject is for 23 days longer than after administering a circular polyribonucleotide alone (e.g., lacking a capped polynucleotide). In some embodiments, the expression of the one or more expression sequences from the complex in the subject is for 24 days longer than after administering a circular polyribonucleotide alone (e.g., lacking a capped polynucleotide). In some embodiments, the expression of the one or more expression sequences from the complex in the subject is for 25 days longer than after administering a circular polyribonucleotide alone (e.g., lacking a capped polynucleotide). In some embodiments, the expression of the one or more expression sequences from the complex in the subject is for 26 days longer than after administering a circular polyribonucleotide alone (e.g., lacking a capped polynucleotide). In some embodiments, the expression of the one or more expression sequences from the complex in the subject is for 27 days longer than after administering a circular polyribonucleotide alone (e.g., lacking a capped polynucleotide). In some embodiments, the expression of the one or more expression sequences from the complex in the subject is for 28 days longer than after administering a circular polyribonucleotide alone (e.g., lacking a capped polynucleotide). In some embodiments, the expression of the one or more expression sequences from the complex in the subject is for 29 days longer than after administering a circular polyribonucleotide alone (e.g., lacking a capped polynucleotide). In some embodiments, the expression of the one or more expression sequences from the complex in the subject is for 30 days longer than after administering a circular polyribonucleotide alone (e.g., lacking a capped polynucleotide). In some embodiments, the expression of the one or more expression sequences from the complex in the subject is for at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months 23 months, 24 months, or more longer than after administering a circular polyribonucleotide alone (e.g., lacking a capped polynucleotide). In some embodiments, the expression of the one or more expression sequences from the complex in the subject is for at least 1 month longer than after administering a circular polyribonucleotide alone (e.g., lacking a capped polynucleotide). In some embodiments, the expression of the one or more expression sequences from the complex in the subject is for at least 2 months longer than after administering a circular polyribonucleotide alone (e.g., lacking a capped polynucleotide). In some embodiments, the expression of the one or more expression sequences from the complex in the subject is for at least 3 months longer than after administering a circular polyribonucleotide alone (e.g., lacking a capped polynucleotide). In some embodiments, the expression of the one or more expression sequences from the complex in the subject is for at least 4 months longer than after administering a circular polyribonucleotide alone (e.g., lacking a capped polynucleotide). In some embodiments, the expression of the one or more expression sequences from the complex in the subject is for at least 5 months longer than after administering a circular polyribonucleotide alone (e.g., lacking a capped polynucleotide). In some embodiments, the expression of the one or more expression sequences from the complex in the subject is for at least 6 months longer than after administering a circular polyribonucleotide alone (e.g., lacking a capped polynucleotide). In some embodiments, the expression of the one or more expression sequences from the complex in the subject is for at least 7 months longer than after administering a circular polyribonucleotide alone (e.g., lacking a capped polynucleotide). In some embodiments, the expression of the one or more expression sequences from the complex in the subject is for at least 8 months longer than after administering a circular polyribonucleotide alone (e.g., lacking a capped polynucleotide). In some embodiments, the expression of the one or more expression sequences from the complex in the subject is for at least 9 months, 10 months longer than after administering a circular polyribonucleotide alone (e.g., lacking a capped polynucleotide). In some embodiments, the expression of the one or more expression sequences from the complex in the subject is for at least 11 months longer than after administering a circular polyribonucleotide alone (e.g., lacking a capped polynucleotide). In some embodiments, the expression of the one or more expression sequences from the complex in the subject is for at least 12 months longer than after administering a circular polyribonucleotide alone (e.g., lacking a capped polynucleotide). In some embodiments, the expression of the one or more expression sequences from the complex in the subject is for at least 13 months longer than after administering a circular polyribonucleotide alone (e.g., lacking a capped polynucleotide). In some embodiments, the expression of the one or more expression sequences from the complex in the subject is for at least 14 months longer than after administering a circular polyribonucleotide alone (e.g., lacking a capped polynucleotide). In some embodiments, the expression of the one or more expression sequences from the complex in the subject is for at least 15 months longer than after administering a circular polyribonucleotide alone (e.g., lacking a capped polynucleotide). In some embodiments, the expression of the one or more expression sequences from the complex in the subject is for at least 16 months longer than after administering a circular polyribonucleotide alone (e.g., lacking a capped polynucleotide). In some embodiments, the expression of the one or more expression sequences from the complex in the subject is for at least 17 months longer than after administering a circular polyribonucleotide alone (e.g., lacking a capped polynucleotide). In some embodiments, the expression of the one or more expression sequences from the complex in the subject is for at least 18 months, 19 months longer than after administering a circular polyribonucleotide alone (e.g., lacking a capped polynucleotide). In some embodiments, the expression of the one or more expression sequences from the complex in the subject is for at least 20 months longer than after administering a circular polyribonucleotide alone (e.g., lacking a capped polynucleotide). In some embodiments, the expression of the one or more expression sequences from the complex in the subject is for at least 21 months longer than after administering a circular polyribonucleotide alone (e.g., lacking a capped polynucleotide). In some embodiments, the expression of the one or more expression sequences from the complex in the subject is for at least 22 months longer than after administering a circular polyribonucleotide alone (e.g., lacking a capped polynucleotide). In some embodiments, the expression of the one or more expression sequences from the complex in the subject is for at least 23 months longer than after administering a circular polyribonucleotide alone (e.g., lacking a capped polynucleotide). In some embodiments, the expression of the one or more expression sequences from the complex in the subject is for at least 24 months longer than after administering a circular polyribonucleotide alone (e.g., lacking a capped polynucleotide). In some embodiments, the expression of the one or more expression sequences from the complex in the subject is for 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months 23 months, 24 months, or more longer than after administering a circular polyribonucleotide alone (e.g., lacking a capped polynucleotide). In some embodiments, the expression of the one or more expression sequences from the complex in the subject is for 1 month longer than after administering a circular polyribonucleotide alone (e.g., lacking a capped polynucleotide). In some embodiments, the expression of the one or more expression sequences from the complex in the subject is for 2 months longer than after administering a circular polyribonucleotide alone (e.g., lacking a capped polynucleotide). In some embodiments, the expression of the one or more expression sequences from the complex in the subject is for 3 months longer than after administering a circular polyribonucleotide alone (e.g., lacking a capped polynucleotide). In some embodiments, the expression of the one or more expression sequences from the complex in the subject is for 4 months longer than after administering a circular polyribonucleotide alone (e.g., lacking a capped polynucleotide). In some embodiments, the expression of the one or more expression sequences from the complex in the subject is for 5 months longer than after administering a circular polyribonucleotide alone (e.g., lacking a capped polynucleotide). In some embodiments, the expression of the one or more expression sequences from the complex in the subject is for 6 months longer than after administering a circular polyribonucleotide alone (e.g., lacking a capped polynucleotide). In some embodiments, the expression of the one or more expression sequences from the complex in the subject is for 7 months longer than after administering a circular polyribonucleotide alone (e.g., lacking a capped polynucleotide). In some embodiments, the expression of the one or more expression sequences from the complex in the subject is for 8 months longer than after administering a circular polyribonucleotide alone (e.g., lacking a capped polynucleotide). In some embodiments, the expression of the one or more expression sequences from the complex in the subject is for 9 months longer than after administering a circular polyribonucleotide alone (e.g., lacking a capped polynucleotide). In some embodiments, the expression of the one or more expression sequences from the complex in the subject is for 10 months longer than after administering a circular polyribonucleotide alone (e.g., lacking a capped polynucleotide). In some embodiments, the expression of the one or more expression sequences from the complex in the subject is for 11 months longer than after administering a circular polyribonucleotide alone (e.g., lacking a capped polynucleotide). In some embodiments, the expression of the one or more expression sequences from the complex in the subject is for 12 months longer than after administering a circular polyribonucleotide alone (e.g., lacking a capped polynucleotide). In some embodiments, the expression of the one or more expression sequences from the complex in the subject is for 13 months longer than after administering a circular polyribonucleotide alone (e.g., lacking a capped polynucleotide). In some embodiments, the expression of the one or more expression sequences from the complex in the subject is for 14 months longer than after administering a circular polyribonucleotide alone (e.g., lacking a capped polynucleotide). In some embodiments, the expression of the one or more expression sequences from the complex in the subject is for 15 months longer than after administering a circular polyribonucleotide alone (e.g., lacking a capped polynucleotide). In some embodiments, the expression of the one or more expression sequences from the complex in the subject is for 16 months longer than after administering a circular polyribonucleotide alone (e.g., lacking a capped polynucleotide). In some embodiments, the expression of the one or more expression sequences from the complex in the subject is for 17 months longer than after administering a circular polyribonucleotide alone (e.g., lacking a capped polynucleotide). In some embodiments, the expression of the one or more expression sequences from the complex in the subject is for 18 months longer than after administering a circular polyribonucleotide alone (e.g., lacking a capped polynucleotide). In some embodiments, the expression of the one or more expression sequences from the complex in the subject is for 19 months longer than after administering a circular polyribonucleotide alone (e.g., lacking a capped polynucleotide). In some embodiments, the expression of the one or more expression sequences from the complex in the subject is for 20 months longer than after administering a circular polyribonucleotide alone (e.g., lacking a capped polynucleotide). In some embodiments, the expression of the one or more expression sequences from the complex in the subject is for 21 months longer than after administering a circular polyribonucleotide alone (e.g., lacking a capped polynucleotide). In some embodiments, the expression of the one or more expression sequences from the complex in the subject is for 22 months longer than after administering a circular polyribonucleotide alone (e.g., lacking a capped polynucleotide). In some embodiments, the expression of the one or more expression sequences from the complex in the subject is for 23 months longer than after administering a circular polyribonucleotide alone (e.g., lacking a capped polynucleotide). In some embodiments, the expression of the one or more expression sequences from the complex in the subject is for 24 months longer than after administering a circular polyribonucleotide alone (e.g., lacking a capped polynucleotide).

### Methods of Treatment

In some aspects, the invention as provided herein comprises a method of treating a subject in need thereof comprising administering a capped polyribonucleotide as provided herein and a circular polyribonucleotide as provided herein to the subject, wherein the administering is effective to treat the subject.

In some other aspects, the invention as provided herein comprises a method of treating a subject in need thereof comprising administering a capped polyribonucleotide bound to a circular polyribonucleotide as provided herein to the subject, wherein the administering is effective to treat the subject.

In some aspects, the invention as provided herein comprises the phamaceutical composition of as described herein for use in a method of treatment of a human or animal body by therapy.

In some aspects, the invention as provided herein comprises the complex as disclosed herein for use as a medicament or a pharmaceutical.

In some aspects, the invention as provided herein comprises the complex as disclosed herein for use in a method of treatment of a human or animal body by therapy.

In some aspects, the invention as provided herein comprises features a use of the complex of as disclosed herein, or the polyribonucleotide as disclosed herein and the circular polyribonucleotide as disclosed herein, in the manufacture of a medicament or a pharmaceutical.

In some aspects, the invention as provided herein comprises a use of the complex as disclosed herein, or the polyribonucleotide as disclosed herein and the circular polyribonucleotide as disclosed herein, in the manufacture of a medicament or a pharmaceutical for treating a human or animal body by therapy.

In some embodiments, the subject is a mammal. In some embodiments, the subject is a human. In some embodiments, the subject is a pet. In some embodiments, the subject is a pet live-stock animal.

In some embodiments, the method comprises administering a pharmaceutical composition comprising a capped polyribonucleotide and a circular polyribonucleotide as provided herein. In some embodiments, the method comprises administering a pharmaceutical composition comprising a polyribonucleotide comprising a 5′ modified guanosine cap and a circular polyribonucleotide. In some embodiments, the method comprises administering a pharmaceutical composition comprising a complex of a capped polyribonucleotide and a circular polyribonucleotide wherein the complex is produced by binding the first binding region of a capped polyribonucleotide as provided herein to a second binding region of a circular polyribonucleotide as provided herein.

In some embodiments, the method comprises administering a first pharmaceutical composition comprising a capped polyribonucleotide and a second pharmaceutical composition comprising a circular polyribonucleotide as provided herein. In some embodiments, the method comprises administering a first pharmaceutical composition comprising a polyribonucleotide comprising a 5′ modified guanosine cap and a second pharmaceutical composition comprising a circular polyribonucleotide. In some embodiments, the first pharmaceutical composition comprising a capped polyribonucleotide and the second pharmaceutical composition comprising a circular polyribonucleotide are administered to a subject in need thereof simultaneously, separately, or consecutively. In some embodiments, the first pharmaceutical composition comprising a polyribonucleotide comprising a 5′ modified guanosine cap and the second pharmaceutical composition comprising a circular polyribonucleotide are administered to a subject in need thereof simultaneously, separately, or consecutively.

In some embodiments, the method further comprises administering to the subject in need thereof a secondary or additional therapeutic agent or therapy in combination with a capped polyribonucleotide as provided herein and a circular polyribonucleotide as provided herein. In some embodiments, the method further comprises administering to the subject in need thereof a secondary or additional therapeutic agent or therapy in combination with a capped polyribonucleotide bound to a circular polyribonucleotide as provided herein.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i. e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition.

By “treating or preventing a disease or a condition” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a condition is one who a medical practitioner has diagnosed as having such a disease or a condition. Diagnosis may be by any suitable means. A patient in whom the development of a disease or a condition is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

Examples of the condition or the disease include, but are not limited to, a proliferative disease, a metabolic disease or disorder, a cardiovascular disease or disorder, an infectious disease, a neurological or neurodegenerative disease or disorder, and an inflammatory disease or disorder.

For instance, examples of a proliferative disease, includes, but is not limited to, a malignant, pre-malignant or benign cancer. Cancers to be treated using the disclosed methods include, for example, a solid tumor, a lymphoma or a leukemia. In one embodiment, a cancer can be, for example, a brain tumor (e.g., a malignant, pre-malignant or benign brain tumor such as, for example, a glioblastoma, an astrocytoma, a meningioma, a medulloblastoma or a peripheral neuroectodermal tumor), a carcinoma (e.g., gall bladder carcinoma, bronchial carcinoma, basal cell carcinoma, adenocarcinoma, squamous cell carcinoma, small cell carcinoma, large cell undifferentiated carcinoma, adenomas, cystadenoma, etc.), a basalioma, a teratoma, a retinoblastoma, a choroidea melanoma, a seminoma, a sarcoma (e.g., Ewing sarcoma, rhabdomyosarcoma, craniopharyngeoma, osteosarcoma, chondrosarcoma, myosarcoma, liposarcoma, fibrosarcoma, leimyosarcoma, Askin’s tumor, lymphosarcoma, neurosarcoma, Kaposi’s sarcoma, dermatofibrosarcoma, angiosarcoma, etc.), a plasmocytoma, a head and neck tumor (e.g., oral, laryngeal, nasopharyngeal, esophageal, etc.), a liver tumor, a kidney tumor, a renal cell tumor, a squamous cell carcinoma, a uterine tumor, a bone tumor, a prostate tumor, a breast tumor including, but not limited to, a breast tumor that is Her2- and/or ER- and/or PR-, a bladder tumor, a pancreatic tumor, an endometrium tumor, a squamous cell carcinoma, a stomach tumor, gliomas, a colorectal tumor, a testicular tumor, a colon tumor, a rectal tumor, an ovarian tumor, a cervical tumor, an eye tumor, a central nervous system tumor (e.g., primary CNS lymphomas, spinal axis tumors, brain stem gliomas, pituitary adenomas, etc.), a thyroid tumor, a lung tumor (e.g., non-small cell lung cancer (NSCLC) or small cell lung cancer), a leukemia or a lymphoma (e.g., cutaneous T-cell lymphomas (CTCL), non-cutaneous peripheral T-cell lymphomas, lymphomas associated with human T-cell lymphotrophic virus (HTLV) such as adult T-cell leukemia/lymphoma (ATLL), B-cell lymphoma, acute non-lymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin’s lymphoma, Burkitt lymphoma, adult T-cell leukemia lymphoma, acute-myeloid leukemia (AML), chronic myeloid leukemia (CML), or hepatocellular carcinoma, etc.), a multiple myeloma, a skin tumor (e.g., basal cell carcinomas, squamous cell carcinomas, melanomas such as malignant melanomas, cutaneous melanomas or intraocular melanomas, Dermatofibrosarcoma protuberans, Merkel cell carcinoma or Kaposi’s sarcoma), a gynecologic tumor (e.g., uterine sarcomas, carcinoma of the fallopian tubes, carcinoma of the endometrium, carcinoma of the cervix, carcinoma of the vagina, carcinoma of the vulva, etc.), Hodgkin’s disease, a cancer of the small intestine, a cancer of the endocrine system (e.g., a cancer of the thyroid, parathyroid or adrenal glands, etc.), a mesothelioma, a cancer of the urethra, a cancer of the penis, tumors related to Gorlin’s syndrome (e.g., medulloblastomas, meningioma, etc.), a tumor of unknown origin; or metastases of any thereto. In some embodiments, the cancer is a lung tumor, a breast tumor, a colon tumor, a colorectal tumor, a head and neck tumor, a liver tumor, a prostate tumor, a glioma, glioblastoma multiforme, a ovarian tumor or a thyroid tumor; or metastases of any thereto. In some other embodiments, the cancer is an endometrial tumor, bladder tumor, multiple myeloma, melanoma, renal tumor, sarcoma, cervical tumor, leukemia, and neuroblastoma.

For another instance, examples of the metabolic disease or disorder include, but are not limited to diabetes, metabolic syndrome, obesity, hyperlipidemia, high cholesterol, arteriosclerosis, hypertension, non-alcoholic steatohepatitis, non-alcoholic fatty liver, non-alcoholic fatty liver disease, hepatic steatosis, and any combination thereof.

For example, the inflammatory disorder or disorder partially or fully results from obesity, metabolic syndrome, an immune disorder, an Neoplasm, an infectious disorder, a chemical agent, an inflammatory bowel disorder, reperfusion injury, necrosis, or combinations thereof. In some embodiments, the inflammatory disorder is an autoimmune disorder, an allergy, a leukocyte defect, graft versus host disease, tissue transplant rejection, or combinations thereof. In some embodiments, the inflammatory disorder is a bacterial infection, a protozoal infection, a protozoal infection, a viral infection, a fungal infection, or combinations thereof. In some embodiments, the inflammatory disorder is Acute disseminated encephalomyelitis; Addison’s disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture’s syndrome; Graves’ disease; Guillain-Barre syndrome; Hashimoto’s disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjëgren’s syndrome; Vasculitis; Vitiligo; Wegener’s granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn’s disorder; Collagenous colitis; Lymphocytic colitis; Ischaemic colitis; Diversion colitis; Behçet’s syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer’s disorder, Parkinson’s disorder, Epilepsy, AIDS dementia, Asthma, Adult respiratory distress syndrome, Bronchitis, Cystic fibrosis, Acute leukocyte-mediated lung injury, Distal proctitis, Wegener’s granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease, abdominal aortic aneurysm (AAA); or combinations thereof.

For another instance, examples of the neurological or neurodegenerative disease or disorder include, but are not limited to, Aarskog syndrome, Alzheimer’s disease, amyotrophic lateral sclerosis (Lou Gehrig’s disease), aphasia, Bell’s Palsy, Creutzfeldt-Jakob disease, cerebrovascular disease, Cornelia de Lange syndrome, epilepsy and other severe seizure disorders, dentatorubral-pallidoluysian atrophy, fragile X syndrome, hypomelanosis of Ito, Joubert syndrome, Kennedy’s disease, Machado-Joseph’s diseases, migraines, Moebius syndrome, myotonic dystrophy, neuromuscular disorders, Guillain-Barre, muscular dystrophy, neuro-oncology disorders, neurofibromatosis, neuro-immunological disorders, multiple sclerosis, pain, pediatric neurology, autism, dyslexia, neuro-otology disorders, Meniere’s disease, Parkinson’s disease and movement disorders, Phenylketonuria, Rubinstein-Taybi syndrome, sleep disorders, spinocerebellar ataxia I, Smith-Lemli-Opitz syndrome, Sotos syndrome, spinal bulbar atrophy, type 1 dominant cerebellar ataxia, Tourette syndrome, tuberous sclerosis complex and William’s syndrome.

### Delivery

Pharmaceutical compositions as described herein can be formulated for example to include a pharmaceutical excipient or carrier. The pharmaceutical compositions described herein may be included in pharmaceutical compositions with a delivery carrier. In some embodiments, the circular polyribonucleotide, the capped polyribonucleotide, or the complex thereof as described herein may be included in a pharmaceutical composition free of any carrier. In some embodiments, the circular polyribonucleotide, the capped polyribonucleotide, or the complex thereof as described herein may be included in a pharmaceutical composition comprising a parenterally acceptable diluent. Methods as disclosed herein include a method of in vivo delivery of the circular polyribonucleotide, the capped polyribonucleotide, or the complex thereof as disclosed herein or a pharmaceutical composition as disclosed herein comprising parenterally administering the circular polyribonucleotide, the capped polyribonucleotide, or the complex thereof as disclosed herein or a pharmaceutical composition as disclosed herein to the cell or tissue of a subject, or to a subject.

Pharmaceutical compositions described herein may be formulated for example to include a pharmaceutical excipient or carrier. A pharmaceutical carrier can be a membrane, lipid bilayer, and/or a polymeric carrier, e.g., a liposome, such as a nanoparticle, e.g., a lipid nanoparticle, and delivered by known methods, such as via partial or full encapsulation of the circular polyribonucleotide, to a subject in need thereof (e.g., a human or non-human agricultural or domestic animal, e.g., cattle, dog, cat, horse, poultry). Such methods include, but are not limited to, transfection (e.g., lipid-mediated, cationic polymers, calcium phosphate, dendrimers); viral delivery (e.g., lentivirus, retrovirus, adenovirus, AAV), fugene, protoplast fusion, exosome-mediated transfer, lipid nanoparticle-mediated transfer, and any combination thereof. Cationic lipid-mediated delivery of proteins enables efficient protein-based genome editing in vitro and in vivo. Nat Biotechnol. 2014 Oct 30;33(1):73-80. Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al.

Additional methods of delivery include electroporation (e.g., using a flow electroporation device) or other methods of membrane disruption (e.g., nucleofection), microinjection, microprojectile bombardment (“gene gun”), direct sonic loading, cell squeezing, optical transfection, impalefection, magnetofection, and any combination thereof. A flow electroporation device, for example, comprises a chamber for containing a suspension of cells to be electorporated, such as the cells (e.g., isolated cells) as described herein, the chamber being at least partially defined by oppositely chargeable electrodes, wherein the thermal resistance of the chamber is less than approximately 110° C. per Watt.

In some embodiments, the circular polyribonucleotide, capped polyribonucleotide, or complex thereof, or pharmaceutical composition as disclosed herein may be delivered as a naked delivery formulation. A naked delivery formulation delivers a circular polyribonucleotide, capped polyribonucleotide, or complex thereof or pharmaceutical composition thereof to a cell without the aid of a carrier and without modification or partial or complete encapsulation of the circular polyribonucleotide, capped polyribonucleotide, or complex thereof.

A naked delivery formulation is a formulation that is free from a carrier and wherein the circular polyribonucleotide, capped polyribonucleotide, or complex thereof, or pharmaceutical composition thereof, is without a covalent modification that binds a moiety that aids in delivery to a cell or without partial or complete encapsulation of the circular polyribonucleotide, capped polyribonucleotide, or complex thereof. In some embodiments, a circular polyribonucleotide, capped polyribonucleotide, or complex thereof without a covalent modification that binds a moiety that aids in delivery to a cell may be a polyribonucleotide that is not covalently bound to a protein, small molecule, a particle, a polymer, or a biopolymer. A circular polyribonucleotide, capped polyriboucleotide, or complex thereof without covalent modification that binds a moiety that aids in delivery to a cell may not contain a modified phosphate group. For example, a circular polyribonucleotide, capped polyribonucleotide, or complex thereof without a covalent modification that binds a moiety that aids in delivery to a cell may not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation may be free of any or all of: transfection reagents, cationic carriers, carbohydrate carriers, nanoparticle carriers, or protein carriers. For example, a naked delivery formulation may be free from phtoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1 -(2,3-dioleoyloxy)propyl]-N,N,N- trimethylammonium chloride (DOTMA), 1-[2-(oleoyloxy)ethyl]-2-oleyl-3-(2-hydroxyethyl)imidazolinium chloride (DOTIM), 2,3-dioleyloxy-N- [2(sperminecarboxamido)ethyl]-N,N-dimethyl-1-propanaminium trifluoroacetate (DOSPA), 3B-[N—(N\N′-Dimethylaminoethane)-carbamoyl]Cholesterol Hydrochloride (DC-Cholesterol HC1), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high- density lipoprotein (HDL), or globulin.

A naked delivery formulation may comprise a non-carrier excipient. In some embodiments, a non-carrier excipient may comprise an inactive ingredient that does not exhibit a cell-penetrating effect. In some embodiments, a non-carrier excipient may comprise a buffer, for example PBS. In some embodiments, a non-carrier excipient may be a solvent, a non-aqueous solvent, a diluent, a suspension aid, a surface active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation may comprise a diluent (e.g., a parenterally acceptable diluent). A diluent may be a liquid diluent or a solid diluent. In some embodiments, a diluent may be an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

The invention is further directed to a host or host cell comprising the the circular polyribonucleotide, capped polyribonucleotide, or complex thereof as described herein. In some embodiments, vertebrate, mammal (e.g., human), or other organism or cell.

In some embodiments, the circular polyribonucleotide, capped polyribonucleotide, or complex thereof is non-immunogenic in the host. In some embodiments, the circular polyribonucleotide, capped polyribonucleotide, or complex thereof has a decreased or fails to produce a response by the host’s immune system as compared to the response triggered by a reference compound, e.g., a linear polynucleotide corresponding to the described circular polyribonucleotide, unmodified circular polyribonucleotide, or a circular polyribonucleotide lacking an encryptogen. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., delivered to or administered to) the circular polyribonucleotide, capped polyribonucleotide, or complex thereof. In some embodiments, the host is a mammal, such as a human. The amount of the circular polyribonucleotide, capped polyribonucleotide, or complex thereof, expression product, or both in the host can be measured at any time after administration. In certain embodiments, a time course of host growth in a culture is determined. If the growth is increased or reduced in the presence of the circular polyribonucleotide, capped polyribonucleotide, or complex thereof, or expression product or both is identified as being effective in increasing or reducing the growth of the host.

### Methods of Delivery

A method of delivering a circular polyribonucleotide, capped polyribonucleotide, or complex thereof as described herein or a pharmaceutical composition thereof as described herein to a cell, tissue or subject, comprises administering the circular polyribonucleotide, capped polyribonucleotide, or complex thereof or the pharmaceutical composition as described herein to the cell, tissue, or subject.

In some embodiments, the method of delivering is an in vivo method. For example, a method of delivering the circular polyribonucleotide, capped polyribonucleotide, or complex thereof or the pharmaceutical composition thereof comprises parenterally administering to a subject in need thereof. In some embodiments, the circular polyribonucleotide is an amount effective to have a biological effect on the cell or tissue in the subject. In some embodiments, the circular polyribonucleotide, capped polyribonucleotide, or complex thereof or the pharmaceutical composition thereof as described herein comprises a carrier. In some embodiments, the circular polyribonucleotide, capped polyribonucleotide, or complex thereof or the pharmaceutical composition thereof as described herein comprises a diluent and is free of any carrier. In some embodiments, parenteral administration is intravenously. In some embodiments, parenteral administration is intramuscularly. In some embodiments, parenteral administration is ophthalmically. In some embodiments, parenteral administration is topically.

In some embodiments, the circular polyribonucleotide, capped polyribonucleotide, or complex thereof or the pharmaceutical composition thereof is administered orally. In some embodiments, the circular polyribonucleotide, capped polyribonucleotide, or complex thereof or the pharmaceutical composition thereof is administered nasally. In some embodiments, the circular polyribonucleotide, capped polyribonucleotide, or complex thereof or the pharmaceutical composition thereof is administered by inhalation. In some embodiments, the circular polyribonucleotide, capped polyribonucleotide, or complex thereof or the pharmaceutical composition thereof is administered topically. In some embodiments, the circular polyribonucleotide, capped polyribonucleotide, or complex thereof or the pharmaceutical composition thereof is administered opthalmically. In some embodiments, the circular polyribonucleotide, capped polyribonucleotide, or complex thereof or the pharmaceutical composition thereof is administered rectally. In some embodiments the circular polyribonucleotide, capped polyribonucleotide, or complex thereof or the pharmaceutical composition thereof is administered by injection. The administration can be systemic administration. The administration can be local administration. In some embodiments, the circular polyribonucleotide, capped polyribonucleotide, or complex thereof or the pharmaceutical composition thereof is administered parenterally. In some embodiments, the circular polyribonucleotide, capped polyribonucleotide, or complex thereof or the pharmaceutical composition thereof is administered intravenously. In some embodiments, the circular polyribonucleotide, capped polyribonucleotide, or complex thereof or the pharmaceutical composition thereof is administered intraarterially. In some embodiments, the circular polyribonucleotide, capped polyribonucleotide, or complex thereof or the pharmaceutical composition thereof is administered intraperotoneally. In some embodiments, the circular polyribonucleotide, capped polyribonucleotide, or complex thereof or the pharmaceutical composition thereof is administered intradermally. In some embodiments, the circular polyribonucleotide, capped polyribonucleotide, or complex thereof or the pharmaceutical composition thereof is administered intracranially. In some embodiments, the circular polyribonucleotide, capped polyribonucleotide, or complex thereof or the pharmaceutical composition thereof is administered intrathecally. In some embodiments, the circular polyribonucleotide, capped polyribonucleotide, or complex thereof or the pharmaceutical composition thereof is administered intralymphaticly. In some embodiments, the circular polyribonucleotide, capped polyribonucleotide, or complex thereof or the pharmaceutical composition thereof is administered subcutaneously. In some embodiments, the circular polyribonucleotide, capped polyribonucleotide, or complex thereof or the pharmaceutical composition thereof is administered intramuscularly. In some embodiments, the circular polyribonucleotide molecule, capped polyribonucleotide, or complex thereof or the pharmaceutical composition thereof is administered via intraocular administration. In some embodiments, the circular polyribonucleotide, capped polyribonucleotide, or complex thereof or the pharmaceutical composition thereof is administered intracochlear (inner ear) administration administration. In some embodiments, the circular polyribonucleotide, capped polyribonucleotide, or complex thereof or the pharmaceutical composition thereof is administered intratracheal administration. In some embodiments, any of the methods of delivery as described herein are performed with a carrier. In some embodiments, the circular polyribonucleotide, capped polyribonucleotide, or complex thereof or the pharmaceutical composition thereof is administered intravenously with a carrier. In some embodiments, the circular polyribonucleotide, capped polyribonucleotide, or complex thereof or the pharmaceutical composition thereof is administered intraarterially with a carrier. In some embodiments, the circular polyribonucleotide, capped polyribonucleotide, or complex thereof or the pharmaceutical composition thereof is administered intraperotoneally with a carrier. In some embodiments, the circular polyribonucleotide, capped polyribonucleotide, or complex thereof or the pharmaceutical composition thereof is administered intradermally with a carrier. In some embodiments, the circular polyribonucleotide, capped polyribonucleotide, or complex thereof or the pharmaceutical composition thereof is administered intracranially with a carrier. In some embodiments, the circular polyribonucleotide, capped polyribonucleotide, or complex thereof or the pharmaceutical composition thereof is administered intrathecally with a carrier. In some embodiments, the circular polyribonucleotide, capped polyribonucleotide, or complex thereof or the pharmaceutical composition thereof is administered intralymphaticly with a carrier. In some embodiments, the circular polyribonucleotide, capped polyribonucleotide, or complex thereof or the pharmaceutical composition thereof is administered subcutaneously with a carrier. In some embodiments, the circular polyribonucleotide, capped polyribonucleotide, or complex thereof or the pharmaceutical composition thereof is administered intramuscularly with a carrier. In some embodiments, the circular polyribonucleotide molecule, capped polyribonucleotide, or complex thereof or the pharmaceutical composition thereof is administered via intraocular administration with a carrier. In some embodiments, the circular polyribonucleotide, capped polyribonucleotide, or complex thereof or the pharmaceutical composition thereof is administered intracochlear (inner ear) administration administration with a carrier. In some embodiments, the circular polyribonucleotide, capped polyribonucleotide, or complex thereof or the pharmaceutical composition thereof is administered intratracheal administration with a carrier. In some embodiments, any methods of delivery as described herein are performed without the aid of a carrier in a naked delivery formulation. In some embodiments, the circular polyribonucleotide, capped polyribonucleotide, or complex thereof or the pharmaceutical composition thereof is administered intravenously without the aid of a carrier in a naked delivery formulation. In some embodiments, the circular polyribonucleotide, capped polyribonucleotide, or complex thereof or the pharmaceutical composition thereof is administered intraarterially without the aid of a carrier in a naked delivery formulation. In some embodiments, the circular polyribonucleotide, capped polyribonucleotide, or complex thereof or the pharmaceutical composition thereof is administered intraperotoneally without the aid of a carrier in a naked delivery formulation. In some embodiments, the circular polyribonucleotide, capped polyribonucleotide, or complex thereof or the pharmaceutical composition thereof is administered intradermally without the aid of a carrier in a naked delivery formulation. In some embodiments, the circular polyribonucleotide, capped polyribonucleotide, or complex thereof or the pharmaceutical composition thereof is administered intracranially without the aid of a carrier in a naked delivery formulation. In some embodiments, the circular polyribonucleotide, capped polyribonucleotide, or complex thereof or the pharmaceutical composition thereof is administered intrathecally without the aid of a carrier in a naked delivery formulation. In some embodiments, the circular polyribonucleotide, capped polyribonucleotide, or complex thereof or the pharmaceutical composition thereof is administered intralymphaticly without the aid of a carrier in a naked delivery formulation. In some embodiments, the circular polyribonucleotide, capped polyribonucleotide, or complex thereof or the pharmaceutical composition thereof is administered subcutaneously without the aid of a carrier in a naked delivery formulation. In some embodiments, the circular polyribonucleotide, capped polyribonucleotide, or complex thereof or the pharmaceutical composition thereof is administered intramuscularly without the aid of a carrier in a naked delivery formulation. In some embodiments, the circular polyribonucleotide molecule, capped polyribonucleotide, or complex thereof or the pharmaceutical composition thereof is administered via intraocular administration without the aid of a carrier in a naked delivery formulation. In some embodiments, the circular polyribonucleotide, capped polyribonucleotide, or complex thereof or the pharmaceutical composition thereof is administered intracochlear (inner ear) administration administration without the aid of a carrier in a naked delivery formulation. In some embodiments, the circular polyribonucleotide, capped polyribonucleotide, or complex thereof or the pharmaceutical composition thereof is administered intratracheal administration without the aid of a carrier in a naked delivery formulation.

In some embodiments, the cell is a eukaryotic cell. In some embodiments, the cell is a mammalian cell. In some embodiments, the cell is a human cell. In some embodiments, the cell is an animal cell. In some embodiments, the cell is an immune cell. In some embodiments, the tissue is a connective tissue, a muscle tissue, a nervous tissue, or an epithelial tissue. In some embodiments, the tissue is an organ (e.g., liver, lung, spleen, kidney, etc.). In some embodiments, the subject is a mammal. In some embodiments, the subject is a human. In some embodiments, the subject is a pet. In some embodiments, the subject is a live-stock animal.

### Cell and Vesicle-Based Carriers

The circular polyribonucleotide, capped polyribonucleotide, or complex thereof as described herein or a pharmaceutical composition thereof as described herein can be administered to a cell in a vesicle or other membrane-based carrier.

In embodiments, the circular polyribonucleotide, capped polyribonucleotide, or complex thereof or the pharmaceutical composition thereof is administered in or via a cell, vesicle or other membrane-based carrier. In one embodiment the circular polyribonucleotide, capped polyribonucleotide, or complex thereof or the pharmaceutical composition thereof can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for a circular polyribonucleotide molecule or the pharmaceutical composition thereof as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the circular polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3- Trimethylammonium-Propane(DOTAP), N-[1 -(2,3-dioleoyloxy)propyl]-N,N,N- trimethylammonium chloride (DOTMA), 1-[2-(oleoyloxy)ethyl]-2-oleyl-3-(2- hydroxyethyl)imidazolinium chloride (DOTIM), 2,3-dioleyloxy-N- [2(sperminecarboxamido)ethyl]-N,N-dimethyl-1-propanaminium trifluoroacetate (DOSPA), 3B-[N— (N\N′-Dimethylaminoethane)-carbamoyl]Cholesterol Hydrochloride (DC-Cholesterol HC1), diheptadecylamidoglycyl spermidine (DOGS), N,N-distearyl-N,N- dimethylammonium bromide (DDAB), N-(1,2-dimyrlstyloxyprop-3-yl)-N,N-dimethyl-N- hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high- density lipoprotein (HDL), or globulin.

Exosomes can also be used as drug delivery vehicles for a circular polyribonucleotide molecule or a pharmaceutical composition thereof described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a circular polyribonucleotide molecule or a pharmaceutical composition thereof described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a circular polyribonucleotide molecule or a pharmaceutical composition thereof described herein.

Virosomes and virus-like particles (VLPs) can also be used as carriers to deliver a circular polyribonucleotide molecule or a pharmaceutical composition thereof described herein to targeted cells.

Plant nanovesicles and plant messenger packs (PMPs), e.g., as described in International Patent Publication Nos. WO2011097480, WO2013070324, WO2017004526, or WO2020041784 can also be used as carriers to deliver the circular RNA as described herein.

Microbubbles can also be used as carriers to deliver a circular polyribonucleotide molecule described herein. Microbubbles can also be used as carriers to deliver a linear polyribonucleotide described herein. See, e.g., US7115583; Beeri, R. et al., Circulation. 2002 Oct 1;106(14):1756-1759; Bez, M. et al., Nat Protoc. 2019 Apr; 14(4): 1015-1026; Hernot, S. et al., Adv Drug Deliv Rev. 2008 Jun 30; 60(10): 1153-1166; Rychak, J.J. et al., Adv Drug Deliv Rev. 2014 Jun; 72: 82-93. In some embodiments, microbubbles are albumin-coated perfluorocarbon microbubbles.

Silk fibroin can also be used as a carrier to deliver a circular polyribonucleotide molecule described herein. See, e.g., Boopathy, A.V. et al., PNAS. 116.33 (2019): 16473-1678; and He, H. et al., ACS Biomater. Sci. Eng. 4.5(2018): 1708-1715.

### Kit

In some aspects, the invention as provided herein comprises a kit comprising a capped polyribonucleotide as provided herein, the circular polyribonucleotide as provided herein and instructions for administering the capped polyribonucleotide and circular polyribonucleotide to a cell.

In some other aspects, the invention as provided herein comprises a kit comprising a complex comprising the capped polyribonucleotide as provided herein bound to the circular polyribonucleotide of as provided herein and instructions for administering the complex to a cell.

### Numbered Embodiments

A pharmaceutical composition comprising:
a. a polyribonucleotide comprising a 5′ modified guanosine cap; and
b. a circular polyribonucleotide.

The pharmaceutical composition of numbered embodiment 1, further comprising a pharmaceutically acceptable excipient.

The pharmaceutical composition of any one of numbered embodiments [1]-[2], wherein the polyribonucleotide comprises a first binding region.

The pharmaceutical composition of any one of numbered embodiments [1]-[3], wherein the circular polyribonucleotide comprises a second binding region.

The pharmaceutical composition of any one of numbered embodiments [1]-[4], wherein the first binding region specifically binds to the second binding region.

A polyribonucleotide comprising a 5′ modified guanosine cap and a first binding region, wherein the first binding region specifically binds to a second binding region of a circular polyribonucleotide.

A circular polyribonucleotide comprising a second binding region, wherein the second binding region specifically binds to a first binding region of a polyribonucleotide and wherein the polyribonucleotide comprises a 5′ modified guanosine cap.

A composition comprising:
a. a polyribonucleotide comprising a 5′ modified guanosine cap structure and first binding region;
b. and
c. a circular polyribonucleotide comprising a second binding region;
d. wherein the first binding region is bound to the second binding region.

The pharmaceutical composition, the polyribonucleotide, the circular polyribonucleotide, or the composition of any one of the preceding numbered embodiments, wherein the polyribonucleotide comprising the 5′ modified guanosine cap drives expression of the expression sequence in the circular polyribonucleotide when the polyribonucleotide is bound to the circular polyribonucleotide

The pharmaceutical composition, the polyribonucleotide, the circular polyribonucleotide, or the composition of any one of the preceding numbered embodiments, wherein the polyribonucleotide is bound to the circular polyribonucleotide by indirect binding.

The pharmaceutical composition, the polyribonucleotide, the circular polyribonucleotide, or the composition of any one of the preceding numbered embodiments, wherein the polyribonucleotide is bound to the circular polyribonucleotide by direct binding.

The pharmaceutical composition, the polyribonucleotide, the circular polyribonucleotide, or the composition of any one of the preceding numbered embodiments, wherein the polyribonucleotide is bound to the circular polyribonucleotide by covalent binding.

The pharmaceutical composition, the polyribonucleotide, the circular polyribonucleotide, or the composition of any one of the preceding numbered embodiments, wherein the polyribonucleotide is bound to the circular polyribonucleotide by noncovalent binding.

The pharmaceutical composition, the polyribonucleotide, the circular polyribonucleotide, or the composition of any one of the preceding numbered embodiments, wherein the first binding region is complementary to the second binding region.

The pharmaceutical composition, the polyribonucleotide, the circular polyribonucleotide, or the composition of any one of the preceding numbered embodiments, wherein the polyribonucleotide recruits a ribosome.

The pharmaceutical composition, the polyribonucleotide, the circular polyribonucleotide, or the composition of any one of the preceding numbered embodiments, wherein the 5′ modified guanosine cap of the polyribonucleotide recruits the ribosome.

The pharmaceutical composition, the polyribonucleotide, the circular polyribonucleotide, or the composition of any one of the preceding numbered embodiments, wherein the circular polyribonucleotide comprises an expression sequence.

The pharmaceutical composition, the polyribonucleotide, the circular polyribonucleotide, or the composition of any one of the preceding numbered embodiments, wherein the polyribonucleotide comprising the 5′ modified guanosine cap drives expression of the expression sequence in the circular polyribonucleotide.

The pharmaceutical composition, the polyribonucleotide, the circular polyribonucleotide, or the composition of any one of the preceding numbered embodiments, wherein the polyribonucleotide further comprises a UTR.

The pharmaceutical composition, the polyribonucleotide, the circular polyribonucleotide, or the composition of any one of the preceding numbered embodiments, wherein the polyribonucleotide comprises a 5′ UTR.

The pharmaceutical composition, the polyribonucleotide, the circular polyribonucleotide, or the composition of any one of the preceding numbered embodiments, wherein the polyribonucleotide comprises a 3′ UTR.

The pharmaceutical composition, the polyribonucleotide, the circular polyribonucleotide, or the composition of any one of the preceding numbered embodiments, wherein the polyribonucleotide comprises a poly A region.

The pharmaceutical composition, the polyribonucleotide, the circular polyribonucleotide, or the composition of any one of the preceding numbered embodiments, wherein the first binding region is a binding region that is 3′ of a UTR.

The pharmaceutical composition, the polyribonucleotide, the circular polyribonucleotide, or the composition of any one of the preceding numbered embodiments, wherein the first binding region comprises from 5 to 100 nucleotides in length.

The pharmaceutical composition, the polyribonucleotide, the circular polyribonucleotide, or the composition of any one of the preceding numbered embodiments, wherein the 5′ modified guanosine cap is a 7-methylguanylate cap.

The pharmaceutical composition, the polyribonucleotide, the circular polyribonucleotide, or the composition of any one of the preceding numbered embodiments, wherein the 5′ modified guanosine cap is an anti-reverse cap analog.

The pharmaceutical composition, the polyribonucleotide, the circular polyribonucleotide, or the composition of any one of the preceding numbered embodiments, wherein the polyribonucleotide comprises one or more of the 5′ modified guanosine cap.

The pharmaceutical composition, the polyribonucleotide, the circular polyribonucleotide, or the composition of any one of the preceding numbered embodiments, wherein the polyribonucleotide is linear.

The pharmaceutical composition, the polyribonucleotide, the circular polyribonucleotide, or the composition of any one of the preceding numbered embodiments, wherein the polyribonucleotide comprises from 5 to 1100 nucleotides in length.

The pharmaceutical composition, the polyribonucleotide, the circular polyribonucleotide, or the composition of any one of the preceding numbered embodiments, wherein the circular polyribonucleotide is an unmodified circular polyribonucleotide.

The pharmaceutical composition, the polyribonucleotide, the circular polyribonucleotide, or the composition of any one of the preceding numbered embodiments, wherein the circular polyribunucleotide comprises a UTR.

The pharmaceutical composition, the polyribonucleotide, the circular polyribonucleotide, or the composition of any one of the preceding numbered embodiments, wherein the circular polyribunucleotide comprises a poly A region.

The pharmaceutical composition, the polyribonucleotide, the circular polyribonucleotide, or the composition of any one of the preceding numbered embodiments, wherein the circular polyribonucleotide comprises an IRES.

The pharmaceutical composition, the polyribonucleotide, the circular polyribonucleotide, or the composition of any one of the preceding numbered embodiments, wherein the circular polyribunucleotide lacks an IRES.

The pharmaceutical composition, the polyribonucleotide, the circular polyribonucleotide, or the composition of any one of the preceding numbered embodiments, wherein the second binding region comprises from 5 to 100 nucleotides in length.

The pharmaceutical composition, the polyribonucleotide, the circular polyribonucleotide, or the composition of any one of the preceding numbered embodiments, wherein the circular polyribonucleotide comprises a stop codon.

The pharmaceutical composition, the polyribonucleotide, the circular polyribonucleotide, or the composition of any one of the preceding numbered embodiments, wherein the circular polyribonucleotide comprises the second binding region located in an untranslated region between the stop and a start codon.

The pharmaceutical composition, the polyribonucleotide, the circular polyribonucleotide, or the composition of any one of the preceding numbered embodiments, wherein the circular polyribonucleotide comprises an encryptogen, regulatory element, replication element, or quasi-double stranded secondary structure.

The pharmaceutical composition, the polyribonucleotide, the circular polyribonucleotide, or the composition of any one of the preceding numbered embodiments, wherein the circular polyribonucleotide comprises a stagger element.

The pharmaceutical composition, the polyribonucleotide, the circular polyribonucleotide, or the composition of any one of the preceding numbered embodiments, wherein the stop codon is between the second binding region and the stagger element.

The pharmaceutical composition, the polyribonucleotide, the circular polyribonucleotide, or the composition of any one of the preceding numbered embodiments, wherein the circular polyribonucleotide comprises a protein translation initiation site.

The pharmaceutical composition, the polyribonucleotide, the circular polyribonucleotide, or the composition of any one of the preceding numbered embodiments, wherein the protein translation initiation site comprises a Kozak sequence.

The pharmaceutical composition, the polyribonucleotide, the circular polyribonucleotide, or the composition of any one of the preceding numbered embodiments, wherein the circular polyribonucleotide comprises from 50 to 20000 nucleotides in length.

A method of producing a complex comprising binding the first binding region of the polyribonucleotide of any one of numbered embodiments [6]-[43] to the second binding region of the circular polyribonucleotide of any one of numbered embodiments [7]-[43], thereby producing the complex.

A method of delivery, comprising
delivering the polyribonucleotide of any one of numbered embodiments [6] or [9]-[43] to a cell, tissue, or subject,
delivering the circular polyribonucleotide of any one of numbered embodiments [7] or [9]-[43] to the cell, tissue, or subject.

A method of delivery, comprising
providing a complex, wherein the first binding region of the polyribonucleotide of any one of numbered embodiments 6 or 9-43 binds to the second binding region of the circular polyribonucleotide of any one of numbered embodiments [7] or [9]-[43] to produce the complex, and
delivering the complex to a cell, tissue, or subject.

A method of expressing one or more expression sequences from a circular polyribonucleotide in a cell, comprising
binding the first binding region of the polyribonucleotide of any one of numbered embodiments [6] or [9]-[43] to the second binding region of the circular polyribonucleotide of any one of numbered embodiments [7] or [9]-[43] to produce a complex,
   a. wherein the circular polyribonucleotide comprises the one or more expression sequences; and
delivering the complex to the cell;
wherein the complex affects expression of the one or more expression sequences in the cell.

A method of expressing one or more expression sequences from a circular polyribonucleotide in a cell, comprising
delivering the polyribonucleotide of any one of numbered embodiments [6] or [9]-[43] to the cell; and
delivering the circular polyribonucleotide of any one of numbered embodiments [7] or [9]-[43] comprising the one or more expression sequences to the cell;
wherein the first binding region binds to the second binding region to produce a complex that affects expression of the one or more expression sequences in the cell.

A method of in vitro expression of one or more expression sequences, comprising:
providing a complex comprising:
   the circular polyribonucleotide of any one of numbered embodiments [7] or [9]-[43] comprising the one or more expression sequences, and the polyribonucleotide of any one of numbered embodiments [6] or [9]-[43],
      a. wherein the first binding region is bound to the second binding region; and
   administering the complex to a cell in vitro,
wherein expression of the one or more expression sequences from the complex in the cell is higher than expression from the circular polyribonucleotide alone.

A method of in vitro expression of one or more expression sequences, comprising:
administering to a cell in vitro:
the circular polyribonucleotide of any one of numbered embodiments [7] or [9]-[43] comprising the one or more expression sequences, and the polyribonucleotide of any one of numbered embodiments 6 or 9-43 to a cell,
wherein the first binding region is bound to the second binding region to form a complex in the cell and the expression of the one or more expression sequences from the complex in the cell is higher than expression from the circular polyribonucleotide alone.

A method of in vivo expression of one or more expression sequences, comprising:
providing a complex comprising:
   a. the circular polyribonucleotide of any one of numbered embodiments [7] or [9]-[4]3 comprising the one or more expression sequences, and
   b. the polyribonucleotide of any one of numbered embodiments [6] or [9]-[43],
   c. wherein the first binding region is bound to the second binding region; and
administering the complex to a cell in vivo,
wherein expression of the one or more expression sequences from the complex in the cell is higher than expression from the circular polyribonucleotide alone.

A method of in vivo expression of one or more expression sequences, comprising:
administering to a cell in vivo:
   a. the circular polyribonucleotide of any one of numbered embodiments [7] or [9]-[43] comprising the one or more expression sequences, and
   b. the polyribonucleotide of any one of numbered embodiments [6] or [9]-[43] to a cell, wherein the first binding region is bound to the second binding region to form a complex in the cell and the expression of the one or more expression sequences from the complex in the cell is higher than expression from the circular polyribonucleotide alone.

A method of expression of one or more expression sequences, comprising:
providing a complex comprising:
   a. the circular polyribonucleotide of any one of numbered embodiments [7] or [9]-[43] comprising the one or more expression sequences, and
   b. the polyribonucleotide of any one of numbered embodiments [6] or [9]-[43],
   c. wherein the first binding region is bound to the second binding region; and
administering the complex to a cell,
wherein expression of the one or more expression sequences from the complex in the cell results in increased protein production compared to expression from the circular polyribonucleotide alone.

A method of expression of one or more expression sequences, comprising:
administering to a cell:
   a. the circular polyribonucleotide of any one of numbered embodiments [7] or [9]-[43] comprising the one or more expression sequences, and
   b. the polyribonucleotide of any one of numbered embodiments [6] or [9]-[43] to a cell, wherein the first binding region is bound to the second binding region to form a complex in the cell and the expression of the one or more expression sequences from the complex in the cell is results in increased protein production than expression from the circular polyribonucleotide alone.

The method of numbered embodiments [53] or [54], wherein the protein production is increased at 1 day after administering compared to a circular polyribonucleotide alone.

A method of expression of one or more expression sequences in a subject, comprising:
providing a complex comprising:
   a. the circular polyribonucleotide of any one of numbered embodiments [7] or [9]-[43] comprising the one or more expression sequences, and
   b. the polyribonucleotide of any one of numbered embodiments [6] or [9]-[43],
   c. wherein the first binding region is bound to the second binding region; and
administering the complex to a cell of the subject,
wherein expression of the one or more expression sequences from the complex in the subject is for at least 6 hours longer than after administering a circular polyribonucleotide alone.

A method of expression of one or more expression sequences in a subject, comprising:
administering to the subject:
   a. the circular polyribonucleotide of any one of numbered embodiments [7] or [9]-[43] comprising the one or more expression sequences, and
   b. the polyribonucleotide of any one of numbered embodiments [6] or [9]-[43], wherein the first binding region is bound to the second binding region to form a complex in the subject and expression of the one or more expression sequences from the complex in a cell of the subject is for at least 6 hours longer than after administering a circular polyribonucleotide alone.

A method of expression of one or more expression sequences in a subject, comprising:
providing a complex comprising:
   a. the circular polyribonucleotide of any one of numbered embodiments [7] or [9]-[43] comprising the one or more expression sequences, and
   b. the polyribonucleotide of any one of numbered embodiments [6] or [9]-[43],
   c. wherein the first binding region is bound to the second binding region;
   and
administering the complex to a cell of the subject,
wherein expression of the one or more expression sequences from the complex in the subject is for at least 6 hours longer than after administering a linear counterpart of the circular polyribonucleotide alone.

A method of expression of one or more expression sequences in a subject, comprising:
administering to the subject:
   a. the circular polyribonucleotide of any one of numbered embodiments [7] or [9]-[43] comprising the one or more expression sequences, and
   b. the polyribonucleotide of any one of numbered embodiments [6] or [9]-[43],
wherein the first binding region is bound to the second binding region to form a complex in the subject and expression of the one or more expression sequences from the complex in a cell of the subject is for at least 6 hours longer than after administering a linear counterpart of the circular polyribonucleotide alone.

The method of any one of numbered embodiments [45]-[59], wherein the cell is a eukaryotic cell.

The method of any one of numbered embodiments [45]-[60], wherein the cell is a mammalian cell.

The method of any one of numbered embodiments [45]-[61], wherein the cell is a human cell.

The method of any one of numbered embodiments [45]-[62], wherein the cell is an immune cell.

A method of treating a subject in need thereof comprising administering the polyribonucleotide of any one of numbered embodiments [6] or [9]-[43] and the circular polyribonucleotide of any one of numbered embodiments [7] or [9]-[43] to the subject.

A method of treating a subject in need thereof comprising administering the polyribonucleotide of any one of numbered embodiments [6] or [9]-[43] bound to the circular polyribonucleotide of any one of numbered embodiments [7]-[43] to the subject.

The method of any one of numbered embodiments [45], [46], [56]-[59], [64], or [65], wherein the subject is a mammal.

The method of any one of numbered embodiments [45], [46], [56]-[59], [64], or [65], wherein the subject is a pet.

The method of any one of numbered embodiments [45], [46], [56]-[59], [64], or [65], wherein the subject is a live-stock animal.

The method of any one of numbered embodiments [45], [46], [56]-[59], [64], or [65], wherein the subject is a human.

A kit comprising:
the polyribonucleotide of any one of numbered embodiments [6] or [9]-[43];
the circular polyribonucleotide of any one of numbered embodiments [7] or [9]-[43]; and instructions for administering the polyribonucleotide and circular polyribonucleotide to a cell.

A kit comprising:
a complex comprising the polyribonucleotide of any one of numbered embodiments [6] or [9]-[43] bound to the circular polyribonucleotide of any one of numbered embodiments [7] or [9]-[43]; and
instructions for administering the complex to a cell.

All references and publications cited herein are hereby incorporated by reference.

The above described embodiments can be combined to achieve the afore-mentioned functional characteristics. This is also illustrated by the below examples which set forth exemplary combinations and functional characteristics achieved.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1: Cap-Dependent Translation of Circular RNA

This Example demonstrates translation of circular RNA annealed with a single stranded linear RNA oligonucleotide encoding a 5′ modified guanosine cap structure.

Translation initiation from RNA molecules typically occurs at the initiation codon (AUG). In eukaryotes, the 40 S ribosomal subunit is recruited to the cap structure at the 5′ end of an mRNA. It then scans for a downstream initiation codon and initiates translation if the AUG is in a preferred surrounding sequence (A/GxxAUGG). Ribosomal scanning process does not proceed via base-by-base inspection of the RNA, but rather that some sections of the mRNA are bypassed during scanning.

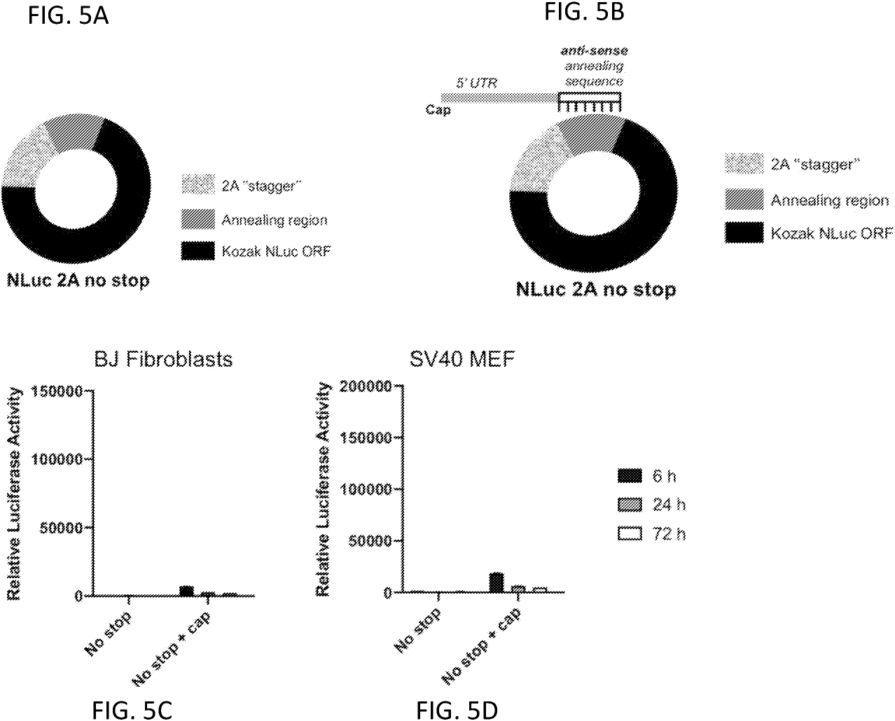

In this example, circular RNA was designed with an ORF encoding Nanoluciferase, a stagger element, an annealing region, and without stop codon (TAA) between the stagger element and annealing region, shown in FIG. 5A.

In this example, the polyribonucleotide comprising a 5′ cap is a linear RNA oligonucleotide that encodes a human alpha globin 5′UTR, a 3′ annealing region complementary to the annealing region of the circular RNA, and a 5′ modified guanosine cap structure generated using co-transcriptional capping with anti-reverse cap analog (ARCA). A schematic of the capped linear RNA oligonucleotide is shown in FIG. 5B.

The circular RNA was generated in vitro as follows: Unmodified linear RNA was synthesized by in vitro transcription using T7 RNA polymerase from a DNA template including the NLuc ORF, stagger element and annealing region described above. Transcribed RNA was purified using a Monarch® RNA cleanup kit (New England Biolabs, T2050), treated with RNA 5′phosphohydrolase (RppH) following the manufacturer’s instructions, and purified again with the Monarch® RNA cleanup kit (New England Biolabs, T2050). RppH treated linear RNA was circularized using a splint DNA and T4 RNA ligase. Circular RNA was purified by urea polyacrylamide gel, eluted in a buffer, ethanol precipitated and resuspended in RNase storage solution (ThermoFisher Scientific, cat# AM7000).

To generate capped single stranded linear RNA oligonucleotide, in vitro transcription was performed in the presence of 7.2 mM of ARCA. Transcribed RNA was purified with an RNA cleanup kit (New England Biolabs, T2050).

To anneal the circular RNA to the capped linear RNA oligonucleotide, capped linear RNA and circular RNA were incubated in buffer at 65° C. for 15 minutes and then gradually cooled to 25° C. RNA annealing was confirmed by agarose gel electrophoresis.

To measure expression efficiency of NLuc from annealed circular RNA-capped linear RNA complex compared to circular RNA only controls, the annealed constructs and the non-annealed controls (circular RNA alone) were transfected into BJ fibroblasts or SV40 MEF cells using a transfection agent. NLuc activity was measured at 6, 24 and 72 hours after transfection. To measure NLuc activity, NLuc reagent (Promega) was added and incubated for 2 minutes to allow lysis of the cell. Lysed cells were read using a luminometer instrument.

Under these conditions, internalized circular RNA annealed with the capped linear RNA oligonucleotide exhibited greater NLuc expression than the circular RNA only counterpart used as a control (FIG. 5C (BJ Fibroblasts); FIG. 5D (SV40 MEF)). Greatest NLuc expression was observed at 6 hours for all samples.

This Example demonstrated that circular RNA annealed with linear RNA oligonucleotides comprising 5′ cap structure can be used to drive expression of functional proteins from circular RNA in cells.

### Example 2: Cap-Dependent Translation of Circular RNA Comprising a Stop Codon

This Example demonstrates in vitro translation of circular RNA annealed with a single stranded linear RNA oligonucleotide encoding a 5′ modified guanosine cap structure.

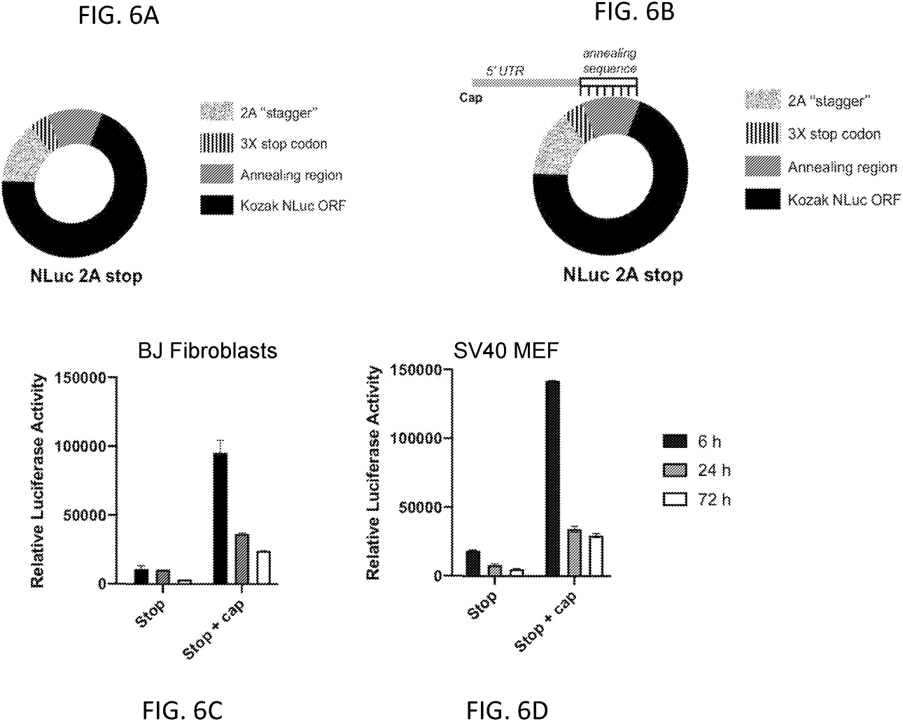

In this example, circular RNA was designed with an ORF encoding Nanoluciferase, a stagger element, an annealing region, with a stop codon (TAA) between the stagger element and annealing region, shown in FIG. 6A.

In this example, the polyribonucleotide comprising a 5′ cap is a linear RNA oligonucleotide that encodes a human alpha globin 5′UTR, a 3′ annealing region complementary to the annealing region of the circular RNA, and a 5′ modified guanosine cap structure generated using co-transcriptional capping with anti-reverse cap analog (ARCA). A schematic of capped linear RNA sequence is shown in FIG. 6B.

The circular RNA was generated in vitro as follows: Unmodified linear RNA was synthesized by in vitro transcription using T7 RNA polymerase from a DNA template including the NLuc ORF, stagger element and annealing region described above. Transcribed RNA was purified using a Monarch® RNA cleanup kit (New England Biolabs, T2050), treated with RNA 5′phosphohydrolase (RppH) following the manufacturer’s instructions, and purified again with the Monarch® RNA cleanup kit (New England Biolabs, T2050). RppH treated linear RNA was circularized using a splint DNA and T4 RNA ligase. Circular RNA was purified by urea polyacrylamide gel, eluted in a buffer, ethanol precipitated and resuspended in RNase storage solution (ThermoFisher Scientific, cat# AM7000).

To generate capped single stranded linear RNA oligonucleotide, in vitro transcription was performed in the presence of 7.2 mM of ARCA. Transcribed RNA was purified with an RNA cleanup kit (New England Biolabs, T2050).

To anneal the circular RNA to the capped linear RNA oligonucleotide, the capped linear RNA and circular RNA were incubated in buffer at 65° C. for 15 minutes and then gradually cooled to 25° C. RNA annealing was confirmed by agarose gel electrophoresis.

To measure expression efficiency of NLuc from annealed circular RNA-capped linear RNA complex compared to circular RNA only controls, the annealed constructs and the non-annealed controls (circular RNA alone) were transfected into BJ fibroblasts or SV40 MEF cells using a transfection agent. NLuc activity was measured at 6, 24 and 72 hours after transfection. To measure NLuc activity, NLuc reagent (Promega) was added and incubated for 2 minutes to allow lysis of the cell. Lysed cells were read using a luminometer instrument.

Under these conditions, internalized circular RNA annealed with the capped linear RNA oligonucleotide exhibited greater NLuc expression than the circular RNA only counterpart used as a control (FIG. 6C (BJ Fibroblasts); FIG. 6D (SV40 MEF)). Greatest NLuc expression was observed at 6 hours for all samples.

This Example demonstrated that circular RNA annealed with linear RNA oligonucleotides comprising 5′ cap structure can be used to drive expression of functional proteins from circular RNA in cells.

### Example 3: Cap-Dependent Translation of Circular RNA

This Example demonstrates translation of circular RNA annealed with a single stranded linear RNA oligonucleotide encoding a 5′ modified guanosine cap structure.

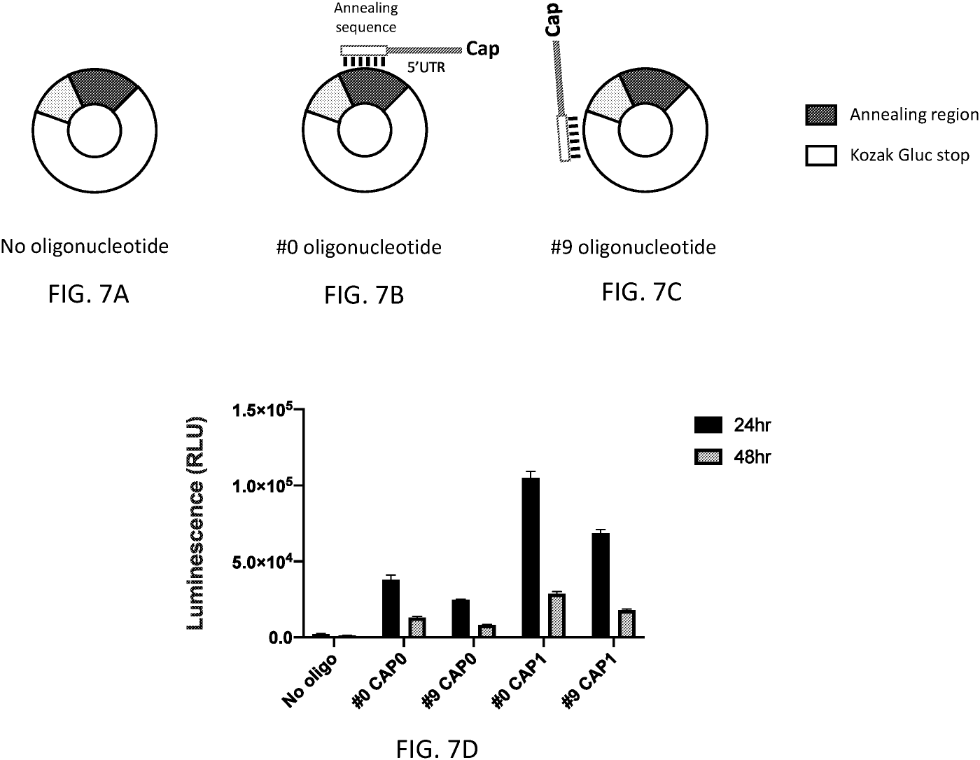

In this example, circular RNA was designed with an ORF encoding Gaussia luciferase, an annealing region, and stop codon (TAA) as shown in FIG. 7A.

In this example, one polyribonucleotide comprising a 5′ cap was a linear RNA oligonucleotide and a 3′ annealing sequence complementary to the annealing region of the circular RNA (Oligo #0). A second polyribonucleotide comprising a 5′ cap in this example was a linear RNA oligonucleotide comprising, a 3′ annealing sequences complementary to nucleotides upstream of a stop codon (TAA) of the Gluc ORF (Oligo #9). Two different cap structures were used for the capped polynucleotides: either a CapO was generated using co-transcriptional capping with anti-reverse cap analog (ARCA) or a Cap 1 was generated using a vaccinia capping system and 2′O-methyltransferase. A schematic of these capped linear RNA oligonucleotides are shown in FIG. 7B and FIG. 7C.

The circular RNA was generated in vitro as follows: Unmodified linear RNA was synthesized by in vitro transcription using T7 RNA polymerase from a DNA template including the GLuc ORF, and annealing region described above. Transcribed RNA was purified using a Monarch® RNA cleanup kit (New England Biolabs, T2050), treated with RNA 5′phosphohydrolase (RppH) following the manufacturer’s instructions, and purified again with the Monarch® RNA cleanup kit (New England Biolabs, T2050). RppH treated linear RNA was circularized using a splint DNA and T4 RNA ligase. Circular RNA was purified by urea polyacrylamide gel, eluted in a buffer, ethanol precipitated and resuspended in RNase-free water

To generate the CapO version of the capped linear polyribonucleotide, in vitro transcription was performed in the presence of 7.2 mM of ARCA. Transcribed RNA was purified with an RNA cleanup kit (New England Biolabs, T2050). To generate the Cap1 version of the capped linear polyribonucleotide, in vitro transcription is performed and purified with an RNA cleanup kit (T2050, NEB). Purified RNA was subjected to one-step capping and 2′-O-methylation to add Cap1 at the 5′ end with vaccinia virus capping enzyme and cap 2′-O-methyltransferase. Capped linear RNA oligonucleotides were purified by urea polyacrylamide gel, eluted in a buffer, ethanol precipitated and resuspended in RNase free water.

To anneal the circular RNA to the capped linear RNA oligonucleotide, 1uM of capped linear RNA and 0.5uM of circular RNA were incubated in bufferat 65° C. for 15 minutes and then gradually cooled to 25° C. Circular RNA without capped linear RNA oligonucleotide was used as negative control.

To measure expression efficiency of GLuc from annealed circular RNA-capped linear RNA complex compared to circular RNA only controls, the annealed constructs with different 5′ end (CAPO or CAP1) and the non-annealed controls (circular RNA alone) were transfected HeLa cells using a transfection agent. Cell culture media was harvested and replaced with fresh media at 24 hour and 48 hour timepoints to measure GLuc activity. Gluc activity was measured using a luminometer instrument.

The results showed that under these conditions, internalized circular RNA annealed with the capped linear RNA oligonucleotide exhibited greater GLuc expression than the circular RNA only counterpart (FIG. 7D). Capped linear RNA oligonucleotide that annealed immediately before the Gluc start codon (oligonucleotide #0) showed better translation enhancement than oligonucleotide that annealed at the 3′ end of Glue ORF (oligonucleotide #9). In addition, Cap1 linear RNA oligonucleotide showed greater translation enhancement than CapO linear RNA oligonucleotide. The highest GLuc expression enhancement was observed at 24 hours for all samples.

This Example demonstrated that circular RNA annealed with linear RNA oligonucleotides comprising 5′ cap structure can be used to drive expression of functional proteins from circular RNA in cells.

### Example 4: Multiple Capped Oligonucleotides Annealing Additively Enhances Translation of Circular RNA

This Example demonstrates capped oligonucleotide annealing to two different regions of circular RNA additively enhances translation from the circular RNA compared to a single oligonucleotide annealing.

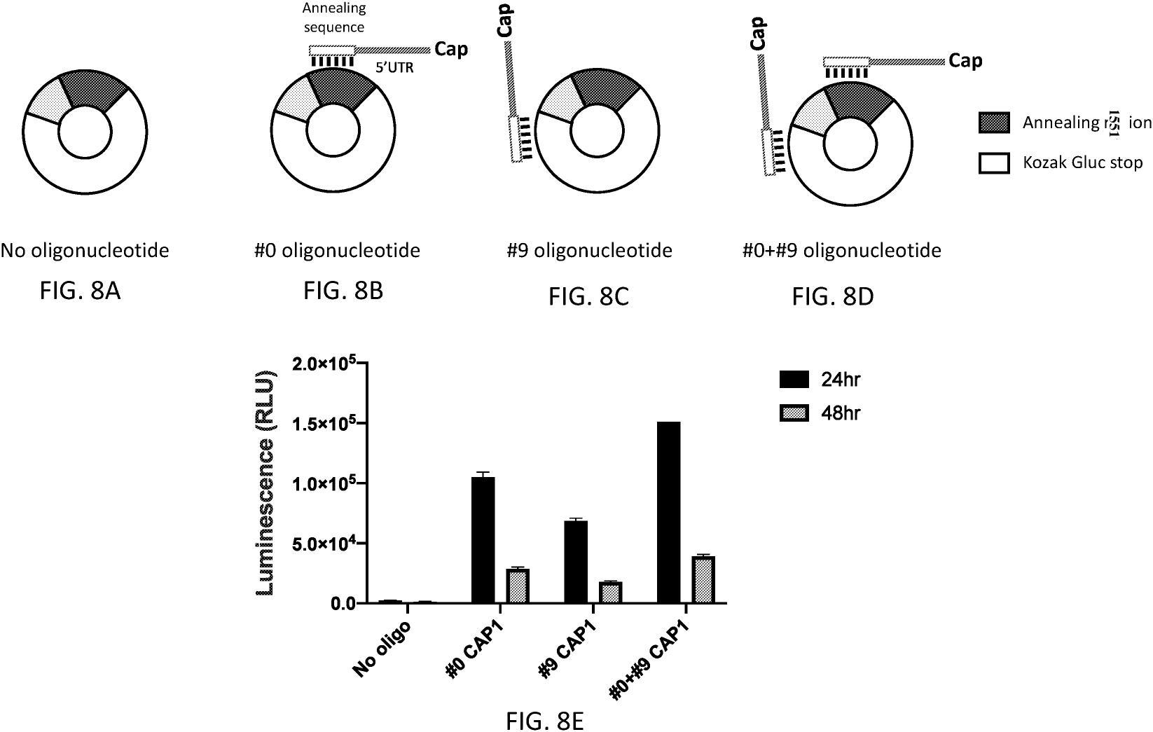

In this example, circular RNA was designed with an ORF encoding Gaussia luciferase, an annealing region, and stop codon (TAA) as shown in FIG. 8A.

In this example, one polyribonucleotide comprising a 5′ cap was a linear RNA oligonucleotide, a 3′ annealing sequence complementary to the annealing region of the circular RNA (Oligo #0). A second polyribonucleotide comprising a 5′ cap in this example was a linear RNA oligonucleotide, a 3′ annealing sequence complementary to nucleotides upstream of the stop codon (TAA) of the Gluc ORF (Oligo #9). The cap structure for the capped polynucleotides was a Cap1 generated using a vaccinia capping system and 2′O-methyltransferase. A schematic of the capped linear RNA oligonucleotides is shown in FIG. 8B, FIG. 8C, and FIG. 8D.

The circular RNA was generated in vitro as follows: Unmodified linear RNA was synthesized by in vitro transcription using T7 RNA polymerase from a DNA template including the GLuc ORF, and annealing region described above. Transcribed RNA was purified using a Monarch® RNA cleanup kit (New England Biolabs, T2050), treated with RNA 5′phosphohydrolase (RppH) following the manufacturer’s instructions, and purified again with the Monarch® RNA cleanup kit (New England Biolabs, T2050). RppH treated linear RNA was circularized using a splint DNA and T4 RNA ligase. Circular RNA was purified by urea polyacrylamide gel, eluted in a buffer, ethanol precipitated and resuspended in RNase-free water

To generate the Cap1 of the capped linear polyribonucleotide, in vitro transcription is performed and purified with an RNA cleanup kit (T2050, NEB). Purified RNA was subjected to one-step capping and 2′-O-methylation to add Cap1 at the 5′ end with vaccinia virus capping enzyme and cap 2′-O-methyltransferase. Capped linear RNA oligonucleotides were purified by urea polyacrylamide gel, eluted in a buffer, ethanol precipitated and resuspended in RNase free water.

To anneal the circular RNA to the capped linear RNA oligonucleotide, 1 uM of capped linear RNA and 0.5 uM of circular RNA were incubated in buffer at 65° C. for 15 minutes and then gradually cooled to 25° C. Capped linear RNA #0 and #9 were mixed with circular RNA individually or combined. Circular RNA without capped linear RNA oligonucleotide was used as negative control.

To measure expression efficiency of GLuc annealed with two capped linear RNAs compared to circular RNA annealed with one capped linear RNA, the annealed constructs and the non-annealed controls (circular RNA alone) were transfected in HeLa cells using a transfection agent. Cell culture media was harvested and replaced with fresh media at 24 hour and 48 hour timepoints to measure GLuc activity. Gluc activity was measured using a luminometer instrument.

The results surprisingly showed that under these conditions, internalized circular RNA annealed with two capped linear RNA oligonucleotide exhibited additive GLuc expression compared to the circular RNA annealed with only one capped linear RNA (FIG. 8E).

This Example demonstrated that more translation enhancement can be achieved by annealing multiple capped linear RNA to circular RNA.

What is claimed is:

1. A pharmaceutical composition comprising:
(a) a polyribonucleotide comprising a 5′ modified guanosine cap and a first binding region;
(b) a circular polyribonucleotide; and
(c) a pharmaceutically acceptable excipient.

2. The pharmaceutical composition of claim 1, wherein the circular polyribonucleotide comprises a second binding region.

3. The pharmaceutical composition of claim 2, wherein the first binding region specifically binds to the second binding region.

4. The pharmaceutical composition of claim 3, wherein the polyribonucleotide comprising the 5′ modified guanosine cap drives expression of the expression sequence in the circular polyribonucleotide when the polyribonucleotide is bound to the circular polyribonucleotide.

5. The pharmaceutical composition of claim 3, wherein the polyribonucleotide is bound to the circular polyribonucleotide by indirect binding.

6. The pharmaceutical composition of claim 3, wherein the polyribonucleotide is bound to the circular polyribonucleotide by direct binding.

7. The pharmaceutical composition of claim 3, wherein the polyribonucleotide is bound to the circular polyribonucleotide by covalent binding.

8. The pharmaceutical composition of claim 3, wherein the polyribonucleotide is bound to the circular polyribonucleotide by noncovalent binding.

9. The pharmaceutical composition of claim 2, wherein the first binding region is complementary to the second binding region.

10. The pharmaceutical composition of any one of claims 1-9 , wherein the polyribonucleotide recruits a ribosome.

11. The pharmaceutical composition of any one of claims 1-9, wherein the 5′ modified guanosine cap of the polyribonucleotide recruits the ribosome.

12. The pharmaceutical composition of any one of claims 1-11, wherein the circular polyribonucleotide comprises an expression sequence.

13. The pharmaceutical composition of claim 12, wherein the polyribonucleotide comprising the 5′ modified guanosine cap drives expression of the expression sequence in the circular polyribonucleotide.

14. The pharmaceutical composition of any one of claims 1-13, wherein the polyribonucleotide further comprises a UTR.

15. The pharmaceutical composition of any one of claims 1-14, wherein the polyribonucleotide comprises a 5′ UTR.

16. The pharmaceutical composition of any one of claims 1-14, wherein the polyribonucleotide comprises a 3′ UTR.

17. The pharmaceutical composition of any one of claims 1-16, wherein the polyribonucleotide comprises a poly A region.

18. The pharmaceutical composition of any one of claims 1-17, wherein the first binding region is a binding region that is 3′ of a UTR.

19. The pharmaceutical composition of any one of claims 1-18, wherein the first binding region comprises from 5 to 100 nucleotides in length.

20. The pharmaceutical composition of any one of claims 1-19, wherein the 5′ modified guanosine cap is a 7-methylguanylate cap.

21. The pharmaceutical composition of any one of claims 1-19, wherein the 5′ modified guanosine cap is an anti-reverse cap analog.

22. The pharmaceutical composition of any one of claims 1-21, wherein the polyribonucleotide comprises one or more of the 5′ modified guanosine cap.

23. The pharmaceutical composition of any one of claims 1-22, wherein the polyribonucleotide is linear.

24. The pharmaceutical composition of any one of claims 1-23, wherein the polyribonucleotide comprises from 5 to 1100 nucleotides in length.

25. The pharmaceutical composition of any one of claims 1-24, wherein the circular polyribonucleotide is an unmodified circular polyribonucleotide.

26. The pharmaceutical composition of any one of claims 1-25, wherein the circular polyribunucleotide comprises a UTR.

27. The pharmaceutical composition of any one of claims 1-26, wherein the circular polyribunucleotide comprises a poly A region.

28. The pharmaceutical composition of any one of claims 1-27, wherein the circular polyribonucleotide comprises an IRES.

29. The pharmaceutical composition of any one of claims 1-27, wherein the circular polyribunucleotide lacks an IRES.

30. The pharmaceutical composition of any one of claims 2-29, wherein the second binding region comprises from 5 to 100 nucleotides in length.

31. The pharmaceutical composition of any one of claims 1-30, wherein the circular polyribonucleotide comprises a stop codon.

32. The pharmaceutical composition of any one of claims 2-29, wherein the circular polyribonucleotide comprises the second binding region located in an untranslated region between the stop and a start codon.

33. The pharmaceutical composition of any one of claims 1-32, wherein the circular polyribonucleotide comprises an encryptogen, regulatory element, replication element, or quasi-double stranded secondary structure.

34. The pharmaceutical composition of any one of claims 1-33, wherein the circular polyribonucleotide comprises a stagger element.

35. The pharmaceutical composition of claim 34, wherein the circular polyribonucleotide comprises a stop codon between the second binding region and the stagger element.

36. The pharmaceutical composition of any one of claims 1-35, wherein the circular polyribonucleotide comprises a protein translation initiation site.

37. The pharmaceutical composition of claim 36, wherein the protein translation initiation site comprises a Kozak sequence.

38. The pharmaceutical composition of any one of claims 1-37, wherein the circular polyribonucleotide comprises from 50 to 20000 nucleotides in length.

39. A pharmaceutical composition comprising
(a) a first polyribonucleotide comprising a 5′ modified guanosine cap and a first binding region;
(b) a second polyribonucleotide comprising a 5′ modified guanosine cap and a third binding region;
(c) a circular polyribonucleotide; and
(d) a pharmaceutically acceptable excipient.

40. The pharmaceutical composition of claim 39, wherein the circular polyribonucleotide comprises a second binding region and a fourth binding region.

41. The pharmaceutical composition of claim 40, wherein the first binding region specifically binds to the second binding region, and the third binding region specifically binds to the fourth binding region.

42. The pharmaceutical composition of claim 41, wherein the first polyribonucleotide and the second polyribonucleotide drive expression of an expression sequence in the circular polyribonucleotide when the polyribonucleotides are bound to the circular polyribonucleotide.

43. The pharmaceutical composition of claim 41, wherein the first polyribonucleotide and the second polyribonucleotide drive increased expression of an expression sequence in the circular polyribonucleotide when the first polyribonucleotide and the second polyribonucleotide are bound to the circular polyribonucleotide compared to expression of an expression sequence in the circular polyribonucleotide when the first polyribonucleotide is bound to the circular polyribonucleotide or compared to expression of an expression sequence in the circular polyribonucleotide when the second polyribonucleotide is bound to the circular polyribonucleotide.

44. A polyribonucleotide comprising a 5′ modified guanosine cap and a first binding region, wherein the first binding region specifically binds to a second binding region of a circular polyribonucleotide.

45. A circular polyribonucleotide comprising a second binding region, wherein the second binding region specifically binds to a first binding region of a polyribonucleotide and wherein the polyribonucleotide comprises a 5′ modified guanosine cap.

46. A complex comprising
the polyribonucleotide of claim 44; and
the circular polyribonucleotide of claim 45;
wherein the first binding region of the polyribonucleotide is bound to the second binding region of the circular polyribonucleotide.

47. A method of producing a complex comprising binding the first binding region of the polyribonucleotide of claim 44 to the second binding region of the circular polyribonucleotide of claim 45, thereby producing the complex.

48. A method of expressing an expression sequence from a circular polyribonucleotide in a cell, comprising delivering the complex of claim 47 to the cell, wherein the circular polyribonucleotide of the complex comprises an expression sequence.

49. The phamaceutical composition of any one of claims 1-43 for use in a method of treatment of a human or animal body by therapy.

50. The complex of claim 46 for use as a medicament or a pharmaceutical.

51. The complex of claim 46 for use in a method of treatment of a human or animal body by therapy.

52. Use of the complex of claim 46, or the polyribonucleotide of claim 44 and the circular polyribonucleotide of claim 45, in the manufacture of a medicament or a pharmaceutical.

53. Use of the complex of claim 46, or the polyribonucleotide of claim 44 and the circular polyribonucleotide of claim 45, in the manufacture of a medicament or a pharmaceutical for treating a human or animal body by therapy.


SEQUENCE LISTING (48 sequences)

SEQ ID NO: 1 - skipped

SEQ ID NO: 2 - protein, 8 aa, Artificial Sequence
  source: /note="Description of Artificial Sequence: Synthetic peptide"
  VARIANT (2)..(2): /replace="Ile"
  MOD_RES (4)..(4): Any amino acid
  SITE (1)..(8): /note="Variant residues given in the sequence have no preference with respect to those in the annotations for variant positions"
DVEXNPGP

SEQ ID NO: 3 - protein, 9 aa, Artificial Sequence
  source: /note="Description of Artificial Sequence: Synthetic peptide"
  VARIANT (1)..(1): /replace="His" or " "
  VARIANT (2)..(2): /replace="Gly" or " "
  VARIANT (3)..(3): /replace="Val" or "Ile" or "Ser" or "Met"
  MOD_RES (5)..(5): Any amino acid
  SITE (1)..(9): /note="Variant residues given in the sequence have no preference with respect to those in the annotations for variant positions"
GDDEXNPGP

SEQ ID NO: 4-5 - skipped (2 entries)

SEQ ID NO: 6 - protein, 9 aa, Artificial Sequence
  source: /note="Description of Artificial Sequence: Synthetic peptide"
GDVESNPGP

SEQ ID NO: 7 - protein, 9 aa, Artificial Sequence
  source: /note="Description of Artificial Sequence: Synthetic peptide"
GDIEENPGP

SEQ ID NO: 8 - protein, 7 aa, Artificial Sequence
  source: /note="Description of Artificial Sequence: Synthetic peptide"
VEPNPGP

SEQ ID NO: 9 - protein, 7 aa, Artificial Sequence
  source: /note="Description of Artificial Sequence: Synthetic peptide"
IETNPGP

SEQ ID NO: 10 - protein, 9 aa, Artificial Sequence
  source: /note="Description of Artificial Sequence: Synthetic peptide"
GDIESNPGP

SEQ ID NO: 11 - protein, 9 aa, Artificial Sequence
  source: /note="Description of Artificial Sequence: Synthetic peptide"
GDVELNPGP

SEQ ID NO: 12 - protein, 9 aa, Artificial Sequence
  source: /note="Description of Artificial Sequence: Synthetic peptide"
GDIETNPGP

SEQ ID NO: 13 - protein, 8 aa, Artificial Sequence
  source: /note="Description of Artificial Sequence: Synthetic peptide"
GDVENPGP

SEQ ID NO: 14 - protein, 9 aa, Artificial Sequence
  source: /note="Description of Artificial Sequence: Synthetic peptide"
GDVEENPGP

SEQ ID NO: 15 - protein, 9 aa, Artificial Sequence
  source: /note="Description of Artificial Sequence: Synthetic peptide"
GDVEQNPGP

SEQ ID NO: 16 - protein, 7 aa, Artificial Sequence
  source: /note="Description of Artificial Sequence: Synthetic peptide"
IESNPGP

SEQ ID NO: 17 - protein, 9 aa, Artificial Sequence
  source: /note="Description of Artificial Sequence: Synthetic peptide"
GDIELNPGP

SEQ ID NO: 18 - protein, 9 aa, Artificial Sequence
  source: /note="Description of Artificial Sequence: Synthetic peptide"
HDIETNPGP

SEQ ID NO: 19 - protein, 9 aa, Artificial Sequence
  source: /note="Description of Artificial Sequence: Synthetic peptide"
HDVETNPGP

SEQ ID NO: 20 - protein, 9 aa, Artificial Sequence
  source: /note="Description of Artificial Sequence: Synthetic peptide"
HDVEMNPGP

SEQ ID NO: 21 - protein, 9 aa, Artificial Sequence
  source: /note="Description of Artificial Sequence: Synthetic peptide"
GDMESNPGP

SEQ ID NO: 22 - protein, 9 aa, Artificial Sequence
  source: /note="Description of Artificial Sequence: Synthetic peptide"
GDVETNPGP

SEQ ID NO: 23 - protein, 9 aa, Artificial Sequence
  source: /note="Description of Artificial Sequence: Synthetic peptide"
GDIEQNPGP

SEQ ID NO: 24 - protein, 8 aa, Artificial Sequence
  source: /note="Description of Artificial Sequence: Synthetic peptide"
DSEFNPGP

SEQ ID NO: 25 - protein, 15 aa, Artificial Sequence
  source: /note="Description of Artificial Sequence: Synthetic peptide"
GLNDIFEAQKIEWHE

SEQ ID NO: 26 - protein, 26 aa, Artificial Sequence
  source: /note="Description of Artificial Sequence: Synthetic peptide"
KRRWKKNFIAVSAANRFKKISSSGAL

SEQ ID NO: 27 - protein, 6 aa, Artificial Sequence
  source: /note="Description of Artificial Sequence: Synthetic peptide"
EEEEEE

SEQ ID NO: 28 - protein, 13 aa, Artificial Sequence
  source: /note="Description of Artificial Sequence: Synthetic peptide"
GAPVPYPDPLEPR

SEQ ID NO: 29 - protein, 8 aa, Artificial Sequence
  source: /note="Description of Artificial Sequence: Synthetic peptide"
DYKDDDDK

SEQ ID NO: 30 - protein, 9 aa, Artificial Sequence
  source: /note="Description of Artificial Sequence: Synthetic peptide"
YPYDVPDYA

SEQ ID NO: 31 - protein, 6 aa, Artificial Sequence
  source: /note="Description of Artificial Sequence: Synthetic 6xHis tag"
HHHHHH

SEQ ID NO: 32 - protein, 10 aa, Artificial Sequence
  source: /note="Description of Artificial Sequence: Synthetic peptide"
EQKLISEEDL

SEQ ID NO: 33 - protein, 18 aa, Artificial Sequence
  source: /note="Description of Artificial Sequence: Synthetic peptide"
TKENPRSNQEESYDDNES

SEQ ID NO: 34 - protein, 15 aa, Artificial Sequence
  source: /note="Description of Artificial Sequence: Synthetic peptide"
KETAAAKFERQHMDS

SEQ ID NO: 35 - protein, 38 aa, Artificial Sequence
  source: /note="Description of Artificial Sequence: Synthetic polypeptide"
MDEKTTGWRGGHVVEGLAGELEQLRARLEHHPQGQREP

SEQ ID NO: 36 - protein, 13 aa, Artificial Sequence
  source: /note="Description of Artificial Sequence: Synthetic peptide"
SLAELLNAGLGGS

SEQ ID NO: 37 - protein, 8 aa, Artificial Sequence
  source: /note="Description of Artificial Sequence: Synthetic peptide"
TQDPSRVG

SEQ ID NO: 38 - protein, 12 aa, Artificial Sequence
  source: /note="Description of Artificial Sequence: Synthetic peptide"
PDRVRAVSHWSS

SEQ ID NO: 39 - protein, 8 aa, Artificial Sequence
  source: /note="Description of Artificial Sequence: Synthetic peptide"
WSHPQFEK

SEQ ID NO: 40 - protein, 6 aa, Artificial Sequence
  source: /note="Description of Artificial Sequence: Synthetic peptide"
CCPGCC

SEQ ID NO: 41 - protein, 10 aa, Artificial Sequence
  source: /note="Description of Artificial Sequence: Synthetic peptide"
EVHTNQDPLD

SEQ ID NO: 42 - protein, 14 aa, Artificial Sequence
  source: /note="Description of Artificial Sequence: Synthetic peptide"
GKPIPNPLLGLDST

SEQ ID NO: 43 - protein, 11 aa, Artificial Sequence
  source: /note="Description of Artificial Sequence: Synthetic peptide"
YTDIEMNRLGK

SEQ ID NO: 44 - protein, 8 aa, Artificial Sequence
  source: /note="Description of Artificial Sequence: Synthetic peptide"
DLYDDDDK

SEQ ID NO: 45 - RNA, 90 nt, Artificial Sequence
  source: /note="Description of Artificial Sequence: Synthetic oligonucleotide"
cgggucggcauggcaucuccaccuccucgcgguccgaccugggcauccgaaggaggacgcacguccacucggauggcuaagggagagcca

SEQ ID NO: 46 - RNA, 88 nt, Artificial Sequence
  source: /note="Description of Artificial Sequence: Synthetic oligonucleotide"
uggccggcauggucccagccuccucgcuggcgccggcugggcaacauuccgaggggaccguccccucgguaauggcgaaugggaccca

SEQ ID NO: 47 - RNA, 96 nt, Artificial Sequence
  source: /note="Description of Artificial Sequence: Synthetic oligonucleotide"
ggcucaucucgacaagaggcggcaguccucaguacucuuacucuuuucuguaaagaggagacugcuggacucgccgcccaaguucgagcaugagcc

SEQ ID NO: 48 - RNA, 74 nt, Artificial Sequence
  source: /note="Description of Artificial Sequence: Synthetic oligonucleotide"
ggcuagaggcggcaguccucaguacucuuacucuuuucuguaaagaggagacugcuggacucgccgcccgagcc